(19) United States
(12) Patent Application Publication
(10) Pub. No.: US 2021/0107959 A1
(43) Pub. Date: Apr. 15, 2021

(54) SINGLE-ARM CO-RECEPTOR FUSION PROTEINS AND USES THEREOF

(71) Applicant: Acceleron Pharma Inc., Cambridge, MA (US)

(72) Inventors: Ravindra Kumar, Acton, MA (US); Asya Grinberg, Lexington, MA (US); Dianne S. Sako, Medford, MA (US)

(21) Appl. No.: 16/959,466

(22) PCT Filed: Jan. 2, 2019
(86) PCT No.: PCT/US2019/012020

§ 371 Date: Jul. 1, 2020

Related U.S. Application Data
(60) Provisional application No. 62/613,340, filed on Jan. 3, 2018.

(51) Int. Cl. C07K 14/495

(52) U.S. Cl. CPC C07K 14/495; A61K 38/00; C07K 2319/30

(57) ABSTRACT

In certain aspects, the disclosure provides single-arm heteromeric polypeptide complexes comprising an extracellular domain of a co-receptor of the TGF-beta superfamily. In some embodiments, the disclosure provides single-arm polypeptide complexes comprising an extracellular domain of a co-receptor selected from: endoglin, betaglycan, Cripto-1, Cryptic, Cryptic family protein 1B, Crim 1, Crim2, BAMBI, BMPER, RGM-A, RGM-B, MuSK, and hemojuvelin. Optionally the complex is a heterodimer. In certain aspects, such polypeptide complexes may be used for the treatment or prevention of various TGF-beta associated conditions, including without limitation diseases and disorders associated with, for example, cancer, muscle, bone, fat, red blood cells, metabolism, fibrosis and other tissues that are affected by one or more ligands of the TGF-beta superfamily.

### RELATED APPLICATION

This application claims the benefit of priority to U.S. Provisional Patent Application No. 62/613,340, filed Jan. 3, 2018, which application is hereby incorporated by reference in its entirety.

### BACKGROUND OF THE INVENTION

The transforming growth factor-beta (TGF-beta) superfamily contains a variety of growth factors that share common sequence elements and structural motifs. These proteins are known to exert biological effects on a large variety of cell types in both vertebrates and invertebrates. Members of the superfamily perform important functions during embryonic development in pattern formation and tissue specification and can influence a variety of differentiation processes, including adipogenesis, myogenesis, chondrogenesis, cardiogenesis, hematopoiesis, neurogenesis, and epithelial cell differentiation. The superfamily is divided into two general phylogenetic clades: the more recently evolved members of the superfamily, which includes TGF-betas, activins, and nodal and the clade of more distantly related proteins of the superfamily, which includes a number of BMPs and GDFs. Hinck (2012) FEBS Letters 586:1860-1870. TGF-beta superfamily members have diverse, often complementary biological effects. By manipulating the activity of a member of the TGF-beta superfamily, it is often possible to cause significant physiological changes in an organism. For example, the Piedmontese and Belgian Blue cattle breeds carry a loss-of-function mutation in the GDF8 (also called myostatin) gene that causes a marked increase in muscle mass. Grobet et al. (1997) Nat Genet., 17(1):71-4. Furthermore, in humans, inactive alleles of GDF8 are associated with increased muscle mass and, reportedly, exceptional strength. Schuelke et al. (2004) N Engl J Med, 350:2682-8.

Changes in muscle, bone, fat, red blood cells, and other tissues may be achieved by enhancing or inhibiting signaling (e.g., SMAD 1, 2, 3, 5, and/or 8) that is mediated by ligands of the TGF-beta superfamily. Thus, there is a need for agents that regulate the activity of various ligands of the TGF-beta superfamily.

### SUMMARY OF THE INVENTION

In part, the disclosure provides heteromultimeric complexes comprising a single TGF-beta superfamily co-receptor polypeptide (e.g., an endoglin, betaglycan, Cripto-1, Cryptic, Cryptic family protein 1B, Crim1, Crim2, BAMBI, BMPER, RGM-A, RGM-B, MuSK, and hemojuvelin polypeptide), including fragments and variants thereof. These constructs may be referred to herein as “single-arm” polypeptide complexes. Optionally, single-arm polypeptide complexes disclosed herein have different ligand-binding specificities/profiles compared to a corresponding homodimeric complex.

Heteromultimeric structures include, for example, heterodimers, heterotrimers, and higher order complexes. Preferably, TGF-beta superfamily co-receptor polypeptides as described herein comprise a ligand-binding domain of the receptor, for example, an extracellular domain of a TGF-beta superfamily co-receptor. Accordingly, in certain aspects, protein complexes described herein comprise a ligand-biding domain of a TGF-beta superfamily co-receptor selected from: endoglin, betaglycan, Cripto-1, Cryptic, Cryptic family protein 1B, Crim1, Crim2, BAMBI, BMPER, RGM-A, RGM-B, MuSK, and hemojuvelin, as well as truncations and variants thereof. Preferably, TGF-beta superfamily co-receptor polypeptides as described herein, as well as protein complexes comprising the same, are soluble. In certain aspects, heteromultimer of the disclosure bind to one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, Müllerian-inhibiting substance (MIS), and Lefty). Optionally, heteromers of the disclosure bind to one or more of these ligands with a K_{D} of less than or equal to 10⁻⁸, 10⁻⁹, 10⁻¹⁰, 10⁻¹¹, or 10⁻¹². In general, heteromultimer complexes of the disclosure antagonize (inhibit) one or more activities of at least one TGF-beta superfamily ligand, and such alterations in activity may be measured using various assays known in the art, including, for example, a cell-based assay as described herein. Preferably, protein complexes of the disclosure exhibit a serum half-life of at least 4, 6, 12, 24, 36, 48, or 72 hours in a mammal (e.g., a mouse or a human). Optionally, protein complexes of the disclosure may exhibit a serum half-life of at least 6, 8, 10, 12, 14, 20, 25, or 30 days in a mammal (e.g., a mouse or a human).

In certain aspects, protein complexes described herein comprise a first polypeptide covalently or non-covalently associated with a second polypeptide wherein the first polypeptide comprises the amino acid sequence of a TGF-beta superfamily co-receptor polypeptide and the amino acid sequence of a first member of an interaction pair and the second polypeptide comprises a second member of the interaction pair and does not contain an amino acid sequence of a TGF-beta superfamily co-receptor polypeptide. Optionally, the second polypeptide comprises, in addition to the second member of the interaction pair, a further polypeptide sequence that is not a TGF-beta superfamily co-receptor polypeptide and may optionally comprise not more than 5, 10, 15, 20, 30, 40, 50, 100, 200, 300, 400 or 500 amino acids. Optionally, the TGF-beta superfamily co-receptor polypeptide is connected directly to the first member of the interaction pair, or an intervening sequence, such as a linker, may be positioned between the amino acid sequence of the TGF-beta superfamily co-receptor polypeptide and the amino acid sequence of the first member of the interaction pair. Examples of linkers include, but are not limited to, the sequences TGGG (SEQ ID NO: 162), TGGGG (SEQ ID NO: 160), SGGGG (SEQ ID NO: 161), SGGG (SEQ ID NO: 163), GGGG (SEQ ID NO: 159), and GGG (SEQ ID NO: 158).

Interaction pairs described herein are designed to promote dimerization or form higher order multimers. In some embodiments, the interaction pair may be any two polypeptide sequences that interact to form a complex, particularly a heterodimeric complex although operative embodiments may also employ an interaction pair that forms a homodimeric complex. The first and second members of the interaction pair may be an asymmetric pair, meaning that the members of the pair preferentially associate with each other rather than self-associate. Accordingly, first and second members of an asymmetric interaction pair may associate to form a heterodimeric complex. Alternatively, the interaction pair may be unguided, meaning that the members of the pair may associate with each other or self-associate without substantial preference and thus may have the same or different amino acid sequences. Accordingly, first and second members of an unguided interaction pair may associate to form a homodimer complex or a heterodimeric complex. Optionally, the first member of the interaction pair (e.g., an asymmetric pair or an unguided interaction pair) associates covalently with the second member of the interaction pair. Optionally, the first member of the interaction pair (e.g., an asymmetric pair or an unguided interaction pair) associates non-covalently with the second member of the interaction pair.

Traditional Fc fusion proteins and antibodies are examples of unguided interaction pairs, whereas a variety of engineered Fc domains have been designed as asymmetric interaction pairs. Therefore, a first member and/or a second member of an interaction pair described herein may comprise a constant domain of an immunoglobulin, including, for example, the Fc portion of an immunoglobulin. Optionally, a first member of an interaction pair may comprise an amino acid sequence that is derived from an Fc domain of an IgG1, IgG2, IgG3, or IgG4 immunoglobulin. For example, the first member of an interaction pair may comprise, consist essentially of, or consist of an amino acid sequence that is at least 70%, 75%, 80%, 85%, 90%, 91%, 92%, 93%, 94%, 95%, 96%, 97%, 98%, 99% or 100% identical to any one of SEQ ID NOs: 200-214, 502, 503, 506, or 507. Optionally, a second member of an interaction pair may comprise an amino acid sequence that is derived from an Fc domain of an IgG1, IgG2, IgG3, or IgG4. For example, the second member of an interaction pair may comprise, consist essentially of, or consist of an amino acid sequence that is at least 80%, 85%, 90%, 91%, 92%, 93%, 94%, 95%, 96%, 97%, 98%, 99% or 100% identical to any one of SEQ ID NOs: 200-214, 502, 503, 506, or 507. In some embodiments, a first member and a second member of an interaction pair comprise Fc domains derived from the same immunoglobulin class and subtype. In other embodiments, a first member and a second member of an interaction pair comprise Fc domains derived from different immunoglobulin classes or subtypes. Optionally, a first member and/or a second member of an interaction pair (e.g., an asymmetric pair or an unguided interaction pair) comprise a modified constant domain of an immunoglobulin, including, for example, a modified Fc portion of an immunoglobulin. For example, protein complexes of the disclosure may comprise a first Fc portion of an IgG comprising an amino acid sequence that is at least 80%, 85%, 90%, 91%, 92%, 93%, 94%, 95%, 96%, 97%, 98%, or 99% identical to an amino acid sequence selected from the group: SEQ ID NOs: 200-214, 502, 503, 506, or 507 and a second Fc portion of an IgG, which may be the same or different from the amino acid sequence of the first modified Fc portion of the IgG, comprising an amino acid sequence that is at least 80%, 85%, 90%, 91%, 92%, 93%, 94%, 95%, 96%, 97%, 98%, or 99% identical to an amino acid sequence selected from the group: SEQ ID NOs: 200-214, 502, 503, 506, or 507.

In some embodiments, the disclosure provides heteromeric polypeptide complexes comprising a single TGF-beta superfamily co-receptor polypeptide, wherein the TGF-beta superfamily receptor polypeptide is derived from an endoglin polypeptide. For example, endoglin polypeptides may comprise of an amino acid sequence that is at least 70%, 75%, 80%, 85%, 90%, 91%, 92%, 93%, 94%, 95%, 96%, 97%, 98%, 99% or 100% identical to an endoglin sequence disclosed herein (e.g., SEQ ID NOs: 1, 2, 5, 6, 9, 10, 500, 501, 504, and 505). Optionally, endoglin polypeptides of the disclosure may be fusion proteins that further comprise one or more portions (domains) that are heterologous to endoglin. For example, an endoglin polypeptide may be fused to a heterologous polypeptide that comprises a multimerization domain, optionally with a linker domain positioned between the endoglin polypeptide and the heterologous polypeptide (e.g., SEQ ID NOs: 500, 501, 504, and 505). In some embodiments, multimerization domains described herein comprise one component of an interaction pair. Heteromeric complexes that comprise an endoglin polypeptide do not comprise a type I receptor, type II receptor, or another co-receptor TGF-beta superfamily polypeptide but may contain additional polypeptides that are not type I receptor, type II receptor, or co-receptor TGF-beta superfamily polypeptides.

In some embodiments, the disclosure provides heteromeric polypeptide complexes comprising a single TGF-beta superfamily co-receptor polypeptide, wherein the TGF-beta superfamily receptor polypeptide is derived from a betaglycan polypeptide. For example, betaglycan polypeptides may comprise of an amino acid sequence that is at least 70%, 75%, 80%, 85%, 90%, 91%, 92%, 93%, 94%, 95%, 96%, 97%, 98%, 99% or 100% identical to an betaglycan sequence disclosed herein (e.g., SEQ ID NOs: 85, 86, 89, 90, 548, 549, 550, or 551). Optionally, betaglycan polypeptides of the disclosure may be fusion proteins that further comprise one or more portions (domains) that are heterologous to endoglin. For example, an betaglycan polypeptide may be fused to a heterologous polypeptide that comprises a multimerization domain, optionally with a linker domain positioned between the betaglycan polypeptide and the heterologous polypeptide (e.g., SEQ ID NOs: 548, 549, 550, or 551). In some embodiments, multimerization domains described herein comprise one component of an interaction pair. Heteromeric complexes that comprise a betaglycan polypeptide do not comprise a type I receptor, type II receptor, or another co-receptor TGF-beta superfamily polypeptide but may contain additional polypeptides that are not type I receptor, type II receptor, or co-receptor TGF-beta superfamily polypeptides.

In some embodiments, the disclosure provides heteromeric polypeptide complexes comprising a single TGF-beta superfamily co-receptor polypeptide, wherein the TGF-beta superfamily receptor polypeptide is derived from a Cripto-1 polypeptide. For example, Cripto-1 polypeptides may comprise of an amino acid sequence that is at least 70%, 75%, 80%, 85%, 90%, 91%, 92%, 93%, 94%, 95%, 96%, 97%, 98%, 99% or 100% identical to an Cripto-1 sequence disclosed herein (e.g., SEQ ID NOs: 13, 14, 17, 18, 508, 509, 510, or 511). Optionally, Cripto-1 polypeptides of the disclosure may be fusion proteins that further comprise one or more portions (domains) that are heterologous to endoglin. For example, a Cripto-1 polypeptide may be fused to a heterologous polypeptide that comprises a multimerization domain, optionally with a linker domain positioned between the Cripto-1 polypeptide and the heterologous polypeptide (e.g., SEQ ID NOs: 508, 509, 510, or 511). In some embodiments, multimerization domains described herein comprise one component of an interaction pair. Heteromeric complexes that comprise an Cripto-1 polypeptide do not comprise a type I receptor, type II receptor, or another co-receptor TGF-beta superfamily polypeptide but may contain additional polypeptides that are not type I receptor, type II receptor, or co-receptor TGF-beta superfamily polypeptides.

In some embodiments, the disclosure provides heteromeric polypeptide complexes comprising a single TGF-beta superfamily co-receptor polypeptide, wherein the TGF-beta superfamily receptor polypeptide is derived from a Cryptic polypeptide. For example, Cryptic polypeptides may comprise of an amino acid sequence that is at least 70%, 75%, 80%, 85%, 90%, 91%, 92%, 93%, 94%, 95%, 96%, 97%, 98%, 99% or 100% identical to a Cryptic sequence disclosed herein (e.g., SEQ ID NOs: 21, 22, 25, 26, 29, 30, 512, 513, 514, or 515). Optionally, Cryptic polypeptides of the disclosure may be fusion proteins that further comprise one or more portions (domains) that are heterologous to endoglin. For example, a Cryptic polypeptide may be fused to a heterologous polypeptide that comprises a multimerization domain, optionally with a linker domain positioned between the Cryptic polypeptide and the heterologous polypeptide (e.g., SEQ ID NOs: 512, 513, 514, or 515). In some embodiments, multimerization domains described herein comprise one component of an interaction pair. Heteromeric complexes that comprise an Cryptic polypeptide do not comprise a type I receptor, type II receptor, or another co-receptor TGF-beta superfamily polypeptide but may contain additional polypeptides that are not type I receptor, type II receptor, or co-receptor TGF-beta superfamily polypeptides.

In some embodiments, the disclosure provides heteromeric polypeptide complexes comprising a single TGF-beta superfamily co-receptor polypeptide, wherein the TGF-beta superfamily receptor polypeptide is derived from a Cryptic family protein 1B polypeptide. For example, Cryptic family protein 1B polypeptides may comprise of an amino acid sequence that is at least 70%, 75%, 80%, 85%, 90%, 91%, 92%, 93%, 94%, 95%, 96%, 97%, 98%, 99% or 100% identical to a Cryptic family protein 1B sequence disclosed herein (e.g., SEQ ID NOs: 33, 34, 516, 517, 518, or 519). Optionally, Cryptic family protein 1B polypeptides of the disclosure may be fusion proteins that further comprise one or more portions (domains) that are heterologous to endoglin. For example, a Cryptic family protein 1B polypeptide may be fused to a heterologous polypeptide that comprises a multimerization domain, optionally with a linker domain positioned between the Cryptic family protein 1B polypeptide and the heterologous polypeptide (e.g., SEQ ID NOs: 516, 517, 518, or 519). In some embodiments, multimerization domains described herein comprise one component of an interaction pair. Heteromeric complexes that comprise a Cryptic family protein 1B polypeptide do not comprise a type I receptor, type II receptor, or another co-receptor TGF-beta superfamily polypeptide but may contain additional polypeptides that are not type I receptor, type II receptor, or co-receptor TGF-beta superfamily polypeptides.

In some embodiments, the disclosure provides heteromeric polypeptide complexes comprising a single TGF-beta superfamily co-receptor polypeptide, wherein the TGF-beta superfamily receptor polypeptide is derived from a Crim1 polypeptide. For example, Crim1 polypeptides may comprise of an amino acid sequence that is at least 70%, 75%, 80%, 85%, 90%, 91%, 92%, 93%, 94%, 95%, 96%, 97%, 98%, 99% or 100% identical to a Crim1 sequence disclosed herein (e.g., SEQ ID NOs: 37, 38, 520, 521, 522, or 523). Optionally, Crim1 polypeptides of the disclosure may be fusion proteins that further comprise one or more portions (domains) that are heterologous to endoglin. For example, a Crim1 polypeptide may be fused to a heterologous polypeptide that comprises a multimerization domain, optionally with a linker domain positioned between the Crim1 polypeptide and the heterologous polypeptide (e.g., SEQ ID NOs: 520, 521, 522, or 523). In some embodiments, multimerization domains described herein comprise one component of an interaction pair. Heteromeric complexes that comprise a CrimI polypeptide do not comprise a type I receptor, type II receptor, or another co-receptor TGF-beta superfamily polypeptide but may contain additional polypeptides that are not type I receptor, type II receptor, or co-receptor TGF-beta superfamily polypeptides.

In some embodiments, the disclosure provides heteromeric polypeptide complexes comprising a single TGF-beta superfamily co-receptor polypeptide, wherein the TGF-beta superfamily receptor polypeptide is derived from a Crim2 polypeptide. For example, Crim2 polypeptides may comprise of an amino acid sequence that is at least 70%, 75%, 80%, 85%, 90%, 91%, 92%, 93%, 94%, 95%, 96%, 97%, 98%, 99% or 100% identical to a Crim2 sequence disclosed herein (e.g., SEQ ID NOs: 41, 42, 45, 46, 524, 525, 526, or 527). Optionally, Crim2 polypeptides of the disclosure may be fusion proteins that further comprise one or more portions (domains) that are heterologous to endoglin. For example, a Crim2 polypeptide may be fused to a heterologous polypeptide that comprises a multimerization domain, optionally with a linker domain positioned between the Crim2 polypeptide and the heterologous polypeptide (e.g., SEQ ID NOs: 524, 525, 526, or 527). In some embodiments, multimerization domains described herein comprise one component of an interaction pair. Heteromeric complexes that comprise a Crim2 polypeptide do not comprise a type I receptor, type II receptor, or another co-receptor TGF-beta superfamily polypeptide but may contain additional polypeptides that are not type I receptor, type II receptor, or co-receptor TGF-beta superfamily polypeptides.

In some embodiments, the disclosure provides heteromeric polypeptide complexes comprising a single TGF-beta superfamily co-receptor polypeptide, wherein the TGF-beta superfamily receptor polypeptide is derived from a BAMBI polypeptide. For example, BAMBI polypeptides may comprise of an amino acid sequence that is at least 70%, 75%, 80%, 85%, 90%, 91%, 92%, 93%, 94%, 95%, 96%, 97%, 98%, 99% or 100% identical to a BAMBI sequence disclosed herein (e.g., SEQ ID NOs: 49, 50, 528, 529, 530, or 531). Optionally, BAMBI polypeptides of the disclosure may be fusion proteins that further comprise one or more portions (domains) that are heterologous to endoglin. For example, a BAMBI polypeptide may be fused to a heterologous polypeptide that comprises a multimerization domain, optionally with a linker domain positioned between the BAMBI polypeptide and the heterologous polypeptide (e.g., SEQ ID NOs: 528, 529, 530, or 531). In some embodiments, multimerization domains described herein comprise one component of an interaction pair. Heteromeric complexes that comprise a BAMBI polypeptide do not comprise a type I receptor, type II receptor, or another co-receptor TGF-beta superfamily polypeptide but may contain additional polypeptides that are not type I receptor, type II receptor, or co-receptor TGF-beta superfamily polypeptides.

In some embodiments, the disclosure provides heteromeric polypeptide complexes comprising a single TGF-beta superfamily co-receptor polypeptide, wherein the TGF-beta superfamily receptor polypeptide is derived from a BMPER polypeptide. For example, BMPER polypeptides may comprise of an amino acid sequence that is at least 70%, 75%, 80%, 85%, 90%, 91%, 92%, 93%, 94%, 95%, 96%, 97%, 98%, 99% or 100% identical to a BMPER sequence disclosed herein (e.g., SEQ ID NOs: 53, 54, 532, 533, 534, or 535). Optionally, BMPER polypeptides of the disclosure may be fusion proteins that further comprise one or more portions (domains) that are heterologous to endoglin. For example, a BMPER polypeptide may be fused to a heterologous polypeptide that comprises a multimerization domain, optionally with a linker domain positioned between the BMPER polypeptide and the heterologous polypeptide (e.g., SEQ ID NOs: 532, 533, 534, or 535). In some embodiments, multimerization domains described herein comprise one component of an interaction pair. Heteromeric complexes that comprise a BMPER polypeptide do not comprise a type I receptor, type II receptor, or another co-receptor TGF-beta superfamily polypeptide but may contain additional polypeptides that are not type I receptor, type II receptor, or co-receptor TGF-beta superfamily polypeptides.

In some embodiments, the disclosure provides heteromeric polypeptide complexes comprising a single TGF-beta superfamily co-receptor polypeptide, wherein the TGF-beta superfamily receptor polypeptide is derived from a RGM-A polypeptide. For example, RGM-A polypeptides may comprise of an amino acid sequence that is at least 70%, 75%, 80%, 85%, 90%, 91%, 92%, 93%, 94%, 95%, 96%, 97%, 98%, 99% or 100% identical to a RGM-A sequence disclosed herein (e.g., SEQ ID NOs: 61, 62, 65, 66, 69, 70, 540, 541, 542, or 543). Optionally, RGM-A polypeptides of the disclosure may be fusion proteins that further comprise one or more portions (domains) that are heterologous to endoglin. For example, a RGM-A polypeptide may be fused to a heterologous polypeptide that comprises a multimerization domain, optionally with a linker domain positioned between the RGM-A polypeptide and the heterologous polypeptide (e.g., SEQ ID NOs: 540, 541, 542, or 543). In some embodiments, multimerization domains described herein comprise one component of an interaction pair. Heteromeric complexes that comprise a RGM-A polypeptide do not comprise a type I receptor, type II receptor, or another co-receptor TGF-beta superfamily polypeptide but may contain additional polypeptides that are not type I receptor, type II receptor, or co-receptor TGF-beta superfamily polypeptides.

In some embodiments, the disclosure provides heteromeric polypeptide complexes comprising a single TGF-beta superfamily co-receptor polypeptide, wherein the TGF-beta superfamily receptor polypeptide is derived from a RGM-B polypeptide. For example, RGM-B polypeptides may comprise of an amino acid sequence that is at least 70%, 75%, 80%, 85%, 90%, 91%, 92%, 93%, 94%, 95%, 96%, 97%, 98%, 99% or 100% identical to an RGM-B sequence disclosed herein (e.g., SEQ ID NOs: 57, 58, 536, 537, 538, or 539). Optionally, RGM-B polypeptides of the disclosure may be fusion proteins that further comprise one or more portions (domains) that are heterologous to endoglin. For example, a RGM-B polypeptide may be fused to a heterologous polypeptide that comprises a multimerization domain, optionally with a linker domain positioned between the RGM-B polypeptide and the heterologous polypeptide (e.g., SEQ ID NOs: 536, 537, 538, or 539). In some embodiments, multimerization domains described herein comprise one component of an interaction pair. Heteromeric complexes that comprise a RGM-B polypeptide do not comprise a type I receptor, type II receptor, or another co-receptor TGF-beta superfamily polypeptide but may contain additional polypeptides that are not type I receptor, type II receptor, or co-receptor TGF-beta superfamily polypeptides.

In some embodiments, the disclosure provides heteromeric polypeptide complexes comprising a single TGF-beta superfamily co-receptor polypeptide, wherein the TGF-beta superfamily receptor polypeptide is derived from a hemojuvelin polypeptide. For example, hemojuvelin polypeptides may comprise of an amino acid sequence that is at least 70%, 75%, 80%, 85%, 90%, 91%, 92%, 93%, 94%, 95%, 96%, 97%, 98%, 99% or 100% identical to a hemojuvelin sequence disclosed herein (e.g., SEQ ID NOs: 73, 74, 77, 78, 81, 82, 544, 545, 546, or 547). Optionally, hemojuvelin polypeptides of the disclosure may be fusion proteins that further comprise one or more portions (domains) that are heterologous to endoglin. For example, a hemojuvelin polypeptide may be fused to a heterologous polypeptide that comprises a multimerization domain, optionally with a linker domain positioned between the hemojuvelin polypeptide and the heterologous polypeptide (e.g., SEQ ID NOs: 544, 545, 546, or 547). In some embodiments, multimerization domains described herein comprise one component of an interaction pair. Heteromeric complexes that comprise a hemojuvelin polypeptide do not comprise a type I receptor, type II receptor, or another co-receptor TGF-beta superfamily polypeptide but may contain additional polypeptides that are not type I receptor, type II receptor, or co-receptor TGF-beta superfamily polypeptides.

In some embodiments, the disclosure provides heteromeric polypeptide complexes comprising a single TGF-beta superfamily co-receptor polypeptide, wherein the TGF-beta superfamily receptor polypeptide is derived from a MuSK polypeptide. For example, MuSK polypeptides may comprise of an amino acid sequence that is at least 70%, 75%, 80%, 85%, 90%, 91%, 92%, 93%, 94%, 95%, 96%, 97%, 98%, 99% or 100% identical to a MuSK sequence disclosed herein (e.g., SEQ ID NOs: 95, 96, 99, 100, 103, 104, 552, 553, 554, or 555). Optionally, MuSK polypeptides of the disclosure may be fusion proteins that further comprise one or more portions (domains) that are heterologous to endoglin. For example, a MuSK polypeptide may be fused to a heterologous polypeptide that comprises a multimerization domain, optionally with a linker domain positioned between the MuSK polypeptide and the heterologous polypeptide (e.g., SEQ ID NOs: 552, 553, 554, or 555). In some embodiments, multimerization domains described herein comprise one component of an interaction pair. Heteromeric complexes that comprise a MuSK polypeptide do not comprise a type I receptor, type II receptor, or another co-receptor TGF-beta superfamily polypeptide but may contain additional polypeptides that are not type I receptor, type II receptor, or co-receptor TGF-beta superfamily polypeptides.

In some embodiments, the TGF-beta superfamily co-receptor polypeptides disclosed herein comprise one or more modified amino acid residues selected from: a glycosylated amino acid, a PEGylated amino acid, a farnesylated amino acid, an acetylated amino acid, a biotinylated amino acid, an amino acid conjugated to a lipid moiety, and an amino acid conjugated to an organic derivatizing agent. In some embodiments, the co-receptor polypeptides described herein are glycosylated and have a glycosylation pattern obtainable from the expression of the polypeptides in a mammalian cell, including, for example, a CHO cell.

In certain aspects the disclosure provides nucleic acids encoding any of the TGF-beta superfamily co-receptor polypeptides described herein, including any fusion proteins comprising members of an interaction pair. Nucleic acids disclosed herein may be operably linked to a promoter for expression, and the disclosure further provides cells transformed with such recombinant polynucleotides. Preferably the cell is a mammalian cell such as a COS cell or a CHO cell.

In certain aspects, the disclosure provides methods for making any of the TGF-beta superfamily co-receptor polypeptides described herein as well as protein complexes comprising such a polypeptide. Such a method may include expressing any of the nucleic acids disclosed herein in a suitable cell (e.g., CHO cell or a COS cell). Such a method may comprise: a) culturing a cell under conditions suitable for expression of a TGF-beta superfamily co-receptor polypeptides described herein, wherein said cell is transformed with a co-receptor polypeptide expression construct; and b) recovering the co-receptor polypeptides so expressed. TGF-beta superfamily co-receptor polypeptides described herein, as well as protein complexes of the same, may be recovered as crude, partially purified, or highly purified fractions using any of the well-known techniques for obtaining protein from cell cultures.

Any of the protein complexes described herein may be incorporated into a pharmaceutical preparation. Optionally, such pharmaceutical preparations are at least 80%, 85%, 90%, 95%, 97%, 98% or 99% pure with respect to other polypeptide components. Optionally, pharmaceutical preparations disclosed herein may comprise one or more additional active agents.

The disclosure further provides methods for use of the protein complexes and pharmaceutical preparations described herein for the treatment or prevention of various TGF-beta associated conditions, including without limitation diseases and disorders associated with, for example, cancer, muscle, bone, fat, red blood cells, metabolism, fibrosis and other tissues that are affected by one or more ligands of the TGF-beta superfamily.

### BRIEF DESCRIPTION OF THE DRAWINGS

FIG. 1 shows a schematic example of a single-arm heteromeric protein complex comprising a co-receptor polypeptide (indicated as “CoR”) (e.g. a polypeptide that is at least 70%, 75%, 80%, 85%, 90%, 91%, 92%, 93%, 94%, 95%, 96%, 97%, 98%, 99% or 100% identical to an extracellular domain of an endoglin, betaglycan, Cripto-1, Cryptic, Cryptic family protein 1B, Crim1, Crim2, BAMBI, BMPER, RGM-A, RGM-B, MuSK, and hemojuvelin protein from humans or other species). In the illustrated embodiment, the co-receptor polypeptide is part of a fusion polypeptide that comprises a first member of an interaction pair (“B”), which associates with a second member of an interaction pair (“C”). In the fusion polypeptide, a linker may be positioned between the co-receptor polypeptide and the corresponding member of the interaction pair. The first and second members of the interaction pair (B, C) may be a guided (asymmetric) pair, meaning that the members of the pair associate preferentially with each other rather than self-associate, or the interaction pair may be unguided, meaning that the members of the pair may associate with each other or self-associate without substantial preference and may have the same or different amino acid sequences. Traditional Fc fusion proteins and antibodies are examples of unguided interaction pairs, whereas a variety of engineered Fc domains have been designed as guided (asymmetric) interaction pairs.

FIG. 2 shows multiple sequence alignment of Fc domains from human IgG isotypes using Clustal 2.1. Hinge regions are indicated by dotted underline. Double underline indicates examples of positions engineered in IgG1 Fc to promote asymmetric chain pairing and the corresponding positions with respect to other isotypes IgG2, IgG3 and IgG4.

### DETAILED DESCRIPTION OF THE INVENTION

### 1. Overview

In part, the present disclosure relates to single-arm heteromultimer complexes comprising a ligand-binding domain of a TGFβ superfamily co-receptor polypeptide, methods of making such single-arm heteromultimer complexes, and uses thereof. As described herein, single-arm heteromultimer complexes may comprise a ligand-binding domain of a TGFβ superfamily co-receptor polypeptide selected from: endoglin, betaglycan, Cripto-1, Cryptic, Cryptic family protein 1B, Crim1, Crim2, BAMBI, BMPER, RGM-A, RGM-B, MuSK, and hemojuvelin. In some embodiments, heteromultimer complexes of the disclosure have an altered profile of binding to TGFβ superfamily ligands relative to a corresponding homomultimer complex.

The TGF-β superfamily is comprised of over thirty secreted factors including TGF-betas, activins, nodals, bone morphogenetic proteins (BMPs), growth and differentiation factors (GDFs), and anti-Mullerian hormone (AMH). See, e.g., Weiss et al. (2013) Developmental Biology, 2(1): 47-63. Members of the superfamily, which are found in both vertebrates and invertebrates, are ubiquitously expressed in diverse tissues and function during the earliest stages of development throughout the lifetime of an animal. Indeed, TGF-β superfamily proteins are key mediators of stem cell self-renewal, gastrulation, differentiation, organ morphogenesis, and adult tissue homeostasis. Consistent with this ubiquitous activity, aberrant TGF-beta superfamily signaling is associated with a wide range of human pathologies including, for example, autoimmune disease, cardiovascular disease, fibrotic disease, and cancer.

Ligands of the TGF-beta superfamily share the same dimeric structure in which the central 3-1/2 turn helix of one monomer packs against the concave surface formed by the beta-strands of the other monomer. The majority of TGF-beta family members are further stabilized by an intermolecular disulfide bond. This disulfide bonds traverses through a ring formed by two other disulfide bonds generating what has been termed a ‘cysteine knot’ motif. See, e.g., Lin et al., (2006) Reproduction 132: 179-190 and Hinck (2012) FEBS Letters 586: 1860-1870.

TGF-beta superfamily signaling is mediated by heteromeric complexes of type I and type II serine/threonine kinase receptors, which phosphorylate and activate downstream SMAD proteins (e.g., SMAD proteins 1, 2, 3, 5, and 8) upon ligand stimulation. See, e.g., Massagud (2000) Nat. Rev. Mol. Cell Biol. 1:169-178. These type I and type II receptors are transmembrane proteins, composed of a ligand-binding extracellular domain with cysteine-rich region, a transmembrane domain, and a cytoplasmic domain with predicted serine/threonine kinase specificity. In general, type I receptors mediate intracellular signaling while the type II receptors are required for binding TGF-beta superfamily ligands. Type I and II receptors form a stable complex after ligand binding, resulting in phosphorylation of type I receptors by type II receptors.

The TGF-beta family can be divided into two phylogenetic branches based on the type I receptors they bind and the Smad proteins they activate. One is the more recently evolved branch, which includes, e.g., the TGF-betas, activins, GDF8, GDF9, GDF11, BMP3 and nodal. The other branch comprises the more distantly related proteins of the superfamily and includes, e.g., BMP2, BMP4, BMP5, BMP6, BMP7, BMP8a, BMP8b, BMP9, BMP10, GDF1, GDF5, GDF6, and GDF7. See, e.g. Hinck (2012) FEBS Letters 586:1860-1870.

TGF-beta isoforms are the founding members of the TGF-beta superfamily, of which there are 3 known isoforms in mammals designated as TGF-beta1, TGF-beta2 and TGF-beta3. Mature bioactive TGF-beta ligands function as homodimers and predominantly signal through the type I receptor ALK5 but have also been found to signal through ALK1 in endothelial cells. See, e.g., Goumans et al. (2003) Mol Cell 12(4): 817-828. TGF-beta1 is the most abundant and ubiquitously expressed isoform. TGF-beta1 is known to have an important role in wound healing, and mice expressing a constitutively active TGF-beta1 transgene develop fibrosis. See e.g., Clouthier et al., (1997) J Clin. Invest. 100(11): 2697-2713. TGF-beta1 is also involved in T cell activation and maintenance of T regulatory cells. See, e.g., Li et al., (2006) Immunity 25(3): 455-471. TGF-beta2 expression was first described in human glioblastoma cells and occurs in neurons and astroglial cells of the embryonic nervous system. TGF-beta2 is also known to suppress interleukin-2-dependent growth of T lymphocytes. TGF-beta3 was initially isolated from a human rhabdomyosarcoma cell line and since has been found in lung adenocarcinoma and kidney carcinoma cell lines. TGF-beta3 is known to be important for palate and lung morphogenesis. See, e.g., Kubiczkova et al., (2012) Journal of Translational Medicine 10:183.

Activins are members of the TGF-beta superfamily that were initially discovered as regulators of follicle-stimulating hormone secretion, but subsequently various reproductive and non-reproductive roles have been characterized. Principal activin forms A, B, and AB are homo/heterodimers of two closely related β subunits (β_{A}β_{A}, β_{B}β_{B}, and β_{A}β_{B}, respectively). The human genome also encodes an activin C and an activin E, which are primarily expressed in the liver, and heterodimeric forms containing β_{C} or β_{E} are also known. In the TGF-beta superfamily, activins are unique and multifunctional factors that can stimulate hormone production in ovarian and placental cells, support neuronal cell survival, influence cell-cycle progress positively or negatively depending on cell type, and induce mesodermal differentiation at least in amphibian embryos. See, e.g., DePaolo et al. (1991) Proc Soc Ep Biol Med. 198:500-512; Dyson et al. (1997) Curr Biol. 7:81-84; and Woodruff (1998) Biochem Pharmacol. 55:953-963. In several tissues, activin signaling is antagonized by its related heterodimer, inhibin. For example, in the regulation of follicle-stimulating hormone (FSH) secretion from the pituitary, activin promotes FSH synthesis and secretion, while inhibin reduces FSH synthesis and secretion. Other proteins that may regulate activin bioactivity and/or bind to activin include follistatin (FS), follistatin-related protein (FSRP, also known as FLRG or FSTL3), and α₂-macroglobulin.

As described herein, agents that bind to “activin A” are agents that specifically bind to the β_{A} subunit, whether in the context of an isolated β_{A} subunit or as a dimeric complex (e.g., a β_{A}β_{A} homodimer or a β_{A}β_{B} heterodimer). In the case of a heterodimer complex (e.g., a β_{A}β_{B} heterodimer), agents that bind to “activin A” are specific for epitopes present within the β_{A} subunit, but do not bind to epitopes present within the non-β_{A} subunit of the complex (e.g., the β_{B} subunit of the complex). Similarly, agents disclosed herein that antagonize (inhibit) “activin A” are agents that inhibit one or more activities as mediated by a β_{A} subunit, whether in the context of an isolated DA subunit or as a dimeric complex (e.g., a β_{A}β_{A} homodimer or a β_{A}β_{B} heterodimer). In the case of β_{A}β_{B} heterodimers, agents that inhibit “activin A” are agents that specifically inhibit one or more activities of the β_{A} subunit but do not inhibit the activity of the non-β_{A} subunit of the complex (e.g., the β_{B} subunit of the complex). This principle applies also to agents that bind to and/or inhibit “activin B”, “activin C”, and “activin E”. Agents disclosed herein that antagonize “activin AB” are agents that inhibit one or more activities as mediated by the β_{A} subunit and one or more activities as mediated by the β_{B} subunit.

The BMPs and GDFs together form a family of cysteine-knot cytokines sharing the characteristic fold of the TGF-beta superfamily. See, e.g., Rider et al. (2010) Biochem J., 429(1):1-12. This family includes, for example, BMP2, BMP4, BMP6, BMP7, BMP2a, BMP3, BMP3b (also known as GDF10), BMP4, BMP5, BMP6, BMP7, BMP8, BMP8a, BMP8b, BMP9 (also known as GDF2), BMP10, BMP11 (also known as GDF11), BMP12 (also known as GDF7), BMP13 (also known as GDF6), BMP14 (also known as GDF5), BMP15, GDF1, GDF3 (also known as VGR2), GDF8 (also known as myostatin), GDF9, GDF15, and decapentaplegic. Besides the ability to induce bone formation, which gave the BMPs their name, the BMP/GDFs display morphogenetic activities in the development of a wide range of tissues. BMP/GDF homo- and hetero-dimers interact with combinations of type I and type II receptor dimers to produce multiple possible signaling complexes, leading to the activation of one of two competing sets of SMAD transcription factors. BMP/GDFs have highly specific and localized functions. These are regulated in a number of ways, including the developmental restriction of BMP/GDF expression and through the secretion of several proteins that bind certain TGF-beta superfamily ligands with high affinity and thereby inhibit ligand activity. Curiously, some of these endogenous antagonists resemble TGF-beta superfamily ligands themselves.

Growth and differentiation factor-8 (GDF8) is also known as myostatin. GDF8 is a negative regulator of skeletal muscle mass and is highly expressed in developing and adult skeletal muscle. The GDF8 null mutation in transgenic mice is characterized by a marked hypertrophy and hyperplasia of skeletal muscle. See, e.g., McPherron et al., Nature (1997) 387:83-90. Similar increases in skeletal muscle mass are evident in naturally occurring mutations of GDF8 in cattle and, strikingly, in humans. See, e.g., Ashmore et al. (1974) Growth, 38:501-507; Swatland and Kieffer, J. Anim. Sci. (1994) 38:752-757; McPherron and Lee, Proc. Natl. Acad. Sci. USA (1997) 94:12457-12461; Kambadur et al., Genome Res. (1997) 7:910-915; and Schuelke et al. (2004) N Engl J Med, 350:2682-8. Studies have also shown that muscle wasting associated with HIV-infection in humans is accompanied by increases in GDF8 protein expression. See, e.g., Gonzalez-Cadavid et al., PNAS (1998) 95:14938-43. In addition, GDF8 can modulate the production of muscle-specific enzymes (e.g., creatine kinase) and modulate myoblast cell proliferation. See, e.g., International Patent Application Publication No. WO 00/43781). The GDF8 propeptide can noncovalently bind to the mature GDF8 domain dimer, inactivating its biological activity. See, e.g., Miyazono et al. (1988) J. Biol. Chem., 263: 6407-6415; Wakefield et al. (1988) J. Biol. Chem., 263; 7646-7654; and Brown et al. (1990) Growth Factors, 3: 35-43. Other proteins which bind to GDF8 or structurally related proteins and inhibit their biological activity include follistatin, and potentially, follistatin-related proteins. See, e.g., Gamer et al. (1999) Dev. Biol., 208: 222-232.

GDF11, also known as BMP11, is a secreted protein that is expressed in the tail bud, limb bud, maxillary and mandibular arches, and dorsal root ganglia during mouse development. See, e.g., McPherron et al. (1999) Nat. Genet., 22: 260-264; and Nakashima et al. (1999) Mech. Dev., 80: 185-189. GDF11 plays a unique role in patterning both mesodermal and neural tissues. See, e.g., Gamer et al. (1999) Dev Biol., 208:222-32. GDF11 was shown to be a negative regulator of chondrogenesis and myogenesis in developing chick limb. See, e.g., Gamer et al. (2001) Dev Biol., 229:407-20. The expression of GDF11 in muscle also suggests its role in regulating muscle growth in a similar way to GDF8. In addition, the expression of GDF11 in brain suggests that GDF11 may also possess activities that relate to the function of the nervous system. Interestingly, GDF11 was found to inhibit neurogenesis in the olfactory epithelium. See, e.g., Wu et al. (2003) Neuron., 37:197-207. Hence, GDF11 may have in vitro and in vivo applications in the treatment of diseases such as muscle diseases and neurodegenerative diseases (e.g., amyotrophic lateral sclerosis).

BMP7, also called osteogenic protein-1 (OP-1), is well known to induce cartilage and bone formation. In addition, BMP7 regulates a wide array of physiological processes. For example, BMP7 may be the osteoinductive factor responsible for the phenomenon of epithelial osteogenesis. It is also found that BMP7 plays a role in calcium regulation and bone homeostasis. Like activin, BMP7 binds to type II receptors, ActRIIA and ActRIIB. However, BMP7 and activin recruit distinct type I receptors into heteromeric receptor complexes. The major BMP7 type I receptor observed was ALK2, while activin bound exclusively to ALK4 (ActRIIB). BMP7 and activin elicited distinct biological responses and activated different SMAD pathways. See, e.g., Macias-Silva et al. (1998) J Biol Chem. 273:25628-36.

Anti-Mullerian hormone (AMH), also known as Mullerian-inhibiting substance (MIS), is a TGF-beta family glycoprotein. One AMH-associated type II receptor has been identified and is designated as AMHRII, or alternatively MISRII. AMH induces regression of the Mullerian ducts in the human male embryo. AMH is expressed in reproductive age women and does not fluctuate with cycle or pregnancy, but was found to gradually decrease as both oocyte quantity and quality decrease, suggesting AMH could serve as a biomarker for ovarian physiology. See e.g. Zec et al., (2011) Biochemia Medica 21(3): 219-30.

In certain aspects, the present invention relates to ENG polypeptides. The protein endoglin (ENG), also known as CD105 and encoded by ENG, is considered a co-receptor for the transforming growth factor-β (TGF-β) superfamily of ligands and is implicated in normal and pathological fibrosis and angiogenesis. Structurally, ENG is a homodimeric cell-surface glycoprotein. It belongs to the zona pellucida (ZP) family of proteins and consists of a short C-terminal cytoplasmic domain, a single hydrophobic transmembrane domain, and a long extracellular domain (ECD) (Gougos et al, 1990, J Biol Chem 265:8361-8364). As determined by electron microscopy, monomeric ENG ECD consists of two ZP regions and an orphan domain located at the N-terminus (Llorca et al, 2007, J Mol Biol 365:694-705).

ENG expression is low in quiescent vascular endothelium but upregulated in endothelial cells of healing wounds, developing embryos, inflammatory tissues, and solid tumors (Dallas et al, 2008, Clin Cancer Res 14:1931-1937). Mice homozygous for null ENG alleles die early in gestation due to defective vascular development (Li et al, 1999, Science 284:1534-1537), whereas heterozygous null ENG mice display angiogenic abnormalities as adults (Jerkic et al, 2006, Cardiovasc Res 69:845-854). In humans, ENG gene mutations have been identified as the cause of hereditary hemorrhagic telangiectasia (Osler-Rendu-Weber syndrome) type-1 (HHT-1), an autosomal dominant form of vascular dysplasia characterized by arteriovenous malformations resulting in direct flow (communication) from artery to vein (arteriovenous shunt) without an intervening capillary bed (McAllister et al, 1994, Nat Genet 8:345-351; Fernandez-L et al, 2006, Clin Med Res 4:66-78). Typical symptoms of patients with HHT include recurrent epistaxis, gastrointestinal hemorrhage, cutaneous and mucocutaneous telangiectases, and arteriovenous malformations in the pulmonary, cerebral, or hepatic vasculature.

As a co-receptor, ENG is thought to modulate responses of other receptors to TGF-β family ligands without direct mediation of ligand signaling by itself. Ligands in the TGF-β family typically signal by binding to a homodimeric type II receptor, which triggers recruitment and transphosphorylation of a homodimeric type I receptor, thereby leading to phosphorylation of Smad proteins responsible for transcriptional activation of specific genes (Massague, 2000, Nat Rev Mol Cell Biol 1:169-178). Based on ectopic cellular expression assays, it has been reported that ENG cannot bind ligands on its own and that its binding to TGF-β1, TGF-β3, activin A, bone morphogenetic protein-2 (BMP-2), and BMP-7 requires the presence of an appropriate type I and/or type II receptor (Barbara et al, 1999, J Biol Chem 274:584-594). Nevertheless, there is evidence that ENG expressed by a fibroblast cell line can bind TGF-β1 (St.-Jacques et al, 1994, Endocrinology 134:2645-2657), and recent results in COS cells indicate that transfected full-length ENG can bind BMP-9 in the absence of transfected type I or type II receptors (Scharpfenecker et al, 2007, J Cell Sci 120:964-972).

In addition to the foregoing, ENG can occur in a soluble form in vivo under certain conditions after proteolytic cleavage of the full-length membrane-bound protein (Hawinkels et al, 2010, Cancer Res 70:4141-4150). Elevated levels of soluble ENG have been observed in the circulation of patients with cancer and preeclampsia (Li et al, 2000, Int J Cancer 89:122-126; Calabro et al, 2003, J Cell Physiol 194:171-175; Venkatesha et al, 2006, Nat Med 12:642-649; Levine et al, 2006, N Engl J Med 355:992-1005). Although the role of endogenous soluble ENG is poorly understood, a protein corresponding to residues 26-437 of the ENG precursor (amino acids 26-437 of SEQ ID NO: 1) has been proposed to act as a scavenger or trap for TGF-β family ligands (Venkatesha et al, 2006, Nat Med 12:642-649; WO-2007/143023), of which only TGF-β1 and TGF-β3 have specifically been implicated.

In certain aspects, the present invention relates to betaglycan polypeptides. Betaglycan, also known as TGFβ receptor type III (TβRIII, TGFβRIII) and encoded by TGFBR3, is a single-pass transmembrane protein consisting of a large extracellular domain, transmembrane domain, and relatively short cytoplasmic domain (43 amino acids). It is thought that betaglycan is not directly involved in signal transduction since its cytoplasmic domain lacks an obvious signaling motif Consistent with a co-receptor role, the presence of betaglycan on the cell surface increases the binding of TGFβ isoforms to their type II receptor (TGFβRII) and increases ligand efficacy in biologic assays (Bilandzic et al., 2011, Mol Cell Endocrinol 339:180-189). This effect is most pronounced for TGFβ2, which binds weakly to TGFβRII in the absence of betaglycan (Lopez-Casillas et al., 1993, 1994). In addition, the extracellular domain of betaglycan is released from some cells in a soluble form whose physiologic role remains to be determined.

Betaglycan can alter signaling by superfamily ligands besides TGFβ. For example, inhibin is capable of binding ActRIIA or ActRIIB and functionally antagonizing activins by preventing recruitment of activin type I receptors. However, inhibin requires the presence of betaglycan for high potency inhibition of activin signaling (Lewis et al., 2000, Nature 404:411-414; Wiater et al., 2009, Mol Endocrinol 23:1033-1042). Betaglycan forms a stable complex with inhibin and activin type II receptors, thus reducing the availability of these receptors to transmit activin signaling (Lewis et al., 2000, Nature 404:411-414). In a similar manner, betaglycan enables inhibin to antagonize the binding of BMPs to ActRIIA, ActRIIB, or BMPRII, thereby inhibiting BMP signaling (Wiater et al., 2003, J Biol Chem 278:7934-7941).

In certain aspects, the present invention relates to EGF-CFC family polypeptides. Members of the epidermal growth factor-Cripto-1/FRL-1/Cryptic (EGF-CFC) family in humans include founder Cripto-1 (encoded by TDGF1) as well as Cryptic protein (encoded by CFC1) and Cryptic family protein 1B (encoded by CFC1B). EGF-CFC genes encode small extracellular proteins that contain a divergent EGF motif and a novel conserved cysteine-rich domain termed the CFC motif, with most sequence similarity occurring in the central EGF and CFC motifs (Shen et al., 2000, Trends Genet 16:303-309). Most EGF-CFC proteins have been shown or predicted to possess a glycosylphosphatidylinositol (GPI) anchor site at the C-terminus. However, soluble extracellular forms of these proteins also exist (see, e.g., Watanabe et al., 2007, J Biol Chem 282:31643-31655).

In certain aspects, the present invention relates to Cripto-1 polypeptides. Cripto-1, also known as Cripto orteratocarcinoma-derived growth factor (TDGF-1), regulates the activity of multiple TGFβ superfamily ligands that signal via the Smad2/3 pathway. Cripto-1 functions as an obligatory cell-surface co-receptor for a subset of ligands including Nodal, GDF1, and GDF3 (Gray et al., 2012, FEBS Lett 586:1836-1845). Cripto-1 acts as a co-receptor for Nodal by recruiting ALK4, leading to formation of an ActRIIB-ALK4-Cripto-Nodal complex for signaling (Rosa, 2002, Sci STKE 2002 (158):pe47; Yan et al., 2002, Mol Cell Biol 22:4439-4449; Blanchet et al., 2008, Sci Signal 1 (45):ra13). This co-receptor function plays essential roles in regulating stem cell differentiation and vertebrate embryogenesis and regulates normal tissue growth and remodeling in adult tissues. See, e.g., Guardiola et al. (2012) Proc Natl Acad Sci USA 109:E3231-E3240. Cripto-1 co-receptor function has also been linked to tumor growth since Nodal signaling plays a key role in promoting tumorigenicity. In addition to facilitating signaling by some ligands, Cripto-1 inhibits receptor activation by activin A, activin B, myostatin (GDF8), and TGFβ (Gray et al., 2003, Proc Natl Acad Sci USA 100:5193-5198; Gray et al., 2006, Mol Cell Biol 26:9268-9278; Guardiola et al., 2012, Proc Natl Acad Sci USA 109:E3231-E3240). It has been shown in a detailed analysis that Cripto-1 forms analogous receptor complexes with Nodal and activin and thereby functions as a noncompetitive activin antagonist (Kelber et al., 2008, J Biol Chem 283:4490-4500).

In certain aspects, the present invention relates to Cryptic and Cryptic family 1B polypeptides. On the basis of phenotypes in double null mutant mice, Cryptic and Cripto-1 have been found to serve partially redundant functions during early embryonic development, and most if not all Nodal activity in early mouse embryogenesis is thought to be dependent on these two EGF-CFC proteins (Chu et al., 2010, Dev Biol 342:63-73). A separate study of mice deficient only in Cryptic has revealed a role for this protein in correct establishment of left-right asymmetry during embryogenesis (Gaio et al., 1999, Curr Biol 9:1339-1342).

In certain aspects, the present invention relates to chordin-related polypeptides. Proteins in this family contain chordin-like cysteine-rich repeat (CRR) motifs of the von Willebrand C (VWC) type which are important for protein binding to superfamily ligands. Such CRRs have a conserved consensus sequence based on ten cysteines (CXₙWX₄CX₂CXCX₆CX₄CX₄₋₆CX₉₋₁₁CCPXC) (Sasai et al., 1994, Cell 79:779-790; Garcia-Abreu et al., 2002, Gene 287:39-47). Examples of chordin-related proteins include BMPER, CRIM1, and CRIM2.

In certain aspects, the present invention relates to BMPER polypeptides. BMP-binding endothelial cell precursor-derived regulator (BMPER) is encoded by BMPER and is the human homolog of Drosophila Crossveinless-2 (CV-2). BMPER is a secreted protein containing five CCR motifs and is reported to be proteolytically cleaved to generate two fragments that are disulfide-linked (Moser et al., 2003, Mol Cell Biol 23:5664-5679; Binnerts et al., 2004, Biochem Biophys Res Commun 315:272-280). Mammalian BMPER was originally identified as an inhibitor of BMP signaling. However, subsequent investigation determined that BMPER can exert biphasic activity depending on concentration, enhancing BMP-mediated signaling at molar concentrations less than that of ligand but inhibiting such signaling at concentrations exceeding those of ligand (Kelley et al., 2009, J Cell Biol 184:597-609). BMPER is implicated in a wide range of BMP-mediated differentiation processes during embryonic development and also implicated as an important postnatal regulator of BMP-mediated vascular inflammation in mice (Pi et al., 2012, Arterioscler Thromb Vase Biol 32:2214-2222).

In certain aspects, the present invention relates to CRIM1 polypeptides. Cysteine-rich motor neuron 1 (CRIM1), also known as “cysteine-rich transmembrane BMP regulator 1”, is encoded by CRIM1. This type I transmembrane protein contains a signal sequence, an extracellular domain (905 amino acids), a transmembrane domain (21 amino acids), and an intracellular domain (76 amino acids). The extracellular domain can also be released from the cell as a soluble form, likely via cleavage of the full protein at the membrane (Wilkinson et al., 2003, J Biol Chem 278:34181-34188), and contains an N-terminal insulin-like growth factor-binding motif and six chordin-like CRR motifs of the VWC type. These CRRs mediate protein binding to superfamily ligands such as TGFβ isoforms, BMP4, and BMP7 (see, e.g., Wilkinson et al., 2003, J Biol Chem 278:34181-34188). CRIM1 inhibits BMP signaling in part by reducing the rate of processing and delivery of BMPs to the cell surface. Studies in transgenic mice expressing a dominant negative (truncated) CRIM1 isoform indicate the importance of CRIM1 for normal development of the eye, central nervous system, and kidney (Pennisi et al., 2007, Dev Dyn 236:502-511; Wilkinson et al., 2007, J Am Soc Nephrol 18:1697-1708).

In certain aspects, the present invention relates to CRIM2 polypeptides. CRIM2 is a secreted protein encoded by the human gene KCP (kielin/chordin-like protein 1), named in recognition of the protein's sequence similarity to Xenopus kielin and mouse chordin. The longest CRIM2 isoform, which is nearly 1500 amino acids in human, contains many CRR motifs of the VWC type. Unlike most inhibitory proteins containing CRR motifs, CRIM2 is a potent enhancer of BMP signaling and is able to increase the affinity of BMP7 for its type I receptor ALK3 and/or enhance the stability of this ligand-receptor complex in mice (Lin et al., 2005, Nat Med 11:387-393). Mice homozygous for a CRIM2 null allele are viable and fertile but are hypersensitive to developing renal interstitial fibrosis, a disease stimulated by TGFβ but inhibited by BMP7. In contrast to the enhancing effect on BMPs, CRIM2 inhibits both activin A-mediated and TGFβ 1-mediated signaling through the Smad2/3 pathway (Lin et al., 2006, Mol Cell Biol 26:4577-4585). These inhibitory effects of CRIM2 are mediated in a paracrine manner, suggesting that direct binding of CRIM2 to TGF 1 or activin A can block interactions of these ligands with prospective receptors. The ability to enhance BMP signaling while suppressing activation by TGFβ and activin indicates an important role for CRIM2 in modulating responses between these antifibrotic and profibrotic cytokines in the initiation and progression of renal interstitial fibrosis.

In certain aspects, the present invention relates to BAMBI polypeptides. The protein named “BMP and activin membrane-bound inhibitor” (BAMBI), also known as “non-metastatic gene A” (NMA), is encoded by BAMBI. BAMBI resembles a type I receptor from the TGFβ superfamily, with an extracellular domain (132 amino acids), a transmembrane domain, and a cytoplasmic domain. However, BAMBI lacks an intracellular kinase domain and has therefore been described as a pseudoreceptor (Onichtchouk et al., 1999, Nature 401:480-485). BAMBI competes with type I receptors to form stable complexes with type II receptors and thereby prevents the formation of active complexes of type I and type II receptors. Additionally, BAMBI cooperates with Smad7 to inhibit ligand-mediated signaling (Yan et al., 2009, J Biol Chem 284:30097-30104). Ligands inhibited by BAMBI include BMPs, activin, and TGFβ. During development, BAMBI is prominent in gastrulation, neurulation, and development of bones and teeth, and is often co-expressed with BMP family members (Onichtchouk et al., 1999, Nature 401:480-485; Knight et al., J Dent Res 80:1895; Paulsen et al., 2011, Proc Natl Acad Sci USA 108:10202-). In the adult, BAMBI modulates processes such as diabetic nephropathy, thrombus formation, response to cardiac overload, and TGFβ-mediated tumor invasiveness (Villar et al., 2013, Biochim Biophys Acta 1832:323-335; Salles-Crawley et al., 2014, Blood 123:2873-2881; Fan et al., 2015, Diabetes 64:2220-2233; Marwitz et al., 2016, Cancer Res 76:3785-3801).

In certain aspects, the present invention relates to repulsive guidance molecule (RGM) polypeptides. RGMs constitute a family of structurally related proteins that have been proposed to act as co-receptors for BMP signaling and also interact with an unrelated transmembrane protein known as neogenin. The three mammalian proteins, RGM-A, RGM-B, and RGM-C, are approximately 50-60% identical in primary amino acid sequence and share structural features such as a proteolytic cleavage site and GPI anchor but undergo distinct biosynthetic and processing steps. Each RGM exhibits a distinct tissue-specific pattern of gene expression (Oldecamp et al., 2004, Gene Expr Patterns 4:283-288) and is thought to serve distinct biologic functions (see below). Soluble RGM proteins, which could form by shedding (Lin et al., 2008, Blood Cells Mol Dis 40:122-131; Tassew et al., 2012, Dev Cell 22:391-402), have been shown to inhibit BMP activity (Lin et al., 2005, Blood 106:2884-2889). A recent structural study reveals that the N-terminal domains of RGMs mimic a key BMP-binding motif of type I superfamily receptors, which could enable membrane-anchored RGMs to compete with type I receptors for BMP binding in a pH-dependent manner and yet eventually enhance BMP signaling from within an endosomal compartment (Healey et al., 2015, Nat Struct Mol Biol 22:458-465; Mueller, 2015, Nat Struct Mol Biol 22:439-440). As determined by surface plasmon resonance, the three RGM proteins exhibit differential binding kinetics for BMPs, which may contribute to their context-specific effects in vivo (Wu et al., 2012, PLOS One 7:e46307).

The protein RGM-A, encoded by RGMA, is expressed in the central nervous system during embryonic development in a largely non-overlapping manner with RGM-B. In the adult, RGM-A is expressed in brain as well as many other tissues, and it has been implicated in cancer, immune regulation, and as a sarcoplasmic protein regulating differentiation and size of skeletal muscle cells (Tian et al., 2013, Mol Reprod Dev 80:700-717; Martins et al., 2014, Cells Tissues Organs 200:326-338). Studies of RGM-A in several cell types in vitro suggest that it increases BMP signaling by facilitating use of ActRIIA by endogenous BMP2 and BMP4 ligands that otherwise prefer signaling through BMPRII (Xia et al., 2007, J Biol Chem 282:18129-18140).

RGM-B, also known as DRAGON and encoded by RGMB. Like RGM-A, RGM-B is expressed in brain as well as many other tissues of the adult. RGM-B knockout mice die several weeks after birth for undetermined reasons (Xia et al., 2011, J Immunol 186:1369-1376). RGM-B binds BMP2 and BMP4 but not BMP7, activin A, or TGFβ isoforms, as determined by surface plasmon resonance, and interacts directly with type I receptors (ALK2, ALK3, and ALK6) and type II receptors (ActRIIA and ActRIIB), as determined by co-immunoprecipitation and blockade with dominant negative receptors (Samad et al., 2005, J Biol Chem 280:14122-14129). The ability of RGM-B to increase BMP signaling requires membrane association through its C-terminal GPI anchor.

The protein RGM-C, also known as hemojuvelin (HJV) and encoded by HFE2, is associated with juvenile hemochromatosis, a rare recessive disease characterized by early-onset systemic iron overload with severe clinical complications. Hemojuvelin is now known to be an essential factor in the regulation of hepcidin, a master regulator of iron homeostasis (Niederkofler et al., 2005, J Clin Invest 115:2180-2186). Hemojuvelin is expressed primarily in liver, consistent with the predominant site of hepcidin regulation, and also in heart and skeletal muscle, where the role of hemojuvelin is unclear. Multiple studies have demonstrated that hemojuvelin regulates hepcidin expression in the liver by altering BMP signaling. Unlike RGM-A and RGM-B, hemojuvelin binds with high affinity to BMP6, a key ligand regulating hepcidin expression (Andriopoulos et al., 2009, Nat Genet 41:482-487), in addition to binding BMP2 and BMP4. On the basis of siRNA knockdown experiments in cell lines and hepatic expression of superfamily proteins, it has been suggested that hemojuvelin promotes endogenous signaling of BMP2, BMP4, and BMP6 through ALK2 or ALK3 and ActRIIA (Xia et al., 2008, Blood 111:5195-5204).

In certain aspects, the present invention relates to MuSK polypeptides. Muscle-associated receptor tyrosine kinase (MuSK), also known as muscle-specific kinase, CMS9, or FADS, is encoded by MUSK. MuSK is a single-pass transmembrane protein originally identified as a receptor tyrosine kinase expressed prominently in embryonic skeletal muscle and at the mature neuromuscular junction (Valenzuela et al., 1995, Neuron 15:573-584). These investigators showed that MuSK expression is induced dramatically throughout the adult myofiber after denervation, blockade of electrical activity, or physical immobilization. Subsequent studies indicate that MuSK is activated by proteins structurally unrelated to the TGFβ superfamily in a complex temporal-spatial manner to promote and maintain clustering of acetylcholine receptors on the postsynaptic side of the neuromuscular junction and to induce differentiation of the presynaptic nerve terminal (Hubbard et al., 2013, Biochim Biophys Acta 1834:2166-2169). Surprisingly, recent studies have revealed that MuSK also serves as a BMP co-receptor which is capable of binding BMPs and type I receptors (ALK3, ALK6) and stimulating BMP signaling by a mechanism independent of MuSK tyrosine kinase activity (Yilmaz et al., 2016, Sci Signal 9:ra87).

The terms used in this specification generally have their ordinary meanings in the art, within the context of this disclosure and in the specific context where each term is used. Certain terms are discussed below or elsewhere in the specification to provide additional guidance to the practitioner in describing the compositions and methods of the disclosure and how to make and use them. The scope or meaning of any use of a term will be apparent from the specific context in which it is used.

The terms “heteromultimer complex”, “heteromer”, or “heteromultimer” is a complex comprising at least a first polypeptide and a second polypeptide, wherein the second polypeptide differs in amino acid sequence from the first polypeptide by at least one amino acid residue. The heteromer can comprise a “heterodimer” formed by the first and second polypeptide or can form higher order structures where polypeptides in addition to the first and second polypeptide are present. Exemplary structures for the heteromultimer include heterodimers, heterotrimers, heterotetramers and further oligomeric structures. Heterodimers are designated herein as X:Y or equivalently as X-Y, where X represents a first polypeptide and Y represents a second polypeptide. Higher-order heteromers and oligomeric structures are designated herein in a corresponding manner. In certain embodiments a heteromultimer is recombinant (e.g., one or more polypeptide components may be a recombinant protein), isolated and/or purified.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions. However, in common usage and in the instant application, the term “homologous,” when modified with an adverb such as “highly,” may refer to sequence similarity and may or may not relate to a common evolutionary origin.

The term “sequence similarity,” in all its grammatical forms, refers to the degree of identity or correspondence between nucleic acid or amino acid sequences that may or may not share a common evolutionary origin.

“Percent (%) sequence identity” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

“Agonize”, in all its grammatical forms, refers to the process of activating a protein and/or gene (e.g., by activating or amplifying that protein's gene expression or by inducing an inactive protein to enter an active state) or increasing a protein's and/or gene's activity.

“Antagonize”, in all its grammatical forms, refers to the process of inhibiting a protein and/or gene (e.g., by inhibiting or decreasing that protein's gene expression or by inducing an active protein to enter an inactive state) or decreasing a protein's and/or gene's activity.

The terms “about” and “approximately” as used in connection with a numerical value throughout the specification and the claims denotes an interval of accuracy, familiar and acceptable to a person skilled in the art. In general, such interval of accuracy is ±10%, Alternatively, and particularly in biological systems, the terms “about” and “approximately” may mean values that are within an order of magnitude, preferably ≤5-fold and more preferably ≤2-fold of a given value.

Numeric ranges disclosed herein are inclusive of the numbers defining the ranges.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

### 2. TGF-Beta Superfamily Co-Receptor Heteromultimers

In part, the disclosure provides recombinant TGF-beta superfamily heteromultimers (heteromultimers) comprising at least one TGF-beta superfamily co-receptor polypeptide, including fragments and variants thereof. In some embodiments, the disclosure relates to a recombinant heteromultimer comprising a TGF-beta superfamily co-receptor polypeptide selected from the group consisting of: endoglin, betaglycan, Cripto-1, Cryptic, Cryptic family protein 1B, Crim1, Crim2, BAMBI, BMPER, RGM-A, RGM-B, hemojuvelin, and MuSK including fragments and variants thereof. Preferably, TGF-beta superfamily co-receptor polypeptides as described herein comprise a ligand-binding domain of the receptor. In some preferred embodiments, polypeptides and heteromultimers of the disclosure are soluble. In certain preferred embodiments, heteromultimers of the disclosure bind to one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, Müllerian-inhibiting substance (MIS), and Lefty). In some embodiments, a heteromultimer may bind to one or more TGF-beta superfamily ligands with a K_{D} of at least 1×10⁻⁷ M (e.g., K_{D} of greater than or equal to 10⁻⁷, 10⁻⁸, 10⁻⁹, 10⁻¹⁰, 10⁻¹¹, or 10⁻¹²). In some embodiments, a heteromultimer of the disclosure has a different TGF-beta superfamily ligand binding and/or inhibition profile (specificity) compared to a corresponding homomultimer. In some embodiments, a heteromultimer of the disclosure may inhibit one or more TGF-beta superfamily ligands (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, Millerian-inhibiting substance (MIS), and Lefty). In some embodiments, a heteromultimer of the disclosure may inhibit signaling of one or more TGF-beta superfamily ligands. For example, in some embodiments, a heteromultimer of the disclosure may inhibit signaling of one or more TGF-beta superfamily ligands in a cell-based assay (e.g., cell-based signaling assays as described herein). In some embodiments, heteromultimers of the disclosure are heterodimers.

The term “endoglin polypeptide” includes polypeptides comprising any naturally occurring endoglin protein (encoded by ENG or one of its nonhuman orthologs) as well as any variants thereof (including mutants, fragments, fusions, and peptidomimetic forms) that retain a useful activity.

The human endoglin isoform 1 precursor protein sequence (NCBI Ref Seq NP_001108225.1) is as follows:

| (SEQ ID NO: 1) | |
|---|---|
| 1 | MDRGTLPLAV ALLLASCSLS PTSLAETVHC DLQPVGPERG EVTYTTSQVS KGCVAQAPNA |
| 61 | ILEVHVLFLE FPTGPSQLEL TLQASKQNGT WPREVLLVLS VNSSVFLHLQ ALGIPLHLAY |
| 121 | NSSLVTFQEP PGVNTTELPS FPKTQILEWA AERGPITSAA ELNDPQSILL RLGQAQGSLS |
| 181 | FCMLEASQDM GRTLEWRPRT PALVRGCHLE GVAGHKEAHI LRVLPGHSAG PRTVTVKVEL |
| 241 | SCAPGDLDAV LILQGPPYVS WLIDANHNMQ IWTTGEYSFK IFPEKNIRGF KLPDTPQGLL |
| 301 | GEARMLNASI VASFVELPLA SIVSLHASSC GGRLQTSPAP IQTTPPKDTC SPELLMSLIQ |
| 361 | TKCADDAMTL VLKKELVAHL KCTITGLTFW DPSCEAEDRG DKFVLRSAYS SCGMQVSASM |
| 421 | ISNEAVVNIL SSSSPQRKKV HCLNMDSLSF QLGLYLSPHF LQASNTIEPG QQSFVQVRVS |
| 481 | PSVSEFLLQL DSCHLDLGPE GGTVELIQGR AAKGNCVSLL SPSPEGDPRF SFLLHFYTVP |
| 541 | 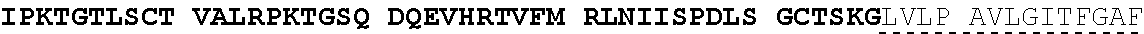 |
| 601 | 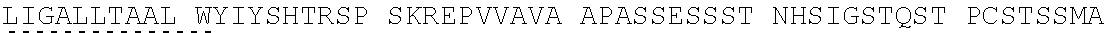 |

The signal peptide is indicated by single underline, the extracellular domain is indicated in bold font, and the transmembrane domain is indicated by dotted underline.

A processed extracellular endoglin polypeptide sequence (isoform 1) is as follows:

(SEQ ID NO: 2)
ETVHCDLQPVGPERGEVTYTTSQVSKGCVAQAPNAILEVHVLFL
EFPTGPSQLELTLQASKQNGTWPREVLLVLSVNSSVFLHLQALG
IPLHLAYNSSLVTFQEPPGVNTTELPSFPKTQILEWAAERGPIT
SAAELNDPQSILLRLGQAQGSLSFCMLEASQDMGRTLEWRPRTP
ALVRGCHLEGVAGHKEAHILRVLPGHSAGPRTVTVKVELSCAPG
DLDAVLILQGPPYVSWLIDANHNMQIWTTGEYSFKIFPEKNIRG
FKLPDTPQGLLGEARMLNASIVASFVELPLASIVSLHASSCGGR
LQTSPAPIQTTPPKDTCSPELLMSLIQTKCADDAMTLVLKKELV
AHLKCTITGLTFWDPSCEAEDRGDKFVLRSAYSSCGMQVSASMI
SNEAVVNILSSSSPQRKKVHCLNMDSLSFQLGLYLSPHFLQASN
TIEPGQQSFVQVRVSPSVSEFLLQLDSCHLDLGPEGGTVELIQG
RAAKGNCVSLLSPSPEGDPRFSFLLHEYTVPIPKTGTLSCTVAL
RPKTGSQDQEVHRTVFMRLNIISPDLSGCTSKG

A nucleic acid sequence encoding unprocessed human ENG isoform 1 precursor protein is shown below (SEQ ID NO: 3), corresponding to nucleotides 419-2392 of NCBI Reference Sequence NM_001114753.2. The signal sequence is underlined.

```
(SEQ ID NO: 3)
      1 ATGGACCGCG GCACGCTCCC TCTGGCTGTT
          GCCCTGCTGC TGGCCAGCTG
    51 CAGCCTCAGC CCCACAAGTC TTGCAGAAAC
          AGTCCATTGT GACCTTCAGC
  101 CTGTGGGCCC CGAGAGGGGC GAGGTGACAT
          ATACCACTAG CCAGGTCTCG
  151 AAGGGCTGCG TGGCTCAGGC CCCCAATGCC
          ATCCTTGAAG TCCATGTCCT
  201 CTTCCTGGAG TTCCCAACGG GCCCGTCACA
          GCTGGAGCTG ACTCTCCAGG
  251 CATCCAAGCA AAATGGCACC TGGCCCCGAG
          AGGTGCTTCT GGTCCTCAGT
  301 GTAAACAGCA GTGTCTTCCT GCATCTCCAG
          GCCCTGGGAA TCCCACTGCA
  351 CTTGGCCTAC AATTCCAGCC TGGTCACCTT
          CCAAGAGCCC CCGGGGGTCA
  401 ACACCACAGA GCTGCCATCC TTCCCCAAGA
          CCCAGATCCT TGAGTGGGCA
  451 GCTGAGAGGG GCCCCATCAC CTCTGCTGCT
          GAGCTGAATG ACCCCCAGAG
  501 CATCCTCCTC CGACTGGGCC AAGCCCAGGG
          GTCACTGTCC TTCTGCATGC
  551 TGGAAGCCAG CCAGGACATG GGCCGCACGC
          TCGAGTGGCG GCCGCGTACT
  601 CCAGCCTTGG TCCGGGGCTG CCACTTGGAA
          GGCGTGGCCG GCCACAAGGA
  651 GGCGCACATC CTGAGGGTCC TGCCGGGCCA
          CTCGGCCGGG CCCCGGACGG
  701 TGACGGTGAA GGTGGAACTG AGCTGCGCAC
          CCGGGGATCT CGATGCCGTC
  751 CTCATCCTGC AGGGTCCCCC CTACGTGTCC
          TGGCTCATCG ACGCCAACCA
  801 CAACATGCAG ATCTGGACCA CTGGAGAATA
          CTCCTTCAAG ATCTTTCCAG
  851 AGAAAAACAT TCGTGGCTTC AAGCTCCCAG
        ACACACCTCA AGGCCTCCTG
  901 GGGGAGGCCC GGATGCTCAA TGCCAGCATT
          GTGGCATCCT TCGTGGAGCT
  951 ACCGCTGGCC AGCATTGTCT CACTTCATGC
          CTCCAGCTGC GGTGGTAGGC
1001 TGCAGACCTC ACCCGCACCG ATCCAGACCA
          CTCCTCCCAA GGACACTTGT
1051 AGCCCGGAGC TGCTCATGTC CTTGATCCAG
          ACAAAGTGTG CCGACGACGC
1101 CATGACCCTG GTACTAAAGA AAGAGCTTGT
          TGCGCATTTG AAGTGCACCA
1151 TCACGGGCCT GACCTTCTGG GACCCCAGCT
          GTGAGGCAGA GGACAGGGGT
1201 GACAAGTTTG TCTTGCGCAG TGCTTACTCC
          AGCTGTGGCA TGCAGGTGTC
1251 AGCAAGTATG ATCAGCAATG AGGCGGTGGT
          CAATATCCTG TCGAGCTCAT
1301 CACCACAGCG GAAAAAGGTG CACTGCCTCA
          ACATGGACAG CCTCTCTTTC
1351 CAGCTGGGCC TCTACCTCAG CCCACACTTC
          CTCCAGGCCT CCAACACCAT
1401 CGAGCCGGGG CAGCAGAGCT TTGTGCAGGT
          CAGAGTGTCC CCATCCGTCT
1451 CCGAGTTCCT GCTCCAGTTA GACAGCTGCC
          ACCTGGACTT GGGGCCTGAG
1501 GGAGGCACCG TGGAACTCAT CCAGGGCCGG
          GCGGCCAAGG GCAACTGTGT
1551 GAGCCTGCTG TCCCCAAGCC CCGAGGGTGA
          CCCGCGCTTC AGCTTCCTCC
1601 TCCACTTCTA CACAGTACCC ATACCCAAAA
          CCGGCACCCT CAGCTGCACG
1651 GTAGCCCTGC GTCCCAAGAC CGGGTCTCAA
          GACCAGGAAG TCCATAGGAC
1701 TGTCTTCATG CGCTTGAACA TCATCAGCCC
          TGACCTGTCT GGTTGCACAA
1751 GCAAAGGCCT CGTCCTGCCC GCCGTGCTGG
          GCATCACCTT TGGTGCCTTC
1801 CTCATCGGGG CCCTGCTCAC TGCTGCACTC
          TGGTACATCT ACTCGCACAC
1851 GCGTTCCCCC AGCAAGCGGG AGCCCGTGGT
          GGCGGTGGCT GCCCCGGCCT
1901 CCTCGGAGAG CAGCAGCACC AACCACAGCA
          TCGGGAGCAC CCAGAGCACC
1951 CCCTGCTCCA CCAGCAGCAT GGCA
```

A nucleic acid sequence encoding a processed extracellular ENG isoform1 polypeptide is as follows (SEQ ID NO: 4):

(SEQ ID NO: 4)
GAAACAGTCCATTGTGACCTTCAGCCTGTGGGCCCCGAGAGGGGCGA
GGTGACATATACCACTAGCCAGGTCTCGAAGGGCTGCGTGGCTCAGG
CCCCCAATGCCATCCTTGAAGTCCATGTCCTCTTCCTGGAGTTCCCA
ACGGGCCCGTCACAGCTGGAGCTGACTCTCCAGGCATCCAAGCAAAA
TGGCACCTGGCCCCGAGAGGTGCTTCTGGTCCTCAGTGTAAACAGCA
GTGTCTTCCTGCATCTCCAGGCCCTGGGAATCCCACTGCACTTGGCC
TACAATTCCAGCCTGGTCACCTTCCAAGAGCCCCCGGGGGTCAACAC
CACAGAGCTGCCATCCTTCCCCAAGACCCAGATCCTTGAGTGGGCAG
CTGAGAGGGGCCCCATCACCTCTGCTGCTGAGCTGAATGACCCCCAG
AGCATCCTCCTCCGACTGGGCCAAGCCCAGGGGTCACTGTCCTTCTG
CATGCTGGAAGCCAGCCAGGACATGGGCCGCACGCTCGAGTGGCGGC
CGCGTACTCCAGCCTTGGTCCGGGGCTGCCACTTGGAAGGCGTGGCC
GGCCACAAGGAGGCGCACATCCTGAGGGTCCTGCCGGGCCACTCGGC
CGGGCCCCGGACGGTGACGGTGAAGGTGGAACTGAGCTGCGCACCCG
GGGATCTCGATGCCGTCCTCATCCTGCAGGGTCCCCCCTACGTGTCC
TGGCTCATCGACGCCAACCACAACATGCAGATCTGGACCACTGGAGA
ATACTCCTTCAAGATCTTTCCAGAGAAAAACATTCGTGGCTTCAAGC
TCCCAGACACACCTCAAGGCCTCCTGGGGGAGGCCCGGATGCTCAAT
GCCAGCATTGTGGCATCCTTCGTGGAGCTACCGCTGGCCAGCATTGT
CTCACTTCATGCCTCCAGCTGCGGTGGTAGGCTGCAGACCTCACCCG
CACCGATCCAGACCACTCCTCCCAAGGACACTTGTAGCCCGGAGCTG
CTCATGTCCTTGATCCAGACAAAGTGTGCCGACGACGCCATGACCCT
GGTACTAAAGAAAGAGCTTGTTGCGCATTTGAAGTGCACCATCACGG
GCCTGACCTTCTGGGACCCCAGCTGTGAGGCAGAGGACAGGGGTGAC
AAGTTTGTCTTGCGCAGTGCTTACTCCAGCTGTGGCATGCAGGTGTC
AGCAAGTATGATCAGCAATGAGGCGGTGGTCAATATCCTGTCGAGCT
CATCACCACAGCGGAAAAAGGTGCACTGCCTCAACATGGACAGCCTC
TCTTTCCAGCTGGGCCTCTACCTCAGCCCACACTTCCTCCAGGCCTC
CAACACCATCGAGCCGGGGCAGCAGAGCTTTGTGCAGGTCAGAGTGT
CCCCATCCGTCTCCGAGTTCCTGCTCCAGTTAGACAGCTGCCACCTG
GACTTGGGGCCTGAGGGAGGCACCGTGGAACTCATCCAGGGCCGGGC
GGCCAAGGGCAACTGTGTGAGCCTGCTGTCCCCAAGCCCCGAGGGTG
ACCCGCGCTTCAGCTTCCTCCTCCACTTCTACACAGTACCCATACCC
AAAACCGGCACCCTCAGCTGCACGGTAGCCCTGCGTCCCAAGACCGG
GTCTCAAGACCAGGAAGTCCATAGGACTGTCTTCATGCGCTTGAACA
TCATCAGCCCTGACCTGTCTGGTTGCACAAGCAAAGGC

The human endoglin isoform 2 precursor protein sequence (NCBI Ref Seq NP_000109.1) is as follows:

| (SEQ ID NO: 5) | |
|---|---|
| 1 | MDRGTLPLAV ALLLASCSLS PTSLAETVHC DLQPVGPERG EVTYTTSQVS KGCVAQAPNA |
| 61 | ILEVHVLFLE FPTGPSQLEL TLQASKQNGT WPREVLLVLS VNSSVFLHLQ ALGIPLHLAY |
| 121 | NSSLVTFQEP PGVNTTELPS FPKTQILEWA AERGPITSAA ELNDPQSILL RLGQAQGSLS |
| 181 | FCMLEASQDM GRTLEWRPRT PALVRGCHLE GVAGHKEAHI LRVLPGHSAG PRTVTVKVEL |
| 241 | SCAPGDLDAV LILQGPPYVS WLIDANHNMQ IWTTGEYSFK IFPEKNIRGF KLPDTPQGLL |
| 301 | GEARMLNASI VASFVELPLA SIVSLHASSC GGRLQTSPAP IQTTPPKDTC SPELLMSLIQ |
| 361 | TKCADDAMTL VLKKELVAHL KCTITGLTFW DPSCEAEDRG DKFVLRSAYS SCGMQVSASM |
| 421 | ISNEAVVNIL SSSSPQRKKV HCLNMDSLSF QLGLYLSPHF LQASNTIEPG QQSFVQVRVS |
| 481 | PSVSEFLLQL DSCHLDLGPE GGTVELIQGR AAKGNCVSLL SPSPEGDPRF SFLLHFYTVP |
| 541 | 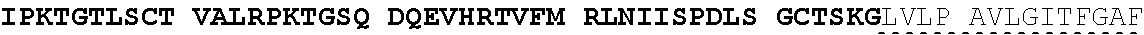 |
| 601 | 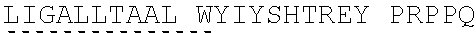 |

The signal peptide is indicated by single underline, the extracellular domain is indicated in bold font, and the transmembrane domain is indicated by doedunderline. The endoglin isoform 2 has a shortened and distinct intracellular domain compared to endoglin isoform 1 and an unchanged extracellular domain compared to endoglin isoform 1.

A processed extracellular endoglin polypeptide sequence (isoform 2) is as follows:

(SEQ ID NO: 6)
ETVHCDLQPVGPERGEVTYTTSQVSKGCVAQAPNAILEVHVLFLEFPT
GPSQLELTLQASKQNGTWPREVLLVLSVNSSVFLHLQALGIPLHLAYN
SSLVTFQEPPGVNTTELPSFPKTQILEWAAERGPITSAAELNDPQSIL
LRLGQAQGSLSFCMLEASQDMGRTLEWRPRTPALVRGCHLEGVAGHKE
AHILRVLPGHSAGPRTVTVKVELSCAPGDLDAVLILQGPPYVSWLIDA
NHNMQIWTTGEYSFKIFPEKNIRGFKLPDTPQGLLGEARMLNASIVAS
FVELPLASIVSLHASSCGGRLQTSPAPIQTTPPKDTCSPELLMSLIQT
KCADDAMTLVLKKELVAHLKCTITGLTFWDPSCEAEDRGDKFVLRSAY
SSCGMQVSASMISNEAVVNILSSSSPQRKKVHCLNMDSLSFQLGLYLS
PHFLQASNTIEPGQQSFVQVRVSPSVSEFLLQLDSCHLDLGPEGGTVE
LIQGRAAKGNCVSLLSPSPEGDPRFSFLLHEYTVPIPKTGTLSCTVAL
RPKTGSQDQEVHRTVFMRLNIISPDLSGCTSKG

A nucleic acid sequence encoding unprocessed human ENG isoform 2 precursor protein is shown below (SEQ ID NO: 7), corresponding to nucleotides 419-2293 of NCBI Reference Sequence NM_000118.3. The signal sequence is underlined.

(SEQ ID NO: 7)
ATGGACCGCGGCACGCTCCCTCTGGCTGTTGCCCTGCTGCTGGCCAGC
TGCAGCCTCAGCCCCACAAGTCTTGCAGAAACAGTCCATTGTGACCTT
CAGCCTGTGGGCCCCGAGAGGGGCGAGGTGACATATACCACTAGCCAG
GTCTCGAAGGGCTGCGTGGCTCAGGCCCCCAATGCCATCCTTGAAGTC
CATGTCCTCTTCCTGGAGTTCCCAACGGGCCCGTCACAGCTGGAGCTG
ACTCTCCAGGCATCCAAGCAAAATGGCACCTGGCCCCGAGAGGTGCTT
CTGGTCCTCAGTGTAAACAGCAGTGTCTTCCTGCATCTCCAGGCCCTG
GGAATCCCACTGCACTTGGCCTACAATTCCAGCCTGGTCACCTTCCAA
GAGCCCCCGGGGGTCAACACCACAGAGCTGCCATCCTTCCCCAAGACC
CAGATCCTTGAGTGGGCAGCTGAGAGGGGCCCCATCACCTCTGCTGCT
GAGCTGAATGACCCCCAGAGCATCCTCCTCCGACTGGGCCAAGCCCAG
GGGTCACTGTCCTTCTGCATGCTGGAAGCCAGCCAGGACATGGGCCGC
ACGCTCGAGTGGCGGCCGCGTACTCCAGCCTTGGTCCGGGGCTGCCAC
TTGGAAGGCGTGGCCGGCCACAAGGAGGCGCACATCCTGAGGGTCCTG
CCGGGCCACTCGGCCGGGCCCCGGACGGTGACGGTGAAGGTGGAACTG
AGCTGCGCACCCGGGGATCTCGATGCCGTCCTCATCCTGCAGGGTCCC
CCCTACGTGTCCTGGCTCATCGACGCCAACCACAACATGCAGATCTGG
ACCACTGGAGAATACTCCTTCAAGATCTTTCCAGAGAAAAACATTCGT
GGCTTCAAGCTCCCAGACACACCTCAAGGCCTCCTGGGGGAGGCCCGG
ATGCTCAATGCCAGCATTGTGGCATCCTTCGTGGAGCTACCGCTGGCC
AGCATTGTCTCACTTCATGCCTCCAGCTGCGGTGGTAGGCTGCAGACC
TCACCCGCACCGATCCAGACCACTCCTCCCAAGGACACTTGTAGCCCG
GAGCTGCTCATGTCCTTGATCCAGACAAAGTGTGCCGACGACGCCATG
ACCCTGGTACTAAAGAAAGAGCTTGTTGCGCATTTGAAGTGCACCATC
ACGGGCCTGACCTTCTGGGACCCCAGCTGTGAGGCAGAGGACAGGGGT
GACAAGTTTGTCTTGCGCAGTGCTTACTCCAGCTGTGGCATGCAGGTG
TCAGCAAGTATGATCAGCAATGAGGCGGTGGTCAATATCCTGTCGAGC
TCATCACCACAGCGGAAAAAGGTGCACTGCCTCAACATGGACAGCCTC
TCTTTCCAGCTGGGCCTCTACCTCAGCCCACACTTCCTCCAGGCCTCC
AACACCATCGAGCCGGGGCAGCAGAGCTTTGTGCAGGTCAGAGTGTCC
CCATCCGTCTCCGAGTTCCTGCTCCAGTTAGACAGCTGCCACCTGGAC
TTGGGGCCTGAGGGAGGCACCGTGGAACTCATCCAGGGCCGGGCGGCC
AAGGGCAACTGTGTGAGCCTGCTGTCCCCAAGCCCCGAGGGTGACCCG
CGCTTCAGCTTCCTCCTCCACTTCTACACAGTACCCATACCCAAAACC
GGCACCCTCAGCTGCACGGTAGCCCTGCGTCCCAAGACCGGGTCTCAA
GACCAGGAAGTCCATAGGACTGTCTTCATGCGCTTGAACATCATCAGC
CCTGACCTGTCTGGTTGCACAAGCAAAGGCCTCGTCCTGCCCGCCGTG
CTGGGCATCACCTTTGGTGCCTTCCTCATCGGGGCCCTGCTCACTGCT
GCACTCTGGTACATCTACTCGCACACGCGTGAGTACCCCAGGCCCCCA
CAG

A nucleic acid sequence encoding a processed extracellular ENG isoform 2 polypeptide is as follows (SEQ ID NO: 8):

(SEQ ID NO: 8)
GAAACAGTCCATTGTGACCTTCAGCCTGTGGGCCCCGAGAGGGGCGAG
GTGACATATACCACTAGCCAGGTCTCGAAGGGCTGCGTGGCTCAGGCC
CCCAATGCCATCCTTGAAGTCCATGTCCTCTTCCTGGAGTTCCCAACG
GGCCCGTCACAGCTGGAGCTGACTCTCCAGGCATCCAAGCAAAATGGC
ACCTGGCCCCGAGAGGTGCTTCTGGTCCTCAGTGTAAACAGCAGTGTC
TTCCTGCATCTCCAGGCCCTGGGAATCCCACTGCACTTGGCCTACAAT
TCCAGCCTGGTCACCTTCCAAGAGCCCCCGGGGGTCAACACCACAGAG
CTGCCATCCTTCCCCAAGACCCAGATCCTTGAGTGGGCAGCTGAGAGG
GGCCCCATCACCTCTGCTGCTGAGCTGAATGACCCCCAGAGCATCCTC
CTCCGACTGGGCCAAGCCCAGGGGTCACTGTCCTTCTGCATGCTGGAA
GCCAGCCAGGACATGGGCCGCACGCTCGAGTGGCGGCCGCGTACTCCA
GCCTTGGTCCGGGGCTGCCACTTGGAAGGCGTGGCCGGCCACAAGGAG
GCGCACATCCTGAGGGTCCTGCCGGGCCACTCGGCCGGGCCCCGGACG
GTGACGGTGAAGGTGGAACTGAGCTGCGCACCCGGGGATCTCGATGCC
GTCCTCATCCTGCAGGGTCCCCCCTACGTGTCCTGGCTCATCGACGCC
AACCACAACATGCAGATCTGGACCACTGGAGAATACTCCTTCAAGATC
TTTCCAGAGAAAAACATTCGTGGCTTCAAGCTCCCAGACACACCTCAA
GGCCTCCTGGGGGAGGCCCGGATGCTCAATGCCAGCATTGTGGCATCC
TTCGTGGAGCTACCGCTGGCCAGCATTGTCTCACTTCATGCCTCCAGC
TGCGGTGGTAGGCTGCAGACCTCACCCGCACCGATCCAGACCACTCCT
CCCAAGGACACTTGTAGCCCGGAGCTGCTCATGTCCTTGATCCAGACA
AAGTGTGCCGACGACGCCATGACCCTGGTACTAAAGAAAGAGCTTGTT
GCGCATTTGAAGTGCACCATCACGGGCCTGACCTTCTGGGACCCCAGC
TGTGAGGCAGAGGACAGGGGTGACAAGTTTGTCTTGCGCAGTGCTTAC
TCCAGCTGTGGCATGCAGGTGTCAGCAAGTATGATCAGCAATGAGGCG
GTGGTCAATATCCTGTCGAGCTCATCACCACAGCGGAAAAAGGTGCAC
TGCCTCAACATGGACAGCCTCTCTTTCCAGCTGGGCCTCTACCTCAGC
CCACACTTCCTCCAGGCCTCCAACACCATCGAGCCGGGGCAGCAGAGC
TTTGTGCAGGTCAGAGTGTCCCCATCCGTCTCCGAGTTCCTGCTCCAG
TTAGACAGCTGCCACCTGGACTTGGGGCCTGAGGGAGGCACCGTGGAA
CTCATCCAGGGCCGGGCGGCCAAGGGCAACTGTGTGAGCCTGCTGTCC
CCAAGCCCCGAGGGTGACCCGCGCTTCAGCTTCCTCCTCCACTTCTAC
ACAGTACCCATACCCAAAACCGGCACCCTCAGCTGCACGGTAGCCCTG
CGTCCCAAGACCGGGTCTCAAGACCAGGAAGTCCATAGGACTGTCTTC
ATGCGCTTGAACATCATCAGCCCTGACCTGTCTGGTTGCACAAGCAAA
GGC

An alternative processed extracellular endoglin polypeptide sequence (from either isoform 1 or isoform 2) is as follows:

(SEQ ID NO: 93)
ETVHCDLQPVGPERGEVTYTTSQVSKGCVAQAPNAILEVHVLFLEFPTGP
SQLELTLQASKQNGTWPREVLLVLSVNSSVFLHLQALGIPLHLAYNSSLV
TFQEPPGVNTTELPSFPKTQILEWAAERGPITSAAELNDPQSILLRLGQA
QGSLSFCMLEASQDMGRTLEWRPRTPALVRGCHLEGVAGHKEAHILRVLP
GHSAGPRTVTVKVELSCAPGDLDAVLILQGPPYVSWLIDANHNMQIWTTG
EYSFKIFPEKNIRGFKLPDTPQGLLGEARMLNASIVASFVELPLASIVSL
HASSCGGRLQTSPAPIQTTPP

A nucleic acid sequence encoding this alternative processed extracellular ENG polypeptide is as follows (SEQ ID NO: 94):

(SEQ ID NO: 94)
GAAACAGTCCATTGTGACCTTCAGCCTGTGGGCCCCGAGAGGGGCGAGGT
GACATATACCACTAGCCAGGTCTCGAAGGGCTGCGTGGCTCAGGCCCCCA
ATGCCATCCTTGAAGTCCATGTCCTCTTCCTGGAGTTCCCAACGGGCCCG
TCACAGCTGGAGCTGACTCTCCAGGCATCCAAGCAAAATGGCACCTGGCC
CCGAGAGGTGCTTCTGGTCCTCAGTGTAAACAGCAGTGTCTTCCTGCATC
TCCAGGCCCTGGGAATCCCACTGCACTTGGCCTACAATTCCAGCCTGGTC
ACCTTCCAAGAGCCCCCGGGGGTCAACACCACAGAGCTGCCATCCTTCCC
CAAGACCCAGATCCTTGAGTGGGCAGCTGAGAGGGGCCCCATCACCTCTG
CTGCTGAGCTGAATGACCCCCAGAGCATCCTCCTCCGACTGGGCCAAGCC
CAGGGGTCACTGTCCTTCTGCATGCTGGAAGCCAGCCAGGACATGGGCCG
CACGCTCGAGTGGCGGCCGCGTACTCCAGCCTTGGTCCGGGGCTGCCACT
TGGAAGGCGTGGCCGGCCACAAGGAGGCGCACATCCTGAGGGTCCTGCCG
GGCCACTCGGCCGGGCCCCGGACGGTGACGGTGAAGGTGGAACTGAGCTG
CGCACCCGGGGATCTCGATGCCGTCCTCATCCTGCAGGGTCCCCCCTACG
TGTCCTGGCTCATCGACGCCAACCACAACATGCAGATCTGGACCACTGGA
GAATACTCCTTCAAGATCTTTCCAGAGAAAAACATTCGTGGCTTCAAGCT
CCCAGACACACCTCAAGGCCTCCTGGGGGAGGCCCGGATGCTCAATGCCA
GCATTGTGGCATCCTTCGTGGAGCTACCGCTGGCCAGCATTGTCTCACTT
CATGCCTCCAGCTGCGGTGGTAGGCTGCAGACCTCACCCGCACCGATCCA
GACCACTCCTCCC

The human endoglin isoform 3 protein sequence (NCBI Ref Seq NP_001265067.1) is as follows:

| (SEQ ID NO: 9) | |
|---|---|
| 1 | MLEASQDMGR TLEWRPRTPA LVRGCHLEGV AGHKEAHILR VLPGHSAGPR TVTVKVELSC |
| 61 | APGDLDAVLI LQGPPYVSWL IDANHNMQIW TTGEYSFKIF PEKNIRGFKL PDTPQGLLGE |
| 121 | ARMLNASIVA SFVELPLASI VSLHASSCGG RLQTSPAPIQ TTPPKDTCSP ELLMSLIQTK |
| 181 | CADDAMTLVL KKELVAHLKC TITGLTFWDP SCEAEDRGDK FVLRSAYSSC GMQVSASMIS |
| 241 | NEAVVNILSS SSPQRKKVHC LNMDSLSFQL GLYLSPHFLQ ASNTIEPGQQ SFVQVRVSPS |
| 301 | VSEFLLQLDS CHLDLGPEGG TVELIQGRAA KGNCVSLLSP SPEGDPRFSF LLHFYTVPIP |
| 361 | 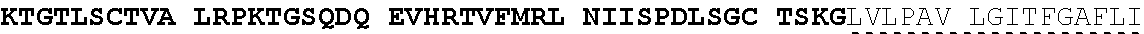 |
| 421 | 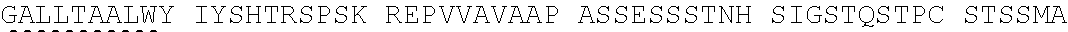 |

The extracellular domain is indicated in bold font, and the transmembrane domain is indicated by dotted underline. The endoglin isoform 3 has a distinct 5′ untranslated region, lacks a portion of the 5′ coding region, and uses a downstream start codon compared to endoglin isoform 1.

A processed extracellular endoglin polypeptide sequence (isoform 3) is as follows:

(SEQ ID NO: 10)
MLEASQDMGRTLEWRPRTPALVRGCHLEGVAGHKEAHILRVLPGHSAGPR
TVTVKVELSCAPGDLDAVLILQGPPYVSWLIDANHNMQIWTTGEYSFKIF
PEKNIRGFKLPDTPQGLLGEARMLNASIVASFVELPLASIVSLHASSCGG
RLQTSPAPIQTTPPKDTCSPELLMSLIQTKCADDAMTLVLKKELVAHLKC
TITGLTFWDPSCEAEDRGDKFVLRSAYSSCGMQVSASMISNEAVVNILSS
SSPQRKKVHCLNMDSLSFQLGLYLSPHFLQASNTIEPGQQSFVQVRVSPS
VSEFLLQLDSCHLDLGPEGGTVELIQGRAAKGNCVSLLSPSPEGDPRFSF
LLHEYTVPIPKTGTLSCTVALRPKTGSQDQEVHRTVFMRLNIISPDLSGC
TSKG

A nucleic acid sequence encoding human ENG isoform 3 protein is shown below (SEQ ID NO: 11), corresponding to nucleotides 705-2132 of NCBI Reference Sequence NM_001278138.1. The transmembrane region is indicated by dotted underline.

(SEQ ID NO: 11)
ATGCTGGAAGCCAGCCAGGACATGGGCCGCACGCTCGAGTGGCGGCCGCGTACTCCAGCCTTGGTCCGGGGCTGC
CACTTGGAAGGCGTGGCCGGCCACAAGGAGGCGCACATCCTGAGGGTCCTGCCGGGCCACTCGGCCGGGCCCCGG
ACGGTGACGGTGAAGGTGGAACTGAGCTGCGCACCCGGGGATCTCGATGCCGTCCTCATCCTGCAGGGTCCCCCC
TACGTGTCCTGGCTCATCGACGCCAACCACAACATGCAGATCTGGACCACTGGAGAATACTCCTTCAAGATCTTT
CCAGAGAAAAACATTCGTGGCTTCAAGCTCCCAGACACACCTCAAGGCCTCCTGGGGGAGGCCCGGATGCTCAAT
GCCAGCATTGTGGCATCCTTCGTGGAGCTACCGCTGGCCAGCATTGTCTCACTTCATGCCTCCAGCTGCGGTGGT
AGGCTGCAGACCTCACCCGCACCGATCCAGACCACTCCTCCCAAGGACACTTGTAGCCCGGAGCTGCTCATGTCC
TTGATCCAGACAAAGTGTGCCGACGACGCCATGACCCTGGTACTAAAGAAAGAGCTTGTTGCGCATTTGAAGTGC
ACCATCACGGGCCTGACCTTCTGGGACCCCAGCTGTGAGGCAGAGGACAGGGGTGACAAGTTTGTCTTGCGCAGT
GCTTACTCCAGCTGTGGCATGCAGGTGTCAGCAAGTATGATCAGCAATGAGGCGGTGGTCAATATCCTGTCGAGC
TCATCACCACAGCGGAAAAAGGTGCACTGCCTCAACATGGACAGCCTCTCTTTCCAGCTGGGCCTCTACCTCAGC
CCACACTTCCTCCAGGCCTCCAACACCATCGAGCCGGGGCAGCAGAGCTTTGTGCAGGTCAGAGTGTCCCCATCC
GTCTCCGAGTTCCTGCTCCAGTTAGACAGCTGCCACCTGGACTTGGGGCCTGAGGGAGGCACCGTGGAACTCATC
CAGGGCCGGGCGGCCAAGGGCAACTGTGTGAGCCTGCTGTCCCCAAGCCCCGAGGGTGACCCGCGCTTCAGCTTC
CTCCTCCACTTCTACACAGTACCCATACCCAAAACCGGCACCCTCAGCTGCACGGTAGCCCTGCGTCCCAAGACC
GGGTCTCAAGACCAGGAAGTCCATAGGACTGTCTTCATGCGCTTGAACATCATCAGCCCTGACCTGTCTGGTTGC

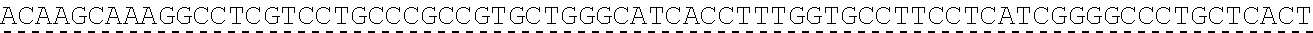

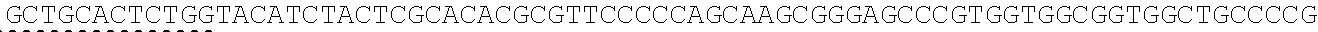

GCCTCCTCGGAGAGCAGCAGCACCAACCACAGCATCGGGAGCACCCAGAGCACCCCCTGCTCCACCAGCAGCATG
GCA

A nucleic acid sequence encoding a processed extracellular ENG isoform 3 polypeptide is as follows (SEQ ID NO: 12):

(SEQ ID NO: 12)
ATGCTGGAAGCCAGCCAGGACATGGGCCGCACGCTCGAGTGGCGGCCGCG
TACTCCAGCCTTGGTCCGGGGCTGCCACTTGGAAGGCGTGGCCGGCCACA
AGGAGGCGCACATCCTGAGGGTCCTGCCGGGCCACTCGGCCGGGCCCCGG
ACGGTGACGGTGAAGGTGGAACTGAGCTGCGCACCCGGGGATCTCGATGC
CGTCCTCATCCTGCAGGGTCCCCCCTACGTGTCCTGGCTCATCGACGCCA
ACCACAACATGCAGATCTGGACCACTGGAGAATACTCCTTCAAGATCTTT
CCAGAGAAAAACATTCGTGGCTTCAAGCTCCCAGACACACCTCAAGGCCT
CCTGGGGGAGGCCCGGATGCTCAATGCCAGCATTGTGGCATCCTTCGTGG
AGCTACCGCTGGCCAGCATTGTCTCACTTCATGCCTCCAGCTGCGGTGGT
AGGCTGCAGACCTCACCCGCACCGATCCAGACCACTCCTCCCAAGGACAC
TTGTAGCCCGGAGCTGCTCATGTCCTTGATCCAGACAAAGTGTGCCGACG
ACGCCATGACCCTGGTACTAAAGAAAGAGCTTGTTGCGCATTTGAAGTGC
ACCATCACGGGCCTGACCTTCTGGGACCCCAGCTGTGAGGCAGAGGACAG
GGGTGACAAGTTTGTCTTGCGCAGTGCTTACTCCAGCTGTGGCATGCAGG
TGTCAGCAAGTATGATCAGCAATGAGGCGGTGGTCAATATCCTGTCGAGC
TCATCACCACAGCGGAAAAAGGTGCACTGCCTCAACATGGACAGCCTCTC
TTTCCAGCTGGGCCTCTACCTCAGCCCACACTTCCTCCAGGCCTCCAACA
CCATCGAGCCGGGGCAGCAGAGCTTTGTGCAGGTCAGAGTGTCCCCATCC
GTCTCCGAGTTCCTGCTCCAGTTAGACAGCTGCCACCTGGACTTGGGGCC
TGAGGGAGGCACCGTGGAACTCATCCAGGGCCGGGCGGCCAAGGGCAACT
GTGTGAGCCTGCTGTCCCCAAGCCCCGAGGGTGACCCGCGCTTCAGCTTC
CTCCTCCACTTCTACACAGTACCCATACCCAAAACCGGCACCCTCAGCTG
CACGGTAGCCCTGCGTCCCAAGACCGGGTCTCAAGACCAGGAAGTCCATA
GGACTGTCTTCATGCGCTTGAACATCATCAGCCCTGACCTGTCTGGTTGC

In certain embodiments, the disclosure relates to heteromultimers that comprise at least one endoglin polypeptide, which includes fragments, functional variants, and modified forms thereof. Preferably, endoglin polypeptides for use in accordance with the disclosure (e.g., heteromultimers comprising an endoglin polypeptide and uses thereof) are soluble (e.g., an extracellular domain of endoglin). In other preferred embodiments, endoglin polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 1, 2, 5, 6, 9, 10, or 93. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-30 (e.g., amino acid residues 26, 27, 28, 29, or 30) of SEQ ID NO: 1, and ends at any one of amino acids 330-346 (e.g., amino acid residues 330, 331, 332, 333, 334, 335, 336, 337, 338, 339, 340, 341, 342, 343, 344, 345, or 346) of SEQ ID NO: 1. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 26-346 of SEQ ID NO: 1. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 30-330 of SEQ ID NO: 1. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 26-330 of SEQ ID NO: 1. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 30-346 of SEQ ID NO: 1. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-30 (e.g., amino acid residues 26, 27, 28, 29, or 30) of SEQ ID NO: 5, and ends at any one of amino acids 330-346 (e.g., amino acid residues 330, 331, 332, 333, 334, 335, 336, 337, 338, 339, 340, 341, 342, 343, 344, 345, or 346) of SEQ ID NO: 5. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 26-346 of SEQ ID NO: 5. In some embodiments, heteromultimers of the disclosure comprise least one endoglin polypeptide that is at least 70%, 75%, 80%, 85%, 90%, 91%, 92%, 93%, 94%, 95%, 96%, 97%, 98%, 99%, or 100% identical to amino acids of 30-330 of SEQ ID NO: 5. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 26-330 of SEQ ID NO: 5. In some embodiments, heteromultimers of the disclosure comprise least one endoglin polypeptide that is at least 70%, 75%, 80%, 85%, 90%, 91%, 92%, 93%, 94%, 95%, 96%, 97%, 98%, 99%, or 100% identical to amino acids of 30-346 of SEQ ID NO: 5. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-25 (e.g., amino acid residues 1, 2, 3, 4, 5, 6, 7, 8, 9, 10, 11, 12, 13, 14, 15, 16, 17, 18, 19, 20, 21, 22, 23, 24, or 25) of SEQ ID NO: 9, and ends at any one of amino acids 148-164 (e.g., amino acid residues 148, 149, 150, 151, 152, 153, 154, 155, 156, 157, 158, 159, 160, 161, 162, 163, or 164) of SEQ ID NO: 9. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 1-164 of SEQ ID NO: 9. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 25-148 of SEQ ID NO: 9. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 1-148 of SEQ ID NO: 9. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 25-164 of SEQ ID NO: 9. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-30 (e.g., amino acid residues 26, 27, 28, 29, or 30) of SEQ ID NO: 1, and ends at any one of amino acids 582-586 (e.g., amino acid residues 582, 583, 584, 585, or 586) of SEQ ID NO: 1. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 26-586 of SEQ ID NO: 501. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 30-582 of SEQ ID NO: 1. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-30 (e.g., amino acid residues 26, 27, 28, 29, or 30) of SEQ ID NO: 5, and ends at any one of amino acids 582-586 (e.g., amino acid residues 582, 583, 584, 585, or 586) of SEQ ID NO: 5. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 26-586 of SEQ ID NO: 5. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 30-582 of SEQ ID NO: 5. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-25 (e.g., amino acid residues 1, 2, 3, 4, 5, 6, 7, 8, 9, 10, 11, 12, 13, 14, 15, 16, 17, 18, 19, 20, 21, 22, 23, 24, or 25) of SEQ ID NO: 9, and ends at any one of amino acids 400-404 (e.g., amino acid residues 400, 401, 402, or 403) of SEQ ID NO: 9. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 1-404 of SEQ ID NO: 9. In some embodiments, heteromultimers of the disclosure comprise at least one endoglin polypeptide that is at least 70%, 75%, 80%, 85%, 90%, 91%, 92%, 93%, 94%, 95%, 96%, 97%, 98%, 99%, or 100% identical to amino acids of 25-400 of SEQ ID NO: 9.

The term “Cripto-1 polypeptide” includes polypeptides comprising any naturally occurring Cripto-1 protein (encoded by TDGF1 or one of its nonhuman orthologs) as well as any variants thereof (including mutants, fragments, fusions, and peptidomimetic forms) that retain a useful activity.

The human Cripto-1 isoform 1 precursor protein sequence (NCBI Ref Seq NP_003203.1) is as follows:

```
(SEQ ID NO: 13)
    1 MDCRKMARFS YSVIWIMAIS KVFELGLVAG LGHQEFARPS RGYLAFRDDS IWPQEEPAIR
  61 PRSSQRVPPM GIQHSKELNR TCCLNGGTCM LGSFCACPPS FYGRNCEHDV RKENCGSVPH
121 DTWLPKKCSL CKCWHGQLRC FPQAFLPGCD GLVMDEHLVA SRTPELPPSA RTTTFMLVGI
181 CLSIQSYY
```

The signal peptide is indicated by single underline.

A processed Cripto-1 isoform 1 polypeptide sequence is as follows:

(SEQ ID NO: 14)
LGHQEFARPSRGYLAFRDDSIWPQEEPAIRPRSSQRVPPMGIQHSKELNR
TCCLNGGTCMLGSFCACPPSFYGRNCEHDVRKENCGSVPHDTWLPKKCSL
CKCWHGQLRCFPQAFLPGCDGLVMDEHLVAS

A nucleic acid sequence encoding unprocessed human Cripto-1 isoform 1 precursor protein is shown below (SEQ ID NO: 15), corresponding to nucleotides 385-948 of NCBI Reference Sequence NM_003212.3. The signal sequence is underlined.

(SEQ ID NO: 15)
ATGGACTGCAGGAAGATGGCCCGCTTCTCTTACAGTGTGATTTGGATCA
TGGCCATTTCTAAAGTCTTTGAACTGGGATTAGTTGCCGGGCTGGGCCA
TCAGGAATTTGCTCGTCCATCTCGGGGATACCTGGCCTTCAGAGATGAC
AGCATTTGGCCCCAGGAGGAGCCTGCAATTCGGCCTCGGTCTTCCCAGC
GTGTGCCGCCCATGGGGATACAGCACAGTAAGGAGCTAAACAGAACCTG
CTGCCTGAATGGGGGAACCTGCATGCTGGGGTCCTTTTGTGCCTGCCCT
CCCTCCTTCTACGGACGGAACTGTGAGCACGATGTGCGCAAAGAGAACT
GTGGGTCTGTGCCCCATGACACCTGGCTGCCCAAGAAGTGTTCCCTGTG
TAAATGCTGGCACGGTCAGCTCCGCTGCTTTCCTCAGGCATTTCTACCC
GGCTGTGATGGCCTTGTGATGGATGAGCACCTCGTGGCTTCCAGGACTC
CAGAACTACCACCGTCTGCACGTACTACCACTTTTATGCTAGTTGGCAT
CTGCCTTTCTATACAAAGCTACTAT

A nucleic acid sequence encoding a processed Cripto-1 isoform 1 is shown below (SEQ ID NO: 16):

(SEQ ID NO: 16)
CTGGGCCATCAGGAATTTGCTCGTCCATCTCGGGGATACCTGGCCTTCAG
AGATGACAGCATTTGGCCCCAGGAGGAGCCTGCAATTCGGCCTCGGTCTT
CCCAGCGTGTGCCGCCCATGGGGATACAGCACAGTAAGGAGCTAAACAGA
ACCTGCTGCCTGAATGGGGGAACCTGCATGCTGGGGTCCTTTTGTGCCTG
CCCTCCCTCCTTCTACGGACGGAACTGTGAGCACGATGTGCGCAAAGAGA
ACTGTGGGTCTGTGCCCCATGACACCTGGCTGCCCAAGAAGTGTTCCCTG
TGTAAATGCTGGCACGGTCAGCTCCGCTGCTTTCCTCAGGCATTTCTACC
CGGCTGTGATGGCCTTGTGATGGATGAGCACCTCGTGGCTTCC

The human Cripto-1 isoform 2 protein sequence (NCBI Ref Seq NP_001167607.1) is as follows:

```
(SEQ ID NO: 17)
    1 MAISKVFELG LVAGLGHQEF ARPSRGYLAF RDDSIWPQEE PAIRPRSSQR VPPMGIQHSK
  61 ELNRTCCLNG GTCMLGSFCA CPPSFYGRNC EHDVRKENCG SVPHDTWLPK KCSLCKCWHG
121 QLRCFPQAFL PGCDGLVMDE HLVASRTPEL PPSARTTTFM LVGICLSIQS YY
```

A mature Cripto-1 polypeptide sequence (isoform 2) is as follows:

(SEQ ID NO: 18)
MAISKVFELGLVAGLGHQEFARPSRGYLAFRDDSIWPQEEPAIRPRSSQR
VPPMGIQHSKELNRTCCLNGGTCMLGSFCACPPSFYGRNCEHDVRKENCG
SVPHDTWLPKKCSLCKCWHGQLRCFPQAFLPGCDGLVMDEHLVAS

A nucleic acid sequence encoding unprocessed human Cripto-1 isoform 2 precursor protein is shown below (SEQ ID NO: 19), corresponding to nucleotides 43-558 of NCBI Reference Sequence NM_001174136.1.

(SEQ ID NO: 19)
ATGGCCATTTCTAAAGTCTTTGAACTGGGATTAGTTGCCGGGCTGGGCCA
TCAGGAATTTGCTCGTCCATCTCGGGGATACCTGGCCTTCAGAGATGACA
GCATTTGGCCCCAGGAGGAGCCTGCAATTCGGCCTCGGTCTTCCCAGCGT
GTGCCGCCCATGGGGATACAGCACAGTAAGGAGCTAAACAGAACCTGCTG
CCTGAATGGGGGAACCTGCATGCTGGGGTCCTTTTGTGCCTGCCCTCCCT
CCTTCTACGGACGGAACTGTGAGCACGATGTGCGCAAAGAGAACTGTGGG
TCTGTGCCCCATGACACCTGGCTGCCCAAGAAGTGTTCCCTGTGTAAATG
CTGGCACGGTCAGCTCCGCTGCTTTCCTCAGGCATTTCTACCCGGCTGTG
ATGGCCTTGTGATGGATGAGCACCTCGTGGCTTCCAGGACTCCAGAACTA
CCACCGTCTGCACGTACTACCACTTTTATGCTAGTTGGCATCTGCCTTTC
TATACAAAGCTACTAT

A nucleic acid sequence encoding a processed human Cripto-1 isoform 2 is shown below (SEQ ID NO: 20):

(SEQ ID NO: 20)
ATGGCCATTTCTAAAGTCTTTGAACTGGGATTAGTTGCCGGGCTGGGCCA
TCAGGAATTTGCTCGTCCATCTCGGGGATACCTGGCCTTCAGAGATGACA
GCATTTGGCCCCAGGAGGAGCCTGCAATTCGGCCTCGGTCTTCCCAGCGT
GTGCCGCCCATGGGGATACAGCACAGTAAGGAGCTAAACAGAACCTGCTG
CCTGAATGGGGGAACCTGCATGCTGGGGTCCTTTTGTGCCTGCCCTCCCT
CCTTCTACGGACGGAACTGTGAGCACGATGTGCGCAAAGAGAACTGTGGG
TCTGTGCCCCATGACACCTGGCTGCCCAAGAAGTGTTCCCTGTGTAAATG
CTGGCACGGTCAGCTCCGCTGCTTTCCTCAGGCATTTCTACCCGGCTGTG
ATGGCCTTGTGATGGATGAGCACCTCGTGGCTTCC

In certain embodiments, the disclosure relates to heteromultimers that comprise at least one Cripto-1 polypeptide, which includes fragments, functional variants, and modified forms thereof. Preferably, Cripto-1 polypeptides for use in accordance with the disclosure (e.g., heteromultimers comprising a Cripto-1 polypeptide and uses thereof) are soluble (e.g., an extracellular domain of Cripto-1). In other preferred embodiments, Cripto-1 polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 13, 14, 17, or 18. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 31-82 (e.g., amino acid residues 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, or 82) of SEQ ID NO: 13, and ends at any one of amino acids 172-188 (e.g., amino acid residues 172, 173, 174, 175, 176, 177, 178, 179, 180, 181, 182, 183, 184, 185, 186, 187, or 188) of SEQ ID NO: 13. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 31-188 of SEQ ID NO: 13. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 63-172 of SEQ ID NO: 13. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 82-172 of SEQ ID NO: 13. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 82-188 of SEQ ID NO: 13. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 31-172 of SEQ ID NO: 13. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 63-188 of SEQ ID NO: 13. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 15-66 (e.g., amino acid residues 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, or 66) of SEQ ID NO: 17, and ends at any one of amino acids 156-172 (e.g., amino acid residues 156, 157, 158, 159, 160, 161, 162, 163, 164, 165, 166, 167, 168, 169, 170, 171, or 172) of SEQ ID NO: 17. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 15-172 of SEQ ID NO: 17. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 47-172 of SEQ ID NO: 17. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 47-156 of SEQ ID NO: 17. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 66-165 of SEQ ID NO: 17. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 15-156 of SEQ ID NO: 17. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 66-172 of SEQ ID NO: 17. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 31-82 (e.g., amino acid residues 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, or 82) of SEQ ID NO: 13, and ends at any one of amino acids 181-188 (e.g., amino acid residues 181, 182, 183, 184, 185, 185, 187, or 188) of SEQ ID NO: 13. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 31-188 of SEQ ID NO: 13. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 82-181 of SEQ ID NO: 13. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-66 (e.g., amino acid residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, or 66) of SEQ ID NO: 17, and ends at any one of amino acids 165-172 (e.g., amino acid residues 165, 166, 167, 168, 169, 170, 171, or 172) of SEQ ID NO: 17. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 1-172 of SEQ ID NO: 17. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 66-165 of SEQ ID NO: 17. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 31-61 of SEQ ID NO: 13. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 63-161 of SEQ ID NO: 13. In some embodiments, heteromultimers of the disclosure comprise at least one Cripto-1 polypeptide that is at least 70%, 75%, 80%, 85%, 90%, 91%, 92%, 93%, 94%, 95%, 96%, 97%, 98%, 99%, or 100% identical to amino acids of 1-145 of SEQ ID NO: 17.

The term “Cryptic polypeptide” includes polypeptides comprising any naturally occurring Cryptic protein (encoded by CFC1 or one of its nonhuman orthologs) as well as any variants thereof (including mutants, fragments, fusions, and peptidomimetic forms) that retain a useful activity.

The human Cryptic isoform 1 precursor protein sequence (NCBI Ref Seq NP_115934.1) is as follows:

```
(SEQ ID NO: 21)
    1 MTWRHHVRLL FTVSLALQII NLGNSYQREK HNGGREEVTK VATQKHRQSP LNWTSSHFGE
  61 VTGSAEGWGP EEPLPYSRAF GEGASARPRC CRNGGTCVLG SFCVCPAHFT GRYCEHDQRR
121 SECGALEHGA WTLRACHLCR CIFGALHCLP LQTPDRCDPK DFLASHAHGP SAGGAPSLLL
181 LLPCALLHRL LRPDAPAHPR SLVPSVLQRE RRPCGRPGLG HRL
```

The signal peptide is indicated by single underline.

A processed Cryptic isoform 1 polypeptide sequence is as follows:

### YQREKHNGGREEVTKVATQKHRQSPLNWTSSHFGEVTGSAEGWGPEEPLPYSRAFGEGASARPRCCRNGGTCVLG SFCVCPAHFTGRYCEHDQRRSECGALEHGAWTLRACHLCRCIFGALHCLPLQTPDRCDPKDFLASHAHG (SEQ ID NO: 22)

A nucleic acid sequence encoding unprocessed human Cryptic isoform 1 precursor protein is shown below (SEQ ID NO: 23), corresponding to nucleotides 289-957 of NCBI Reference Sequence NM_032545.3. The signal sequence is underlined.

(SEQ ID NO: 23)
ATGACCTGGAGGCACCATGTCAGGCTTCTGTTTACGGTCAGTTTGGCAT
TACAGATCATCAATTTGGGAAACAGCTATCAAAGAGAGAAACATAACGG
CGGTAGAGAGGAAGTCACCAAGGTTGCCACTCAGAAGCACCGACAGTCA
CCGCTCAACTGGACCTCCAGTCATTTCGGAGAGGTGACTGGGAGCGCCG
AGGGCTGGGGGCCGGAGGAGCCGCTCCCCTACTCCCGGGCTTTCGGAGA
GGGTGCGTCCGCGCGGCCGCGCTGCTGCAGGAACGGCGGTACCTGCGTG
CTGGGCAGCTTCTGCGTGTGCCCGGCCCACTTCACCGGCCGCTACTGCG
AGCATGACCAGAGGCGCAGTGAATGCGGCGCCCTGGAGCACGGAGCCTG
GACCCTCCGCGCCTGCCACCTCTGCAGGTGCATCTTCGGGGCCCTGCAC
TGCCTCCCCCTCCAGACGCCTGACCGCTGTGACCCGAAAGACTTCCTGG
CCTCCCACGCTCACGGGCCGAGCGCCGGGGGCGCGCCCAGCCTGCTACT
CTTGCTGCCCTGCGCACTCCTGCACCGCCTCCTGCGCCCGGATGCGCCC
GCGCACCCTCGGTCCCTGGTCCCTTCCGTCCTCCAGCGGGAGCGGCGCC
CCTGCGGAAGGCCGGGACTTGGGCATCGCCTT

A nucleic acid sequence encoding a processed human Cryptic isoform 1 is shown below (SEQ ID NO: 24):

(SEQ ID NO: 24)
TATCAAAGAGAGAAACATAACGGCGGTAGAGAGGAAGTCACCAAGGTTG
CCACTCAGAAGCACCGACAGTCACCGCTCAACTGGACCTCCAGTCATTT
CGGAGAGGTGACTGGGAGCGCCGAGGGCTGGGGGCCGGAGGAGCCGCTC
CCCTACTCCCGGGCTTTCGGAGAGGGTGCGTCCGCGCGGCCGCGCTGCT
GCAGGAACGGCGGTACCTGCGTGCTGGGCAGCTTCTGCGTGTGCCCGGC
CCACTTCACCGGCCGCTACTGCGAGCATGACCAGAGGCGCAGTGAATGC
GGCGCCCTGGAGCACGGAGCCTGGACCCTCCGCGCCTGCCACCTCTGCA
GGTGCATCTTCGGGGCCCTGCACTGCCTCCCCCTCCAGACGCCTGACCG
CTGTGACCCGAAAGACTTCCTGGCCTCCCACGCTCACGGG

The human Cryptic isoform 2 precursor protein sequence (NCBI Ref Seq NP_001257349.1) is as follows:

```
(SEQ ID NO: 25)
    1 MTWRHHVRLL FTVSLALQII NLGNSYQREK
        HNGGREEVTK VATQKHRQSP LNWTSSHFGE
  61 VTGSAEGWGP EEPLPYSRAF GEVNAAPWST
        EPGPSAPATS AGASSGPCTA SPSRRLTAVT
121 RKTSWPPTLT GRAPGARPAC YSCCPAHSCT
        ASCARMRPRT LGPWSLPSSS GSGAPAEGRD
181 LGIAFNFLCC K
```

The signal peptide is indicated by single underline.

A processed Cryptic isoform 2 polypeptide sequence is as follows:

(SEQ ID NO: 26)
YQREKHNGGREEVTKVATQKHRQSPLNWTSSHFGEVTGSAEGWGPEEPL
PYSRAFGEVNAAPWSTEPGPSAPATSAGASSGPCTASPSRRLTAVTRKT
SWPPTLTGRAPGARPACYSCCPAHSCTASCARMRPRTLGPWSLPSSSGS
GAPAEGRDLGIAFNFLCCK

A nucleic acid sequence encoding unprocessed human Cryptic isoform 2 precursor protein is shown below (SEQ ID NO: 27), corresponding to nucleotides 289-861 of NCBI Reference Sequence NM_001270420.1. The signal sequence is underlined.

(SEQ ID NO: 27)
ATGACCTGGAGGCACCATGTCAGGCTTCTGTTTACGGTCAGTTTGGCA
TTACAGATCATCAATTTGGGAAACAGCTATCAAAGAGAGAAACATAAC
GGCGGTAGAGAGGAAGTCACCAAGGTTGCCACTCAGAAGCACCGACAG
TCACCGCTCAACTGGACCTCCAGTCATTTCGGAGAGGTGACTGGGAGC
GCCGAGGGCTGGGGGCCGGAGGAGCCGCTCCCCTACTCCCGGGCTTTC
GGAGAGGTGAATGCGGCGCCCTGGAGCACGGAGCCTGGACCCTCCGCG
CCTGCCACCTCTGCAGGTGCATCTTCGGGGCCCTGCACTGCCTCCCCC
TCCAGACGCCTGACCGCTGTGACCCGAAAGACTTCCTGGCCTCCCACG
CTCACGGGCCGAGCGCCGGGGGCGCGCCCAGCCTGCTACTCTTGCTGC
CCTGCGCACTCCTGCACCGCCTCCTGCGCCCGGATGCGCCCGCGCACC
CTCGGTCCCTGGTCCCTTCCGTCCTCCAGCGGGAGCGGCGCCCCTGCG
GAAGGCCGGGACTTGGGCATCGCCTTTAATTTTCTATGTTGTAAA

A nucleic acid sequence encoding processed Cryptic isoform 2 is shown below (SEQ ID NO: 28):

(SEQ ID NO: 28)
TATCAAAGAGAGAAACATAACGGCGGTAGAGAGGAAGTCACCAAGG
TTGCCACTCAGAAGCACCGACAGTCACCGCTCAACTGGACCTCCAG
TCATTTCGGAGAGGTGACTGGGAGCGCCGAGGGCTGGGGGCCGGAG
GAGCCGCTCCCCTACTCCCGGGCTTTCGGAGAGGTGAATGCGGCGC
CCTGGAGCACGGAGCCTGGACCCTCCGCGCCTGCCACCTCTGCAGG
TGCATCTTCGGGGCCCTGCACTGCCTCCCCCTCCAGACGCCTGACC
GCTGTGACCCGAAAGACTTCCTGGCCTCCCACGCTCACGGGCCGAG
CGCCGGGGGCGCGCCCAGCCTGCTACTCTTGCTGCCCTGCGCACTC
CTGCACCGCCTCCTGCGCCCGGATGCGCCCGCGCACCCTCGGTCCC
TGGTCCCTTCCGTCCTCCAGCGGGAGCGGCGCCCCTGCGGAAGGCC
GGGACTTGGGCATCGCCTTTAATTTTCTATGTTGTAAA

The human Cryptic isoform 3 precursor protein sequence (NCBI Ref Seq NP_001257350.1) is as follows:

```
(SEQ ID NO: 29)
    1 MTWRHHVRLL FTVSLALQII NLGNSYQREK
        HNGGREEVTK VATQKHRQSP LNWTSSHFGE
  61 VTGSAEGWGP EEPLPYSRAF GEDPKDFLAS
        HAHGPSAGGA PSLLLLLPCA LLHRLLRPDA
121 PAHPRSLVPS VLQRERRPCG RPGLGHRL
```

The signal peptide is indicated by single underline.

A processed Cryptic isoform 3 polypeptide sequence is as follows:

(SEQ ID NO: 30)
YQREKHNGGREEVTKVATQKHRQSPLNWTSSHFGE
VTGSAEGWGPEEPLPYSRAFGEDPKDFLASHAHG

A nucleic acid sequence encoding unprocessed human Cryptic isoform 3 precursor protein is shown below (SEQ ID NO: 31), corresponding to nucleotides 289-732 of NCBI Reference Sequence NM_001270421.1. The signal sequence is underlined.

(SEQ ID NO: 31)
ATGACCTGGAGGCACCATGTCAGGCTTCTGTTTACGGTCAGTTTGGC
ATTACAGATCATCAATTTGGGAAACAGCTATCAAAGAGAGAAACATA
ACGGCGGTAGAGAGGAAGTCACCAAGGTTGCCACTCAGAAGCACCGA
CAGTCACCGCTCAACTGGACCTCCAGTCATTTCGGAGAGGTGACTGG
GAGCGCCGAGGGCTGGGGGCCGGAGGAGCCGCTCCCCTACTCCCGGG
CTTTCGGAGAGGACCCGAAAGACTTCCTGGCCTCCCACGCTCACGGG
CCGAGCGCCGGGGGCGCGCCCAGCCTGCTACTCTTGCTGCCCTGCGC
ACTCCTGCACCGCCTCCTGCGCCCGGATGCGCCCGCGCACCCTCGGT
CCCTGGTCCCTTCCGTCCTCCAGCGGGAGCGGCGCCCCTGCGGAAGG
CCGGGACTTGGGCATCGCCTT

A nucleic acid sequence encoding a processed Cryptic isoform 3 is shown below (SEQ ID NO: 32):

(SEQ ID NO: 32)
TATCAAAGAGAGAAACATAACGGCGGTAGAGAGGAAGTCACCAAGGTT
GCCACTCAGAAGCACCGACAGTCACCGCTCAACTGGACCTCCAGTCAT
TTCGGAGAGGTGACTGGGAGCGCCGAGGGCTGGGGGCCGGAGGAGCCG
CTCCCCTACTCCCGGGCTTTCGGAGAGGACCCGAAAGACTTCCTGGCC
TCCCACGCTCACGGG

In certain embodiments, the disclosure relates to heteromultimers that comprise at least one Cryptic polypeptide, which includes fragments, functional variants, and modified forms thereof. Preferably, Cryptic polypeptides for use in accordance with the disclosure (e.g., heteromultimers comprising a Cryptic polypeptide and uses thereof) are soluble (e.g., an extracellular domain of Cryptic). In other preferred embodiments, Cryptic polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 21, 22, 25, 26, 29, or 30. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-90 (e.g., amino acid residues 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90) of SEQ ID NO: 21, and ends at any one of amino acids 157-223 (e.g., amino acid residues 157, 158, 159, 160, 161, 162, 163, 164, 165, 166, 167, 168, 169, 170, 171, 172, 173, 174, 175, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 126, 217, 218, 219, 220, 221, 222, or 223) of SEQ ID NO: 21. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-223 of SEQ ID NO: 21. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-157 of SEQ ID NO: 21. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 90-157 of SEQ ID NO: 21. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-169 of SEQ ID NO: 21. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 90-169 of SEQ ID NO: 21. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 90-223 of SEQ ID NO: 21. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-82 of SEQ ID NO: 21. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-30 (e.g., amino acid residues 26, 27, 28, 29, or 30) of SEQ ID NO: 25, and ends at any one of amino acids 82-191 (e.g., amino acid residues 82, 83, 84, 85, 86, 57, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 178, 179, 180, 181, 182, 183, 184, 185, 186, 187, 188, 189, 190, or 191) of SEQ ID NO: 25. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-82 of SEQ ID NO: 25. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-191 of SEQ ID NO: 25. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 30-82 of SEQ ID NO: 25. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 30-191 of SEQ ID NO: 25. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-30 (e.g., amino acid residues 26, 27, 28, 29, or 30) of SEQ ID NO: 29, and ends at any one of amino acids 82-148 (e.g., amino acid residues 82, 83, 84, 85, 86, 57, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, or 148) of SEQ ID NO: 29. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-148 of SEQ ID NO: 29. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-82 of SEQ ID NO: 29. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 30-148 of SEQ ID NO: 29. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 30-82 of SEQ ID NO: 29. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-90 (e.g., amino acid residues 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90) of SEQ ID NO: 21, and ends at any one of amino acids 214-223 (e.g., amino acid residues 214, 215, 126, 217, 218, 219, 220, 221, 222, or 223) of SEQ ID NO: 21. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-223 of SEQ ID NO: 21. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 109-223 of SEQ ID NO: 21. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-108 (e.g., amino acid residues 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, or 108) of SEQ ID NO: 25, and ends at any one of amino acids 189-191 (e.g., amino acid residues 189, 190, or 191) of SEQ ID NO: 25. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-191 of SEQ ID NO: 25. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 108-189 of SEQ ID NO: 25. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-109 (e.g., amino acid residues 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108 or 109) of SEQ ID NO: 29, and ends at any one of amino acids 139-148 (e.g., amino acid residues 139, 140, 141, 142, 143, 144, 145, 146, 147, or 148) of SEQ ID NO: 29. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-148 of SEQ ID NO: 29. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 109-139 of SEQ ID NO: 29. In some embodiments, heteromultimers of the disclosure comprise at least one Cryptic polypeptide that is at least 70%, 75%, 80%, 85%, 90%, 91%, 92%, 93%, 94%, 95%, 96%, 97%, 98%, 99%, or 100% identical to amino acids of 26-94 of SEQ ID NO: 29.

The term “Cryptic family protein 1B polypeptide” includes polypeptides comprising any naturally occurring Cryptic family protein 1B protein (encoded by CFC1B or one of its nonhuman orthologs) as well as any variants thereof (including mutants, fragments, fusions, and peptidomimetic forms) that retain a useful activity.

The human Cryptic family protein 1B precursor protein sequence (NCBI Ref Seq NP_001072998.1) is as follows:

```
(SEQ ID NO: 33)
    1 MTWRHHVRLL FTVSLALQII NLGNSYQREK
        HNGGREEVTK VATQKHRQSP LNWTSSHFGE
  61 VTGSAEGWGP EEPLPYSWAF GEGASARPRC
        CRNGGTCVLG SFCVCPAHFT GRYCEHDQRR
121 SECGALEHGA WTLRACHLCR CIFGALHCLP
        LQTPDRCDPK DFLASHAHGP SAGGAPSLLL
181 LLPCALLHRL LRPDAPAHPR SLVPSVLQRE
        RRPCGRPGLG HRL
```

The signal peptide is indicated by single underline.

A processed Cryptic family protein 1B polypeptide sequence is as follows:

(SEQ ID NO: 34)
YQREKHNGGREEVTKVATQKHRQSPLNWTSSHFGEVTGSAEGWGPEEP
LPYSWAFGEGASARPRCCRNGGTCVLGSECVCPAHFTGRYCEHDQRRS
ECGALEHGAWTLRACHLCRCIFGALHCLPLQTPDRCDPKDFLASHAHG

A nucleic acid sequence encoding unprocessed human Cryptic family protein 1B precursor protein is shown below (SEQ ID NO: 35), corresponding to nucleotides 392-1060 of NCBI Reference Sequence NM_001079530.1. The signal sequence is underlined.

( SEQ ID NO: 35)
ATGACCTGGAGGCACCATGTCAGGCTTCTGTTTACGGTCAGTTTGGC
ATTACAGATCATCAATTTGGGAAACAGCTATCAAAGAGAGAAACATA
ACGGCGGTAGAGAGGAAGTCACCAAGGTTGCCACTCAGAAGCACCGA
CAGTCACCGCTCAACTGGACCTCCAGTCATTTCGGAGAGGTGACTGG
GAGCGCCGAGGGCTGGGGGCCGGAGGAGCCGCTCCCATACTCCTGGG
CTTTCGGAGAGGGTGCGTCCGCGCGGCCGCGCTGCTGCAGGAACGGC
GGTACCTGCGTGCTGGGCAGCTTCTGCGTGTGCCCGGCCCACTTCAC
CGGCCGCTACTGCGAGCATGACCAGAGGCGCAGTGAATGCGGCGCCC
TGGAGCACGGAGCCTGGACCCTCCGCGCCTGCCACCTCTGCAGGTGC
ATCTTCGGGGCCCTGCACTGCCTCCCCCTCCAGACGCCTGACCGCTG
TGACCCGAAAGACTTCCTGGCCTCCCACGCTCACGGGCCGAGCGCCG
GGGGCGCGCCCAGCCTGCTACTCTTGCTGCCCTGCGCACTCCTGCAC
CGCCTCCTGCGCCCGGATGCGCCCGCGCACCCTCGGTCCCTGGTCCC
TTCCGTCCTCCAGCGGGAGCGGCGCCCCTGCGGAAGGCCGGGACTTG
GGCATCGCCTT

A nucleic acid sequence encoding a processed Cryptic family protein 1B is shown below (SEQ ID NO: 36):

(SEQ ID NO: 36)
TATCAAAGAGAGAAACATAACGGCGGTAGAGAGGAAGTCACCAAGGT
TGCCACTCAGAAGCACCGACAGTCACCGCTCAACTGGACCTCCAGTC
ATTTCGGAGAGGTGACTGGGAGCGCCGAGGGCTGGGGGCCGGAGGAG
CCGCTCCCATACTCCTGGGCTTTCGGAGAGGGTGCGTCCGCGCGGCC
GCGCTGCTGCAGGAACGGCGGTACCTGCGTGCTGGGCAGCTTCTGCG
TGTGCCCGGCCCACTTCACCGGCCGCTACTGCGAGCATGACCAGAGG
CGCAGTGAATGCGGCGCCCTGGAGCACGGAGCCTGGACCCTCCGCGC
CTGCCACCTCTGCAGGTGCATCTTCGGGGCCCTGCACTGCCTCCCCC
TCCAGACGCCTGACCGCTGTGACCCGAAAGACTTCCTGGCCTCCCAC
GCTCACGGG

In certain embodiments, the disclosure relates to heteromultimers that comprise at least one Cryptic family protein 1B polypeptide, which includes fragments, functional variants, and modified forms thereof. Preferably, Cryptic family protein 1B polypeptides for use in accordance with the disclosure (e.g., heteromultimers comprising a Cryptic family protein 1B polypeptide and uses thereof) are soluble (e.g., an extracellular domain of Cryptic family protein 1B). In other preferred embodiments, Cryptic family protein 1B polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 33 or 34. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-30 (e.g., amino acid residues 26, 27, 28, 29, or 30) of SEQ ID NO: 33, and ends at any one of amino acids 82-223 (e.g., amino acid residues 82, 83, 84, 85, 86, 57, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 126, 217, 218, 219, 220, 221, 222, or 223) of SEQ ID NO: 33. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to amino acids of 26-223 of SEQ ID NO: 33. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to amino acids of 26-82 of SEQ ID NO: 33. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to amino acids of 30-82 of SEQ ID NO: 33. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to amino acids of 30-223 of SEQ ID NO: 33. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to amino acids of 26-169 of SEQ ID NO: 33. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to amino acids of 30-169 of SEQ ID NO: 33. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-90 (e.g., amino acid residues 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90) of SEQ ID NO: 33, and ends at any one of amino acids 214-223 (e.g., amino acid residues 214, 215, 126, 217, 218, 219, 220, 221, 222, or 223) of SEQ ID NO: 33. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to amino acids of 26-223 of SEQ ID NO: 33. In some embodiments, heteromultimers of the disclosure comprise at least one Cryptic family protein 1B polypeptide that is at least 70%, 75%, 80%, 85%, 90%, 91%, 92%, 93%, 94%, 95%, 96%, 97%, 98%, 99%, or 100% identical to amino acids of 90-214 of SEQ ID NO: 33.

The term “CRIM1 polypeptide” includes polypeptides comprising any naturally occurring polypeptide of a CRIM1 protein (encoded by CRIM1 or one of its nonhuman orthologs) as well as any variants thereof (including mutants, fragments, fusions, and peptidomimetic forms) that retain a useful activity.

The human CRIM1 precursor protein sequence (NCBI Ref Seq NP_057525.1) is as follows:

| (SEQ ID NO: 37) | |
|---|---|
| 1 | MYLVAGDRGL AGCGHLLVSL LGLLLLLARS GTRALVCLPC DESKCEEPRN CPGSIVQGVC |
| 61 | GCCYTCASQR NESCGGTFGI YGTCDRGLRC VIRPPLNGDS LTEYEAGVCE DENWTDDQLL |
| 121 | GFKPCNENLI AGCNIINGKC ECNTIRTCSN PFEFPSQDMC LSALKRIEEE KPDCSKARCE |
| 181 | VQFSPRCPED SVLIEGYAPP GECCPLPSRC VCNPAGCLRK VCQPGNLNIL VSKASGKPGE |
| 241 | CCDLYECKPV FGVDCRTVEC PPVQQTACPP DSYETQVRLT ADGCCTLPTR CECLSGLCGF |
| 301 | PVCEVGSTPR IVSRGDGTPG KCCDVFECVN DTKPACVFNN VEYYDGDMFR MDNCRFCRCQ |
| 361 | GGVAICFTAQ CGEINCERYY VPEGECCPVC EDPVYPFNNP AGCYANGLIL AHGDRWREDD |
| 421 | CTFCQCVNGE RHCVATVCGQ TCTNPVKVPG ECCPVCEEPT IITVDPPACG ELSNCTLTGK |
| 481 | DCINGFKRDH NGCRTCQCIN TEELCSERKQ GCTLNCPFGF LTDAQNCEIC ECRPRPKKCR |
| 541 | PIICDKYCPL GLLKNKHGCD ICRCKKCPEL SCSKICPLGF QQDSHGCLIC KCREASASAG |
| 601 | PPILSGTCLT VDGHHHKNEE SWHDGCRECY CLNGREMCAL ITCPVPACGN PTIHPGQCCP |
| 661 | SCADDFVVQK PELSTPSICH APGGEYFVEG ETWNIDSCTQ CTCHSGRVLC ETEVCPPLLC |
| 721 | QNPSRTQDSC CPQCTDQPFR PSLSRNNSVP NYCKNDEGDI FLAAESWKPD VCTSCICIDS |
| 781 | VISCFSESCP SVSCERPVLR KGQCCPYCIE DTIPKKVVCH FSGKAYADEE RWDLDSCTHC |
| 841 | YCLQGQTLCS TVSCPPLPCV EPINVEGSCC PMCPEMYVPE PTNIPIEKTN HRGEVDLEVP |
| 901 | 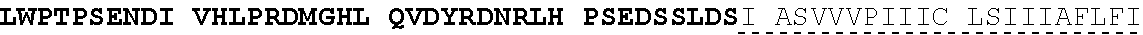 |
| 961 | NQKKQWIPLL CWYRTPTKPS SLNNQLVSVD CKKGTRVQVD SSQRMLRIAE PDARFSGFYS |
| 1021 | MQKQNHLQAD NFYQTV |

The signal peptide is indicated by a single underline, the extracellular domain is indicated by bold, and the transmembrane domain is indicated by dotted underline.

A mature CRIM1 sequence is as follows:

( SEQ ID NO: 38)
LVCLPCDESKCEEPRNCPGSIVQGVCGCCYTCASQRNESCGGTFGIY
GTCDRGLRCVIRPPLNGDSLTEYEAGVCEDENWTDDQLLGFKPCNEN
LIAGCNIINGKCECNTIRTCSNPFEFPSQDMCLSALKRIEEEKPDCS
KARCEVQFSPRCPEDSVLIEGYAPPGECCPLPSRCVCNPAGCLRKVC
QPGNLNILVSKASGKPGECCDLYECKPVFGVDCRTVECPPVQQTACP
PDSYETQVRLTADGCCTLPTCECLSGLCGFPVCEVGSTPRIVSRGDG
TPGKCCDVFECVNDTKPACVENNVEYYDGDMERMDNCRECRCQGGVA
ICETAQCGEINCERYYVPEGECCPVCEDPVYPENNPAGCYANGLILA
HGDRWREDDCTFCQCVNGERHCVATVCGQTCTNPVKVPGECCPVCEE
PTIITVDPPACGELSNCTLTGKDCINGFKRDHNGCRTCQCINTEELC
SERKQGCTLNCPFGFLTDAQNCEICECRPRPKKCRPIICDKYCPLGL
LKNKHGCDICRCKKCPELSCSKICPLGFQQDSHGCLICKCREASASA
GPPILSGTCLTVDGHHHKNEESWHDGCRECYCLNGREMCALITCPVP
ACGNPTIHPGQCCPSCADDFVVQKPELSTPSICHAPGGEYFVEGETW
NIDSCTQCTCHSGRVLCETEVCPPLLCQNPSRTQDSCCPQCTDQPFR
PSLSRNNSVPNYCKNDEGDIFLAAESWKPDVCTSCICIDSVISCFSE
SCPSVSCERPVLRKGQCCPYCIEDTIPKKVVCHFSGKAYADEERWDL
DSCTHCYCLQGQTLCSTVSCPPLPCVEPINVEGSCCPMCPEMYVPEP
TNIPIEKTNHRGEVDLEVPLWPTPSENDIVHLPRDMGHLQVDYRDNR
LHPSEDSSLDS

A nucleic acid sequence encoding unprocessed human CRIM1 precursor protein is shown below (SEQ ID NO: 39), corresponding to nucleotides 67-3174 of NCBI Reference Sequence NM_016441.2. The signal sequence is indicated by solid underline and the transmembrane region by dotted underline.

(SEQ ID NO: 39)
ATGTACTTGGTGGCGGGGGACAGGGGGTTGGCCGGCTGCGGGCACCTCCTGGTCTCGCTGCTGGGGCTGCTGCTG
CTGCTGGCGCGCTCCGGCACCCGGGCGCTGGTCTGCCTGCCCTGTGACGAGTCCAAGTGCGAGGAGCCCAGGAAC
TGCCCGGGGAGCATCGTGCAGGGCGTCTGCGGCTGCTGCTACACGTGCGCCAGCCAGAGGAACGAGAGCTGCGGC
GGCACCTTCGGGATTTACGGAACCTGCGACCGGGGGCTGCGTTGTGTCATCCGCCCCCCGCTCAATGGCGACTCC
CTCACCGAGTACGAAGCGGGCGTTTGCGAAGATGAGAACTGGACTGATGACCAACTGCTTGGTTTTAAACCATGC
AATGAAAACCTTATTGCTGGCTGCAATATAATCAATGGGAAATGTGAATGTAACACCATTCGAACCTGCAGCAAT
CCCTTTGAGTTTCCAAGTCAGGATATGTGCCTTTCAGCTTTAAAGAGAATTGAAGAAGAGAAGCCAGATTGCTCC
AAGGCCCGCTGTGAAGTCCAGTTCTCTCCACGTTGTCCTGAAGATTCTGTTCTGATCGAGGGTTATGCTCCTCCT
GGGGAGTGCTGTCCCTTACCCAGCCGCTGCGTGTGCAACCCCGCAGGCTGTCTGCGCAAAGTCTGCCAGCCGGGA
AACCTGAACATACTAGTGTCAAAAGCCTCAGGGAAGCCGGGAGAGTGCTGTGACCTCTATGAGTGCAAACCAGTT
TTCGGCGTGGACTGCAGGACTGTGGAATGCCCTCCTGTTCAGCAGACCGCGTGTCCCCCGGACAGCTATGAAACT
CAAGTCAGACTAACTGCAGATGGTTGCTGTACTTTGCCAACAAGATGCGAGTGTCTCTCTGGCTTATGTGGTTTC
CCCGTGTGTGAGGTGGGATCCACTCCCCGCATAGTCTCTCGTGGCGATGGGACACCTGGAAAGTGCTGTGATGTC
TTTGAATGTGTTAATGATACAAAGCCAGCCTGCGTATTTAACAATGTGGAATATTATGATGGAGACATGTTTCGA
ATGGACAACTGTCGGTTCTGTCGATGCCAAGGGGGCGTTGCCATCTGCTTCACCGCCCAGTGTGGTGAGATAAAC
TGCGAGAGGTACTACGTGCCCGAAGGAGAGTGCTGCCCAGTGTGTGAAGATCCAGTGTATCCTTTTAATAATCCC
GCTGGCTGCTATGCCAATGGCCTGATCCTTGCCCACGGAGACCGGTGGCGGGAAGACGACTGCACATTCTGCCAG
TGCGTCAACGGTGAACGCCACTGCGTTGCGACCGTCTGCGGACAGACCTGCACAAACCCTGTGAAAGTGCCTGGG
GAGTGTTGCCCTGTGTGCGAAGAACCAACCATCATCACAGTTGATCCACCTGCATGTGGGGAGTTATCAAACTGC
ACTCTGACAGGGAAGGACTGCATTAATGGTTTCAAACGCGATCACAATGGTTGTCGGACCTGTCAGTGCATAAAC
ACCGAGGAACTATGTTCAGAACGTAAACAAGGCTGCACCTTGAACTGTCCCTTCGGTTTCCTTACTGATGCCCAA
AACTGTGAGATCTGTGAGTGCCGCCCAAGGCCCAAGAAGTGCAGACCCATAATCTGTGACAAGTATTGTCCACTT
GGATTGCTGAAGAATAAGCACGGCTGTGACATCTGTCGCTGTAAGAAATGTCCAGAGCTCTCATGCAGTAAGATC
TGCCCCTTGGGTTTCCAGCAGGACAGTCACGGCTGTCTTATCTGCAAGTGCAGAGAGGCCTCTGCTTCAGCTGGG
CCACCCATCCTGTCGGGCACTTGTCTCACCGTGGATGGTCATCATCATAAAAATGAGGAGAGCTGGCACGATGGG
TGCCGGGAATGCTACTGTCTCAATGGACGGGAAATGTGTGCCCTGATCACCTGCCCGGTGCCTGCCTGTGGCAAC
CCCACCATTCACCCTGGACAGTGCTGCCCATCATGTGCAGATGACTTTGTGGTGCAGAAGCCAGAGCTCAGTACT
CCCTCCATTTGCCACGCCCCTGGAGGAGAATACTTTGTGGAAGGAGAAACGTGGAACATTGACTCCTGTACTCAG
TGCACCTGCCACAGCGGACGGGTGCTGTGTGAGACAGAGGTGTGCCCACCGCTGCTCTGCCAGAACCCCTCACGC
ACCCAGGATTCCTGCTGCCCACAGTGTACAGATCAACCTTTTCGGCCTTCCTTGTCCCGCAATAACAGCGTACCT
AATTACTGCAAAAATGATGAAGGGGATATATTCCTGGCAGCTGAGTCCTGGAAGCCTGACGTTTGTACCAGCTGC
ATCTGCATTGATAGCGTAATTAGCTGTTTCTCTGAGTCCTGCCCTTCTGTATCCTGTGAAAGACCTGTCTTGAGA
AAAGGCCAGTGTTGTCCCTACTGCATAGAAGACACAATTCCAAAGAAGGTGGTGTGCCACTTCAGTGGGAAGGCC
TATGCCGACGAGGAGCGGTGGGACCTTGACAGCTGCACCCACTGCTACTGCCTGCAGGGCCAGACCCTCTGCTCG
ACCGTCAGCTGCCCCCCTCTGCCCTGTGTTGAGCCCATCAACGTGGAAGGAAGTTGCTGCCCAATGTGTCCAGAA
ATGTATGTCCCAGAACCAACCAATATACCCATTGAGAAGACAAACCATCGAGGAGAGGTTGACCTGGAGGTTCCC
CTGTGGCCCACGCCTAGTGAAAATGATATCGTCCATCTCCCTAGAGATATGGGTCACCTCCAGGTAGATTACAGA

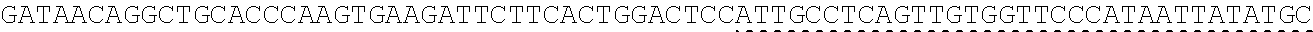

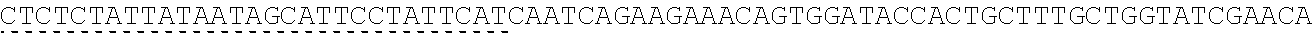

CCAACTAAGCCTTCTTCCTTAAATAATCAGCTAGTATCTGTGGACTGCAAGAAAGGAACCAGAGTCCAGGTGGAC
AGTTCCCAGAGAATGCTAAGAATTGCAGAACCAGATGCAAGATTCAGTGGCTTCTACAGCATGCAAAAACAGAAC
CATCTACAGGCAGACAATTTCTACCAAACAGTG

A nucleic acid sequence encoding processed extracellular human CRIM1 is shown below (SEQ ID NO: 40):

(SEQ ID NO: 40)
CTGGTCTGCCTGCCCTGTGACGAGTCCAAGTGCGAGGAGCCCAGGAAC
TGCCCGGGGAGCATCGTGCAGGGCGTCTGCGGCTGCTGCTACACGTGC
GCCAGCCAGAGGAACGAGAGCTGCGGCGGCACCTTCGGGATTTACGGA
ACCTGCGACCGGGGGCTGCGTTGTGTCATCCGCCCCCCGCTCAATGGC
GACTCCCTCACCGAGTACGAAGCGGGCGTTTGCGAAGATGAGAACTGG
ACTGATGACCAACTGCTTGGTTTTAAACCATGCAATGAAAACCTTATT
GCTGGCTGCAATATAATCAATGGGAAATGTGAATGTAACACCATTCGA
ACCTGCAGCAATCCCTTTGAGTTTCCAAGTCAGGATATGTGCCTTTCA
GCTTTAAAGAGAATTGAAGAAGAGAAGCCAGATTGCTCCAAGGCCCGC
TGTGAAGTCCAGTTCTCTCCACGTTGTCCTGAAGATTCTGTTCTGATC
GAGGGTTATGCTCCTCCTGGGGAGTGCTGTCCCTTACCCAGCCGCTGC
GTGTGCAACCCCGCAGGCTGTCTGCGCAAAGTCTGCCAGCCGGGAAAC
CTGAACATACTAGTGTCAAAAGCCTCAGGGAAGCCGGGAGAGTGCTGT
GACCTCTATGAGTGCAAACCAGTTTTCGGCGTGGACTGCAGGACTGTG
GAATGCCCTCCTGTTCAGCAGACCGCGTGTCCCCCGGACAGCTATGAA
ACTCAAGTCAGACTAACTGCAGATGGTTGCTGTACTTTGCCAACAAGA
TGCGAGTGTCTCTCTGGCTTATGTGGTTTCCCCGTGTGTGAGGTGGGA
TCCACTCCCCGCATAGTCTCTCGTGGCGATGGGACACCTGGAAAGTGC
TGTGATGTCTTTGAATGTGTTAATGATACAAAGCCAGCCTGCGTATTT
AACAATGTGGAATATTATGATGGAGACATGTTTCGAATGGACAACTGT
CGGTTCTGTCGATGCCAAGGGGGCGTTGCCATCTGCTTCACCGCCCAG
TGTGGTGAGATAAACTGCGAGAGGTACTACGTGCCCGAAGGAGAGTGC
TGCCCAGTGTGTGAAGATCCAGTGTATCCTTTTAATAATCCCGCTGGC
TGCTATGCCAATGGCCTGATCCTTGCCCACGGAGACCGGTGGCGGGAA
GACGACTGCACATTCTGCCAGTGCGTCAACGGTGAACGCCACTGCGTT
GCGACCGTCTGCGGACAGACCTGCACAAACCCTGTGAAAGTGCCTGGG
GAGTGTTGCCCTGTGTGCGAAGAACCAACCATCATCACAGTTGATCCA
CCTGCATGTGGGGAGTTATCAAACTGCACTCTGACAGGGAAGGACTGC
ATTAATGGTTTCAAACGCGATCACAATGGTTGTCGGACCTGTCAGTGC
ATAAACACCGAGGAACTATGTTCAGAACGTAAACAAGGCTGCACCTTG
AACTGTCCCTTCGGTTTCCTTACTGATGCCCAAAACTGTGAGATCTGT
GAGTGCCGCCCAAGGCCCAAGAAGTGCAGACCCATAATCTGTGACAAG
TATTGTCCACTTGGATTGCTGAAGAATAAGCACGGCTGTGACATCTGT
CGCTGTAAGAAATGTCCAGAGCTCTCATGCAGTAAGATCTGCCCCTTG
GGTTTCCAGCAGGACAGTCACGGCTGTCTTATCTGCAAGTGCAGAGAG
GCCTCTGCTTCAGCTGGGCCACCCATCCTGTCGGGCACTTGTCTCACC
GTGGATGGTCATCATCATAAAAATGAGGAGAGCTGGCACGATGGGTGC
CGGGAATGCTACTGTCTCAATGGACGGGAAATGTGTGCCCTGATCACC
TGCCCGGTGCCTGCCTGTGGCAACCCCACCATTCACCCTGGACAGTGC
TGCCCATCATGTGCAGATGACTTTGTGGTGCAGAAGCCAGAGCTCAGT
ACTCCCTCCATTTGCCACGCCCCTGGAGGAGAATACTTTGTGGAAGGA
GAAACGTGGAACATTGACTCCTGTACTCAGTGCACCTGCCACAGCGGA
CGGGTGCTGTGTGAGACAGAGGTGTGCCCACCGCTGCTCTGCCAGAAC
CCCTCACGCACCCAGGATTCCTGCTGCCCACAGTGTACAGATCAACCT
TTTCGGCCTTCCTTGTCCCGCAATAACAGCGTACCTAATTACTGCAAA
AATGATGAAGGGGATATATTCCTGGCAGCTGAGTCCTGGAAGCCTGAC
GTTTGTACCAGCTGCATCTGCATTGATAGCGTAATTAGCTGTTTCTCT
GAGTCCTGCCCTTCTGTATCCTGTGAAAGACCTGTCTTGAGAAAAGGC
CAGTGTTGTCCCTACTGCATAGAAGACACAATTCCAAAGAAGGTGGTG
TGCCACTTCAGTGGGAAGGCCTATGCCGACGAGGAGCGGTGGGACCTT
GACAGCTGCACCCACTGCTACTGCCTGCAGGGCCAGACCCTCTGCTCG
ACCGTCAGCTGCCCCCCTCTGCCCTGTGTTGAGCCCATCAACGTGGAA
GGAAGTTGCTGCCCAATGTGTCCAGAAATGTATGTCCCAGAACCAACC
AATATACCCATTGAGAAGACAAACCATCGAGGAGAGGTTGACCTGGAG
GTTCCCCTGTGGCCCACGCCTAGTGAAAATGATATCGTCCATCTCCCT
AGAGATATGGGTCACCTCCAGGTAGATTACAGAGATAACAGGCTGCAC
CCAAGTGAAGATTCTTCACTGGACTCC

In certain embodiments, the disclosure relates to heteromultimers that comprise at least one CRIM1 polypeptide, which includes fragments, functional variants, and modified forms thereof. Preferably, CRIM1 polypeptides for use in accordance with the disclosure (e.g., heteromultimers comprising a CRIM1 polypeptide and uses thereof) are soluble (e.g., an extracellular domain of CRIM1). In other preferred embodiments, CRIM1 polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one CRIM1 polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 37 or 38. In some embodiments, heteromultimers of the disclosure comprise at least one CRIM1 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 35-37 (e.g., amino acid residues 35, 36, or 37) of SEQ ID NO: 37, and ends at any one of amino acids 873-939 (e.g., amino acid residues 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, or 939) of SEQ ID NO: 37. In some embodiments, heteromultimers of the disclosure comprise at least one CRIM1 polypeptide that is at least 70%, 75%, 80%, 85%, 90%, 91%, 92%, 93%, 94%, 95%, 96%, 97%, 98%, 99%, or 100% identical to amino acids of 35-939 of SEQ ID NO: 37. In some embodiments, heteromultimers of the disclosure comprise at least one CRIM1 polypeptide that is at least 70%, 75%, 80%, 85%, 90%, 91%, 92%, 93%, 94%, 95%, 96%, 97%, 98%, 99%, or 100% identical to amino acids of 37-939 of SEQ ID NO: 37. In some embodiments, heteromultimers of the disclosure comprise at least one CRIM1 polypeptide that is at least 70%, 75%, 80%, 85%, 90%, 91%, 92%, 93%, 94%, 95%, 96%, 97%, 98%, 99%, or 100% identical to amino acids of 35-873 of SEQ ID NO: 37. In some embodiments, heteromultimers of the disclosure comprise at least one CRIM1 polypeptide that is at least 70%, 75%, 80%, 85%, 90%, 91%, 92%, 93%, 94%, 95%, 96%, 97%, 98%, 99%, or 100% identical to amino acids of 37-939 of SEQ ID NO: 37.

The term “CRIM2 polypeptide” includes polypeptides comprising any naturally occurring CRIM2 protein (encoded by KCP or one of its nonhuman orthologs) as well as any variants thereof (including mutants, fragments, fusions, and peptidomimetic forms) that retain a useful activity.

A human CRIM2 isoform 1 precursor protein sequence (NCBI Ref Seq NP_001129386.1) is as follows:

```
(SEQ ID NO: 41)
      1 MAGVGAAALS LLLHLGALAL AAGAEGGAVP
          REPPGQQTTA HSSVLAGNSQ EQWHPLREWL
    61 GRLEAAVMEL REQNKDLQTR VRQLESCECH
          PASPQCWGLG RAWPEGARWE PDACTACVCQ
  121 DGAAHCGPQA HLPHCRGCSQ NGQTYGNGET
          FSPDACTTCR CLTGAVQCQG PSCSELNCLE
  181 SCTPPGECCP ICCTEGGSHW EHGQEWTTPG
          DPCRICRCLE GHIQCRQREC ASLCPYPARP
  241 LPGTCCPVCD GCFLNGREHR SGEPVGSGDP
          CSHCRCANGS VQCEPLPCPP VPCRHPGKIP
  301 GQCCPVCDGC EYQGHQYQSQ ETFRLQERGL
          CVRCSCQAGE VSCEEQECPV TPCALPASGR
  361 QLCPACELDG EEFAEGVQWE PDGRPCTACV
          CQDGVPKCGA VLCPPAPCQH PTQPPGACCP
  421 SCDSCTYHSQ VYANGQNFTD ADSPCHACHC
          QDGTVTCSLV DCPPTTCARP QSGPGQCCPR
  481 CPDCILEEEV FVDGESFSHP RDPCQECRCQ
          EGHAHCQPRP CPRAPCAHPL PGTCCPNDCS
  541 GCAFGGKEYP SGADFPHPSD PCRLCRCLSG
          NVQCLARRCV PLPCPEPVLL PGECCPQCPA
  601 PAGCPRPGAA HARHQEYFSP PGDPCRRCLC
          LDGSVSCQRL PCPPAPCAHP RQGPCCPSCD
  661 GCLYQGKEFA SGERFPSPTA ACHLCLCWEG
          SVSCEPKACA PALCPFPARG DCCPDCDGCE
  721 YLGESYLSNQ EFPDPREPCN LCTCLGGFVT
          CGRRPCEPPG CSHPLIPSGH CCPTCQGCRY
  781 HGVTTASGET LPDPLDPTCS LCTCQEGSMR
          CQKKPCPPAL CPHPSPGPCF CPVCHSCLSQ
  841 GREHQDGEEF EGPAGSCEWC RCQAGQVSCV
          RLQCPPLPCK LQVTERGSCC PRCRGCLAHG
  901 EEHPEGSRWV PPDSACSSCV CHEGVVTCAR
          IQCISSCAQP RQGPHDCCPQ CSDCEHEGRK
  961 YEPGESFQPG ADPCEVCICE PQPEGPPSLR
          CHRRQCPSLV GCPPSQLLPP GPQHCCPTCA
1021 EALSNCSEGL LGSELAPPDP CYTCQCQDLT
          WLCIHQACPE LSCPLSERHT PPGSCCPVCR
1081 APTQSCVHQG REVASGERWT VDTCTSCSCM
          AGTVRCQSQR CSPLSCGPDK APALSPGSCC
1141 PRCLPRPASC MAFGDPHYRT FDGRLLHFQG
          SCSYVLAKDC HSGDFSVHVT NDDRGRSGVA
1201 WTQEVAVLLG DMAVRLLQDG AVTVDGHPVA
          LPFLQEPLLY VELRGHTVIL HAQPGLQVLW
1261 DGQSQVEVSV PGSYQGRTCG LCGNFNGFAQ
          DDLQGPEGLL LPSEAAFGNS WQVSEGLWPG
1321 RPCSAGREVD PCRAAGYRAR REANARCGVL
          KSSPFSRCHA VVPPEPFFAA CVYDLCACGP
1381 GSSADACLCD ALEAYASHCR QAGVTPTWRG
          PTLCVVGCPL ERGFVFDECG PPCPRTCFNQ
1441 HIPLGELAAH CVRPCVPGCQ CPAGLVEHEA
          HCIPPEACPQ VLLTGDQPLG ARPSPSREPQ
1501 ETP
```

The signal peptide is indicated by single underline.

A processed CRIM2 isoform 1 polypeptide sequence is as follows:

(SEQ ID NO: 42)
GAVPREPPGQQTTAHSSVLAGNSQEQWHPLREWLGRLEAAVMELREQNK
DLQTRVRQLESCECHPASPQCWGLGRAWPEGARWEPDACTACVCQDGAA
HCGPQAHLPHCRGCSQNGQTYGNGETFSPDACTTCRCLTGAVQCQGPSC
SELNCLESCTPPGECCPICCTEGGSHWEHGQEWTTPGDPCRICRCLEGH
IQCRQRECASLCPYPARPLPGTCCPVCDGCFLNGREHRSGEPVGSGDPC
SHCRCANGSVQCEPLPCPPVPCRHPGKIPGQCCPVCDGCEYQGHQYQSQ
ETFRLQERGLCVRCSCQAGEVSCEEQECPVTPCALPASGRQLCPACELD
GEEFAEGVQWEPDGRPCTACVCQDGVPKCGAVLCPPAPCQHPTQPPGAC
CPSCDSCTYHSQVYANGQNFTDADSPCHACHCQDGTVTCSLVDCPPTTC
ARPQSGPGQCCPRCPDCILEEEVFVDGESFSHPRDPCQECRCQEGHAHC
QPRPCPRAPCAHPLPGTCCPNDCSGCAFGGKEYPSGADFPHPSDPCRLC
RCLSGNVQCLARRCVPLPCPEPVLLPGECCPQCPAPAGCPRPGAAHARH
QEYFSPPGDPCRRCLCLDGSVSCQRLPCPPAPCAHPRQGPCCPSCDGCL
YQGKEFASGERFPSPTAACHLCLCWEGSVSCEPKACAPALCPFPARGDC
CPDCDGCEYLGESYLSNQEFPDPREPCNLCTCLGGFVTCGRRPCEPPGC
SHPLIPSGHCCPTCQGCRYHGVTTASGETLPDPLDPTCSLCTCQEGSMR
CQKKPCPPALCPHPSPGPCFCPVCHSCLSQGREHQDGEEFEGPAGSCEW
CRCQAGQVSCVRLQCPPLPCKLQVTERGSCCPRCRGCLAHGEEHPEGSR
WVPPDSACSSCVCHEGVVTCARTQCISSCAQPRQGPHDCCPQCSDCEHE
GRKYEPGESFQPGADPCEVCICEPQPEGPPSLRCHRRQCPSLVGCPPSQ
LLPPGPQHCCPTCAEALSNCSEGLLGSELAPPDPCYTCQCQDLTWLCIH
QACPELSCPLSERHTPPGSCCPVCRAPTQSCVHQGREVASGERWTVDTC
TSCSCMAGTVRCQSQRCSPLSCGPDKAPALSPGSCCPRCLPRPASCMAF
GDPHYRTFDGRLLHFQGSCSYVLAKDCHSGDFSVHVTNDDRGRSGVAWT
QEVAVLLGDMAVRLLQDGAVTVDGHPVALPFLQEPLLYVELRGHTVILH
AQPGLQVLWDGQSQVEVSVPGSYQGRTCGLCGNFNGFAQDDLQGPEGLL
LPSEAAFGNSWQVSEGLWPGRPCSAGREVDPCRAAGYRARREANARCGV
LKSSPFSRCHAVVPPEPFFAACVYDLCACGPGSSADACLCDALEAYASH
CRQAGVTPTWRGPTLCVVGCPLERGFVFDECGPPCPRTCFNQHIPLGEL
AAHCVRPCVPGCQCPAGLVEHEAHCIPPEACPQVLLTGDQPLGARPSPS
REPQETP

A nucleic acid sequence encoding unprocessed human CRIM2 isoform 1 precursor protein is shown below (SEQ ID NO: 43), corresponding to nucleotides 44-4552 of NCBI Reference Sequence NM_001135914.1. The signal sequence is underlined.

(SEQ ID NO: 43)
ATGGCCGGGGTCGGGGCCGCTGCGCTGTCCCTTCTCCTGCACCTCGGGG
CCCTGGCGCTGGCCGCGGGCGCGGAAGGTGGGGCTGTCCCCAGGGAGCC
CCCTGGGCAGCAGACAACTGCCCATTCCTCAGTCCTTGCTGGGAACTCC
CAGGAGCAGTGGCACCCCCTGCGAGAGTGGCTGGGGCGACTGGAGGCTG
CAGTGATGGAGCTCAGAGAACAGAATAAGGACCTGCAGACGAGGGTGAG
GCAGCTGGAGTCCTGTGAGTGCCACCCTGCATCTCCCCAGTGCTGGGGG
CTGGGGCGTGCCTGGCCCGAGGGGGCACGCTGGGAGCCTGACGCCTGCA
CAGCCTGCGTCTGCCAGGATGGGGCCGCTCACTGTGGCCCCCAAGCACA
CCTGCCCCATTGCAGGGGCTGCAGCCAAAATGGCCAGACCTACGGCAAC
GGGGAGACCTTCTCCCCAGATGCCTGCACCACCTGCCGCTGTCTGACAG
GAGCCGTGCAGTGCCAGGGGCCCTCGTGTTCAGAGCTCAACTGCTTGGA
GAGCTGCACCCCACCTGGGGAGTGCTGCCCCATCTGCTGCACAGAAGGT
GGCTCTCACTGGGAACATGGCCAAGAGTGGACAACACCTGGGGACCCCT
GCCGAATCTGCCGGTGCCTGGAGGGTCACATCCAGTGCCGCCAGCGAGA
ATGTGCCAGCCTGTGTCCATACCCAGCCCGGCCCCTCCCAGGCACCTGC
TGCCCTGTGTGTGATGGCTGTTTCCTAAACGGGCGGGAGCACCGCAGCG
GGGAGCCTGTGGGCTCAGGGGACCCCTGCTCGCACTGCCGCTGTGCTAA
TGGGAGTGTCCAGTGTGAGCCTCTGCCCTGCCCGCCAGTGCCCTGCAGA
CACCCAGGCAAGATCCCTGGGCAGTGCTGCCCTGTCTGCGATGGCTGTG
AGTACCAGGGACACCAGTATCAGAGCCAGGAGACCTTCAGACTCCAAGA
GCGGGGCCTCTGTGTCCGCTGCTCCTGCCAGGCTGGCGAGGTCTCCTGT
GAGGAGCAGGAGTGCCCAGTCACCCCCTGTGCCCTGCCTGCCTCTGGCC
GCCAGCTCTGCCCAGCCTGTGAGCTGGATGGAGAGGAGTTTGCTGAGGG
AGTCCAGTGGGAGCCTGATGGTCGGCCCTGCACCGCCTGCGTCTGTCAA
GATGGGGTACCCAAGTGCGGGGCTGTGCTCTGCCCCCCAGCCCCCTGCC
AGCACCCCACCCAGCCCCCTGGTGCCTGCTGCCCCAGCTGTGACAGCTG
CACCTACCACAGCCAAGTGTATGCCAATGGGCAGAACTTCACGGATGCA
GACAGCCCTTGCCATGCCTGCCACTGTCAGGATGGAACTGTGACATGCT
CCTTGGTTGACTGCCCTCCCACGACCTGTGCCAGGCCCCAGAGTGGACC
AGGCCAGTGTTGCCCCAGGTGCCCAGACTGCATCCTGGAGGAAGAGGTG
TTTGTGGACGGCGAGAGCTTCTCCCACCCCCGAGACCCCTGCCAGGAGT
GCCGATGCCAGGAAGGCCATGCCCACTGCCAGCCTCGCCCCTGCCCCAG
GGCCCCCTGTGCCCACCCGCTGCCTGGGACCTGCTGCCCGAACGACTGC
AGCGGCTGTGCCTTTGGCGGGAAAGAGTACCCCAGCGGAGCGGACTTCC
CCCACCCCTCTGACCCCTGCCGTCTGTGTCGCTGTCTGAGCGGCAACGT
GCAGTGCCTGGCCCGCCGCTGCGTGCCGCTGCCCTGTCCAGAGCCTGTC
CTGCTGCCGGGAGAGTGCTGCCCGCAGTGCCCAGCCCCCGCCGGCTGCC
CACGGCCCGGCGCGGCCCACGCCCGCCACCAGGAGTACTTCTCCCCGCC
CGGCGATCCCTGCCGCCGCTGCCTCTGCCTCGACGGCTCCGTGTCCTGC
CAGCGGCTGCCCTGCCCGCCCGCGCCCTGCGCGCACCCGCGCCAGGGGC
CTTGCTGCCCCTCCTGCGACGGCTGCCTGTACCAGGGGAAGGAGTTTGC
CAGCGGGGAGCGCTTCCCATCGCCCACTGCTGCCTGCCACCTCTGCCTT
TGCTGGGAGGGCAGCGTGAGCTGCGAGCCCAAGGCATGTGCCCCTGCAC
TGTGCCCCTTCCCTGCCAGGGGCGACTGCTGCCCTGACTGTGATGGCTG
TGAGTACCTGGGGGAGTCCTACCTGAGTAACCAGGAGTTCCCAGACCCC
CGAGAACCCTGCAACCTGTGTACCTGTCTTGGAGGCTTCGTGACCTGCG
GCCGCCGGCCCTGTGAGCCTCCGGGCTGCAGCCACCCACTCATCCCCTC
TGGGCACTGCTGCCCGACCTGCCAGGGATGCCGCTACCATGGCGTCACT
ACTGCCTCCGGAGAGACCCTTCCTGACCCACTTGACCCTACCTGCTCCC
TCTGCACCTGCCAGGAAGGTTCCATGCGCTGCCAGAAGAAGCCATGTCC
CCCAGCTCTCTGCCCCCACCCCTCTCCAGGCCCCTGCTTCTGCCCTGTT
TGCCACAGCTGTCTCTCTCAGGGCCGGGAGCACCAGGATGGGGAGGAGT
TTGAGGGACCAGCAGGCAGCTGTGAGTGGTGTCGCTGTCAGGCTGGCCA
GGTCAGCTGTGTGCGGCTGCAGTGCCCACCCCTTCCCTGCAAGCTCCAG
GTCACCGAGCGGGGGAGCTGCTGCCCTCGCTGCAGAGGCTGCCTGGCTC
ATGGGGAAGAGCACCCCGAAGGCAGTAGATGGGTGCCCCCCGACAGTGC
CTGCTCCTCCTGTGTGTGTCACGAGGGCGTCGTCACCTGTGCACGCATC
CAGTGCATCAGCTCTTGCGCCCAGCCCCGCCAAGGGCCCCATGACTGCT
GTCCTCAATGCTCTGACTGTGAGCATGAGGGCCGGAAGTACGAGCCTGG
GGAGAGCTTCCAGCCTGGGGCAGACCCCTGTGAAGTGTGCATCTGCGAG
CCACAGCCTGAGGGGCCTCCCAGCCTTCGCTGTCACCGGCGGCAGTGTC
CCAGCCTGGTGGGCTGCCCCCCCAGCCAGCTCCTGCCCCCTGGGCCCCA
GCACTGCTGTCCCACCTGTGCCGAGGCCTTGAGTAACTGTTCAGAGGGC
CTGCTGGGATCTGAGCTAGCCCCACCAGACCCCTGCTACACGTGCCAGT
GCCAGGACCTGACATGGCTCTGCATCCACCAGGCTTGTCCTGAGCTCAG
CTGTCCCCTCTCAGAGCGCCACACTCCCCCTGGGAGCTGCTGCCCCGTA
TGCCGGGCTCCCACCCAGTCCTGCGTGCACCAGGGCCGTGAGGTGGCCT
CTGGAGAGCGCTGGACTGTGGACACCTGCACCAGCTGCTCCTGCATGGC
GGGCACCGTGCGTTGCCAGAGCCAGCGCTGCTCACCGCTCTCGTGTGGC
CCCGACAAGGCCCCTGCCCTGAGTCCTGGCAGCTGCTGCCCCCGCTGCC
TGCCTCGGCCCGCTTCCTGCATGGCCTTCGGAGACCCCCATTACCGCAC
CTTCGACGGCCGCCTGCTGCACTTCCAGGGCAGTTGCAGCTATGTGCTG
GCCAAGGACTGCCACAGCGGGGACTTCAGTGTGCACGTGACCAATGATG
ACCGGGGCCGGAGCGGTGTGGCCTGGACCCAGGAGGTGGCGGTGCTGCT
GGGAGACATGGCCGTGCGGCTGCTGCAGGACGGGGCAGTCACGGTGGAT
GGGCACCCGGTGGCCTTGCCCTTCCTGCAGGAGCCGCTGCTGTATGTGG
AGCTGCGAGGACACACTGTGATCCTGCACGCCCAGCCCGGGCTCCAGGT
GCTGTGGGATGGGCAGTCCCAGGTGGAGGTGAGCGTACCTGGCTCCTAC
CAGGGCCGGACTTGTGGGCTCTGTGGGAACTTCAATGGCTTTGCCCAGG
ACGATCTGCAGGGCCCTGAGGGGCTGCTCCTGCCCTCGGAGGCTGCGTT
TGGGAATAGCTGGCAGGTCTCAGAGGGGCTGTGGCCTGGCCGGCCCTGT
TCTGCAGGCCGAGAGGTGGATCCGTGCCGGGCAGCAGGTTACCGTGCCA
GGCGTGAGGCCAATGCCCGGTGTGGGGTGCTGAAGTCCTCCCCATTCAG
TCGCTGCCATGCTGTGGTGCCACCGGAGCCCTTCTTTGCCGCCTGTGTG
TATGACCTGTGTGCCTGTGGCCCTGGCTCCTCCGCTGATGCCTGCCTCT
GTGATGCCCTGGAAGCCTACGCCAGTCACTGTCGCCAGGCAGGAGTGAC
ACCTACCTGGCGAGGCCCCACGCTGTGTGTGGTAGGCTGCCCCCTGGAG
CGTGGCTTCGTGTTTGATGAGTGCGGCCCACCCTGTCCCCGCACCTGCT
TCAATCAGCATATCCCCCTGGGGGAGCTGGCAGCCCACTGCGTGAGGCC
CTGCGTGCCCGGCTGCCAGTGCCCTGCAGGCCTGGTGGAGCATGAGGCC
CACTGCATCCCACCCGAGGCCTGCCCCCAAGTCCTGCTCACTGGAGACC
AGCCACTTGGTGCTCGGCCCAGCCCCAGCCGGGAGCCCCAGGAGACACC
C

A nucleic acid sequence encoding a processed human CRIM2 isoform 1 is shown below (SEQ ID NO: 44):

(SEQ ID NO: 44)
GGGGCTGTCCCCAGGGAGCCCCCTGGGCAGCAGACAACTGCCCATTCCT
CAGTCCTTGCTGGGAACTCCCAGGAGCAGTGGCACCCCCTGCGAGAGTG
GCTGGGGCGACTGGAGGCTGCAGTGATGGAGCTCAGAGAACAGAATAAG
GACCTGCAGACGAGGGTGAGGCAGCTGGAGTCCTGTGAGTGCCACCCTG
CATCTCCCCAGTGCTGGGGGCTGGGGCGTGCCTGGCCCGAGGGGGCACG
CTGGGAGCCTGACGCCTGCACAGCCTGCGTCTGCCAGGATGGGGCCGCT
CACTGTGGCCCCCAAGCACACCTGCCCCATTGCAGGGGCTGCAGCCAAA
ATGGCCAGACCTACGGCAACGGGGAGACCTTCTCCCCAGATGCCTGCAC
CACCTGCCGCTGTCTGACAGGAGCCGTGCAGTGCCAGGGGCCCTCGTGT
TCAGAGCTCAACTGCTTGGAGAGCTGCACCCCACCTGGGGAGTGCTGCC
CCATCTGCTGCACAGAAGGTGGCTCTCACTGGGAACATGGCCAAGAGTG
GACAACACCTGGGGACCCCTGCCGAATCTGCCGGTGCCTGGAGGGTCAC
ATCCAGTGCCGCCAGCGAGAATGTGCCAGCCTGTGTCCATACCCAGCCC
GGCCCCTCCCAGGCACCTGCTGCCCTGTGTGTGATGGCTGTTTCCTAAA
CGGGCGGGAGCACCGCAGCGGGGAGCCTGTGGGCTCAGGGGACCCCTGC
TCGCACTGCCGCTGTGCTAATGGGAGTGTCCAGTGTGAGCCTCTGCCCT
GCCCGCCAGTGCCCTGCAGACACCCAGGCAAGATCCCTGGGCAGTGCTG
CCCTGTCTGCGATGGCTGTGAGTACCAGGGACACCAGTATCAGAGCCAG
GAGACCTTCAGACTCCAAGAGCGGGGCCTCTGTGTCCGCTGCTCCTGCC
AGGCTGGCGAGGTCTCCTGTGAGGAGCAGGAGTGCCCAGTCACCCCCTG
TGCCCTGCCTGCCTCTGGCCGCCAGCTCTGCCCAGCCTGTGAGCTGGAT
GGAGAGGAGTTTGCTGAGGGAGTCCAGTGGGAGCCTGATGGTCGGCCCT
GCACCGCCTGCGTCTGTCAAGATGGGGTACCCAAGTGCGGGGCTGTGCT
CTGCCCCCCAGCCCCCTGCCAGCACCCCACCCAGCCCCCTGGTGCCTGC
TGCCCCAGCTGTGACAGCTGCACCTACCACAGCCAAGTGTATGCCAATG
GGCAGAACTTCACGGATGCAGACAGCCCTTGCCATGCCTGCCACTGTCA
GGATGGAACTGTGACATGCTCCTTGGTTGACTGCCCTCCCACGACCTGT
GCCAGGCCCCAGAGTGGACCAGGCCAGTGTTGCCCCAGGTGCCCAGACT
GCATCCTGGAGGAAGAGGTGTTTGTGGACGGCGAGAGCTTCTCCCACCC
CCGAGACCCCTGCCAGGAGTGCCGATGCCAGGAAGGCCATGCCCACTGC
CAGCCTCGCCCCTGCCCCAGGGCCCCCTGTGCCCACCCGCTGCCTGGGA
CCTGCTGCCCGAACGACTGCAGCGGCTGTGCCTTTGGCGGGAAAGAGTA
CCCCAGCGGAGCGGACTTCCCCCACCCCTCTGACCCCTGCCGTCTGTGT
CGCTGTCTGAGCGGCAACGTGCAGTGCCTGGCCCGCCGCTGCGTGCCGC
TGCCCTGTCCAGAGCCTGTCCTGCTGCCGGGAGAGTGCTGCCCGCAGTG
CCCAGCCCCCGCCGGCTGCCCACGGCCCGGCGCGGCCCACGCCCGCCAC
CAGGAGTACTTCTCCCCGCCCGGCGATCCCTGCCGCCGCTGCCTCTGCC
TCGACGGCTCCGTGTCCTGCCAGCGGCTGCCCTGCCCGCCCGCGCCCTG
CGCGCACCCGCGCCAGGGGCCTTGCTGCCCCTCCTGCGACGGCTGCCTG
TACCAGGGGAAGGAGTTTGCCAGCGGGGAGCGCTTCCCATCGCCCACTG
CTGCCTGCCACCTCTGCCTTTGCTGGGAGGGCAGCGTGAGCTGCGAGCC
CAAGGCATGTGCCCCTGCACTGTGCCCCTTCCCTGCCAGGGGCGACTGC
TGCCCTGACTGTGATGGCTGTGAGTACCTGGGGGAGTCCTACCTGAGTA
ACCAGGAGTTCCCAGACCCCCGAGAACCCTGCAACCTGTGTACCTGTCT
TGGAGGCTTCGTGACCTGCGGCCGCCGGCCCTGTGAGCCTCCGGGCTGC
AGCCACCCACTCATCCCCTCTGGGCACTGCTGCCCGACCTGCCAGGGAT
GCCGCTACCATGGCGTCACTACTGCCTCCGGAGAGACCCTTCCTGACCC
ACTTGACCCTACCTGCTCCCTCTGCACCTGCCAGGAAGGTTCCATGCGC
TGCCAGAAGAAGCCATGTCCCCCAGCTCTCTGCCCCCACCCCTCTCCAG
GCCCCTGCTTCTGCCCTGTTTGCCACAGCTGTCTCTCTCAGGGCCGGGA
GCACCAGGATGGGGAGGAGTTTGAGGGACCAGCAGGCAGCTGTGAGTGG
TGTCGCTGTCAGGCTGGCCAGGTCAGCTGTGTGCGGCTGCAGTGCCCAC
CCCTTCCCTGCAAGCTCCAGGTCACCGAGCGGGGGAGCTGCTGCCCTCG
CTGCAGAGGCTGCCTGGCTCATGGGGAAGAGCACCCCGAAGGCAGTAGA
TGGGTGCCCCCCGACAGTGCCTGCTCCTCCTGTGTGTGTCACGAGGGCG
TCGTCACCTGTGCACGCATCCAGTGCATCAGCTCTTGCGCCCAGCCCCG
CCAAGGGCCCCATGACTGCTGTCCTCAATGCTCTGACTGTGAGCATGAG
GGCCGGAAGTACGAGCCTGGGGAGAGCTTCCAGCCTGGGGCAGACCCCT
GTGAAGTGTGCATCTGCGAGCCACAGCCTGAGGGGCCTCCCAGCCTTCG
CTGTCACCGGCGGCAGTGTCCCAGCCTGGTGGGCTGCCCCCCCAGCCAG
CTCCTGCCCCCTGGGCCCCAGCACTGCTGTCCCACCTGTGCCGAGGCCT
TGAGTAACTGTTCAGAGGGCCTGCTGGGATCTGAGCTAGCCCCACCAGA
CCCCTGCTACACGTGCCAGTGCCAGGACCTGACATGGCTCTGCATCCAC
CAGGCTTGTCCTGAGCTCAGCTGTCCCCTCTCAGAGCGCCACACTCCCC
CTGGGAGCTGCTGCCCCGTATGCCGGGCTCCCACCCAGTCCTGCGTGCA
CCAGGGCCGTGAGGTGGCCTCTGGAGAGCGCTGGACTGTGGACACCTGC
ACCAGCTGCTCCTGCATGGCGGGCACCGTGCGTTGCCAGAGCCAGCGCT
GCTCACCGCTCTCGTGTGGCCCCGACAAGGCCCCTGCCCTGAGTCCTGG
CAGCTGCTGCCCCCGCTGCCTGCCTCGGCCCGCTTCCTGCATGGCCTTC
GGAGACCCCCATTACCGCACCTTCGACGGCCGCCTGCTGCACTTCCAGG
GCAGTTGCAGCTATGTGCTGGCCAAGGACTGCCACAGCGGGGACTTCAG
TGTGCACGTGACCAATGATGACCGGGGCCGGAGCGGTGTGGCCTGGACC
CAGGAGGTGGCGGTGCTGCTGGGAGACATGGCCGTGCGGCTGCTGCAGG
ACGGGGCAGTCACGGTGGATGGGCACCCGGTGGCCTTGCCCTTCCTGCA
GGAGCCGCTGCTGTATGTGGAGCTGCGAGGACACACTGTGATCCTGCAC
GCCCAGCCCGGGCTCCAGGTGCTGTGGGATGGGCAGTCCCAGGTGGAGG
TGAGCGTACCTGGCTCCTACCAGGGCCGGACTTGTGGGCTCTGTGGGAA
CTTCAATGGCTTTGCCCAGGACGATCTGCAGGGCCCTGAGGGGCTGCTC
CTGCCCTCGGAGGCTGCGTTTGGGAATAGCTGGCAGGTCTCAGAGGGGC
TGTGGCCTGGCCGGCCCTGTTCTGCAGGCCGAGAGGTGGATCCGTGCCG
GGCAGCAGGTTACCGTGCCAGGCGTGAGGCCAATGCCCGGTGTGGGGTG
CTGAAGTCCTCCCCATTCAGTCGCTGCCATGCTGTGGTGCCACCGGAGC
CCTTCTTTGCCGCCTGTGTGTATGACCTGTGTGCCTGTGGCCCTGGCTC
CTCCGCTGATGCCTGCCTCTGTGATGCCCTGGAAGCCTACGCCAGTCAC
TGTCGCCAGGCAGGAGTGACACCTACCTGGCGAGGCCCCACGCTGTGTG
TGGTAGGCTGCCCCCTGGAGCGTGGCTTCGTGTTTGATGAGTGCGGCCC
ACCCTGTCCCCGCACCTGCTTCAATCAGCATATCCCCCTGGGGGAGCTG
GCAGCCCACTGCGTGAGGCCCTGCGTGCCCGGCTGCCAGTGCCCTGCAG
GCCTGGTGGAGCATGAGGCCCACTGCATCCCACCCGAGGCCTGCCCCCA
AGTCCTGCTCACTGGAGACCAGCCACTTGGTGCTCGGCCCAGCCCCAGC
CGGGAGCCCCAGGAGACACCC

A human CRIM2 isoform 2 precursor protein sequence (NCBI Ref Seq NP_955381.2) is as follows:

| (SEQ ID NO: 45) | |
|---|---|
| 1 | MAGVGAAALS LLLHLGALAL AAGAEGGAVP REPPGQQTTA HSSVLAGNSQ EQWHPLREWL |
| 61 | GRLEAAVMEL REQNKDLQTR VRQLESCECH PASPQCWGLG RAWPEGARWE PDACTACVCQ |
| 121 | DGAAHCGPQA HLPHCRGCSQ NGQTYGNGET FSPDACTTCR CLEGTITCNQ KPCPRGPCPE |
| 181 | PGACCPHCKP GCDYEGQLYE EGVTFLSSSN PCLQCTCLRS RVRCMALKCP PSPCPEPVLR |
| 241 | PGHCCPTCQG CTEGGSHWEH GQEWTTPGDP CRICRCLEGH IQCRQRECAS LCPYPARPLP |
| 301 | GTCCPVCDGC FLNGREHRSG EPVGSGDPCS HCRCANGSVQ CEPLPCPPVP CRHPGKIPGQ |
| 361 | CCPVCDGCEY QGHQYQSQET FRLQERGLCV RCSCQAGEVS CEEQECPVTP CALPASGRQL |
| 421 | CPACELDGEE FAEGVQWEPD GRPCTACVCQ DGVPKCGAVL CPPAPCQHPT QPPGACCPSC |
| 481 | DSCTYHSQVY ANGQNFTDAD SPCHACHCQD GTVTCSLVDC PPTTCARPQS GPGQCCPRCP |
| 541 | DCILEEEVFV DGESFSHPRD PCQECRCQEG HAHCQPRPCP RAPCAHPLPG TCCPNDCSGC |
| 601 | AFGGKEYPSG ADFPHPSDPC RLCRCLSGNV QCLARRCVPL PCPEPVLLPG ECCPQCPAAP |
| 661 | APAGCPRPGA AHARHQEYFS PPGDPCRRCL CLDGSVSCQR LPCPPAPCAH PRQGPCCPSC |
| 721 | DGCLYQGKEF ASGERFPSPT AACHLCLCWE GSVSCEPKAC APALCPFPAR GDCCPDCDGE |
| 781 | GHGIGSCRGG MRETRGLGQN NLYCPRVDLK YLLQ |

A processed CRIM2 isoform 2 sequence is as follows:

(SEQ ID NO: 46)
AEGGAVPREPPGQQTTAHSSVLAGNSQEQWHPLREWLGRLEAAVMELRE
QNKDLQTRVRQLESCECHPASPQCWGLGRAWPEGARWEPDACTACVCQD
GAAHCGPQAHLPHCRGCSQNGQTYGNGETFSPDACTTCRCLEGTITCNQ
KPCPRGPCPEPGACCPHCKPGCDYEGQLYEEGVTFLSSSNPCLQCTCLR
SRVRCMALKCPPSPCPEPVLRPGHCCPTCQGCTEGGSHWEHGQEWTTPG
DPCRICRCLEGHIQCRQRECASLCPYPARPLPGTCCPVCDGCFLNGREH
RSGEPVGSGDPCSHCRCANGSVQCEPLPCPPVPCRHPGKIPGQCCPVCD
GCEYQGHQYQSQETFRLQERGLCVRCSCQAGEVSCEEQECPVTPCALPA
SGRQLCPACELDGEEFAEGVQWEPDGRPCTACVCQDGVPKCGAVLCPPA
PCQHPTQPPGACCPSCDSCTYHSQVYANGQNFTDADSPCHACHCQDGTV
TCSLVDCPPTTCARPQSGPGQCCPRCPDCILEEEVFVDGESFSHPRDPC
QECRCQEGHAHCQPRPCPRAPCAHPLPGTCCPNDCSGCAFGGKEYPSGA
DFPHPSDPCRLCRCLSGNVQCLARRCVPLPCPEPVLLPGECCPQCPAAP
APAGCPRPGAAHARHQEYFSPPGDPCRRCLCLDGSVSCQRLPCPPAPCA
HPRQGPCCPSCDGCLYQGKEFASGERFPSPTAACHLCLCWEGSVSCEPK
ACAPALCPFPARGDCCPDCDGEGHGIGSCRGGMRETRGLGQNNLYCPRV
DLKYLLQ

A nucleic acid sequence encoding an unprocessed human CRIM2 isoform 2 precursor protein is shown below (SEQ ID NO: 47), corresponding to nucleotides 44-2485 of NCBI Reference Sequence NM_199349.2. The signal sequence is underlined.

(SEQ ID NO: 47)
ATGGCCGGGGTCGGGGCCGCTGCGCTGTCCCTTCTCCTGCACCTCGGGG
CCCTGGCGCTGGCCGCGGGCGCGGAAGGTGGGGCTGTCCCCAGGGAGCC
CCCTGGGCAGCAGACAACTGCCCATTCCTCAGTCCTTGCTGGGAACTCC
CAGGAGCAGTGGCACCCCCTGCGAGAGTGGCTGGGGCGACTGGAGGCTG
CAGTGATGGAGCTCAGAGAACAGAATAAGGACCTGCAGACGAGGGTGAG
GCAGCTGGAGTCCTGTGAGTGCCACCCTGCATCTCCCCAGTGCTGGGGG
CTGGGGCGTGCCTGGCCCGAGGGGGCACGCTGGGAGCCTGACGCCTGCA
CAGCCTGCGTCTGCCAGGATGGGGCCGCTCACTGTGGCCCCCAAGCACA
CCTGCCCCATTGCAGGGGCTGCAGCCAAAATGGCCAGACCTACGGCAAC
GGGGAGACCTTCTCCCCAGATGCCTGCACCACCTGCCGCTGTCTGGAAG
GTACCATCACTTGCAACCAGAAGCCATGCCCAAGAGGACCCTGCCCTGA
GCCAGGAGCATGCTGCCCGCACTGTAAGCCAGGCTGTGATTATGAGGGG
CAGCTTTATGAGGAGGGGGTCACCTTCCTGTCCAGCTCCAACCCTTGTC
TACAGTGCACCTGCCTGAGGAGCCGAGTTCGCTGCATGGCCCTGAAGTG
CCCGCCTAGCCCCTGCCCAGAGCCAGTGCTGAGGCCTGGGCACTGCTGC
CCAACCTGCCAAGGCTGCACAGAAGGTGGCTCTCACTGGGAACATGGCC
AAGAGTGGACAACACCTGGGGACCCCTGCCGAATCTGCCGGTGCCTGGA
GGGTCACATCCAGTGCCGCCAGCGAGAATGTGCCAGCCTGTGTCCATAC
CCAGCCCGGCCCCTCCCAGGCACCTGCTGCCCTGTGTGTGATGGCTGTT
TCCTAAACGGGCGGGAGCACCGCAGCGGGGAGCCTGTGGGCTCAGGGGA
CCCCTGCTCGCACTGCCGCTGTGCTAATGGGAGTGTCCAGTGTGAGCCT
CTGCCCTGCCCGCCAGTGCCCTGCAGACACCCAGGCAAGATCCCTGGGC
AGTGCTGCCCTGTCTGCGATGGCTGTGAGTACCAGGGACACCAGTATCA
GAGCCAGGAGACCTTCAGACTCCAAGAGCGGGGCCTCTGTGTCCGCTGC
TCCTGCCAGGCTGGCGAGGTCTCCTGTGAGGAGCAGGAGTGCCCAGTCA
CCCCCTGTGCCCTGCCTGCCTCTGGCCGCCAGCTCTGCCCAGCCTGTGA
GCTGGATGGAGAGGAGTTTGCTGAGGGAGTCCAGTGGGAGCCTGATGGT
CGGCCCTGCACCGCCTGCGTCTGTCAAGATGGGGTACCCAAGTGCGGGG
CTGTGCTCTGCCCCCCAGCCCCCTGCCAGCACCCCACCCAGCCCCCTGG
TGCCTGCTGCCCCAGCTGTGACAGCTGCACCTACCACAGCCAAGTGTAT
GCCAATGGGCAGAACTTCACGGATGCAGACAGCCCTTGCCATGCCTGCC
ACTGTCAGGATGGAACTGTGACATGCTCCTTGGTTGACTGCCCTCCCAC
GACCTGTGCCAGGCCCCAGAGTGGACCAGGCCAGTGTTGCCCCAGGTGC
CCAGACTGCATCCTGGAGGAAGAGGTGTTTGTGGACGGCGAGAGCTTCT
CCCACCCCCGAGACCCCTGCCAGGAGTGCCGATGCCAGGAAGGCCATGC
CCACTGCCAGCCTCGCCCCTGCCCCAGGGCCCCCTGTGCCCACCCGCTG
CCTGGGACCTGCTGCCCGAACGACTGCAGCGGCTGTGCCTTTGGCGGGA
AAGAGTACCCCAGCGGAGCGGACTTCCCCCACCCCTCTGACCCCTGCCG
TCTGTGTCGCTGTCTGAGCGGCAACGTGCAGTGCCTGGCCCGCCGCTGC
GTGCCGCTGCCCTGTCCAGAGCCTGTCCTGCTGCCGGGAGAGTGCTGCC
CGCAGTGCCCAGCCGCCCCAGCCCCCGCCGGCTGCCCACGGCCCGGCGC
GGCCCACGCCCGCCACCAGGAGTACTTCTCCCCGCCCGGCGATCCCTGC
CGCCGCTGCCTCTGCCTCGACGGCTCCGTGTCCTGCCAGCGGCTGCCCT
GCCCGCCCGCGCCCTGCGCGCACCCGCGCCAGGGGCCTTGCTGCCCCTC
CTGCGACGGCTGCCTGTACCAGGGGAAGGAGTTTGCCAGCGGGGAGCGC
TTCCCATCGCCCACTGCTGCCTGCCACCTCTGCCTTTGCTGGGAGGGCA
GCGTGAGCTGCGAGCCCAAGGCATGTGCCCCTGCACTGTGCCCCTTCCC
TGCCAGGGGCGACTGCTGCCCTGACTGTGATGGTGAGGGTCATGGGATA
GGGAGCTGCCGGGGTGGGATGCGGGAGACCAGAGGGCTGGGTCAGAATA
ATCTTTACTGCCCTAGGGTGGATCTAAAATATTTATTACAG

A nucleic acid sequence encoding a processed CRIM2 isoform 2 is shown below (SEQ ID NO: 48):

(SEQ ID NO: 48)
GCGGAAGGTGGGGCTGTCCCCAGGGAGCCCCCTGGGCAGCAGACAACTGC
CCATTCCTCAGTCCTTGCTGGGAACTCCCAGGAGCAGTGGCACCCCCTGC
GAGAGTGGCTGGGGCGACTGGAGGCTGCAGTGATGGAGCTCAGAGAACAG
AATAAGGACCTGCAGACGAGGGTGAGGCAGCTGGAGTCCTGTGAGTGCCA
CCCTGCATCTCCCCAGTGCTGGGGGCTGGGGCGTGCCTGGCCCGAGGGGG
CACGCTGGGAGCCTGACGCCTGCACAGCCTGCGTCTGCCAGGATGGGGCC
GCTCACTGTGGCCCCCAAGCACACCTGCCCCATTGCAGGGGCTGCAGCCA
AAATGGCCAGACCTACGGCAACGGGGAGACCTTCTCCCCAGATGCCTGCA
CCACCTGCCGCTGTCTGGAAGGTACCATCACTTGCAACCAGAAGCCATGC
CCAAGAGGACCCTGCCCTGAGCCAGGAGCATGCTGCCCGCACTGTAAGCC
AGGCTGTGATTATGAGGGGCAGCTTTATGAGGAGGGGGTCACCTTCCTGT
CCAGCTCCAACCCTTGTCTACAGTGCACCTGCCTGAGGAGCCGAGTTCGC
TGCATGGCCCTGAAGTGCCCGCCTAGCCCCTGCCCAGAGCCAGTGCTGAG
GCCTGGGCACTGCTGCCCAACCTGCCAAGGCTGCACAGAAGGTGGCTCTC
ACTGGGAACATGGCCAAGAGTGGACAACACCTGGGGACCCCTGCCGAATC
TGCCGGTGCCTGGAGGGTCACATCCAGTGCCGCCAGCGAGAATGTGCCAG
CCTGTGTCCATACCCAGCCCGGCCCCTCCCAGGCACCTGCTGCCCTGTGT
GTGATGGCTGTTTCCTAAACGGGCGGGAGCACCGCAGCGGGGAGCCTGTG
GGCTCAGGGGACCCCTGCTCGCACTGCCGCTGTGCTAATGGGAGTGTCCA
GTGTGAGCCTCTGCCCTGCCCGCCAGTGCCCTGCAGACACCCAGGCAAGA
TCCCTGGGCAGTGCTGCCCTGTCTGCGATGGCTGTGAGTACCAGGGACAC
CAGTATCAGAGCCAGGAGACCTTCAGACTCCAAGAGCGGGGCCTCTGTGT
CCGCTGCTCCTGCCAGGCTGGCGAGGTCTCCTGTGAGGAGCAGGAGTGCC
CAGTCACCCCCTGTGCCCTGCCTGCCTCTGGCCGCCAGCTCTGCCCAGCC
TGTGAGCTGGATGGAGAGGAGTTTGCTGAGGGAGTCCAGTGGGAGCCTGA
TGGTCGGCCCTGCACCGCCTGCGTCTGTCAAGATGGGGTACCCAAGTGCG
GGGCTGTGCTCTGCCCCCCAGCCCCCTGCCAGCACCCCACCCAGCCCCCT
GGTGCCTGCTGCCCCAGCTGTGACAGCTGCACCTACCACAGCCAAGTGTA
TGCCAATGGGCAGAACTTCACGGATGCAGACAGCCCTTGCCATGCCTGCC
ACTGTCAGGATGGAACTGTGACATGCTCCTTGGTTGACTGCCCTCCCACG
ACCTGTGCCAGGCCCCAGAGTGGACCAGGCCAGTGTTGCCCCAGGTGCCC
AGACTGCATCCTGGAGGAAGAGGTGTTTGTGGACGGCGAGAGCTTCTCCC
ACCCCCGAGACCCCTGCCAGGAGTGCCGATGCCAGGAAGGCCATGCCCAC
TGCCAGCCTCGCCCCTGCCCCAGGGCCCCCTGTGCCCACCCGCTGCCTGG
GACCTGCTGCCCGAACGACTGCAGCGGCTGTGCCTTTGGCGGGAAAGAGT
ACCCCAGCGGAGCGGACTTCCCCCACCCCTCTGACCCCTGCCGTCTGTGT
CGCTGTCTGAGCGGCAACGTGCAGTGCCTGGCCCGCCGCTGCGTGCCGCT
GCCCTGTCCAGAGCCTGTCCTGCTGCCGGGAGAGTGCTGCCCGCAGTGCC
CAGCCGCCCCAGCCCCCGCCGGCTGCCCACGGCCCGGCGCGGCCCACGCC
CGCCACCAGGAGTACTTCTCCCCGCCCGGCGATCCCTGCCGCCGCTGCCT
CTGCCTCGACGGCTCCGTGTCCTGCCAGCGGCTGCCCTGCCCGCCCGCGC
CCTGCGCGCACCCGCGCCAGGGGCCTTGCTGCCCCTCCTGCGACGGCTGC
CTGTACCAGGGGAAGGAGTTTGCCAGCGGGGAGCGCTTCCCATCGCCCAC
TGCTGCCTGCCACCTCTGCCTTTGCTGGGAGGGCAGCGTGAGCTGCGAGC
CCAAGGCATGTGCCCCTGCACTGTGCCCCTTCCCTGCCAGGGGCGACTGC
TGCCCTGACTGTGATGGTGAGGGTCATGGGATAGGGAGCTGCCGGGGTGG
GATGCGGGAGACCAGAGGGCTGGGTCAGAATAATCTTTACTGCCCTAGGG
TGGATCTAAAATATTTATTACAG

In certain embodiments, the disclosure relates to heteromultimers that comprise at least one CRIM2 polypeptide, which includes fragments, functional variants, and modified forms thereof. Preferably, CRIM2 polypeptides for use in accordance with the disclosure (e.g., heteromultimers comprising a CRIM2 polypeptide and uses thereof) are soluble (e.g., an extracellular domain of CRIM2). In other preferred embodiments, CRIM2 polypeptides for use in accordance with the disclosure bind to and/or inhibit (antagonize) activity (e.g., Smad signaling) of one or more TGF-beta superfamily ligands. In some embodiments, heteromultimers of the disclosure at least one CRIM2 polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 41, 42, 45, or 46. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 26-138 (e.g., amino acid residues 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, and 138) of SEQ ID NO: 41, and ends at any one of amino acids 1298-1503 (e.g., amino acid residues 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1335, 1336, 1337, 1338, 1339, 1340, 1341, 1342, 1343, 1344, 1345, 1346, 1347, 1348, 1349, 1350, 1351, 1352, 1353, 1354, 1355, 1356, 1357, 1358, 1359, 1360, 1361, 1362, 1363, 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, 1406, 1407, 1408, 1409, 1410, 1411, 1412, 1413, 1414, 1415, 1416, 1417, 1418, 1419, 1420, 1421, 1422, 1423, 1424, 1425, 1426, 1427, 1428, 1429, 1430, 1431, 1432, 1433, 1434, 1435, 1435, 1436, 1437, 1438, 1439, 1440, 1441, 1442, 1443, 1444, 1445, 1446, 1447, 1448, 1349, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, or 1503) of SEQ ID NO: 41. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 26-1298 of SEQ ID NO: 41. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 26-1503 of SEQ ID NO: 41. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 138-1298 of SEQ ID NO: 41. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 138-1503 of SEQ ID NO: 41. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 24-138 (e.g., amino acid residues 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, or 138) of SEQ ID NO: 45, and ends at any one of amino acids 539-814 (e.g., amino acid residues 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 414, 615, 616, 617, 618, 619, 620, 621, 622, 623, 624, 625, 626, 627, 628, 629, 630, 631, 632, 633, 634, 635, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 405, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, or 814) of SEQ ID NO: 45. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 24-539 of SEQ ID NO: 45. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 24-814 of SEQ ID NO: 45. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 138-539 of SEQ ID NO: 45. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 138-814 of SEQ ID NO: 45. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 27-87 (e.g., amino acid residues 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, and 87) of SEQ ID NO: 41, and ends at any one of amino acids 1478-1503 (e.g., amino acid residues 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, or 1503) of SEQ ID NO: 41. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 27-1503 of SEQ ID NO: 41. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 87-1478 of SEQ ID NO: 41. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 24-87 (e.g., amino acid residues 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, and 87) of SEQ ID NO: 45, and ends at any one of amino acids 804-814 (e.g., amino acid residues 804, 805, 806, 807, 808, 809, 810, 811, 812, 813, or 814) of SEQ ID NO: 45. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 24-814 of SEQ ID NO: 45. In some embodiments, heteromultimers of the disclosure comprise at least one CRIM2 polypeptide that is at least 70%, 75%, 80%, 85%, 90%, 91%, 92%, 93%, 94%, 95%, 96%, 97%, 98%, 99%, or 100% identical to amino acids of 87-804 of SEQ ID NO: 45.

The term “BAMBI polypeptide” includes polypeptides comprising any naturally occurring BAMBI protein (encoded by BAMBI or one of its nonhuman orthologs) as well as any variants thereof (including mutants, fragments, fusions, and peptidomimetic forms) that retain a useful activity.

The human BAMBI precursor protein sequence (NCBI Ref Seq NP_036474.1) is as follows:

| (SEQ ID NO: 49) | |
|---|---|
| 1 | MDRHSSYIFI WLQLELCAMA VLLTKGEIRC YCDAAHCVAT GYMCKSELSA CFSRLLDPQN |
| 61 | SNSPLTHGCL DSLASTTDIC QAKQARNHSG TTIPTLECCH EDMCNYRGLH DVLSPPRGEA |
| 121 | 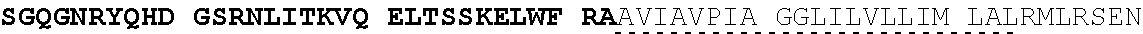 |
| 181 | KRLQDQRQQM LSRLHYSFHG HHSKKGQVAK LDLECMVPVS GHENCCLTCD KMRQADLSND |
| 241 | KILSLVHWGM YSGHGKLEFV |

The signal peptide is indicated by single underline, the extracellular domain is indicated in bold font, and the transmembrane domain is indicated by dotted underline.

A processed BAMBI polypeptide sequence is as follows:

(SEQ ID NO: 50)
VLLTKGEIRCYCDAAHCVATGYMCKSELSACFSRLLDPQNSNSPLTHGCL
DSLASTTDICQAKQARNHSGTTIPTLECCHEDMCNYRGLHDVLSPPRGEA
SGQGNRYQHDGSRNLITKVQELTSSKELWFRA

A nucleic acid sequence encoding unprocessed human BAMBI precursor protein is shown below (SEQ ID NO: 51), corresponding to nucleotides 404-1183 of NCBI Reference Sequence NM_012342.2. The signal sequence is indicated by solid underline and the transmembrane domain by dotted underline.

(SEQ ID NO: 51)
ATGGATCGCCACTCCAGCTACATCTTCATCTGGCTGCAGCTGGAGCTCTGCGCCATGGCCGTGCTGCTCACCAAA
GGTGAAATTCGATGCTACTGTGATGCTGCCCACTGTGTAGCCACTGGTTATATGTGTAAATCTGAGCTCAGCGCC
TGCTTCTCTAGACTTCTTGATCCTCAGAACTCAAATTCCCCACTCACCCATGGCTGCCTGGACTCTCTTGCAAGC
ACGACAGACATCTGCCAAGCCAAACAGGCCCGAAACCACTCTGGCACCACCATACCCACATTGGAATGCTGTCAT
GAAGACATGTGCAATTACAGAGGGCTGCACGATGTTCTCTCTCCTCCCAGGGGTGAGGCCTCAGGACAAGGAAAC
AGGTATCAGCATGATGGTAGCAGAAACCTTATCACCAAGGTGCAGGAGCTGACTTCTTCCAAAGAGTTGTGGTTC

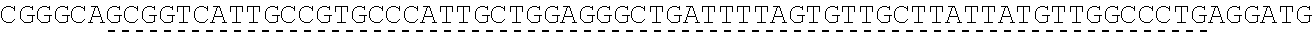

CTTCGAAGTGAAAATAAGAGGCTGCAGGATCAGCGGCAACAGATGCTCTCCCGTTTGCACTACAGCTTTCACGGA
CACCATTCCAAAAAGGGGCAGGTTGCAAAGTTAGACTTGGAATGCATGGTGCCGGTCAGTGGGCACGAGAACTGC
TGTCTGACCTGTGATAAAATGAGACAAGCAGACCTCAGCAACGATAAGATCCTCTCGCTTGTTCACTGGGGCATG
TACAGTGGGCACGGGAAGCTGGAATTCGTA

A nucleic acid sequence encoding a processed extracellular BAMBI is shown below (SEQ ID NO: 52):

(SEQ ID NO: 52)
GTGCTGCTCACCAAAGGTGAAATTCGATGCTACTGTGATGCTGCCCACT
GTGTAGCCACTGGTTATATGTGTAAATCTGAGCTCAGCGCCTGCTTCTC
TAGACTTCTTGATCCTCAGAACTCAAATTCCCCACTCACCCATGGCTGC
CTGGACTCTCTTGCAAGCACGACAGACATCTGCCAAGCCAAACAGGCCC
GAAACCACTCTGGCACCACCATACCCACATTGGAATGCTGTCATGAAGA
CATGTGCAATTACAGAGGGCTGCACGATGTTCTCTCTCCTCCCAGGGGT
GAGGCCTCAGGACAAGGAAACAGGTATCAGCATGATGGTAGCAGAAACC
TTATCACCAAGGTGCAGGAGCTGACTTCTTCCAAAGAGTTGTGGTTCCG
GGCA

In certain embodiments, the disclosure relates to heteromultimers that comprise at least one BAMBI polypeptide, which includes fragments, functional variants, and modified forms thereof. Preferably, BAMBI polypeptides for use in accordance with the disclosure (e.g., heteromultimers comprising a BAMBI polypeptide and uses thereof) are soluble (e.g., an extracellular domain of BAMBI). In other preferred embodiments, BAMBI polypeptides for use in accordance with disclosure bind to and/or inhibit (antagonize) activity (e.g., Smad signaling) of one or more TGF-beta superfamily ligands. In some embodiments, heteromultimers of the disclosure comprise at least one BAMBI polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 49 or 50. In some embodiments, heteromultimers of the disclosure comprise at least one BAMBI polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-30 (e.g., amino acid residues 21, 22, 23, 24, 25, 26, 27, 28, 29, or 30) of SEQ ID NO: 49, and ends at any one of amino acids 104-152 (e.g., amino acid residues 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, or 152) of SEQ ID NO: 49. In some embodiments, heteromultimers of the disclosure comprise at least one BAMBI polypeptide that is at least 70%, 75%, 80%, 85%, 90%, 91%, 92%, 93%, 94%, 95%, 96%, 97%, 98%, 99%, or 100% identical to amino acids of 21-104 of SEQ ID NO: 49. In some embodiments, heteromultimers of the disclosure comprise at least one BAMBI polypeptide that is at least 70%, 75%, 80%, 85%, 90%, 91%, 92%, 93%, 94%, 95%, 96%, 97%, 98%, 99%, or 100% identical to amino acids of 21-152 of SEQ ID NO: 49. In some embodiments, heteromultimers of the disclosure comprise at least one BAMBI polypeptide that is at least 70%, 75%, 80%, 85%, 90%, 91%, 92%, 93%, 94%, 95%, 96%, 97%, 98%, 99%, or 100% identical to amino acids of 30-104 of SEQ ID NO: 49. In some embodiments, heteromultimers of the disclosure comprise at least one BAMBI polypeptide that is at least 70%, 75%, 80%, 85%, 90%, 91%, 92%, 93%, 94%, 95%, 96%, 97%, 98%, 99%, or 100% identical to amino acids of 30-152 of SEQ ID NO: 49. In some embodiments, heteromultimers of the disclosure comprise at least one BAMBI polypeptide that is at least 70%, 75%, 80%, 85%, 90%, 91%, 92%, 93%, 94%, 95%, 96%, 97%, 98%, 99%, or 100% identical to amino acids of 27-152 of SEQ ID NO: 49.

The term “BMPER polypeptide” includes polypeptides comprising any naturally occurring BMPER protein (encoded by BMPER or one of its nonhuman orthologs) as well as any variants thereof (including mutants, fragments, fusions, and peptidomimetic forms) that retain a useful activity.

A human BMPER precursor protein sequence (NCBI Ref Seq NP_597725.1) is as follows:

| (SEQ ID NO: 53) | |
|---|---|
| 1 | MLWFSGVGAL AERYCRRSPG ITCCVLLLLN CSGVPMSLAS SFLTGSVAKC ENEGEVLQIP |
| 61 | FITDNPCIMC VCLNKEVTCK REKCPVLSRD CALAIKQRGA CCEQCKGCTY EGNTYNSSFK |
| 121 | WQSPAEPCVL RQCQEGVVTE SGVRCVVHCK NPLEHLGMCC PTCPGCVFEG VQYQEGEEFQ |
| 181 | PEGSKCTKCS CTGGRTQCVR EVCPILSCPQ HLSHIPPGQC CPKCLGQRKV FDLPFGSCLF |
| 241 | RSDVYDNGSS FLYDNCTACT CRDSTVVCKR KCSHPGGCDQ GQEGCCEECL LRVPPEDIKV |
| 301 | CKFGNKIFQD GEMWSSINCT ICACVKGRTE CRNKQCIPIS SCPQGKILNR KGCCPICTEK |
| 361 | PGVCTVFGDP HYNTFDGRTF NFQGTCQYVL TKDCSSPASP FQVLVKNDAR RTRSFSWTKS |
| 421 | VELVLGESRV SLQQHLTVRW NGSRIALPCR APHFHIDLDG YLLKVTTKAG LEISWDGDSF |
| 481 | VEVMAAPHLK GKLCGLCGNY NGHKRDDLIG GDGNFKFDVD DFAESWRVES NEFCNRPQRK |
| 541 | PVPELCQGTV KVKLRAHREC QKLKSWEFQT CHSTVDYATF YRSCVTDMCE CPVHKNCYCE |
| 601 | SFLAYTRACQ REGIKVHWEP QQNCAATQCK HGAVYDTCGP GCIKTCDNWN EIGPCNKPCV |
| 661 | AGCHCPANLV LHKGRCIKPV LCPQR |

The signal peptide is indicated by a single underline.

A mature BMPER polypeptide sequence is as follows:

(SEQ ID NO: 54)
SSFLTGSVAKCENEGEVLQIPFITDNPCIMCVCLNKEVTCKREKCPVLSR
DCALAIKQRGACCEQCKGCTYEGNTYNSSFKWQSPAEPCVLRQCQEGVVT
ESGVRCVVHCKNPLEHLGMCCPTCPGCVFEGVQYQEGEEFQPEGSKCTKC
SCTGGRTQCVREVCPILSCPQHLSHIPPGQCCPKCLGQRKVFDLPFGSCL
FRSDVYDNGSSFLYDNCTACTCRDSTVVCKRKCSHPGGCDQGQEGCCEEC
LLRVPPEDIKVCKFGNKIFQDGEMWSSINCTICACVKGRTECRNKQCIPI
SSCPQGKILNRKGCCPICTEKPGVCTVFGDPHYNTFDGRTFNFQGTCQYV
LTKDCSSPASPFQVLVKNDARRTRSFSWTKSVELVLGESRVSLQQHLTVR
WNGSRIALPCRAPHFHIDLDGYLLKVTTKAGLEISWDGDSFVEVMAAPHL
KGKLCGLCGNYNGHKRDDLIGGDGNEKEDVDDFAESWRVESNEFCNRPQR
KPVPELCQGTVKVKLRAHRECQKLKSWEFQTCHSTVDYATFYRSCVTDMC
ECPVHKNCYCESFLAYTRACQREGIKVHWEPQQNCAATQCKHGAVYDTCG
PGCIKTCDNWNEIGPCNKPCVAGCHCPANLVLHKGRCIKPVLCPQR

A nucleic acid sequence encoding unprocessed human BMPER precursor protein is shown below (SEQ ID NO: 55), corresponding to nucleotides 375-2429 of NCBI Reference Sequence NM_133468.4. The signal sequence is underlined.

(SEQ ID NO: 55)
ATGCTCTGGTTCTCCGGCGTCGGGGCTCTGGCTGAGCGTTACTGCCGCC
GCTCGCCTGGGATTACGTGCTGCGTCTTGCTGCTACTCAATTGCTCGGG
GGTCCCCATGTCTCTGGCTTCCTCCTTCTTGACAGGTTCTGTTGCAAAA
TGTGAAAATGAAGGTGAAGTCCTCCAGATTCCATTTATCACAGACAACC
CTTGCATAATGTGTGTCTGCTTGAACAAGGAAGTGACATGTAAGAGAGA
GAAGTGCCCCGTGCTGTCCCGAGACTGTGCCCTGGCCATCAAGCAGAGG
GGAGCCTGTTGTGAACAGTGCAAAGGTTGCACCTATGAAGGAAATACCT
ATAACAGCTCCTTCAAATGGCAGAGCCCGGCTGAGCCTTGTGTTCTACG
CCAGTGCCAGGAGGGCGTTGTCACAGAGTCTGGGGTGCGCTGTGTTGTT
CATTGTAAAAACCCTTTGGAGCATCTGGGAATGTGCTGCCCCACATGTC
CAGGCTGTGTGTTTGAGGGTGTGCAGTATCAAGAAGGGGAGGAATTTCA
GCCAGAAGGAAGCAAATGTACCAAGTGTTCCTGCACTGGAGGCAGGACA
CAATGTGTGAGAGAAGTCTGTCCCATTCTCTCCTGTCCCCAGCACCTTA
GTCACATACCCCCAGGACAGTGCTGCCCCAAATGTTTGGGTCAGAGGAA
AGTGTTTGACCTCCCTTTTGGGAGCTGCCTCTTTCGAAGTGATGTTTAT
GACAATGGATCCTCATTTCTGTACGATAACTGCACAGCTTGTACCTGCA
GGGACTCTACTGTGGTTTGCAAGAGGAAGTGCTCCCACCCTGGTGGCTG
TGACCAAGGCCAGGAGGGCTGTTGTGAAGAGTGCCTCCTACGAGTGCCC
CCAGAAGACATCAAAGTATGCAAATTTGGCAACAAGATTTTCCAGGATG
GAGAGATGTGGTCCTCTATCAATTGTACCATCTGTGCTTGTGTGAAAGG
CAGGACGGAGTGTCGCAATAAGCAGTGCATTCCCATCAGTAGCTGCCCA
CAGGGCAAAATTCTCAACAGAAAAGGATGCTGTCCTATTTGCACTGAAA
AGCCCGGCGTTTGCACGGTGTTTGGAGATCCCCACTACAACACTTTTGA
CGGTCGGACATTTAACTTTCAGGGGACGTGTCAGTACGTTTTGACAAAA
GACTGCTCCTCCCCTGCCTCGCCCTTCCAGGTGCTGGTGAAGAACGACG
CCCGCCGGACACGCTCCTTCTCGTGGACCAAGTCGGTGGAGCTGGTGCT
GGGCGAGAGCAGGGTCAGCCTGCAGCAGCACCTCACCGTGCGCTGGAAC
GGCTCGCGCATCGCGCTCCCCTGCCGCGCGCCACACTTCCACATCGACC
TGGATGGCTACCTCTTGAAAGTGACCACCAAAGCAGGTTTGGAAATATC
TTGGGATGGAGACAGTTTTGTAGAAGTCATGGCTGCGCCGCATCTCAAG
GGCAAGCTCTGTGGTCTTTGTGGCAACTACAATGGACATAAACGTGATG
ACTTAATTGGTGGAGATGGAAACTTCAAGTTTGATGTGGATGACTTTGC
TGAATCTTGGAGGGTGGAGTCCAATGAGTTCTGCAACAGACCTCAGAGA
AAGCCAGTGCCTGAACTGTGTCAAGGGACAGTCAAGGTAAAGCTCCGGG
CCCATCGAGAATGCCAAAAGCTCAAATCCTGGGAGTTTCAGACCTGCCA
CTCGACTGTGGACTACGCCACTTTCTACCGGTCCTGTGTGACAGACATG
TGTGAATGTCCAGTCCATAAAAACTGTTATTGCGAGTCATTTTTGGCAT
ATACCCGGGCCTGCCAGAGAGAGGGCATCAAAGTCCACTGGGAGCCTCA
GCAGAATTGTGCAGCCACCCAGTGTAAGCATGGTGCTGTGTACGATACC
TGTGGTCCGGGATGTATCAAGACGTGTGACAACTGGAATGAAATTGGTC
CATGCAACAAGCCGTGCGTTGCTGGGTGCCACTGTCCAGCAAACTTGGT
CCTTCACAAGGGAAGGTGCATCAAGCCAGTCCTTTGTCCCCAGCGG

A nucleic acid sequence encoding a processed BMPER is shown below (SEQ ID NO: 56):

(SEQ ID NO: 56)
TCCTCCTTCTTGACAGGTTCTGTTGCAAAATGTGAAAATGAAGGTGAAG
TCCTCCAGATTCCATTTATCACAGACAACCCTTGCATAATGTGTGTCTG
CTTGAACAAGGAAGTGACATGTAAGAGAGAGAAGTGCCCCGTGCTGTCC
CGAGACTGTGCCCTGGCCATCAAGCAGAGGGGAGCCTGTTGTGAACAGT
GCAAAGGTTGCACCTATGAAGGAAATACCTATAACAGCTCCTTCAAATG
GCAGAGCCCGGCTGAGCCTTGTGTTCTACGCCAGTGCCAGGAGGGCGTT
GTCACAGAGTCTGGGGTGCGCTGTGTTGTTCATTGTAAAAACCCTTTGG
AGCATCTGGGAATGTGCTGCCCCACATGTCCAGGCTGTGTGTTTGAGGG
TGTGCAGTATCAAGAAGGGGAGGAATTTCAGCCAGAAGGAAGCAAATGT
ACCAAGTGTTCCTGCACTGGAGGCAGGACACAATGTGTGAGAGAAGTCT
GTCCCATTCTCTCCTGTCCCCAGCACCTTAGTCACATACCCCCAGGACA
GTGCTGCCCCAAATGTTTGGGTCAGAGGAAAGTGTTTGACCTCCCTTTT
GGGAGCTGCCTCTTTCGAAGTGATGTTTATGACAATGGATCCTCATTTC
TGTACGATAACTGCACAGCTTGTACCTGCAGGGACTCTACTGTGGTTTG
CAAGAGGAAGTGCTCCCACCCTGGTGGCTGTGACCAAGGCCAGGAGGGC
TGTTGTGAAGAGTGCCTCCTACGAGTGCCCCCAGAAGACATCAAAGTAT
GCAAATTTGGCAACAAGATTTTCCAGGATGGAGAGATGTGGTCCTCTAT
CAATTGTACCATCTGTGCTTGTGTGAAAGGCAGGACGGAGTGTCGCAAT
AAGCAGTGCATTCCCATCAGTAGCTGCCCACAGGGCAAAATTCTCAACA
GAAAAGGATGCTGTCCTATTTGCACTGAAAAGCCCGGCGTTTGCACGGT
GTTTGGAGATCCCCACTACAACACTTTTGACGGTCGGACATTTAACTTT
CAGGGGACGTGTCAGTACGTTTTGACAAAAGACTGCTCCTCCCCTGCCT
CGCCCTTCCAGGTGCTGGTGAAGAACGACGCCCGCCGGACACGCTCCTT
CTCGTGGACCAAGTCGGTGGAGCTGGTGCTGGGCGAGAGCAGGGTCAGC
CTGCAGCAGCACCTCACCGTGCGCTGGAACGGCTCGCGCATCGCGCTCC
CCTGCCGCGCGCCACACTTCCACATCGACCTGGATGGCTACCTCTTGAA
AGTGACCACCAAAGCAGGTTTGGAAATATCTTGGGATGGAGACAGTTTT
GTAGAAGTCATGGCTGCGCCGCATCTCAAGGGCAAGCTCTGTGGTCTTT
GTGGCAACTACAATGGACATAAACGTGATGACTTAATTGGTGGAGATGG
AAACTTCAAGTTTGATGTGGATGACTTTGCTGAATCTTGGAGGGTGGAG
TCCAATGAGTTCTGCAACAGACCTCAGAGAAAGCCAGTGCCTGAACTGT
GTCAAGGGACAGTCAAGGTAAAGCTCCGGGCCCATCGAGAATGCCAAAA
GCTCAAATCCTGGGAGTTTCAGACCTGCCACTCGACTGTGGACTACGCC
ACTTTCTACCGGTCCTGTGTGACAGACATGTGTGAATGTCCAGTCCATA
AAAACTGTTATTGCGAGTCATTTTTGGCATATACCCGGGCCTGCCAGAG
AGAGGGCATCAAAGTCCACTGGGAGCCTCAGCAGAATTGTGCAGCCACC
CAGTGTAAGCATGGTGCTGTGTACGATACCTGTGGTCCGGGATGTATCA
AGACGTGTGACAACTGGAATGAAATTGGTCCATGCAACAAGCCGTGCGT
TGCTGGGTGCCACTGTCCAGCAAACTTGGTCCTTCACAAGGGAAGGTGC
ATCAAGCCAGTCCTTTGTCCCCAGCGG

In certain embodiments, the disclosure relates to heteromultimers that comprise at least one BMPER polypeptide, which includes fragments, functional variants, and modified forms thereof. Preferably, BMPER polypeptides for use in accordance with the disclosure (e.g., heteromultimers comprising a BMPER polypeptide and uses thereof) are soluble (e.g., an extracellular domain of BMPER). In other preferred embodiments, BMPER polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 53 or 54. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 40-50 (e.g., amino acid residues 40, 41, 42, 43, 44, 45, 46, 47, 48, 49, or 50) of SEQ ID NO: 53, and ends at any one of amino acids 364-369 (e.g., amino acid residues 364, 365, 366, 367, 368, or 369) of SEQ ID NO: 53. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 (e.g., amino acid residues 370, 371, 372, 373, 374, 375, 376, 377, 378, 379, 380, 381, 382, 383, 284, 385, or 386) of SEQ ID NO: 53, and ends at any one of amino acids 682-685 (e.g., amino acid residues 682, 683, 684, or 685) of SEQ ID NO: 53. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 (e.g., amino acid residues 39, 40, 41, 42, 43, 44, 45, 46, 47, 48, 49, or 50) of SEQ ID NO: 53, and ends at any one of amino acids 682-685 (e.g., amino acid residues 682, 683, 684, or 685) of SEQ ID NO: 53. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 39-364 of SEQ ID NO: 53. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 39-369 of SEQ ID NO: 53. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 39-682 of SEQ ID NO: 53. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 39-685 of SEQ ID NO: 53. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 50-364 of SEQ ID NO: 53. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 50-369 of SEQ ID NO: 53. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 50-682 of SEQ ID NO: 53. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 50-685 of SEQ ID NO: 53. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 370-682 of SEQ ID NO: 53. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 370-685 of SEQ ID NO: 53. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 386-682 of SEQ ID NO: 53. In some embodiments, heteromultimers of the disclosure comprise at least one BMPER polypeptide that is at least 70%, 75%, 80%, 85%, 90%, 91%, 92%, 93%, 94%, 95%, 96%, 97%, 98%, 99%, or 100% identical to amino acids of 386-685 of SEQ ID NO: 53. In some embodiments, heteromultimers of the disclosure comprise at least a BMPER protein, wherein the BMPER protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 (e.g., amino acid residues 39, 40, 41, 42, 43, 44, 45, 46, 47, 48, 49, or 50) of SEQ ID NO: 53, and ends at any one of amino acids 364-369 (e.g., amino acid residues 364, 365, 366, 367, 368, or 369) of SEQ ID NO: 53, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 (e.g., amino acid residues 370, 371, 372, 373, 374, 375, 376, 377, 378, 379, 380, 381, 382, 383, 284, 385, or 386) of SEQ ID NO: 53, and ends at any one of amino acids 682-685 (e.g., amino acid residues 682, 683, 684, or 685) of SEQ ID NO: 53. In some embodiments, heteromultimers of the disclosure comprise at least one single chain ligand trap that comprises a first BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 39-50 (e.g., amino acid residues 39, 40, 41, 42, 43, 44, 45, 46, 47, 48, 49, or 50) of SEQ ID NO: 53, and ends at any one of amino acids 364-369 (e.g., amino acid residues 364, 365, 366, 367, 368, or 369) of SEQ ID NO: 53, and second BMPER polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 370-386 (e.g., amino acid residues 370, 371, 372, 373, 374, 375, 376, 377, 378, 379, 380, 381, 382, 383, 284, 385, or 386) of SEQ ID NO: 53, and ends at any one of amino acids 682-685 (e.g., amino acid residues 682, 683, 684, or 685) of SEQ ID NO: 53.

The term “RGM-B polypeptide” includes polypeptides comprising any naturally occurring RGM-B protein (encoded by RGMB or one of its nonhuman orthologs) as well as any variants thereof (including mutants, fragments, fusions, and peptidomimetic forms) that retain a useful activity.

A human RGM-B precursor protein sequence (NCBI Ref Seq NP_001012779.2) is as follows:

| (SEQ ID NO: 57) | |
|---|---|
| 1 | MIRKKRKRSA PPGPCRSHGP RPATAPAPPP SPEPTRPAWT GMGLRAAPSS AAAAAAEVEQ |
| 61 | RRSPGLCPPP LELLLLLLFS LGLLHAGDCQ QPAQCRIQKC TTDFVSLTSH LNSAVDGFDS |
| 121 | EFCKALRAYA GCTQRTSKAC RGNLVYHSAV LGISDLMSQR NCSKDGPTSS TNPEVTHDPC |
| 181 | NYHSHAGARE HRRGDQNPPS YLFCGLFGDP HLRTFKDNFQ TCKVEGAWPL IDNNYLSVQV |
| 241 | TNVPVVPGSS ATATNKITII FKAHHECTDQ KVYQAVTDDL PAAFVDGTTS GGDSDAKSLR |
| 301 | IVERESGHYV EMHARYIGTT VFVRQVGRYL TLAIRMPEDL AMSYEESQDL QLCVNGCPLS |
| 361 | ERIDDGQGQV SAILGHSLPR TSLVQAWPGY TLETANTQCH EKMPVKDIYF QSCVFDLLTT |
| 421 | GDANFTAAAH SALEDVEALH PRKERWHIFP SSGNGTPRGG SDLSVSLGLT CLILIVFL |

The signal peptide is indicated by single underline.

A processed RGM-B polypeptide sequence is as follows:

(SEQ ID NO: 58)
GDCQQPAQCRIQKCTTDFVSLTSHLNSAVDGFDSEFCKALRAYAGCTQRT
SKACRGNLVYHSAVLGISDLMSQRNCSKDGPTSSTNPEVTHDPCNYHSHA
GAREHRRGDQNPPSYLFCGLFGDPHLRTFKDNFQTCKVEGAWPLIDNNYL
SVQVTNVPVVPGSSATATNKITIIFKAHHECTDQKVYQAVTDDLPAAFVD
GTTSGGDSDAKSLRIVERESGHYVEMHARYIGTTVFVRQVGRYLTLAIRM
PEDLAMSYEESQDLQLCVNGCPLSERIDDGQGQVSAILGHSLPRTSLVQA
WPGYTLETANTQCHEKMPVKDIYFQSCVFDLLTTGDANFTAAAHSALEDV
EALHPRKERWHIFPSS

A nucleic acid sequence encoding unprocessed human RGM-B precursor protein is shown below (SEQ ID NO: 59), corresponding to nucleotides 403-1836 of NCBI Reference Sequence NM_001012761.2. The signal sequence is underlined.

(SEQ ID NO: 59)
ATGATAAGGAAGAAGAGGAAGCGAAGCGCGCCCCCCGGCCCATGCCGCA
GCCACGGGCCCAGACCCGCCACGGCGCCCGCGCCGCCGCCCTCGCCGGA
GCCCACGAGACCTGCATGGACGGGCATGGGCTTGAGAGCAGCACCTTCC
AGCGCCGCCGCTGCCGCCGCCGAGGTTGAGCAGCGCCGCAGCCCCGGGC
TCTGCCCCCCGCCGCTGGAGCTGCTGCTGCTGCTGCTGTTCAGCCTCGG
GCTGCTCCACGCAGGTGACTGCCAACAGCCAGCCCAATGTCGAATCCAG
AAATGCACCACGGACTTCGTGTCCCTGACTTCTCACCTGAACTCTGCCG
TTGACGGCTTTGACTCTGAGTTTTGCAAGGCCTTGCGTGCCTATGCTGG
CTGCACCCAGCGAACTTCAAAAGCCTGCCGTGGCAACCTGGTATACCAT
TCTGCCGTGTTGGGTATCAGTGACCTCATGAGCCAGAGGAATTGTTCCA
AGGATGGACCCACATCCTCTACCAACCCCGAAGTGACCCATGATCCTTG
CAACTATCACAGCCACGCTGGAGCCAGGGAACACAGGAGAGGGGACCAG
AACCCTCCCAGTTACCTTTTTTGTGGCTTGTTTGGAGATCCTCACCTCA
GAACTTTCAAGGATAACTTCCAAACATGCAAAGTAGAAGGGGCCTGGCC
ACTCATAGATAATAATTATCTTTCAGTTCAAGTGACAAACGTACCTGTG
GTCCCTGGATCCAGTGCTACTGCTACAAATAAGATCACTATTATCTTCA
AAGCCCACCATGAGTGTACAGATCAGAAAGTCTACCAAGCTGTGACAGA
TGACCTGCCGGCCGCCTTTGTGGATGGCACCACCAGTGGTGGGGACAGC
GATGCCAAGAGCCTGCGTATCGTGGAAAGGGAGAGTGGCCACTATGTGG
AGATGCACGCCCGCTATATAGGGACCACAGTGTTTGTGCGGCAGGTGGG
TCGCTACCTGACCCTTGCCATCCGTATGCCTGAAGACCTGGCCATGTCC
TACGAGGAGAGCCAGGACCTGCAGCTGTGCGTGAACGGCTGCCCCCTGA
GTGAACGCATCGATGACGGGCAGGGCCAGGTGTCTGCCATCCTGGGACA
CAGCCTGCCTCGCACCTCCTTGGTGCAGGCCTGGCCTGGCTACACACTG
GAGACTGCCAACACTCAATGCCATGAGAAGATGCCAGTGAAGGACATCT
ATTTCCAGTCCTGTGTCTTCGACCTGCTCACCACTGGTGATGCCAACTT
TACTGCCGCAGCCCACAGTGCCTTGGAGGATGTGGAGGCCCTGCACCCA
AGGAAGGAACGCTGGCACATTTTCCCCAGCAGTGGCAATGGGACTCCCC
GTGGAGGCAGTGATTTGTCTGTCAGTCTAGGACTCACCTGCTTGATCCT
TATCGTGTTTTTG

A nucleic acid sequence encoding a processed RGM-B is shown below (SEQ ID NO: 60):

(SEQ ID NO: 60)
GGTGACTGCCAACAGCCAGCCCAATGTCGAATCCAGAAATGCACCACGG
ACTTCGTGTCCCTGACTTCTCACCTGAACTCTGCCGTTGACGGCTTTGA
CTCTGAGTTTTGCAAGGCCTTGCGTGCCTATGCTGGCTGCACCCAGCGA
ACTTCAAAAGCCTGCCGTGGCAACCTGGTATACCATTCTGCCGTGTTGG
GTATCAGTGACCTCATGAGCCAGAGGAATTGTTCCAAGGATGGACCCAC
ATCCTCTACCAACCCCGAAGTGACCCATGATCCTTGCAACTATCACAGC
CACGCTGGAGCCAGGGAACACAGGAGAGGGGACCAGAACCCTCCCAGTT
ACCTTTTTTGTGGCTTGTTTGGAGATCCTCACCTCAGAACTTTCAAGGA
TAACTTCCAAACATGCAAAGTAGAAGGGGCCTGGCCACTCATAGATAAT
AATTATCTTTCAGTTCAAGTGACAAACGTACCTGTGGTCCCTGGATCCA
GTGCTACTGCTACAAATAAGATCACTATTATCTTCAAAGCCCACCATGA
GTGTACAGATCAGAAAGTCTACCAAGCTGTGACAGATGACCTGCCGGCC
GCCTTTGTGGATGGCACCACCAGTGGTGGGGACAGCGATGCCAAGAGCC
TGCGTATCGTGGAAAGGGAGAGTGGCCACTATGTGGAGATGCACGCCCG
CTATATAGGGACCACAGTGTTTGTGCGGCAGGTGGGTCGCTACCTGACC
CTTGCCATCCGTATGCCTGAAGACCTGGCCATGTCCTACGAGGAGAGCC
AGGACCTGCAGCTGTGCGTGAACGGCTGCCCCCTGAGTGAACGCATCGA
TGACGGGCAGGGCCAGGTGTCTGCCATCCTGGGACACAGCCTGCCTCGC
ACCTCCTTGGTGCAGGCCTGGCCTGGCTACACACTGGAGACTGCCAACA
CTCAATGCCATGAGAAGATGCCAGTGAAGGACATCTATTTCCAGTCCTG
TGTCTTCGACCTGCTCACCACTGGTGATGCCAACTTTACTGCCGCAGCC
CACAGTGCCTTGGAGGATGTGGAGGCCCTGCACCCAAGGAAGGAACGCT
GGCACATTTTCCCCAGCAGT

In certain embodiments, the disclosure relates to heteromultimers that comprise at least one RGM-B polypeptide, which includes fragments, functional variants, and modified forms thereof. Preferably, RGM-B polypeptides for use in accordance with the disclosure (e.g., heteromultimers comprising a RGM-B polypeptide and uses thereof) are soluble (e.g., an extracellular domain of RGM-B). In other preferred embodiments, RGM-B polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one RGM-B polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 57 or 58. In some embodiments, heteromultimers of the disclosure comprise at least one RGM-B polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-87 (e.g., amino acid residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, or 87) of SEQ ID NO: 57, and ends at any one of amino acids 452-478 (e.g., amino acid residues 452, 453, 454, 455, 456, 457, 458, 459, 460, 461, 462, 463, 464, 465, 466, 467, 468, 469, 470, 471, 472, 473, 474, 475, 476, 477, or 478) of SEQ ID NO: 57. In some embodiments, heteromultimers of the disclosure comprise at least one RGM-B polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 (e.g., amino acid residues 210, 211, 212, 213, 214, 215, 216, 217, 218, 219, 220, 221, or 222) of SEQ ID NO: 57, and ends at any one of amino acids 413-452 (e.g., amino acid residues 413, 414, 415, 416, 417, 418, 419, 420, 421, 422, 423, 424, 425, 426, 427, 428, 429, 430, 431, 432, 433, 434, 435, 435, 436, 437, 438, 439, 440, 441, 442, 443, 444, 445, 446, 447, 448, 449, 450, 451, or 452) of SEQ ID NO: 57. In some embodiments, heteromultimers of the disclosure comprise at least one RGM-B polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 (e.g., amino acid residues 87, 88, 89, 90, 91, 92, 93, 94 or 95) of SEQ ID NO: 57, and ends at any one of amino acids 204-209 (e.g., amino acid residues 204, 205, 206, 207, 208, or 209) of SEQ ID NO: 57. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 1-452 of SEQ ID NO: 57. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 87-204 of SEQ ID NO: 57. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 87-209 of SEQ ID NO: 57. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 95-204 of SEQ ID NO: 57. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 95-209 of SEQ ID NO: 57. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 210-413 of SEQ ID NO: 57. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 210-452 of SEQ ID NO: 57. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 222-413 of SEQ ID NO: 57. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 222-452 of SEQ ID NO: 57. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 87-413 of SEQ ID NO: 57. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 87-452 of SEQ ID NO: 57. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 95-413 of SEQ ID NO: 57. In some embodiments, heteromultimers of the disclosure comprise of at least one RGM-B polypeptide that is at least 70%, 75%, 80%, 85%, 90%, 91%, 92%, 93%, 94%, 95%, 96%, 97%, 98%, 99%, or 100% identical to amino acids of 95-452 of SEQ ID NO: 57. In some embodiments, heteromultimers of the disclosure comprise at least a RGM-B protein, wherein the RGM-B protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 (e.g., amino acid residues 87, 88, 89, 90, 91, 92, 93, 94 or 95) of SEQ ID NO: 57, and ends at any one of amino acids 204-209 (e.g., amino acid residues 204, 205, 206, 207, 208, or 209) of SEQ ID NO: 57, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 (e.g., amino acid residues 210, 211, 212, 213, 214, 215, 216, 217, 218, 219, 220, 221, or 222) of SEQ ID NO: 57, and ends at any one of amino acids 413-452 (e.g., amino acid residues 413, 414, 415, 416, 417, 418, 419, 420, 421, 422, 423, 424, 425, 426, 427, 428, 429, 430, 431, 432, 433, 434, 435, 435, 436, 437, 438, 439, 440, 441, 442, 443, 444, 445, 446, 447, 448, 449, 450, 451, or 452) of SEQ ID NO: 57. In some embodiments, heteromultimers of the disclosure comprise at least one single chain ligand trap that comprises a first RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 87-95 (e.g., amino acid residues 87, 88, 89, 90, 91, 92, 93, 94 or 95) of SEQ ID NO: 57, and ends at any one of amino acids 204-209 (e.g., amino acid residues 204, 205, 206, 207, 208, or 209) of SEQ ID NO: 57, and second RGM-B polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 210-222 (e.g., amino acid residues 210, 211, 212, 213, 214, 215, 216, 217, 218, 219, 220, 221, or 222) of SEQ ID NO: 57, and ends at any one of amino acids 413-452 (e.g., amino acid residues 413, 414, 415, 416, 417, 418, 419, 420, 421, 422, 423, 424, 425, 426, 427, 428, 429, 430, 431, 432, 433, 434, 435, 435, 436, 437, 438, 439, 440, 441, 442, 443, 444, 445, 446, 447, 448, 449, 450, 451, or 452) of SEQ ID NO: 57. In some embodiments, heteromultimers of the disclosure comprise at least one RGM-B polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 87-89 (e.g., amino acid residues 87, 88, or 89) of SEQ ID NO: 57, and ends at any one of amino acids 471-478 (e.g., amino acid residues 471, 472, 473, 474, 475, 476, 477, or 478) of SEQ ID NO: 57. In some embodiments, heteromultimers of the disclosure comprise at least one RGM-B polypeptide that is at least 70%, 75%, 80%, 85%, 90%, 91%, 92%, 93%, 94%, 95%, 96%, 97%, 98%, 99%, or 100% identical to amino acids 87-478 of SEQ ID NO: 57. In some embodiments, heteromultimers of the disclosure comprise at least one RGM-B polypeptide that is at least 70%, 75%, 80%, 85%, 90%, 91%, 92%, 93%, 94%, 95%, 96%, 97%, 98%, 99%, or 100% identical to amino acids 89-471 of SEQ ID NO: 57.

The term “RGM-A polypeptide” includes polypeptides comprising any naturally occurring RGM-A protein (encoded by RGMA or one of its nonhuman orthologs) as well as any variants thereof (including mutants, fragments, fusions, and peptidomimetic forms) that retain a useful activity.

A human RGM-A isoform 1 precursor protein sequence (NCBI Ref Seq NP_001159755.1) is as follows:

| (SEQ ID NO: 61) | |
|---|---|
| 1 | MGGLGPRRAG TSRERLVVTG RAGWMGMGRG AGRSALGFWP TLAFLLCSFP AATSPCKILK |
| 61 | CNSEFWSATS GSHAPASDDT PEFCAALRSY ALCTRRTART CRGDLAYHSA VHGIEDLMSQ |
| 121 | HNCSKDGPTS QPRLRTLPPA GDSQERSDSP EICHYEKSFH KHSATPNYTH CGLFGDPHLR |
| 181 | TFTDRFQTCK VQGAWPLIDN NYLNVQVTNT PVLPGSAATA TSKLTIIFKN FQECVDQKVY |
| 241 | QAEMDELPAA FVDGSKNGGD KHGANSLKIT EKVSGQHVEI QAKYIGTTIV VRQVGRYLTF |
| 301 | AVRMPEEVVN AVEDWDSQGL YLCLRGCPLN QQIDFQAFHT NAEGTGARRL AAASPAPTAP |
| 361 | ETFPYETAVA KCKEKLPVED LYYQACVFDL LTTGDVNFTL AAYYALEDVK MLHSNKDKLH |
| 421 | LYERTRDLPG RAAAGLPLAP RPLLGALVPL LALLPVFC |

The signal peptide is indicated by solid underline.

A processed RGM-A isoform 1 polypeptide sequence is as follows:

(SEQ ID NO: 62)
CKILKCNSEFWSATSGSHAPASDDTPEFCAALRSYALCTRRTARTCR
GDLAYHSAVHGIEDLMSQHNCSKDGPTSQPRLRTLPPAGDSQERSDS
PEICHYEKSFHKHSATPNYTHCGLFGDPHLRTFTDRFQTCKVQGAWP
LIDNNYLNVQVTNTPVLPGSAATATSKLTIIFKNFQECVDQKVYQAE
MDELPAAFVDGSKNGGDKHGANSLKITEKVSGQHVEIQAKYIGTTIV
VRQVGRYLTFAVRMPEEVVNAVEDWDSQGLYLCLRGCPLNQQIDFQA
FHTNAEGTGARRLAAASPAPTAPETFPYETAVAKCKEKLPVEDLYYQ
ACVFDLLTTGDVNFTLAAYYALEDVKMLHS

A nucleic acid sequence encoding unprocessed human RGM-A isoform 1 precursor protein is shown below (SEQ ID NO: 63), corresponding to nucleotides 232-1605 of NCBI Reference Sequence NM_001166283.1. The signal sequence is underlined.

(SEQ ID NO: 63)
ATGGGTGGCCTGGGGCCACGACGGGCGGGAACCTCGAGGGAGAGGCTA
GTGGTAACAGGCCGAGCTGGATGGATGGGTATGGGGAGAGGGGCAGGA
CGTTCAGCCCTGGGATTCTGGCCGACCCTCGCCTTCCTTCTCTGCAGC
TTCCCCGCAGCCACCTCCCCGTGCAAGATCCTCAAGTGCAACTCTGAG
TTCTGGAGCGCCACGTCGGGCAGCCACGCCCCAGCCTCAGACGACACC
CCCGAGTTCTGTGCAGCCTTGCGCAGCTACGCCCTGTGCACGCGGCGG
ACGGCCCGCACCTGCCGGGGTGACCTGGCCTACCACTCGGCCGTCCAT
GGCATAGAGGACCTCATGAGCCAGCACAACTGCTCCAAGGATGGCCCC
ACCTCGCAGCCACGCCTGCGCACGCTCCCACCGGCCGGAGACAGCCAG
GAGCGCTCGGACAGCCCCGAGATCTGCCATTACGAGAAGAGCTTTCAC
AAGCACTCGGCCACCCCCAACTACACGCACTGTGGCCTCTTCGGGGAC
CCACACCTCAGGACTTTCACCGACCGCTTCCAGACCTGCAAGGTGCAG
GGCGCCTGGCCGCTCATCGACAATAATTACCTGAACGTGCAGGTCACC
AACACGCCTGTGCTGCCCGGCTCAGCGGCCACTGCCACCAGCAAGCTC
ACCATCATCTTCAAGAACTTCCAGGAGTGTGTGGACCAGAAGGTGTAC
CAGGCTGAGATGGACGAGCTCCCGGCCGCCTTCGTGGATGGCTCTAAG
AACGGTGGGGACAAGCACGGGGCCAACAGCCTGAAGATCACTGAGAAG
GTGTCAGGCCAGCACGTGGAGATCCAGGCCAAGTACATCGGCACCACC
ATCGTGGTGCGCCAGGTGGGCCGCTACCTGACCTTTGCCGTCCGCATG
CCAGAGGAAGTGGTCAATGCTGTGGAGGACTGGGACAGCCAGGGTCTC
TACCTCTGCCTGCGGGGCTGCCCCCTCAACCAGCAGATCGACTTCCAG
GCCTTCCACACCAATGCTGAGGGCACCGGTGCCCGCAGGCTGGCAGCC
GCCAGCCCTGCACCCACAGCCCCCGAGACCTTCCCATACGAGACAGCC
GTGGCCAAGTGCAAGGAGAAGCTGCCGGTGGAGGACCTGTACTACCAG
GCCTGCGTCTTCGACCTCCTCACCACGGGCGACGTGAACTTCACACTG
GCCGCCTACTACGCGTTGGAGGATGTCAAGATGCTCCACTCCAACAAA
GACAAACTGCACCTGTATGAGAGGACTCGGGACCTGCCAGGCAGGGCG
GCTGCGGGGCTGCCCCTGGCCCCCCGGCCCCTCCTGGGCGCCCTCGTC
CCGCTCCTGGCCCTGCTCCCTGTGTTCTGC

A nucleic acid sequence encoding a processed RGM-A isoform 1 is shown below (SEQ ID NO: 64):

(SEQ ID NO: 64)
TGCAAGATCCTCAAGTGCAACTCTGAGTTCTGGAGCGCCACGTCGGGCA
GCCACGCCCCAGCCTCAGACGACACCCCCGAGTTCTGTGCAGCCTTGCG
CAGCTACGCCCTGTGCACGCGGCGGACGGCCCGCACCTGCCGGGGTGAC
CTGGCCTACCACTCGGCCGTCCATGGCATAGAGGACCTCATGAGCCAGC
ACAACTGCTCCAAGGATGGCCCCACCTCGCAGCCACGCCTGCGCACGCT
CCCACCGGCCGGAGACAGCCAGGAGCGCTCGGACAGCCCCGAGATCTGC
CATTACGAGAAGAGCTTTCACAAGCACTCGGCCACCCCCAACTACACGC
ACTGTGGCCTCTTCGGGGACCCACACCTCAGGACTTTCACCGACCGCTT
CCAGACCTGCAAGGTGCAGGGCGCCTGGCCGCTCATCGACAATAATTAC
CTGAACGTGCAGGTCACCAACACGCCTGTGCTGCCCGGCTCAGCGGCCA
CTGCCACCAGCAAGCTCACCATCATCTTCAAGAACTTCCAGGAGTGTGT
GGACCAGAAGGTGTACCAGGCTGAGATGGACGAGCTCCCGGCCGCCTTC
GTGGATGGCTCTAAGAACGGTGGGGACAAGCACGGGGCCAACAGCCTGA
AGATCACTGAGAAGGTGTCAGGCCAGCACGTGGAGATCCAGGCCAAGTA
CATCGGCACCACCATCGTGGTGCGCCAGGTGGGCCGCTACCTGACCTTT
GCCGTCCGCATGCCAGAGGAAGTGGTCAATGCTGTGGAGGACTGGGACA
GCCAGGGTCTCTACCTCTGCCTGCGGGGCTGCCCCCTCAACCAGCAGAT
CGACTTCCAGGCCTTCCACACCAATGCTGAGGGCACCGGTGCCCGCAGG
CTGGCAGCCGCCAGCCCTGCACCCACAGCCCCCGAGACCTTCCCATACG
AGACAGCCGTGGCCAAGTGCAAGGAGAAGCTGCCGGTGGAGGACCTGTA
CTACCAGGCCTGCGTCTTCGACCTCCTCACCACGGGCGACGTGAACTTC
ACACTGGCCGCCTACTACGCGTTGGAGGATGTCAAGATGCTCCACTCC

A human RGM-A isoform 2 precursor protein sequence (NCBI Ref Seq NP_001159758.1) is as follows:

| (SEQ ID NO: 65) | |
|---|---|
| 1 | MGMGRGAGRS ALGFWPTLAF LLCSFPAATS PCKILKCNSE FWSATSGSHA PASDDTPEFC |
| 61 | AALRSYALCT RRTARTCRGD LAYHSAVHGI EDLMSQHNCS KDGPTSQPRL RTLPPAGDSQ |
| 121 | ERSDSPEICH YEKSFHKHSA TPNYTHCGLF GDPHLRTFTD RFQTCKVQGA WPLIDNNYLN |
| 181 | VQVTNTPVLP GSAATATSKL TIIFKNFQEC VDQKVYQAEM DELPAAFVDG SKNGGDKHGA |
| 241 | NSLKITEKVS GQHVEIQAKY IGTTIVVRQV GRYLTFAVRM PEEVVNAVED WDSQGLYLCL |
| 301 | RGCPLNQQID FQAFHTNAEG TGARRLAAAS PAPTAPETFP YETAVAKCKE KLPVEDLYYQ |
| 361 | ACVFDLLTTG DVNFTLAAYY ALEDVKMLHS NKDKLHLYER TRDLPGRAAA GLPLAPRPLL |
| 421 | GALVPLLALL PVFC |

The signal peptide is indicated by solid underline.

A mature RGM-A isoform 2 sequence is as follows:

(SEQ ID NO: 66)
CKILKCNSEFWSATSGSHAPASDDTPEFCAALRSYALCTRRTARTCR
GDLAYHSAVHGIEDLMSQHNCSKDGPTSQPRLRTLPPAGDSQERSDS
PEICHYEKSFHKHSATPNYTHCGLFGDPHLRTFTDRFQTCKVQGAWP
LIDNNYLNVQVTNTPVLPGSAATATSKLTIIFKNFQECVDQKVYQAE
MDELPAAFVDGSKNGGDKHGANSLKITEKVSGQHVEIQAKYIGTTIV
VRQVGRYLTFAVRMPEEVVNAVEDWDSQGLYLCLRGCPLNQQIDFQA
FHTNAEGTGARRLAAASPAPTAPETFPYETAVAKCKEKLPVEDLYYQ
ACVFDLLTTGDVNFTLAAYYALEDVKMLHS

A nucleic acid sequence encoding unprocessed human RGM-A isoform 2 precursor protein is shown below (SEQ ID NO: 67), corresponding to nucleotides 164-1465 of NCBI Reference Sequence NM_001166286.1. The signal sequence is underlined.

(SEQ ID NO: 67)
ATGGGTATGGGGAGAGGGGCAGGACGTTCAGCCCTGGGATTCTGGCC
GACCCTCGCCTTCCTTCTCTGCAGCTTCCCCGCAGCCACCTCCCCGT
GCAAGATCCTCAAGTGCAACTCTGAGTTCTGGAGCGCCACGTCGGGC
AGCCACGCCCCAGCCTCAGACGACACCCCCGAGTTCTGTGCAGCCTT
GCGCAGCTACGCCCTGTGCACGCGGCGGACGGCCCGCACCTGCCGGG
GTGACCTGGCCTACCACTCGGCCGTCCATGGCATAGAGGACCTCATG
AGCCAGCACAACTGCTCCAAGGATGGCCCCACCTCGCAGCCACGCCT
GCGCACGCTCCCACCGGCCGGAGACAGCCAGGAGCGCTCGGACAGCC
CCGAGATCTGCCATTACGAGAAGAGCTTTCACAAGCACTCGGCCACC
CCCAACTACACGCACTGTGGCCTCTTCGGGGACCCACACCTCAGGAC
TTTCACCGACCGCTTCCAGACCTGCAAGGTGCAGGGCGCCTGGCCGC
TCATCGACAATAATTACCTGAACGTGCAGGTCACCAACACGCCTGTG
CTGCCCGGCTCAGCGGCCACTGCCACCAGCAAGCTCACCATCATCTT
CAAGAACTTCCAGGAGTGTGTGGACCAGAAGGTGTACCAGGCTGAGA
TGGACGAGCTCCCGGCCGCCTTCGTGGATGGCTCTAAGAACGGTGGG
GACAAGCACGGGGCCAACAGCCTGAAGATCACTGAGAAGGTGTCAGG
CCAGCACGTGGAGATCCAGGCCAAGTACATCGGCACCACCATCGTGG
TGCGCCAGGTGGGCCGCTACCTGACCTTTGCCGTCCGCATGCCAGAG
GAAGTGGTCAATGCTGTGGAGGACTGGGACAGCCAGGGTCTCTACCT
CTGCCTGCGGGGCTGCCCCCTCAACCAGCAGATCGACTTCCAGGCCT
TCCACACCAATGCTGAGGGCACCGGTGCCCGCAGGCTGGCAGCCGCC
AGCCCTGCACCCACAGCCCCCGAGACCTTCCCATACGAGACAGCCGT
GGCCAAGTGCAAGGAGAAGCTGCCGGTGGAGGACCTGTACTACCAGG
CCTGCGTCTTCGACCTCCTCACCACGGGCGACGTGAACTTCACACTG
GCCGCCTACTACGCGTTGGAGGATGTCAAGATGCTCCACTCCAACAA
AGACAAACTGCACCTGTATGAGAGGACTCGGGACCTGCCAGGCAGGG
CGGCTGCGGGGCTGCCCCTGGCCCCCCGGCCCCTCCTGGGCGCCCTC
GTCCCGCTCCTGGCCCTGCTCCCTGTGTTCTGC

A nucleic acid sequence encoding a processed RGM-A isoform 2 is shown below (SEQ ID NO: 68):

(SEQ ID NO: 68)
TGCAAGATCCTCAAGTGCAACTCTGAGTTCTGGAGCGCCACGTCGGG
CAGCCACGCCCCAGCCTCAGACGACACCCCCGAGTTCTGTGCAGCCT
TGCGCAGCTACGCCCTGTGCACGCGGCGGACGGCCCGCACCTGCCGG
GGTGACCTGGCCTACCACTCGGCCGTCCATGGCATAGAGGACCTCAT
GAGCCAGCACAACTGCTCCAAGGATGGCCCCACCTCGCAGCCACGCC
TGCGCACGCTCCCACCGGCCGGAGACAGCCAGGAGCGCTCGGACAGC
CCCGAGATCTGCCATTACGAGAAGAGCTTTCACAAGCACTCGGCCAC
CCCCAACTACACGCACTGTGGCCTCTTCGGGGACCCACACCTCAGGA
CTTTCACCGACCGCTTCCAGACCTGCAAGGTGCAGGGCGCCTGGCCG
CTCATCGACAATAATTACCTGAACGTGCAGGTCACCAACACGCCTGT
GCTGCCCGGCTCAGCGGCCACTGCCACCAGCAAGCTCACCATCATCT
TCAAGAACTTCCAGGAGTGTGTGGACCAGAAGGTGTACCAGGCTGAG
ATGGACGAGCTCCCGGCCGCCTTCGTGGATGGCTCTAAGAACGGTGG
GGACAAGCACGGGGCCAACAGCCTGAAGATCACTGAGAAGGTGTCAG
GCCAGCACGTGGAGATCCAGGCCAAGTACATCGGCACCACCATCGTG
GTGCGCCAGGTGGGCCGCTACCTGACCTTTGCCGTCCGCATGCCAGA
GGAAGTGGTCAATGCTGTGGAGGACTGGGACAGCCAGGGTCTCTACC
TCTGCCTGCGGGGCTGCCCCCTCAACCAGCAGATCGACTTCCAGGCC
TTCCACACCAATGCTGAGGGCACCGGTGCCCGCAGGCTGGCAGCCGC
CAGCCCTGCACCCACAGCCCCCGAGACCTTCCCATACGAGACAGCCG
TGGCCAAGTGCAAGGAGAAGCTGCCGGTGGAGGACCTGTACTACCAG
GCCTGCGTCTTCGACCTCCTCACCACGGGCGACGTGAACTTCACACT
GGCCGCCTACTACGCGTTGGAGGATGTCAAGATGCTCCACTCC

A human RGM-A isoform 3 precursor protein sequence (NCBI Ref Seq NP_064596.2) is as follows:

| (SEQ ID NO: 69) | |
|---|---|
| 1 | MQPPRERLVV TGRAGWMGMG RGAGRSALGF WPTLAFLLCS FPAATSPCKI LKCNSEFWSA |
| 61 | TSGSHAPASD DTPEFCAALR SYALCTRRTA RTCRGDLAYH SAVHGIEDLM SQHNCSKDGP |
| 121 | TSQPRLRTLP PAGDSQERSD SPEICHYEKS FHKHSATPNY THCGLFGDPH LRTFTDRFQT |
| 181 | CKVQGAWPLI DNNYLNVQVT NTPVLPGSAA TATSKLTIIF KNFQECVDQK VYQAEMDELP |
| 241 | AAFVDGSKNG GDKHGANSLK ITEKVSGQHV EIQAKYIGTT IVVRQVGRYL TFAVRMPEEV |
| 301 | VNAVEDWDSQ GLYLCLRGCP LNQQIDFQAF HTNAEGTGAR RLAAASPAPT APETFPYETA |
| 361 | VAKCKEKLPV EDLYYQACVF DLLTTGDVNF TLAAYYALED VKMLHSNKDK LHLYERTRDL |
| 421 | PGRAAAGLPL APRPLLGALV PLLALLPVFC |

The signal peptide is indicated by solid underline.

A mature RGM-A isoform 3 sequence is as follows:

(SEQ ID NO: 70)
CKILKCNSEFWSATSGSHAPASDDTPEFCAALRSYALCTRRTARTCR
GDLAYHSAVHGIEDLMSQHNCSKDGPTSQPRLRTLPPAGDSQERSDS
PEICHYEKSFHKHSATPNYTHCGLFGDPHLRTFTDRFQTCKVQGAWP
LIDNNYLNVQVTNTPVLPGSAATATSKLTIIFKNFQECVDQKVYQAE
MDELPAAFVDGSKNGGDKHGANSLKITEKVSGQHVEIQAKYIGTTIV
VRQVGRYLTFAVRMPEEVVNAVEDWDSQGLYLCLRGCPLNQQIDFQA
FHTNAEGTGARRLAAASPAPTAPETFPYETAVAKCKEKLPVEDLYYQ
ACVEDLLTTGDVNFTLAAYYALEDVEMLHS

A nucleic acid sequence encoding unprocessed RGM-A isoform 3 precursor protein is shown below (SEQ ID NO: 71), corresponding to nucleotides 283-1632 of NCBI Reference Sequence NM_020211.2. The signal sequence is underlined.

(SEQ ID NO: 71)
ATGCAGCCGCCAAGGGAGAGGCTAGTGGTAACAGGCCGAGCTGGATGGA
TGGGTATGGGGAGAGGGGCAGGACGTTCAGCCCTGGGATTCTGGCCGAC
CCTCGCCTTCCTTCTCTGCAGCTTCCCCGCAGCCACCTCCCCGTGCAAG
ATCCTCAAGTGCAACTCTGAGTTCTGGAGCGCCACGTCGGGCAGCCACG
CCCCAGCCTCAGACGACACCCCCGAGTTCTGTGCAGCCTTGCGCAGCTA
CGCCCTGTGCACGCGGCGGACGGCCCGCACCTGCCGGGGTGACCTGGCC
TACCACTCGGCCGTCCATGGCATAGAGGACCTCATGAGCCAGCACAACT
GCTCCAAGGATGGCCCCACCTCGCAGCCACGCCTGCGCACGCTCCCACC
GGCCGGAGACAGCCAGGAGCGCTCGGACAGCCCCGAGATCTGCCATTAC
GAGAAGAGCTTTCACAAGCACTCGGCCACCCCCAACTACACGCACTGTG
GCCTCTTCGGGGACCCACACCTCAGGACTTTCACCGACCGCTTCCAGAC
CTGCAAGGTGCAGGGCGCCTGGCCGCTCATCGACAATAATTACCTGAAC
GTGCAGGTCACCAACACGCCTGTGCTGCCCGGCTCAGCGGCCACTGCCA
CCAGCAAGCTCACCATCATCTTCAAGAACTTCCAGGAGTGTGTGGACCA
GAAGGTGTACCAGGCTGAGATGGACGAGCTCCCGGCCGCCTTCGTGGAT
GGCTCTAAGAACGGTGGGGACAAGCACGGGGCCAACAGCCTGAAGATCA
CTGAGAAGGTGTCAGGCCAGCACGTGGAGATCCAGGCCAAGTACATCGG
CACCACCATCGTGGTGCGCCAGGTGGGCCGCTACCTGACCTTTGCCGTC
CGCATGCCAGAGGAAGTGGTCAATGCTGTGGAGGACTGGGACAGCCAGG
GTCTCTACCTCTGCCTGCGGGGCTGCCCCCTCAACCAGCAGATCGACTT
CCAGGCCTTCCACACCAATGCTGAGGGCACCGGTGCCCGCAGGCTGGCA
GCCGCCAGCCCTGCACCCACAGCCCCCGAGACCTTCCCATACGAGACAG
CCGTGGCCAAGTGCAAGGAGAAGCTGCCGGTGGAGGACCTGTACTACCA
GGCCTGCGTCTTCGACCTCCTCACCACGGGCGACGTGAACTTCACACTG
GCCGCCTACTACGCGTTGGAGGATGTCAAGATGCTCCACTCCAACAAAG
ACAAACTGCACCTGTATGAGAGGACTCGGGACCTGCCAGGCAGGGCGGC
TGCGGGGCTGCCCCTGGCCCCCCGGCCCCTCCTGGGCGCCCTCGTCCCG
CTCCTGGCCCTGCTCCCTGTGTTCTGC

A nucleic acid sequence encoding processed RGM-A isoform 3 is shown below (SEQ ID NO: 72):

(SEQ ID NO: 72)
TGCAAGATCCTCAAGTGCAACTCTGAGTTCTGGAGCGCCACGTCGGGCA
GCCACGCCCCAGCCTCAGACGACACCCCCGAGTTCTGTGCAGCCTTGCG
CAGCTACGCCCTGTGCACGCGGCGGACGGCCCGCACCTGCCGGGGTGAC
CTGGCCTACCACTCGGCCGTCCATGGCATAGAGGACCTCATGAGCCAGC
ACAACTGCTCCAAGGATGGCCCCACCTCGCAGCCACGCCTGCGCACGCT
CCCACCGGCCGGAGACAGCCAGGAGCGCTCGGACAGCCCCGAGATCTGC
CATTACGAGAAGAGCTTTCACAAGCACTCGGCCACCCCCAACTACACGC
ACTGTGGCCTCTTCGGGGACCCACACCTCAGGACTTTCACCGACCGCTT
CCAGACCTGCAAGGTGCAGGGCGCCTGGCCGCTCATCGACAATAATTAC
CTGAACGTGCAGGTCACCAACACGCCTGTGCTGCCCGGCTCAGCGGCCA
CTGCCACCAGCAAGCTCACCATCATCTTCAAGAACTTCCAGGAGTGTGT
GGACCAGAAGGTGTACCAGGCTGAGATGGACGAGCTCCCGGCCGCCTTC
GTGGATGGCTCTAAGAACGGTGGGGACAAGCACGGGGCCAACAGCCTGA
AGATCACTGAGAAGGTGTCAGGCCAGCACGTGGAGATCCAGGCCAAGTA
CATCGGCACCACCATCGTGGTGCGCCAGGTGGGCCGCTACCTGACCTTT
GCCGTCCGCATGCCAGAGGAAGTGGTCAATGCTGTGGAGGACTGGGACA
GCCAGGGTCTCTACCTCTGCCTGCGGGGCTGCCCCCTCAACCAGCAGAT
CGACTTCCAGGCCTTCCACACCAATGCTGAGGGCACCGGTGCCCGCAGG
CTGGCAGCCGCCAGCCCTGCACCCACAGCCCCCGAGACCTTCCCATACG
AGACAGCCGTGGCCAAGTGCAAGGAGAAGCTGCCGGTGGAGGACCTGTA
CTACCAGGCCTGCGTCTTCGACCTCCTCACCACGGGCGACGTGAACTTC
ACACTGGCCGCCTACTACGCGTTGGAGGATGTCAAGATGCTCCACTCC

In certain embodiments, the disclosure relates to heteromultimers that comprise at least one RGM-A polypeptide, which includes fragments, functional variants, and modified forms thereof. Preferably, RGM-A polypeptides for use in accordance with the disclosure (e.g., heteromultimers comprising a RGM-A polypeptide and uses thereof) are soluble (e.g., an extracellular domain of RGM-A). In other preferred embodiments, RGM-A polypeptides for use in accordance with the disclosure bind to and/or inhibit (antagonize) activity (e.g., Smad signaling) of one or more TGF-beta superfamily ligands. In some embodiments, heteromultimers of the disclosure comprise at least one RGM-A polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 61, 62, 65, 66, 69, or 70. In some embodiments, heteromultimers of the disclosure comprise at least one RGM-A polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-177 (e.g., amino acid residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, or 177) of SEQ ID NO: 61, and ends at any one of amino acids 430-458 (e.g., amino acid residues 430, 431, 432, 433, 434, 435, 435, 436, 437, 438, 439, 440, 441, 442, 443, 444, 445, 446, 447, 448, 449, 450, 451, 452, 453, 454, 455, 456, 457, or 458) of SEQ ID NO: 61. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 1-430 of SEQ ID NO: 61. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 1-458 of SEQ ID NO: 61. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 177-430 of SEQ ID NO: 61. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 177-458 of SEQ ID NO: 61. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 56-430 of SEQ ID NO: 61. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 56-458 of SEQ ID NO: 61. In some embodiments, heteromultimers of the disclosure comprise at least one RGM-A polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-153 (e.g., amino acid residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 135, 136, 137, 138, 139, 140, 141, 142, 143, 144, 145, 146, 147, 148, 149, 150, 151, 152, or 153) of SEQ ID NO: 65, and ends at any one of amino acids 406-434 (e.g., amino acid residues 406, 407, 408, 409, 410, 411, 412, 413, 414, 415, 416, 417, 418, 419, 420, 421, 422, 423, 424, 425, 426, 427, 428, 429, 430, 431, 432, 433, 434) of SEQ ID NO: 65. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 1-406 of SEQ ID NO: 65. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 153-406 of SEQ ID NO: 65. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 1-434 of SEQ ID NO: 65. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 153-434 of SEQ ID NO: 65. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 32-406 of SEQ ID NO: 65. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 32-434 of SEQ ID NO: 65. In some embodiments, heteromultimers of the disclosure comprise at least one RGM-A polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-169 (e.g., amino acid residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169) of SEQ ID NO: 69, and ends at any one of amino acids 422-450 (e.g., amino acid residues 422, 423, 424, 425, 426, 427, 428, 429, 430, 431, 432, 433, 434, 435, 435, 436, 437, 438, 439, 440, 441, 442, 443, 444, 445, 446, 447, 448, 449, 450) of SEQ ID NO: 69. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 1-422 of SEQ ID NO: 69. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 169-422 of SEQ ID NO: 69. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 1-450 of SEQ ID NO: 69. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 169-450 of SEQ ID NO: 69. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 48-422 of SEQ ID NO: 69. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 48-450 of SEQ ID NO: 69. In some embodiments, heteromultimers of the disclosure comprise at least one RGM-A polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 56-61 (e.g., amino acid residues 56, 57, 58, 59, 60, or 61) of SEQ ID NO: 61, and ends at any one of amino acids 366-458 (e.g., amino acid residues 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, or 458) of SEQ ID NO: 61. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 56-458 of SEQ ID NO: 61. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 61-366 of SEQ ID NO: 61. In some embodiments, heteromultimers of the disclosure comprise at least one RGM-A polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 32-37 (e.g., amino acid residues 32, 33, 34, 35, 36, or 37) of SEQ ID NO: 65, and ends at any one of amino acids 362-434 (e.g., amino acid residues 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, or 434) of SEQ ID NO: 65. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 32-434 of SEQ ID NO: 65. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 37-362 of SEQ ID NO: 65. In some embodiments, heteromultimers of the disclosure comprise at least one RGM-A polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 48-53 (e.g., amino acid residues 48, 49, 50, 51, 52, or 53) of SEQ ID NO: 69, and ends at any one of amino acids 378-450 (e.g., amino acid residues 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450) of SEQ ID NO: 69. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 48-450 of SEQ ID NO: 69. In some embodiments, heteromultimers of the disclosure comprise of at least one RGM-A polypeptide that is at least 70%, 75%, 80%, 85%, 90%, 91%, 92%, 93%, 94%, 95%, 96%, 97%, 98%, 99%, or 100% identical to amino acids of 53-378 of SEQ ID NO: 69.

The term “hemojuvelin polypeptide” includes polypeptides comprising any naturally occurring hemojuvelin protein (encoded by HFE2 or one of its nonhuman orthologs) as well as any variants thereof (including mutants, fragments, fusions, and peptidomimetic forms) that retain a useful activity.

The human hemojuvelin isoform A precursor protein sequence (NCBI Ref Seq NP_998818.1) is as follows:

| (SEQ ID NO: 73) | |
|---|---|
| 1 | MGEPGQSPSP RSSHGSPPTL STLTLLLLLC GHAHSQCKIL RCNAEYVSST LSLRGGGSSG |
| 61 | ALRGGGGGGR GGGVGSGGLC RALRSYALCT RRTARTCRGD LAFHSAVHGI EDLMIQHNCS |
| 121 | RQGPTAPPPP RGPALPGAGS GLPAPDPCDY EGRFSRLHGR PPGFLHCASF GDPHVRSFHH |
| 181 | HFHTCRVQGA WPLLDNDFLF VQATSSPMAL GANATATRKL TIIFKNMQEC IDQKVYQAEV |
| 241 | DNLPVAFEDG SINGGDRPGG SSLSIQTANP GNHVEIQAAY IGTTIIIRQT AGQLSFSIKV |
| 301 | AEDVAMAFSA EQDLQLCVGG CPPSQRLSRS ERNRRGAITI DTARRLCKEG LPVEDAYFHS |
| 361 | CVFDVLISGD PNFTVAAQAA LEDARAFLPD LEKLHLFPSD AGVPLSSATL LAPLLSGLFV |
| 421 | LWLCIQ |

The signal peptide is indicated by single underline.

A processed hemojuvelin isoform A polypeptide sequence is as follows:

(SEQ ID NO: 74)
QCKILRCNAEYVSSTLSLRGGGSSGALRGGGGGGRGGGVGSGGLCRAL
RSYALCTRRTARTCRGDLAFHSAVHGIEDLMIQHNCSRQGPTAPPPPR
GPALPGAGSGLPAPDPCDYEGRFSRLHGRPPGFLHCASFGDPHVRSFH
HHFHTCRVQGAWPLLDNDFLFVQATSSPMALGANATATRKLTIIFKNM
QECIDQKVYQAEVDNLPVAFEDGSINGGDRPGGSSLSIQTANPGNHVE
IQAAYIGTTIIIRQTAGQLSFSIKVAEDVAMAFSAEQDLQLCVGGCPP
SQRLSRSERNRRGAITIDTARRLCKEGLPVEDAYFHSCVFDVLISGDP
NFTVAAQAALEDARAFLPDLEKLHLFPSD

A nucleic acid sequence encoding unprocessed human hemojuvelin isoform A precursor protein is shown below (SEQ ID NO: 75), corresponding to nucleotides 326-1603 of NCBI Reference Sequence NM_213653.3. The signal sequence is underlined.

(SEQ ID NO: 75)
ATGGGGGAGCCAGGCCAGTCCCCTAGTCCCAGGTCCTCCCATGGCAGT
CCCCCAACTCTAAGCACTCTCACTCTCCTGCTGCTCCTCTGTGGACAT
GCTCATTCTCAATGCAAGATCCTCCGCTGCAATGCTGAGTACGTATCG
TCCACTCTGAGCCTTAGAGGTGGGGGTTCATCAGGAGCACTTCGAGGA
GGAGGAGGAGGAGGCCGGGGTGGAGGGGTGGGCTCTGGCGGCCTCTGT
CGAGCCCTCCGCTCCTATGCGCTCTGCACTCGGCGCACCGCCCGCACC
TGCCGCGGGGACCTCGCCTTCCATTCGGCGGTACATGGCATCGAAGAC
CTGATGATCCAGCACAACTGCTCCCGCCAGGGCCCTACAGCCCCTCCC
CCGCCCCGGGGCCCCGCCCTTCCAGGCGCGGGCTCCGGCCTCCCTGCC
CCGGACCCTTGTGACTATGAAGGCCGGTTTTCCCGGCTGCATGGTCGT
CCCCCGGGGTTCTTGCATTGCGCTTCCTTCGGGGACCCCCATGTGCGC
AGCTTCCACCATCACTTTCACACATGCCGTGTCCAAGGAGCTTGGCCT
CTACTGGATAATGACTTCCTCTTTGTCCAAGCCACCAGCTCCCCCATG
GCGTTGGGGGCCAACGCTACCGCCACCCGGAAGCTCACCATCATATTT
AAGAACATGCAGGAATGCATTGATCAGAAGGTGTATCAGGCTGAGGTG
GATAATCTTCCTGTAGCCTTTGAAGATGGTTCTATCAATGGAGGTGAC
CGACCTGGGGGATCCAGTTTGTCGATTCAAACTGCTAACCCTGGGAAC
CATGTGGAGATCCAAGCTGCCTACATTGGCACAACTATAATCATTCGG
CAGACAGCTGGGCAGCTCTCCTTCTCCATCAAGGTAGCAGAGGATGTG
GCCATGGCCTTCTCAGCTGAACAGGACCTGCAGCTCTGTGTTGGGGGG
TGCCCTCCAAGTCAGCGACTCTCTCGATCAGAGCGCAATCGTCGGGGA
GCTATAACCATTGATACTGCCAGACGGCTGTGCAAGGAAGGGCTTCCA
GTGGAAGATGCTTACTTCCATTCCTGTGTCTTTGATGTTTTAATTTCT
GGTGATCCCAACTTTACCGTGGCAGCTCAGGCAGCACTGGAGGATGCC
CGAGCCTTCCTGCCAGACTTAGAGAAGCTGCATCTCTTCCCCTCAGAT
GCTGGGGTTCCTCTTTCCTCAGCAACCCTCTTAGCTCCACTCCTTTCT
GGGCTCTTTGTTCTGTGGCTTTGCATTCAG

A nucleic acid sequence encoding a processed hemojuvelin isoform A is shown below (SEQ ID NO: 76):

(SEQ ID NO: 76)
CAATGCAAGATCCTCCGCTGCAATGCTGAGTACGTATCGTCCACTCTG
AGCCTTAGAGGTGGGGGTTCATCAGGAGCACTTCGAGGAGGAGGAGGA
GGAGGCCGGGGTGGAGGGGTGGGCTCTGGCGGCCTCTGTCGAGCCCTC
CGCTCCTATGCGCTCTGCACTCGGCGCACCGCCCGCACCTGCCGCGGG
GACCTCGCCTTCCATTCGGCGGTACATGGCATCGAAGACCTGATGATC
CAGCACAACTGCTCCCGCCAGGGCCCTACAGCCCCTCCCCCGCCCCGG
GGCCCCGCCCTTCCAGGCGCGGGCTCCGGCCTCCCTGCCCCGGACCCT
TGTGACTATGAAGGCCGGTTTTCCCGGCTGCATGGTCGTCCCCCGGGG
TTCTTGCATTGCGCTTCCTTCGGGGACCCCCATGTGCGCAGCTTCCAC
CATCACTTTCACACATGCCGTGTCCAAGGAGCTTGGCCTCTACTGGAT
AATGACTTCCTCTTTGTCCAAGCCACCAGCTCCCCCATGGCGTTGGGG
GCCAACGCTACCGCCACCCGGAAGCTCACCATCATATTTAAGAACATG
CAGGAATGCATTGATCAGAAGGTGTATCAGGCTGAGGTGGATAATCTT
CCTGTAGCCTTTGAAGATGGTTCTATCAATGGAGGTGACCGACCTGGG
GGATCCAGTTTGTCGATTCAAACTGCTAACCCTGGGAACCATGTGGAG
ATCCAAGCTGCCTACATTGGCACAACTATAATCATTCGGCAGACAGCT
GGGCAGCTCTCCTTCTCCATCAAGGTAGCAGAGGATGTGGCCATGGCC
TTCTCAGCTGAACAGGACCTGCAGCTCTGTGTTGGGGGGTGCCCTCCA
AGTCAGCGACTCTCTCGATCAGAGCGCAATCGTCGGGGAGCTATAACC
ATTGATACTGCCAGACGGCTGTGCAAGGAAGGGCTTCCAGTGGAAGAT
GCTTACTTCCATTCCTGTGTCTTTGATGTTTTAATTTCTGGTGATCCC
AACTTTACCGTGGCAGCTCAGGCAGCACTGGAGGATGCCCGAGCCTTC
CTGCCAGACTTAGAGAAGCTGCATCTCTTCCCCTCAGAT

A human hemojuvelin isoform B protein sequence (NCBI Ref Seq NP_660320.3) is as follows:

```
(SEQ ID NO: 77)
    1 MIQHNCSRQG PTAPPPPRGP ALPGAGSGLP
        APDPCDYEGR FSRLHGRPPG FLHCASFGDP
  61 HVRSFHHHFH TCRVQGAWPL LDNDFLFVQA
        TSSPMALGAN ATATRKLTII FKNMQECIDQ
121 KVYQAEVDNL PVAFEDGSIN GGDRPGGSSL
        SIQTANPGNH VEIQAAYIGT TIIIRQTAGQ
181 LSFSIKVAED VAMAFSAEQD LQLCVGGCPP
        SQRLSRSERN RRGAITIDTA RRLCKEGLPV
241 EDAYFHSCVF DVLISGDPNF TVAAQAALED
        ARAFLPDLEK LHLFPSDAGV PLSSATLLAP
301 LLSGLFVLWL CIQ
```

A processed hemojuvelin isoform B polypeptide sequence is as follows:

MIQHNCSRQGPTAPPPPRGPALPGAGSGLPAPDPCDYEGRFSRLHGRPPGFLHCASFGDPHVRSFHHHFHTCRVQ GAWPLLDNDFLFVQATSSPMALGANATATRKLTIIFKNMQECIDQKVYQAEVDNLPVAFEDGSINGGDRPGGSSL SIQTANPGNHVEIQAAYIGTTIIIRQTAGQLSFSIKVAEDVAMAFSAEQDLQLCVGGCPPSQRLSRSERNRRGAI TIDTARRLCKEGLPVEDAYFHSCVFDVLISGDPNFTVAAQAALEDARAFLPDLEKLHLFPSD (SEQ ID NO: 78)

A nucleic acid sequence encoding human hemojuvelin isoform B precursor protein is shown below (SEQ ID NO: 79), corresponding to nucleotides 479-1417 of NCBI Reference Sequence NM_145277.4.

(SEQ ID NO: 79)
ATGATCCAGCACAACTGCTCCCGCCAGGGCCCTACAGCCCCTCCCCCGC
CCCGGGGCCCCGCCCTTCCAGGCGCGGGCTCCGGCCTCCCTGCCCCGGA
CCCTTGTGACTATGAAGGCCGGTTTTCCCGGCTGCATGGTCGTCCCCCG
GGGTTCTTGCATTGCGCTTCCTTCGGGGACCCCCATGTGCGCAGCTTCC
ACCATCACTTTCACACATGCCGTGTCCAAGGAGCTTGGCCTCTACTGGA
TAATGACTTCCTCTTTGTCCAAGCCACCAGCTCCCCCATGGCGTTGGGG
GCCAACGCTACCGOCACCCGGAAGCTCACCATCATATTTAAGAACATGC
AGGAATGOATTGATCAGAAGGTGTATCAGGCTGAGGTGGATAATCTTCC
TGTAGCCTTTGAAGATGGTTCTATCAATGGAGGTGACCGACCTGGGGGA
TCCAGTTTGTCGATTCAAACTGCTAACCCTGGGAACCATGTGGAGATCC
AAGCTGCCTACATTGGCACAACTATAATCATTCGGCAGACAGCTGGGCA
GCTCTCCTTCTCCATCAAGGTAGCAGAGGATGTGGCCATGGCCTTCTCA
GCTGAACAGGACCTGCAGCTCTGTGTTGGGGGGTGCCCTCCAAGTCAGC
GACTCTCTCGATCAGAGCGCAATCGTCGGGGAGCTATAACCATTGATAC
TGCCAGACGGCTGTGCAAGGAAGGGCTTCCAGTGGAAGATGCTTACTTC
CATTCCTGTGTCTTTGATGTTTTAATTTCTGGTGATCCCAACTTTACCG
TGGCAGCTCAGGCAGCACTGGAGGATGCCCGAGCCTTCCTGCCAGACTT
AGAGAAGCTGCATCTCTTCCCCTCAGATGCTGGGGTTCCTCTTTCCTCA
GCAACCCTCTTAGCTCCACTCCTTTCTGGGCTCTTTGTTCTGTGGCTTT
GCATTCAG

A nucleic acid sequence encoding a processed hemojuvelin isoform B is shown below (SEQ ID NO: 80):

(SEQ ID NO: 80)
ATGATCCAGCACAACTGCTCCCGCCAGGGCCCTACAGCCCCTCCCCC
GCCCCGGGGCCCCGCCCTTCCAGGCGCGGGCTCCGGCCTCCCTGCCC
CGGACCCTTGTGACTATGAAGGCCGGTTTTCCCGGCTGCATGGTCGT
CCCCCGGGGTTCTTGCATTGCGCTTCCTTCGGGGACCCCCATGTGCG
CAGCTTCCACCATCACTTTCACACATGCCGTGTCCAAGGAGCTTGGC
CTCTACTGGATAATGACTTCCTCTTTGTCCAAGCCACCAGCTCCCCC
ATGGCGTTGGGGGCCAACGCTACCGOCACCCGGAAGCTCACCATCAT
ATTTAAGAACATGCAGGAATGOATTGATCAGAAGGTGTATCAGGCTG
AGGTGGATAATCTTCCTGTAGCCTTTGAAGATGGTTCTATCAATGGA
GGTGACCGACCTGGGGGATCCAGTTTGTCGATTCAAACTGCTAACCC
TGGGAACCATGTGGAGATCCAAGCTGCCTACATTGGCACAACTATAA
TCATTCGGCAGACAGCTGGGCAGCTCTCCTTCTCCATCAAGGTAGCA
GAGGATGTGGCCATGGCCTTCTCAGCTGAACAGGACCTGCAGCTCTG
TGTTGGGGGGTGCCCTCCAAGTCAGCGACTCTCTCGATCAGAGCGCA
ATCGTCGGGGAGCTATAACCATTGATACTGCCAGACGGCTGTGCAAG
GAAGGGCTTCCAGTGGAAGATGCTTACTTCCATTCCTGTGTCTTTGA
TGTTTTAATTTCTGGTGATCCCAACTTTACCGTGGCAGCTCAGGCAG
CACTGGAGGATGCCCGAGCCTTCCTGCCAGACTTAGAGAAGCTGCAT
CTCTTCCCCTCAGAT

A human hemojuvelin isoform C protein sequence (NCBI Ref Seq NP_973733.1) is as follows:

```
(SEQ ID NO: 81)
    1 MQECIDQKVY QAEVDNLPVA FEDGSINGGD RPGGSSLSIQ
        TANPGNHVEI QAAYIGTTII
  61 IRQTAGQLSF SIKVAEDVAM AFSAEQDLQL CVGGCPPSQR
        LSRSERNRRG AITIDTARRL
121 CKEGLPVEDA YFHSCVFDVL ISGDPNFTVA AQAALEDARA
        FLPDLEKLHL FPSD
```

A processed hemojuvelin isoform C polypeptide sequence is as follows:

(SEQ ID NO: 82)
MQECIDQKVYQAEVDNLPVAFEDGSINGGDRPGGSSLSIQTANPGNHV
EIQAAYIGTTIIIRQTAGQLSFSIKVAEDVAMAFSAEQDLQLCVGGCP
PSQRLSRSERNRRGAITIDTARRLCKEGLPVEDAYFHSCVFDVLISGD
PNFTVAAQAALEDARAFLPDLEKLHLFPSD

A nucleic acid sequence encoding human hemojuvelin isoform C protein is shown below (SEQ ID NO: 83), corresponding to nucleotides 295-894 of NCBI Reference Sequence NM_202004.3.

(SEQ ID NO: 83)
ATGCAGGAATGCATTGATCAGAAGGTGTATCAGGCTGAGGTGGATAAT
CTTCCTGTAGCCTTTGAAGATGGTTCTATCAATGGAGGTGACCGACCT
GGGGGATCCAGTTTGTCGATTCAAACTGCTAACCCTGGGAACCATGTG
GAGATCCAAGCTGCCTACATTGGCACAACTATAATCATTCGGCAGACA
GCTGGGCAGCTCTCCTTCTCCATCAAGGTAGCAGAGGATGTGGCCATG
GCCTTCTCAGCTGAACAGGACCTGCAGCTCTGTGTTGGGGGGTGCCCT
CCAAGTCAGCGACTCTCTCGATCAGAGCGCAATCGTCGGGGAGCTATA
ACCATTGATACTGCCAGACGGCTGTGCAAGGAAGGGCTTCCAGTGGAA
GATGCTTACTTCCATTCCTGTGTCTTTGATGTTTTAATTTCTGGTGAT
CCCAACTTTACCGTGGCAGCTCAGGCAGCACTGGAGGATGCCCGAGCC
TTCCTGCCAGACTTAGAGAAGCTGCATCTCTTCCCCTCAGATGCTGGG
GTTCCTCTTTCCTCAGCAACCCTCTTAGCTCCACTCCTTTCTGGGCTC
TTTGTTCTGTGGCTTTGCATTCAG

A nucleic acid sequence encoding a processed hemojuvelin isoform C is shown below (SEQ ID NO: 84):

(SEQ ID NO: 84)
ATGCAGGAATGCATTGATCAGAAGGTGTATCAGGCTGAGGTGGATAAT
CTTCCTGTAGCCTTTGAAGATGGTTCTATCAATGGAGGTGACCGACCT
GGGGGATCCAGTTTGTCGATTCAAACTGCTAACCCTGGGAACCATGTG
GAGATCCAAGCTGCCTACATTGGCACAACTATAATCATTCGGCAGACA
GCTGGGCAGCTCTCCTTCTCCATCAAGGTAGCAGAGGATGTGGCCATG
GCCTTCTCAGCTGAACAGGACCTGCAGCTCTGTGTTGGGGGGTGCCCT
CCAAGTCAGCGACTCTCTCGATCAGAGCGCAATCGTCGGGGAGCTATA
ACCATTGATACTGCCAGACGGCTGTGCAAGGAAGGGCTTCCAGTGGAA
GATGCTTACTTCCATTCCTGTGTCTTTGATGTTTTAATTTCTGGTGAT
CCCAACTTTACCGTGGCAGCTCAGGCAGCACTGGAGGATGCCCGAGCC
TTCCTGCCAGACTTAGAGAAGCTGCATCTCTTCCCCTCAGAT

In certain embodiments, the disclosure relates to heteromultimers that comprise at least one hemojuvelin polypeptide, which includes fragments, functional variants, and modified forms thereof. Preferably, hemojuvelin polypeptides for use in accordance with the disclosure (e.g., heteromultimers comprising a hemojuvelin polypeptide and uses thereof) are soluble (e.g., an extracellular domain of hemojuvelin). In other preferred embodiments, hemojuvelin polypeptides for use in accordance with disclosure bind to and/or inhibit (antagonize) activity (e.g., Smad signaling) of one or more TGF-beta superfamily ligands. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 73, 74, 77, 78, 81, or 82. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-36 (e.g., amino acid residues 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 35, or 36) of SEQ ID NO: 73, and ends at any one of amino acids 400-426 (e.g., amino acid residues 400, 401, 402, 403, 404, 405, 406, 407, 408, 409, 410, 411, 412, 413, 414, 415, 416, 417, 418, 419, 420, 421, 422, 423, 424, 425, or 426) of SEQ ID NO: 73. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 (e.g., amino acid residues 36, 37, 38, 39, 40, 41, or 42) of SEQ ID NO: 73, and ends at any one of amino acids 167-172 (e.g., amino acid residues 167, 168, 169, 170, 171, or 172) of SEQ ID NO: 73. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 (e.g., amino acid residues 173, 174, 175, 176, 177, 178, 179, 180, 181, 182, 183, 184, or 185) of SEQ ID NO: 73, and ends at any one of amino acids 361-400 (e.g., amino acid residues 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400) of SEQ ID NO: 73. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-400 of SEQ ID NO: 73. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-426 of SEQ ID NO: 73. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 36-400 of SEQ ID NO: 73. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 36-426 of SEQ ID NO: 73. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 36-167 of SEQ ID NO: 73. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 36-172 of SEQ ID NO: 73. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 42-167 of SEQ ID NO: 73. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 42-172 of SEQ ID NO: 73. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 173-361 of SEQ ID NO: 73. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 173-400 of SEQ ID NO: 73. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 185-361 of SEQ ID NO: 73. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 185-400 of SEQ ID NO: 73. In some embodiments, heteromultimers of the disclosure comprise at least one hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 (e.g., amino acid residues 36, 37, 38, 39, 40, 41, or 42) of SEQ ID NO: 73, and ends at any one of amino acids 167-172 (e.g., amino acid residues 167, 168, 169, 170, 171, or 172) of SEQ ID NO: 73, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 (e.g., amino acid residues 173, 174, 175, 176, 177, 178, 179, 180, 181, 182, 183, 184, or 185) of SEQ ID NO: 73, and ends at any one of amino acids 361-400 (e.g., amino acid residues 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400) of SEQ ID NO: 73. In some embodiments, heteromultimers of the disclosure comprise at least one single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 36-42 (e.g., amino acid residues 36, 37, 38, 39, 40, 41, or 42) of SEQ ID NO: 73, and ends at any one of amino acids 167-172 (e.g., amino acid residues 167, 168, 169, 170, 171, or 172) of SEQ ID NO: 73, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 173-185 (e.g., amino acid residues 173, 174, 175, 176, 177, 178, 179, 180, 181, 182, 183, 184, or 185) of SEQ ID NO: 73, and ends at any one of amino acids 361-400 (e.g., amino acid residues 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400) of SEQ ID NO: 73. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 (e.g., amino acid residues 1, 2, 3, 4, 5, or 6) of SEQ ID NO: 77, and ends at any one of amino acids 287-313 (e.g., amino acid residues 287, 288, 289, 300, 301, 302, 303, 304, 305, 306, 307, 308, 309, 310, 311, 312, or 313) of SEQ ID NO: 77. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 (e.g., amino acid residues 1, 2, 3, 4, 5, or 6) of SEQ ID NO: 77, and ends at any one of amino acids 54-59 (e.g., amino acid residues 54, 55, 56, 57, 58, or 59) of SEQ ID NO: 77. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 (e.g., amino acid residues 60, 61, 62, 63, 64, 65, 66, 67, 68, 69, 70, 71, or 72) of SEQ ID NO: 77, and ends at any one of amino acids 248-287 (e.g., amino acid residues 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, or 287) of SEQ ID NO: 77. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-287 of SEQ ID NO: 77. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-313 of SEQ ID NO: 77. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 6-287 of SEQ ID NO: 77. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 6-313 of SEQ ID NO: 77. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-54 of SEQ ID NO: 77. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-59 of SEQ ID NO: 77. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 6-54 of SEQ ID NO: 77. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 6-59 of SEQ ID NO: 77. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 60-248 of SEQ ID NO: 77. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 60-287 of SEQ ID NO: 77. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 72-248 of SEQ ID NO: 77. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 72-287 of SEQ ID NO: 77. In some embodiments, heteromultimers of the disclosure comprise at least one hemojuvelin protein, wherein the hemojuvelin protein is a dimer comprising a first polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 (e.g., amino acid residues 1, 2, 3, 4, 5, or 6) of SEQ ID NO: 77, and ends at any one of amino acids 54-59 (e.g., amino acid residues 54, 55, 56, 57, 58, or 59) of SEQ ID NO: 77, and second polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 (e.g., amino acid residues 60, 61, 62, 63, 64, 65, 66, 67, 68, 69, 70, 71, or 72) of SEQ ID NO: 77, and ends at any one of amino acids 248-287 (e.g., amino acid residues 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, or 287) of SEQ ID NO: 77. In some embodiments, heteromultimers of the disclosure comprise at least one single chain ligand trap that comprises a first hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 1-6 (e.g., amino acid residues 1, 2, 3, 4, 5, or 6) of SEQ ID NO: 77, and ends at any one of amino acids 54-59 (e.g., amino acid residues 54, 55, 56, 57, 58, or 59) of SEQ ID NO: 77, and second hemojuvelin polypeptide domain that is at least 70%, 75%, 80%, 85%, 90%, 91%, 92%, 93%, 94%, 95%, 96%, 97%, 98%, 99%, or 100% identical to a polypeptide that begins at any one of amino acids of 60-72 (e.g., amino acid residues 60, 61, 62, 63, 64, 65, 66, 67, 68, 69, 70, 71, or 72) of SEQ ID NO: 77, and ends at any one of amino acids 248-287 (e.g., amino acid residues 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, or 287) of SEQ ID NO: 77. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 1-4 (e.g., amino acid residues 1, 2, 3, or 4) of SEQ ID NO: 81, and ends at any one of amino acids 135-200 (e.g., amino acid residues 135, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200) of SEQ ID NO: 81. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-135 of SEQ ID NO: 81. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-200 of SEQ ID NO: 81. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 4-135 of SEQ ID NO: 81. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 4-200 of SEQ ID NO: 81. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-174 of SEQ ID NO: 81. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 4-174 of SEQ ID NO: 81. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 36-37 (e.g., amino acid residues 36 or 37) of SEQ ID NO: 73, and ends at any one of amino acids 424-426 (e.g., amino acid residues 424, 425, or 426) of SEQ ID NO: 73. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 36-426 of SEQ ID NO: 73. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 37-424 of SEQ ID NO: 73. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 36-400 of SEQ ID NO: 73. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to a polypeptide that begins at anyone of amino acids of 1-4 (e.g., amino acid residues 1, 2, 3, or 4) of SEQ ID NO: 82, and ends at any one of amino acids 135-174 (e.g., amino acid residues 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, or 174) of SEQ ID NO: 82. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-174 of SEQ ID NO: 82. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 4-135 of SEQ ID NO: 82. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-174 of SEQ ID NO: 82. In some embodiments, heteromultimers of the disclosure comprise at least one hemojuvelin polypeptide that is at least 70%, 75%, 80%, 85%, 90%, 91%, 92%, 93%, 94%, 95%, 96%, 97%, 98%, 99%, or 100% identical to a polypeptide that begins at anyone of amino acids of 1-6 (e.g., amino acid residues 1, 2, 3, 4, 5, or 6) of SEQ ID NO: 77, and ends at any one of amino acids 311-313 (e.g., amino acid residues 311, 312, or 313) of SEQ ID NO: 77. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-313 of SEQ ID NO: 77. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 6-311 of SEQ ID NO: 77. In some embodiments, heteromultimers of the disclosure comprise of at least one hemojuvelin polypeptide that is at least 70%, 75%, 80%, 85%, 90%, 91%, 92%, 93%, 94%, 95%, 96%, 97%, 98%, 99%, or 100% identical to amino acids of 1-127 of SEQ ID NO: 77.

The term “betaglycan polypeptide” includes polypeptides comprising any naturally is occurring betaglycan protein (encoded by TGFBR3 or one of its nonhuman orthologs) as well as any variants thereof (including mutants, fragments, fusions, and peptidomimetic forms) that retain a useful activity.

The human betaglycan isoform A precursor protein sequence (NCBI Ref Seq NP_003234.2) is as follows:

| (SEQ ID NO: 85) | |
|---|---|
| 1 | MTSHYVIAIF ALMSSCLATA GPEPGALCEL SPVSASHPVQ ALMESFTVLS GCASRGTTGL |
| 61 | PQEVHVLNLR TAGQGPGQLQ REVTLHLNPI SSVHIHHKSV VFLLNSPHPL VWHLKTERLA |
| 121 | TGVSRLFLVS EGSVVQFSSA NFSLTAETEE RNFPHGNEHL LNWAREEYGA VISFTELKIA |
| 181 | RNIYIKVGED QVFPPKCNIG KNFLSLNYLA EYLQPKAAEG CVMSSQPQNE EVHIIELITP |
| 241 | NSNPYSAFQV DITIDIRPSQ EDLEVVKNLI LILKCKKSVN WVIKSFDVKG SLKIIAPNSI |
| 301 | GFGKESERSM TMTKSIRDDI PSTQGNLVKW ALDNGYSPIT SYTMAPVANR FHLRLENNAE |
| 361 | EMGDEEVHTI PPELRILLDP GALPALQNPP IRGGEGQNGG LPFPFPDISR RVWNEEGEDG |
| 421 | LPRPKDPVIP SIQLFPGLRE PEEVQGSVDI ALSVKCDNEK MIVAVEKDSF QASGYSGMDV |
| 481 | TLLDPTCKAK MNGTHFVLES PLNGCGTRPR WSALDGVVYY NSIVIQVPAL GDSSGWPDGY |
| 541 | EDLESGDNGF PGDMDEGDAS LFTRPEIVVF NCSLQQVRNP SSFQEQPHGN ITFNMELYNT |
| 601 | DLFLVPSQGV FSVPENGHVY VEVSVTKAEQ ELGFAIQTCF ISPYSNPDRM SHYTIIENIC |
| 661 | PKDESVKFYS PKRVHFPIPQ ADMDKKRFSF VFKPVFNTSL LFLQCELTLC TKMEKHPQKL |
| 721 | PKCVPPDEAC TSLDASIIWA MMQNKKTFTK PLAVIHHEAE SKEKGPSMEE PNPISPPIFH |
| 781 | 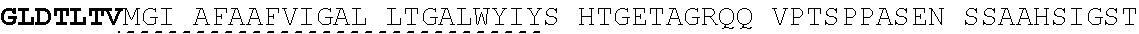 |
| 841 | QSTPCSSSST A |

The signal peptide is indicated by single underline, the extracellular domain is indicated in bold font, and the transmembrane domain is indicated by dotted-underline. This isoform differs from betaglycan isoform B by insertion of a single alanine indicated above by double underline.

A processed betaglycan isoform A polypeptide sequence is as follows:

(SEQ ID NO: 86)
GPEPGALCELSPVSASHPVQALMESFTVLSGCASRGTTGLPQEVHVLNL
RTAGQGPGQLQREVTLHLNPISSVHIHHKSVVFLLNSPHPLVWHLKTER
LATGVSRLFLVSEGSVVQFSSANFSLTAETEERNFPHGNEHLLNWARKE
YGAVTSFTELKIARNIYIKVGEDQVFPPKCNIGKNFLSLNYLAEYLQPK
AAEGCVMSSQPQNEEVHIIELITPNSNPYSAFQVDITIDIRPSQEDLEV
VKNLILILKCKKSVNWVIKSFDVKGSLKIIAPNSIGFGKESERSMTMTK
SIRDDIPSTQGNLVKWALDNGYSPITSYTMAPVANRFHLRLENNAEEMG
DEEVHTIPPELRILLDPGALPALQNPPIRGGEGQNGGLPFPFPDISRRV
WNEEGEDGLPRPKDPVIPSIQLFPGLREPEEVQGSVDIALSVKCDNEKM
IVAVEKDSFQASGYSGMDVTLLDPTCKAKMNGTHFVLESPLNGCGTRPR
WSALDGVVYYNSIVIQVPALGDSSGWPDGYEDLESGDNGFPGDMDEGDA
SLFTRPEIVVFNCSLQQVRNPSSFQEQPHGNITFNMELYNTDLFLVPSQ
GVFSVPENGHVYVEVSVTKAEQELGFAIQTCFISPYSNPDRMSHYTIIE
NICPKDESVKFYSPKRVHFPIPQADMDKKRFSFVFKPVFNTSLLFLQCE
LTLCTKMEKHPQKLPKCVPPDEACTSLDASIIWAMMQNKKTFTKPLAVI
HHEAESKEKGPSMKEPNPISPPIFHGLDTLTV

A nucleic acid sequence encoding the unprocessed precursor protein of human betaglycan isoform A is shown below (SEQ ID NO: 87), corresponding to nucleotides 516-3068 of NCBI Reference Sequence NM_003243.4. The signal sequence is indicated by solid underline and the transmembrane region by dotted underline.

(SEQ ID NO: 87)
ATGACTTCCCATTATGTGATTGCCATCTTTGCCCTGATGAGCTCCTGTTTAGCCACTGCAGGTCCAGAGCCTGGT
GCACTGTGTGAACTGTCACCTGTCAGTGCCTCCCATCCTGTCCAGGCCTTGATGGAGAGCTTCACTGTTTTGTCA
GGCTGTGCCAGCAGAGGCACAACTGGGCTGCCACAGGAGGTGCATGTCCTGAATCTCCGCACTGCAGGCCAGGGG
CCTGGCCAGCTACAGAGAGAGGTCACACTTCACCTGAATCCCATCTCCTCAGTCCACATCCACCACAAGTCTGTT
GTGTTCCTGCTCAACTCCCCACACCCCCTGGTGTGGCATCTGAAGACAGAGAGACTTGCCACTGGGGTCTCCAGA
CTGTTTTTGGTGTCTGAGGGTTCTGTGGTCCAGTTTTCATCAGCAAACTTCTCCTTGACAGCAGAAACAGAAGAA
AGGAACTTCCCCCATGGAAATGAACATCTGTTAAATTGGGCCCGAAAAGAGTATGGAGCAGTTACTTCATTCACC
GAACTCAAGATAGCAAGAAACATTTATATTAAAGTGGGGGAAGATCAAGTGTTCCCTCCAAAGTGCAACATAGGG
AAGAATTTTCTCTCACTCAATTACCTTGCTGAGTACCTTCAACCCAAAGCAGCAGAAGGGTGTGTGATGTCCAGC
CAGCCCCAGAATGAGGAAGTACACATCATCGAGCTAATCACCCCCAACTCTAACCCCTACAGTGCTTTCCAGGTG
GATATAACAATTGATATAAGACCTTCTCAAGAGGATCTTGAAGTGGTCAAAAATCTCATCCTGATCTTGAAGTGC
AAAAAGTCTGTCAACTGGGTGATCAAATCTTTTGATGTTAAGGGAAGCCTGAAAATTATTGCTCCTAACAGTATT
GGCTTTGGAAAAGAGAGTGAAAGATCTATGACAATGACCAAATCAATAAGAGATGACATTCCTTCAACCCAAGGG
AATCTGGTGAAGTGGGCTTTGGACAATGGCTATAGTCCAATAACTTCATACACAATGGCTCCTGTGGCTAATAGA
TTTCATCTTCGGCTTGAAAATAATGCAGAGGAGATGGGAGATGAGGAAGTCCACACTATTCCTCCTGAGCTACGG
ATCCTGCTGGACCCTGGTGCCCTGCCTGCCCTGCAGAACCCGCCCATCCGGGGAGGGGAAGGCCAAAATGGAGGC
CTTCCGTTTCCTTTCCCAGATATTTCCAGGAGAGTCTGGAATGAAGAGGGAGAAGATGGGCTCCCTCGGCCAAAG
GACCCTGTCATTCCCAGCATACAACTGTTTCCTGGTCTCAGAGAGCCAGAAGAGGTGCAAGGGAGCGTGGATATT
GCCCTGTCTGTCAAATGTGACAATGAGAAGATGATCGTGGCTGTAGAAAAAGATTCTTTTCAGGCCAGTGGCTAC
TCGGGGATGGACGTCACCCTGTTGGATCCTACCTGCAAGGCCAAGATGAATGGCACACACTTTGTTTTGGAGTCT
CCTCTGAATGGCTGCGGTACTCGGCCCCGGTGGTCAGCCCTTGATGGTGTGGTCTACTATAACTCCATTGTGATA
CAGGTTCCAGCCCTTGGGGACAGTAGTGGTTGGCCAGATGGTTATGAAGATCTGGAGTCAGGTGATAATGGATTT
CCGGGAGATATGGATGAAGGAGATGCTTCCCTGTTCACCCGACCTGAAATCGTGGTGTTTAATTGCAGCCTTCAG
CAGGTGAGGAACCCCAGCAGCTTCCAGGAACAGCCCCACGGAAACATCACCTTCAACATGGAGCTATACAACACT
GACCTCTTTTTGGTGCCCTCCCAGGGCGTCTTCTCTGTGCCAGAGAATGGACACGTTTATGTTGAGGTATCTGTT
ACTAAGGCTGAACAAGAACTGGGATTTGCCATCCAAACGTGCTTTATCTCTCCATATTCGAACCCTGATAGGATG
TCTCATTACACCATTATTGAGAATATTTGTCCTAAAGATGAATCTGTGAAATTCTACAGTCCCAAGAGAGTGCAC
TTTCCTATCCCGCAAGCTGACATGGATAAGAAGCGATTCAGCTTTGTCTTCAAGCCTGTCTTCAACACCTCACTG
CTCTTTCTACAGTGTGAGCTGACGCTGTGTACGAAGATGGAGAAGCACCCCCAGAAGTTGCCTAAGTGTGTGCCT
CCTGACGAAGCCTGCACCTCGCTGGACGCCTCGATAATCTGGGCCATGATGCAGAATAAGAAGACGTTCACTAAG
CCCCTTGCTGTGATCCACCATGAAGCAGAATCTAAAGAAAAAGGTCCAAGCATGAAGGAACCAAATCCAATTTCT

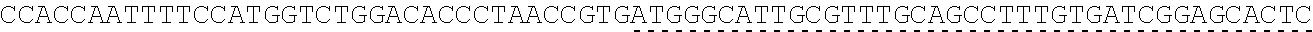

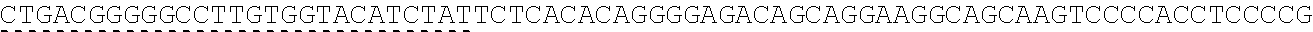

CCAGCCTCGGAAAACAGCAGTGCTGCCCACAGCATCGGCAGCACGCAGAGCACGCCTTGCTCCAGCAGCAGCACG
GCC

A nucleic acid sequence encoding a processed extracellular domain of betaglycan isoform A is shown below (SEQ ID NO: 88):

(SEQ ID NO: 88)
GGTCCAGAGCCTGGTGCACTGTGTGAACTGTCACCTGTCAGTGCCTCC
CATCCTGTCCAGGCCTTGATGGAGAGCTTCACTGTTTTGTCAGGCTGT
GCCAGCAGAGGCACAACTGGGCTGCCACAGGAGGTGCATGTCCTGAAT
CTCCGCACTGCAGGCCAGGGGCCTGGCCAGCTACAGAGAGAGGTCACA
CTTCACCTGAATCCCATCTCCTCAGTCCACATCCACCACAAGTCTGTT
GTGTTCCTGCTCAACTCCCCACACCCCCTGGTGTGGCATCTGAAGACA
GAGAGACTTGCCACTGGGGTCTCCAGACTGTTTTTGGTGTCTGAGGGT
TCTGTGGTCCAGTTTTCATCAGCAAACTTCTCCTTGACAGCAGAAACA
GAAGAAAGGAACTTCCCCCATGGAAATGAACATCTGTTAAATTGGGCC
CGAAAAGAGTATGGAGCAGTTACTTCATTCACCGAACTCAAGATAGCA
AGAAACATTTATATTAAAGTGGGGGAAGATCAAGTGTTCCCTCCAAAG
TGCAACATAGGGAAGAATTTTCTCTCACTCAATTACCTTGCTGAGTAC
CTTCAACCCAAAGCAGCAGAAGGGTGTGTGATGTCCAGCCAGCCCCAG
AATGAGGAAGTACACATCATCGAGCTAATCACCCCCAACTCTAACCCC
TACAGTGCTTTCCAGGTGGATATAACAATTGATATAAGACCTTCTCAA
GAGGATCTTGAAGTGGTCAAAAATCTCATCCTGATCTTGAAGTGCAAA
AAGTCTGTCAACTGGGTGATCAAATCTTTTGATGTTAAGGGAAGCCTG
AAAATTATTGCTCCTAACAGTATTGGCTTTGGAAAAGAGAGTGAAAGA
TCTATGACAATGACCAAATCAATAAGAGATGACATTCCTTCAACCCAA
GGGAATCTGGTGAAGTGGGCTTTGGACAATGGCTATAGTCCAATAACT
TCATACACAATGGCTCCTGTGGCTAATAGATTTCATCTTCGGCTTGAA
AATAATGCAGAGGAGATGGGAGATGAGGAAGTCCACACTATTCCTCCT
GAGCTACGGATCCTGCTGGACCCTGGTGCCCTGCCTGCCCTGCAGAAC
CCGCCCATCCGGGGAGGGGAAGGCCAAAATGGAGGCCTTCCGTTTCCT
TTCCCAGATATTTCCAGGAGAGTCTGGAATGAAGAGGGAGAAGATGGG
CTCCCTCGGCCAAAGGACCCTGTCATTCCCAGCATACAACTGTTTCCT
GGTCTCAGAGAGCCAGAAGAGGTGCAAGGGAGCGTGGATATTGCCCTG
TCTGTCAAATGTGACAATGAGAAGATGATCGTGGCTGTAGAAAAAGAT
TCTTTTCAGGCCAGTGGCTACTCGGGGATGGACGTCACCCTGTTGGAT
CCTACCTGCAAGGCCAAGATGAATGGCACACACTTTGTTTTGGAGTCT
CCTCTGAATGGCTGCGGTACTCGGCCCCGGTGGTCAGCCCTTGATGGT
GTGGTCTACTATAACTCCATTGTGATACAGGTTCCAGCCCTTGGGGAC
AGTAGTGGTTGGCCAGATGGTTATGAAGATCTGGAGTCAGGTGATAAT
GGATTTCCGGGAGATATGGATGAAGGAGATGCTTCCCTGTTCACCCGA
CCTGAAATCGTGGTGTTTAATTGCAGCCTTCAGCAGGTGAGGAACCCC
AGCAGCTTCCAGGAACAGCCCCACGGAAACATCACCTTCAACATGGAG
CTATACAACACTGACCTCTTTTTGGTGCCCTCCCAGGGCGTCTTCTCT
GTGCCAGAGAATGGACACGTTTATGTTGAGGTATCTGTTACTAAGGCT
GAACAAGAACTGGGATTTGCCATCCAAACGTGCTTTATCTCTCCATAT
TCGAACCCTGATAGGATGTCTCATTACACCATTATTGAGAATATTTGT
CCTAAAGATGAATCTGTGAAATTCTACAGTCCCAAGAGAGTGCACTTT
CCTATCCCGCAAGCTGACATGGATAAGAAGCGATTCAGCTTTGTCTTC
AAGCCTGTCTTCAACACCTCACTGCTCTTTCTACAGTGTGAGCTGACG
CTGTGTACGAAGATGGAGAAGCACCCCCAGAAGTTGCCTAAGTGTGTG
CCTCCTGACGAAGCCTGCACCTCGCTGGACGCCTCGATAATCTGGGCC
ATGATGCAGAATAAGAAGACGTTCACTAAGCCCCTTGCTGTGATCCAC
CATGAAGCAGAATCTAAAGAAAAAGGTCCAAGCATGAAGGAACCAAAT
CCAATTTCTCCACCAATTTTCCATGGTCTGGACACCCTAACCGTG

A human betaglycan isoform B precursor protein sequence (NCBI Ref Seq NP_001182612.1) is as follows:

| (SEQ ID NO: 89) | |
|---|---|
| 1 | MTSHYVIAIF ALMSSCLATA GPEPGALCEL SPVSASHPVQ ALMESFTVLS GCASRGTTGL |
| 61 | PQEVHVLNLR TAGQGPGQLQ REVTLHLNPI SSVHIHHKSV VFLLNSPHPL VWHLKTERLA |
| 121 | TGVSRLFLVS EGSVVQFSSA NFSLTAETEE RNFPHGNEHL LNWAREEYGA VISFTELKIA |
| 181 | RNIYIKVGED QVFPPKCNIG KNFLSLNYLA EYLQPKAAEG CVMSSQPQNE EVHIIELITP |
| 241 | NSNPYSAFQV DITIDIRPSQ EDLEVVKNLI LILKCKKSVN WVIKSFDVKG SLKIIAPNSI |
| 301 | GFGKESERSM TMTKSIRDDI PSTQGNLVKW ALDNGYSPIT SYTMAPVANR FHLRLENNEE |
| 361 | MGDEEVHTIP PELRILLDPG ALPALQNPPI RGGEGQNGGL PFPFPDISRR VWNEEGEDGL |
| 421 | PRPKDPVIPS IQLFPGLREP EEVQGSVDIA LSVKCDNEKM IVAVEKDSFQ ASGYSGMDVT |
| 481 | LLDPTCKAKM NGTHFVLESP LNGCGTRPRW SALDGVVYYN SIVIQVPALG DSSGWPDGYE |
| 541 | DLESGDNGFP GDMDEGDASL FTRPEIVVFN CSLQQVRNPS SFQEQPHGNI TFNMELYNTD |
| 601 | LFLVPSQGVF SVPENGHVYV EVSVTKAEQE LGFAIQTCFI SPYSNPDRMS HYTIIENICP |
| 661 | KDESVKFYSP KRVHFPIPQA DMDKKRFSFV FKFVFNTSLL FLQCELTLCT KMEKHPQKLP |
| 721 | KCVPPDEACT SLDASIIWAM MQNKKTFTKP LAVIHHEAES KEKGPSMKEP NPISPPIFHG |
| 781 | 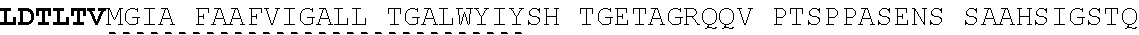 |
| 841 | STPCSSSSTA |

The signal peptide is indicated by single underline, the extracellular domain is indicated in bold font, and the transmembrane domain is indicated by dotted underline.

A processed betaglycan isoform B polypeptide sequence is as follows:

(SEQ ID NO: 90)
GPEPGALCELSPVSASHPVQALMESFTVLSGCASRGT
TGLPQEVHVLNLRTAGQGPGQLQREVTLHLNPISSVH
IHHKSVVFLLNSPHPLVWHLKTERLATGVSRLFLVSE
GSVVQFSSANFSLTAETEERNFPHGNEHLLNWARKEY
GAVTSFTELKIARNIYIKVGEDQVFPPKCNIGKNFLS
LNYLAEYLQPKAAEGCVMSSQPQNEEVHIIELITPNS
NPYSAFQVDITIDIRPSQEDLEVVKNLILILKCKKSV
NWVIKSFDVKGSLKIIAPNSIGFGKESERSMTMTKSI
RDDIPSTQGNLVKWALDNGYSPITSYTMAPVANRFHL
RLENNEEMGDEEVHTIPPELRILLDPGALPALQNPPI
RGGEGQNGGLPFPFPDISRRVWNEEGEDGLPRPKDPV
IPSIQLFPGLREPEEVQGSVDIALSVKCDNEKMIVAV
EKDSFQASGYSGMDVTLLDPTCKAKMNGTHFVLESPL
NGCGTRPRWSALDGVVYYNSIVIQVPALGDSSGWPDG
YEDLESGDNGFPGDMDEGDASLFTRPEIVVENCSLQQ
VRNPSSFQEQPHGNITFNMELYNTDLFLVPSQGVESV
PENGHVYVEVSVTKAEQELGFAIQTCFISPYSNPDRM
SHYTIIENICPKDESVKFYSPKRVHFPIPQADMDKKR
FSFVFKPVFNTSLLFLQCELTLCTKMEKHPQKLPKCV
PPDEACTSLDASIIWAMMQNKKTFTKPLAVIHHEAES
KEKGPSMKEPNPISPPIFHGLDTLTV

A nucleic acid sequence encoding the unprocessed precursor protein of human betaglycan isoform B is shown below (SEQ ID NO: 91), corresponding to nucleotides 516-3065 of NCBI Reference Sequence NM_001195683.1. The signal sequence is indicated by solid underline and the transmembrane region by dotted underline.

(SEQ ID NO: 91)
ATGACTTCCCATTATGTGATTGCCATCTTTGCCCTGATGAGCTCCTGTTTAGCCACTGCAGGTCCAGAGCCTGGT
GCACTGTGTGAACTGTCACCTGTCAGTGCCTCCCATCCTGTCCAGGCCTTGATGGAGAGCTTCACTGTTTTGTCA
GGCTGTGCCAGCAGAGGCACAACTGGGCTGCCACAGGAGGTGCATGTCCTGAATCTCCGCACTGCAGGCCAGGGG
CCTGGCCAGCTACAGAGAGAGGTCACACTTCACCTGAATCCCATCTCCTCAGTCCACATCCACCACAAGTCTGTT
GTGTTCCTGCTCAACTCCCCACACCCCCTGGTGTGGCATCTGAAGACAGAGAGACTTGCCACTGGGGTCTCCAGA
CTGTTTTTGGTGTCTGAGGGTTCTGTGGTCCAGTTTTCATCAGCAAACTTCTCCTTGACAGCAGAAACAGAAGAA
AGGAACTTCCCCCATGGAAATGAACATCTGTTAAATTGGGCCCGAAAAGAGTATGGAGCAGTTACTTCATTCACC
GAACTCAAGATAGCAAGAAACATTTATATTAAAGTGGGGGAAGATCAAGTGTTCCCTCCAAAGTGCAACATAGGG
AAGAATTTTCTCTCACTCAATTACCTTGCTGAGTACCTTCAACCCAAAGCAGCAGAAGGGTGTGTGATGTCCAGC
CAGCCCCAGAATGAGGAAGTACACATCATCGAGCTAATCACCCCCAACTCTAACCCCTACAGTGCTTTCCAGGTG
GATATAACAATTGATATAAGACCTTCTCAAGAGGATCTTGAAGTGGTCAAAAATCTCATCCTGATCTTGAAGTGC
AAAAAGTCTGTCAACTGGGTGATCAAATCTTTTGATGTTAAGGGAAGCCTGAAAATTATTGCTCCTAACAGTATT
GGCTTTGGAAAAGAGAGTGAAAGATCTATGACAATGACCAAATCAATAAGAGATGACATTCCTTCAACCCAAGGG
AATCTGGTGAAGTGGGCTTTGGACAATGGCTATAGTCCAATAACTTCATACACAATGGCTCCTGTGGCTAATAGA
TTTCATCTTCGGCTTGAAAATAATGAGGAGATGGGAGATGAGGAAGTCCACACTATTCCTCCTGAGCTACGGATC
CTGCTGGACCCTGGTGCCCTGCCTGCCCTGCAGAACCCGCCCATCCGGGGAGGGGAAGGCCAAAATGGAGGCCTT
CCGTTTCCTTTCCCAGATATTTCCAGGAGAGTCTGGAATGAAGAGGGAGAAGATGGGCTCCCTCGGCCAAAGGAC
CCTGTCATTCCCAGCATACAACTGTTTCCTGGTCTCAGAGAGCCAGAAGAGGTGCAAGGGAGCGTGGATATTGCC
CTGTCTGTCAAATGTGACAATGAGAAGATGATCGTGGCTGTAGAAAAAGATTCTTTTCAGGCCAGTGGCTACTCG
GGGATGGACGTCACCCTGTTGGATCCTACCTGCAAGGCCAAGATGAATGGCACACACTTTGTTTTGGAGTCTCCT
CTGAATGGCTGCGGTACTCGGCCCCGGTGGTCAGCCCTTGATGGTGTGGTCTACTATAACTCCATTGTGATACAG
GTTCCAGCCCTTGGGGACAGTAGTGGTTGGCCAGATGGTTATGAAGATCTGGAGTCAGGTGATAATGGATTTCCG
GGAGATATGGATGAAGGAGATGCTTCCCTGTTCACCCGACCTGAAATCGTGGTGTTTAATTGCAGCCTTCAGCAG
GTGAGGAACCCCAGCAGCTTCCAGGAACAGCCCCACGGAAACATCACCTTCAACATGGAGCTATACAACACTGAC
CTCTTTTTGGTGCCCTCCCAGGGCGTCTTCTCTGTGCCAGAGAATGGACACGTTTATGTTGAGGTATCTGTTACT
AAGGCTGAACAAGAACTGGGATTTGCCATCCAAACGTGCTTTATCTCTCCATATTCGAACCCTGATAGGATGTCT
CATTACACCATTATTGAGAATATTTGTCCTAAAGATGAATCTGTGAAATTCTACAGTCCCAAGAGAGTGCACTTT
CCTATCCCGCAAGCTGACATGGATAAGAAGCGATTCAGCTTTGTCTTCAAGCCTGTCTTCAACACCTCACTGCTC
TTTCTACAGTGTGAGCTGACGCTGTGTACGAAGATGGAGAAGCACCCCCAGAAGTTGCCTAAGTGTGTGCCTCCT
GACGAAGCCTGCACCTCGCTGGACGCCTCGATAATCTGGGCCATGATGCAGAATAAGAAGACGTTCACTAAGCCC
CTTGCTGTGATCCACCATGAAGCAGAATCTAAAGAAAAAGGTCCAAGCATGAAGGAACCAAATCCAATTTCTCCA

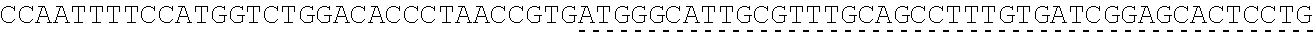

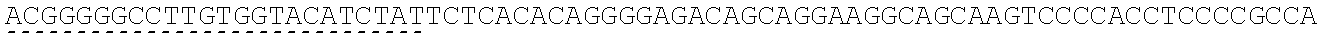

GCCTCGGAAAACAGCAGTGCTGCCCACAGCATCGGCAGCACGCAGAGCACGCCTTGCTCCAGCAGCAGCACGGCC

A nucleic acid sequence encoding a processed extracellular domain of betaglycan isoform B is shown below (SEQ ID NO: 92):

(SEQ ID NO: 92)
GGTCCAGAGCCTGGTGCACTGTGTGAACTGTCACCTG
TCAGTGCCTCCCATCCTGTCCAGGCCTTGATGGAGAG
CTTCACTGTTTTGTCAGGCTGTGCCAGCAGAGGCACA
ACTGGGCTGCCACAGGAGGTGCATGTCCTGAATCTCC
GCACTGCAGGCCAGGGGCCTGGCCAGCTACAGAGAGA
GGTCACACTTCACCTGAATCCCATCTCCTCAGTCCAC
ATCCACCACAAGTCTGTTGTGTTCCTGCTCAACTCCC
CACACCCCCTGGTGTGGCATCTGAAGACAGAGAGACT
TGCCACTGGGGTCTCCAGACTGTTTTTGGTGTCTGAG
GGTTCTGTGGTCCAGTTTTCATCAGCAAACTTCTCCT
TGACAGCAGAAACAGAAGAAAGGAACTTCCCCCATGG
AAATGAACATCTGTTAAATTGGGCCCGAAAAGAGTAT
GGAGCAGTTACTTCATTCACCGAACTCAAGATAGCAA
GAAACATTTATATTAAAGTGGGGGAAGATCAAGTGTT
CCCTCCAAAGTGCAACATAGGGAAGAATTTTCTCTCA
CTCAATTACCTTGCTGAGTACCTTCAACCCAAAGCAG
CAGAAGGGTGTGTGATGTCCAGCCAGCCCCAGAATGA
GGAAGTACACATCATCGAGCTAATCACCCCCAACTCT
AACCCCTACAGTGCTTTCCAGGTGGATATAACAATTG
ATATAAGACCTTCTCAAGAGGATCTTGAAGTGGTCAA
AAATCTCATCCTGATCTTGAAGTGCAAAAAGTCTGTC
AACTGGGTGATCAAATCTTTTGATGTTAAGGGAAGCC
TGAAAATTATTGCTCCTAACAGTATTGGCTTTGGAAA
AGAGAGTGAAAGATCTATGACAATGACCAAATCAATA
AGAGATGACATTCCTTCAACCCAAGGGAATCTGGTGA
AGTGGGCTTTGGACAATGGCTATAGTCCAATAACTTC
ATACACAATGGCTCCTGTGGCTAATAGATTTCATCTT
CGGCTTGAAAATAATGAGGAGATGGGAGATGAGGAAG
TCCACACTATTCCTCCTGAGCTACGGATCCTGCTGGA
CCCTGGTGCCCTGCCTGCCCTGCAGAACCCGCCCATC
CGGGGAGGGGAAGGCCAAAATGGAGGCCTTCCGTTTC
CTTTCCCAGATATTTCCAGGAGAGTCTGGAATGAAGA
GGGAGAAGATGGGCTCCCTCGGCCAAAGGACCCTGTC
ATTCCCAGCATACAACTGTTTCCTGGTCTCAGAGAGC
CAGAAGAGGTGCAAGGGAGCGTGGATATTGCCCTGTC
TGTCAAATGTGACAATGAGAAGATGATCGTGGCTGTA
GAAAAAGATTCTTTTCAGGCCAGTGGCTACTCGGGGA
TGGACGTCACCCTGTTGGATCCTACCTGCAAGGCCAA
GATGAATGGCACACACTTTGTTTTGGAGTCTCCTCTG
AATGGCTGCGGTACTCGGCCCCGGTGGTCAGCCCTTG
ATGGTGTGGTCTACTATAACTCCATTGTGATACAGGT
TCCAGCCCTTGGGGACAGTAGTGGTTGGCCAGATGGT
TATGAAGATCTGGAGTCAGGTGATAATGGATTTCCGG
GAGATATGGATGAAGGAGATGCTTCCCTGTTCACCCG
ACCTGAAATCGTGGTGTTTAATTGCAGCCTTCAGCAG
GTGAGGAACCCCAGCAGCTTCCAGGAACAGCCCCACG
GAAACATCACCTTCAACATGGAGCTATACAACACTGA
CCTCTTTTTGGTGCCCTCCCAGGGCGTCTTCTCTGTG
CCAGAGAATGGACACGTTTATGTTGAGGTATCTGTTA
CTAAGGCTGAACAAGAACTGGGATTTGCCATCCAAAC
GTGCTTTATCTCTCCATATTCGAACCCTGATAGGATG
TCTCATTACACCATTATTGAGAATATTTGTCCTAAAG
ATGAATCTGTGAAATTCTACAGTCCCAAGAGAGTGCA
CTTTCCTATCCCGCAAGCTGACATGGATAAGAAGCGA
TTCAGCTTTGTCTTCAAGCCTGTCTTCAACACCTCAC
TGCTCTTTCTACAGTGTGAGCTGACGCTGTGTACGAA
GATGGAGAAGCACCCCCAGAAGTTGCCTAAGTGTGTG
CCTCCTGACGAAGCCTGCACCTCGCTGGACGCCTCGA
TAATCTGGGCCATGATGCAGAATAAGAAGACGTTCAC
TAAGCCCCTTGCTGTGATCCACCATGAAGCAGAATCT
AAAGAAAAAGGTCCAAGCATGAAGGAACCAAATCCAA
TTTCTCCACCAATTTTCCATGGTCTGGACACCCTAAC
CGTG

In certain embodiments, the disclosure relates to heteromultimers that comprise at least one betaglycan polypeptide, which includes fragments, functional variants, and modified forms thereof. Preferably, betaglycan polypeptides for use in accordance with inventions of the disclosure (e.g., heteromultimers comprising a betaglycan polypeptide and uses thereof) are soluble (e.g., an extracellular domain of betaglycan). In other preferred embodiments, betaglycan polypeptides for use in accordance with the inventions of the disclosure bind to and/or inhibit (antagonize) activity (e.g., Smad signaling) of one or more TGF-beta superfamily ligands. In some embodiments, heteromultimers of the disclosure comprise of at least one betaglycan polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 85, 86, 89, or 90. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-28 (e.g., amino acid residues 21, 22, 23, 24, 25, 26, 27, or 28) of SEQ ID NO: 85, and ends at any one of amino acids 381-787 (e.g., amino acid residues 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, or 787) of SEQ ID NO: 85. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-381 of SEQ ID NO: 85. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-787 of SEQ ID NO: 85. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-381 of SEQ ID NO: 85. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-787 of SEQ ID NO: 85. In some embodiments, heteromultimers of the disclosure comprise of at least one betaglycan polypeptide that is at least 70%, 75%, 80%, 85%, 90%, 91%, 92%, 93%, 94%, 95%, 96%, 97%, 98%, 99%, or 100% identical to amino acids of 21-781 of SEQ ID NO: 85. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-781 of SEQ ID NO: 85. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-28 (e.g., amino acid residues 21, 22, 23, 24, 25, 26, or 27) of SEQ ID NO: 89, and ends at any one of amino acids 380-786 (e.g., amino acid residues 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, or 786) of SEQ ID NO: 89. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-380 of SEQ ID NO: 89. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-786 of SEQ ID NO: 89. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-380 of SEQ ID NO: 89. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-786 of SEQ ID NO: 89. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-780 of SEQ ID NO: 89. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-780 of SEQ ID NO: 89. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-28 (e.g., amino acid residues 21, 22, 23, 24, 25, 26, 27, or 28) of SEQ ID NO: 85, and ends at any one of amino acids 730-787 (e.g., amino acid residues 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, or 787) of SEQ ID NO: 85. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-787 of SEQ ID NO: 85. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-730 of SEQ ID NO: 85. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-28 (e.g., amino acid residues 21, 22, 23, 24, 25, 26, 27, or 28) of SEQ ID NO: 85, and ends at any one of amino acids 730-787 (e.g., amino acid residues 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, or 787) of SEQ ID NO: 85. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-787 of SEQ ID NO: 85. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-730 of SEQ ID NO: 85. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-28 (e.g., amino acid residues 21, 22, 23, 24, 25, 26, 27, or 28) of SEQ ID NO: 85, and ends at any one of amino acids 730-787 (e.g., amino acid residues 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, or 786) of SEQ ID NO: 85. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 21-786 of SEQ ID NO: 85. In some embodiments, heteromultimers of the disclosure comprise at least one betaglycan polypeptide that is at least 70%, 75%, 80%, 85%, 90%, 91%, 92%, 93%, 94%, 95%, 96%, 97%, 98%, 99%, or 100% identical to amino acids of 28-729 of SEQ ID NO: 85.

The term “MuSK polypeptide” includes polypeptides comprising any naturally occurring MuSK protein (encoded by MUSK or one of its nonhuman orthologs) as well as any variants thereof (including mutants, fragments, fusions, and peptidomimetic forms) that retain a useful activity.

A human MuSK isoform 1 precursor protein sequence (NCBI Reference Sequence NP_005583.1) is as follows:

| (SEQ ID NO: 95) | |
|---|---|
| 1 | MRELVNIPLV HILTLVAFSG TEKLPKAPVI TTPLETVDAL VEEVATFMCA |
| 51 | VESYPQPEIS WTRNKILIKL FDTRYSIREN GQLLTILSVE DSDDGIYCCT |
| 101 | ANNGVGGAVE SCGALQVKMK PKITRPPINV KIIEGLKAVL PCTTMGNPKP |
| 151 | SVSWIKGDSP LRENSRIAVL ESGSLRIHNV QKEDAGQYRC VAKNSLGTAY |
| 201 | SKVVKLEVEV FARILRAPES HNVTFGSFVT LHCTATGIPV PTITWIENGN |
| 251 | AVSSGSIQES VKDRVIDSRL QLFITKPGLY TCIATNKHGE KFSTAKAAAT |
| 301 | ISIAEWSKPQ KDNKGYCAQY RGEVCNAVLA KDALVFLNTS YADPEEAQEL |
| 351 | LVHTAWNELK VVSPVCRPAA EALLCNHIFQ ECSPGVVPTP IPICREYCLA |
| 401 | VKELFCAKEW LVMEEKTHRG LYRSEMHLLS VPECSKLPSM HWDPTACARL |
| 451 | 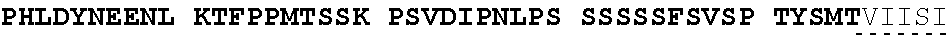 |
| 501 | 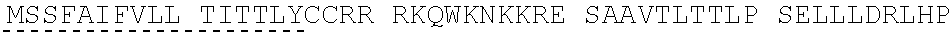 |
| 551 | NPMYQRMPLL LNPKLLSLEY PRNNIEYVRD IGEGAFGRVF QARAPGLLPY |
| 601 | EPFTMVAVKM LKEEASADMQ ADFQREAALM AEFDNPNIVK LLGVCAVGKP |
| 651 | MCLLFEYMAY GDLNEFLRSM SPHTVCSLSH SDLSMRAQVS SPGPPPLSCA |
| 701 | EQLCIARQVA AGMAYLSERK FVHRDLATRN CLVGENMVVK IADFGLSRNI |
| 751 | YSADYYKANE NDAIPIRWMP PESIFYNRYT TESDVWAYGV VLWEIFSYGL |
| 801 | QPYYGMAHEE VIYYVRDGNI LSCPENCPVE LYNLMRLCWS KLPADRPSFT |
| 851 | SIHRILERMC ERAEGTVSV |

The signal peptide is indicated by single underline, the extracellular domain is indicated in bold font, and the transmembrane domain is indicated by dotted underline. This isoform is the longest of human MuSK isoforms 1, 2, and 3.

A processed MuSK isoform 1 polypeptide sequence (SEQ ID NO: 96) is as follows:

```
(SEQ ID NO: 96)
    1 GTEKLPKAPV ITTPLETVDA LVEEVATFMC
        AVESYPQPEI SWTRNKILIK
  51 LFDTRYSIRE NGQLLTILSV EDSDDGIYCC
        TANNGVGGAV ESCGALQVKM
101 KPKITRPPIN VKIIEGLKAV LPCTTMGNPK
        PSVSWIKGDS PLRENSRIAV
151 LESGSLRIHN VQKEDAGQYR CVAKNSLGTA
        YSKVVKLEVE VFARILRAPE
201 SHNVTFGSFV TLHCTATGIP VPTITWIENG
        NAVSSGSIQE SVKDRVIDSR
251 LQLFITKPGL YTCIATNKHG EKFSTAKAAA
        TISIAEWSKP QKDNKGYCAQ
301 YRGEVCNAVL AKDALVFLNT SYADPEEAQE
        LLVHTAWNEL KVVSPVCRPA
351 AEALLCNHIF QECSPGVVPT PIPICREYCL
        AVKELFCAKE WLVMEEKTHR
401 GLYRSEMHLL SVPECSKLPS MHWDPTACAR
        LPHLDYNKEN LKTFPPMTSS
451 KPSVDIPNLP SSSSSSFSVS PTYSMT
```

A nucleic acid sequence encoding the unprocessed precursor protein of human MuSK isoform 1 is shown below (SEQ ID NO: 97), corresponding to nucleotides 135-2744 of NCBI Reference Sequence NM_005592.3. The signal sequence is indicated by solid underline and the transmembrane region by dotted underline.

(SEQ ID NO: 97)
ATGAGAGAGCTCGTCAACATTCCACTGGTACATATTCTTACTCTGGTTGCCTTCAGCGGAACTGAGAAACTTCCA
AAAGCTCCTGTCATCACCACTCCTCTTGAAACAGTGGATGCCTTAGTTGAAGAAGTGGCTACTTTCATGTGTGCA
GTGGAATCCTACCCCCAGCCTGAGATTTCCTGGACTAGAAATAAAATTCTCATTAAACTCTTTGACACCCGGTAC
AGCATCCGGGAGAATGGGCAGCTCCTCACCATCCTGAGTGTGGAAGACAGTGATGATGGCATTTACTGCTGCACG
GCCAACAATGGTGTGGGAGGAGCTGTGGAGAGTTGTGGAGCCCTGCAAGTGAAGATGAAACCTAAAATAACTCGT
CCTCCCATAAATGTGAAAATAATAGAGGGATTAAAAGCAGTCCTACCATGTACTACAATGGGTAATCCCAAACCA
TCAGTGTCTTGGATAAAGGGAGACAGCCCTCTCAGGGAAAATTCCCGAATTGCAGTTCTTGAATCTGGGAGCTTG
AGGATTCATAACGTACAAAAGGAAGATGCAGGACAGTATCGATGTGTGGCAAAAAACAGCCTCGGGACAGCATAT
TCCAAAGTGGTGAAGCTGGAAGTTGAGGTTTTTGCCAGGATCCTGCGGGCTCCTGAATCCCACAATGTCACCTTT
GGCTCCTTTGTGACCCTGCACTGTACAGCAACAGGCATTCCTGTCCCCACCATCACCTGGATTGAAAACGGAAAT
GCTGTTTCTTCTGGGTCCATTCAAGAGAGTGTGAAAGACCGAGTGATTGACTCAAGACTGCAGCTGTTTATCACC
AAGCCAGGACTCTACACATGCATAGCTACCAATAAGCATGGGGAGAAGTTCAGTACTGCCAAGGCTGCAGCCACC
ATCAGCATAGCAGAATGGAGTAAACCACAGAAAGATAACAAAGGCTACTGCGCCCAGTACAGAGGGGAGGTGTGT
AATGCAGTCCTGGCAAAAGATGCTCTTGTTTTTCTCAACACCTCCTATGCGGACCCTGAGGAGGCCCAAGAGCTA
CTGGTCCACACGGCCTGGAATGAACTGAAAGTAGTGAGCCCAGTCTGCCGGCCAGCTGCTGAGGCTTTGTTGTGT
AACCACATCTTCCAGGAGTGCAGTCCTGGAGTAGTGCCTACTCCTATTCCCATTTGCAGAGAGTACTGCTTGGCA
GTAAAGGAGCTCTTCTGCGCAAAAGAATGGCTGGTAATGGAAGAGAAGACCCACAGAGGACTCTACAGATCCGAG
ATGCATTTGCTGTCCGTGCCAGAATGCAGCAAGCTTCCCAGCATGCATTGGGACCCCACGGCCTGTGCCAGACTG
CCACATCTAGATTATAACAAAGAAAACCTAAAAACATTCCCACCAATGACGTCCTCAAAGCCAAGTGTGGACATT

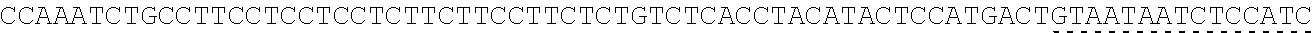

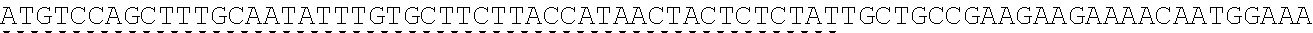

AATAAGAAAAGAGAATCAGCAGCAGTAACCCTCACCACACTGCCTTCTGAGCTCTTACTAGATAGACTTCATCCC
AACCCCATGTACCAGAGGATGCCGCTCCTTCTGAACCCCAAATTGCTCAGCCTGGAGTATCCAAGGAATAACATT
GAATATGTGAGAGACATCGGAGAGGGAGCGTTTGGAAGGGTGTTTCAAGCAAGGGCACCAGGCTTACTTCCCTAT
GAACCTTTCACTATGGTGGCAGTAAAGATGCTCAAAGAAGAAGCCTCGGCAGATATGCAAGCGGACTTTCAGAGG
GAGGCAGCCCTCATGGCAGAATTTGACAACCCTAACATTGTGAAGCTATTAGGAGTGTGTGCTGTCGGGAAGCCA
ATGTGCCTGCTCTTTGAATACATGGCCTATGGTGACCTCAATGAGTTCCTCCGCAGCATGTCCCCTCACACCGTG
TGCAGCCTCAGTCACAGTGACTTGTCTATGAGGGCTCAGGTCTCCAGCCCTGGGCCCCCACCCCTCTCCTGTGCT
GAGCAGCTTTGCATTGCCAGGCAGGTGGCAGCTGGCATGGCTTACCTCTCAGAACGTAAGTTTGTTCACCGAGAT
TTAGCCACCAGGAACTGCCTGGTGGGCGAGAACATGGTGGTGAAAATTGCCGACTTTGGCCTCTCCAGGAACATC
TACTCAGCAGACTACTACAAAGCTAATGAAAACGACGCTATCCCTATCCGTTGGATGCCACCAGAGTCCATTTTT
TATAACCGCTACACTACAGAGTCTGATGTGTGGGCCTATGGCGTGGTCCTCTGGGAGATCTTCTCCTATGGCCTG
CAGCCCTACTATGGGATGGCCCATGAGGAGGTCATTTACTACGTGCGAGATGGCAACATCCTCTCCTGCCCTGAG
AACTGCCCCGTGGAGCTGTACAATCTCATGCGTCTATGTTGGAGCAAGCTGCCTGCAGACAGACCCAGTTTCACC
AGTATTCACCGAATTCTGGAACGCATGTGTGAGAGGGCAGAGGGAACTGTGAGTGTC

A nucleic acid sequence encoding a processed extracellular domain of MuSK isoform 1 is shown below (SEQ ID NO: 98):

(SEQ ID NO: 98)
GGAACTGAGAAACTTCCAAAAGCTCCTGTCATCACCACTCCTCTTGAAA
CAGTGGATGCCTTAGTTGAAGAAGTGGCTACTTTCATGTGTGCAGTGGA
ATCCTACCCCCAGCCTGAGATTTCCTGGACTAGAAATAAAATTCTCATT
AAACTCTTTGACACCCGGTACAGCATCCGGGAGAATGGGCAGCTCCTCA
CCATCCTGAGTGTGGAAGACAGTGATGATGGCATTTACTGCTGCACGGC
CAACAATGGTGTGGGAGGAGCTGTGGAGAGTTGTGGAGCCCTGCAAGTG
AAGATGAAACCTAAAATAACTCGTCCTCCCATAAATGTGAAAATAATAG
AGGGATTAAAAGCAGTCCTACCATGTACTACAATGGGTAATCCCAAACC
ATCAGTGTCTTGGATAAAGGGAGACAGCCCTCTCAGGGAAAATTCCCGA
ATTGCAGTTCTTGAATCTGGGAGCTTGAGGATTCATAACGTACAAAAGG
AAGATGCAGGACAGTATCGATGTGTGGCAAAAAACAGCCTCGGGACAGC
ATATTCCAAAGTGGTGAAGCTGGAAGTTGAGGTTTTTGCCAGGATCCTG
CGGGCTCCTGAATCCCACAATGTCACCTTTGGCTCCTTTGTGACCCTGC
ACTGTACAGCAACAGGCATTCCTGTCCCCACCATCACCTGGATTGAAAA
CGGAAATGCTGTTTCTTCTGGGTCCATTCAAGAGAGTGTGAAAGACCGA
GTGATTGACTCAAGACTGCAGCTGTTTATCACCAAGCCAGGACTCTACA
CATGCATAGCTACCAATAAGCATGGGGAGAAGTTCAGTACTGCCAAGGC
TGCAGCCACCATCAGCATAGCAGAATGGAGTAAACCACAGAAAGATAAC
AAAGGCTACTGCGCCCAGTACAGAGGGGAGGTGTGTAATGCAGTCCTGG
CAAAAGATGCTCTTGTTTTTCTCAACACCTCCTATGCGGACCCTGAGGA
GGCCCAAGAGCTACTGGTCCACACGGCCTGGAATGAACTGAAAGTAGTG
AGCCCAGTCTGCCGGCCAGCTGCTGAGGCTTTGTTGTGTAACCACATCT
TCCAGGAGTGCAGTCCTGGAGTAGTGCCTACTCCTATTCCCATTTGCAG
AGAGTACTGCTTGGCAGTAAAGGAGCTCTTCTGCGCAAAAGAATGGCTG
GTAATGGAAGAGAAGACCCACAGAGGACTCTACAGATCCGAGATGCATT
TGCTGTCCGTGCCAGAATGCAGCAAGCTTCCCAGCATGCATTGGGACCC
CACGGCCTGTGCCAGACTGCCACATCTAGATTATAACAAAGAAAACCTA
AAAACATTCCCACCAATGACGTCCTCAAAGCCAAGTGTGGACATTCCAA
ATCTGCCTTCCTCCTCCTCTTCTTCCTTCTCTGTCTCACCTACATACTC
CATGACT

A human MuSK isoform 2 precursor protein sequence (NCBI Reference Sequence NP_001159752.1) is as follows:

| (SEQ ID NO: 99) | |
|---|---|
| 1 | MRELVNIPLV HILTLVAFSG TEKLPKAPVI TTPLETVDAL VEEVATFMCA |
| 51 | VESYPQPEIS WTRNKILIKL FDTRYSIREN GQLLTILSVE DSDDGIYCCT |
| 101 | ANNGVGGAVE SCGALQVKMK PKITRPPINV KIIEGLKAVL PCTTMGNPKP |
| 151 | SVSWIKGDSP LRENSRIAVL ESGSLRIHNV QKEDAGQYRC VAKNSLGTAY |
| 201 | SKVVKLEVEE ESEPEQDTKV FARILRAPES HNVITGSFVT LHCTATGIPV |
| 251 | PTITWIENGN AVSSGSIQES VKDRVIDSRL QLFITKPGLY TCIATNKHGE |
| 301 | KFSTAKAAAT ISIAEWREYC LAVKELFCAK EWLVMEEKTH RGLYRSEMHL |
| 351 | LSVPECSKLP SMHWDPTACA RLPHLAFPPM TSSKPSVDIP NLPSSSSSSF |
| 401 | 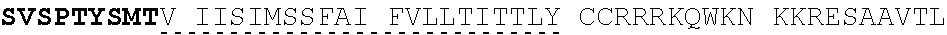 |
| 451 | TTLPSELLLD RLHPNPMYQR MPLLLNPKLL SLEYPRNNIE YVRDIGEGAF |
| 501 | GRVFQARAPG LLPYEPFTMV AVKMLKEEAS ADMQADFQRE AALMAEFDNP |
| 551 | NIVKLLGVCA VGKPMCLLFE YMAYGDLNEF LRSMSPHTVC SLSHSDLSMR |
| 601 | AQVSSPGPPP LSCAEQLCIA RQVAAGMAYL SERKFVHRDL ATRNCLVGEN |
| 651 | MVVKIADFGL SRNIYSADYY KANENDAIPI RWMPPESIFY NRYTTESDVW |
| 701 | AYGVVLWEIF SYGLQPYYGM AHEEVIYYVR DGNILSCPEN CPVELYNLMR |
| 751 | LCWSKLPADR PSFTSIHRIL ERMCERAEGT VSV |

The signal peptide is indicated by single underline, the extracellular domain is indicated in bold font, and the transmembrane domain is indicated by dotted underline. This variant contains an alternate in-frame exon and lacks an alternate in-frame exon in the middle portion of the coding region compared to variant 1. The encoded isoform 2 is shorter than isoform 1.

A mature MuSK isoform 2 polypeptide sequence (SEQ ID NO: 100) is as follows:

| (SEQ ID NO: 100) | |
|---|---|
| 1 | GTEKLPKAPV ITTPLETVDA LVEEVATFMC AVESYPQPEI SWTRNKILIK |
| 51 | LFDTRYSIRE NGQLLTILSV EDSDDGIYCC TANNGVGGAV ESCGALQVKM |
| 101 | KPKITRPPIN VKIIEGLKAV LPCTTMGNPK PSVSWIKGDS PLRENSRIAV |
| 151 | LESGSLRIHN VQKEDAGQYR CVAKNSLGTA YSKVVKLEVE EESEPEQDTK |
| 201 | VFARILRAPE SHNVTFGSFV TLHCTATGIP VPTITWIENG NAVSSGSIQE |
| 251 | SVKDRVIDSR LQLFITKPGL YTCIATNKHG EKFSTAKAAA TISIAEWREY |
| 301 | CLAVKELFCA KEWLVMEEKT HRGLYRSEMH LLSVPECSKL PSMHWDPTAC |
| 351 | ARLPHLAFPP MTSSKPSVDI PNLPSSSSSS FSVSPTYSMT |

A nucleic acid sequence encoding the unprocessed precursor protein of human MuSK isoform 2 is shown below (SEQ ID NO: 101), corresponding to nucleotides 135-2483 of NCBI Reference Sequence NM_001166280.1. The signal sequence is indicated by solid underline and the transmembrane region by dotted underline.

ATGAGAGAGCTCGTCAACATTCCACTGGTACATATTCTTACTCTGGTTGCCTTCAGCGGAACTGAGAAACTTCCA
AAAGCTCCTGTCATCACCACTCCTCTTGAAACAGTGGATGCCTTAGTTGAAGAAGTGGCTACTTTCATGTGTGCA
GTGGAATCCTACCCCCAGCCTGAGATTTCCTGGACTAGAAATAAAATTCTCATTAAACTCTTTGACACCCGGTAC
AGCATCCGGGAGAATGGGCAGCTCCTCACCATCCTGAGTGTGGAAGACAGTGATGATGGCATTTACTGCTGCACG
GCCAACAATGGTGTGGGAGGAGCTGTGGAGAGTTGTGGAGCCCTGCAAGTGAAGATGAAACCTAAAATAACTCGT
CCTCCCATAAATGTGAAAATAATAGAGGGATTAAAAGCAGTCCTACCATGTACTACAATGGGTAATCCCAAACCA
TCAGTGTCTTGGATAAAGGGAGACAGCCCTCTCAGGGAAAATTCCCGAATTGCAGTTCTTGAATCTGGGAGCTTG
AGGATTCATAACGTACAAAAGGAAGATGCAGGACAGTATCGATGTGTGGCAAAAAACAGCCTCGGGACAGCATAT
TCCAAAGTGGTGAAGCTGGAAGTTGAGGAAGAAAGTGAACCCGAACAAGATACTAAAGTTTTTGCCAGGATCCTG
CGGGCTCCTGAATCCCACAATGTCACCTTTGGCTCCTTTGTGACCCTGCACTGTACAGCAACAGGCATTCCTGTC
CCCACCATCACCTGGATTGAAAACGGAAATGCTGTTTCTTCTGGGTCCATTCAAGAGAGTGTGAAAGACCGAGTG
ATTGACTCAAGACTGCAGCTGTTTATCACCAAGCCAGGACTCTACACATGCATAGCTACCAATAAGCATGGGGAG
AAGTTCAGTACTGCCAAGGCTGCAGCCACCATCAGCATAGCAGAATGGAGAGAGTACTGCTTGGCAGTAAAGGAG
CTCTTCTGCGCAAAAGAATGGCTGGTAATGGAAGAGAAGACCCACAGAGGACTCTACAGATCCGAGATGCATTTG
CTGTCCGTGCCAGAATGCAGCAAGCTTCCCAGCATGCATTGGGACCCCACGGCCTGTGCCAGACTGCCACATCTA
GCATTCCCACCAATGACGTCCTCAAAGCCAAGTGTGGACATTCCAAATCTGCCTTCCTCCTCCTCTTCTTCCTTC

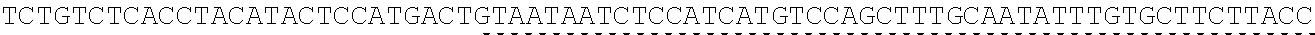

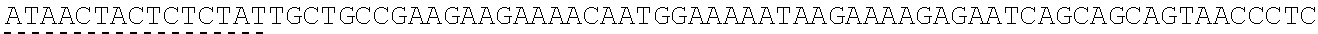

ACCACACTGCCTTCTGAGCTCTTACTAGATAGACTTCATCCCAACCCCATGTACCAGAGGATGCCGCTCCTTCTG
AACCCCAAATTGCTCAGCCTGGAGTATCCAAGGAATAACATTGAATATGTGAGAGACATCGGAGAGGGAGCGTTT
GGAAGGGTGTTTCAAGCAAGGGCACCAGGCTTACTTCCCTATGAACCTTTCACTATGGTGGCAGTAAAGATGCTC
AAAGAAGAAGCCTCGGCAGATATGCAAGCGGACTTTCAGAGGGAGGCAGCCCTCATGGCAGAATTTGACAACCCT
AACATTGTGAAGCTATTAGGAGTGTGTGCTGTCGGGAAGCCAATGTGCCTGCTCTTTGAATACATGGCCTATGGT
GACCTCAATGAGTTCCTCCGCAGCATGTCCCCTCACACCGTGTGCAGCCTCAGTCACAGTGACTTGTCTATGAGG
GCTCAGGTCTCCAGCCCTGGGCCCCCACCCCTCTCCTGTGCTGAGCAGCTTTGCATTGCCAGGCAGGTGGCAGCT
GGCATGGCTTACCTCTCAGAACGTAAGTTTGTTCACCGAGATTTAGCCACCAGGAACTGCCTGGTGGGCGAGAAC
ATGGTGGTGAAAATTGCCGACTTTGGCCTCTCCAGGAACATCTACTCAGCAGACTACTACAAAGCTAATGAAAAC
GACGCTATCCCTATCCGTTGGATGCCACCAGAGTCCATTTTTTATAACCGCTACACTACAGAGTCTGATGTGTGG
GCCTATGGCGTGGTCCTCTGGGAGATCTTCTCCTATGGCCTGCAGCCCTACTATGGGATGGCCCATGAGGAGGTC
ATTTACTACGTGCGAGATGGCAACATCCTCTCCTGCCCTGAGAACTGCCCCGTGGAGCTGTACAATCTCATGCGT
CTATGTTGGAGCAAGCTGCCTGCAGACAGACCCAGTTTCACCAGTATTCACCGAATTCTGGAACGCATGTGTGAG
AGGGCAGAGGGAACTGTGAGTGTC (SEQ ID NO: 101)

A nucleic acid sequence encoding a processed extracellular domain of MuSK isoform 2 is shown below (SEQ ID NO: 102):

(SEQ ID NO: 102)
GGAACTGAGAAACTTCCAAAAGCTCCTGTCATCACCACTCCTCTTGAAA
CAGTGGATGCCTTAGTTGAAGAAGTGGCTACTTTCATGTGTGCAGTGGA
ATCCTACCCCCAGCCTGAGATTTCCTGGACTAGAAATAAAATTCTCATT
AAACTCTTTGACACCCGGTACAGCATCCGGGAGAATGGGCAGCTCCTCA
CCATCCTGAGTGTGGAAGACAGTGATGATGGCATTTACTGCTGCACGGC
CAACAATGGTGTGGGAGGAGCTGTGGAGAGTTGTGGAGCCCTGCAAGTG
AAGATGAAACCTAAAATAACTCGTCCTCCCATAAATGTGAAAATAATAG
AGGGATTAAAAGCAGTCCTACCATGTACTACAATGGGTAATCCCAAACC
ATCAGTGTCTTGGATAAAGGGAGACAGCCCTCTCAGGGAAAATTCCCGA
ATTGCAGTTCTTGAATCTGGGAGCTTGAGGATTCATAACGTACAAAAGG
AAGATGCAGGACAGTATCGATGTGTGGCAAAAAACAGCCTCGGGACAGC
ATATTCCAAAGTGGTGAAGCTGGAAGTTGAGGAAGAAAGTGAACCCGAA
CAAGATACTAAAGTTTTTGCCAGGATCCTGCGGGCTCCTGAATCCCACA
ATGTCACCTTTGGCTCCTTTGTGACCCTGCACTGTACAGCAACAGGCAT
TCCTGTCCCCACCATCACCTGGATTGAAAACGGAAATGCTGTTTCTTCT
GGGTCCATTCAAGAGAGTGTGAAAGACCGAGTGATTGACTCAAGACTGC
AGCTGTTTATCACCAAGCCAGGACTCTACACATGCATAGCTACCAATAA
GCATGGGGAGAAGTTCAGTACTGCCAAGGCTGCAGCCACCATCAGCATA
GCAGAATGGAGAGAGTACTGCTTGGCAGTAAAGGAGCTCTTCTGCGCAA
AAGAATGGCTGGTAATGGAAGAGAAGACCCACAGAGGACTCTACAGATC
CGAGATGCATTTGCTGTCCGTGCCAGAATGCAGCAAGCTTCCCAGCATG
CATTGGGACCCCACGGCCTGTGCCAGACTGCCACATCTAGCATTCCCAC
CAATGACGTCCTCAAAGCCAAGTGTGGACATTCCAAATCTGCCTTCCTC
CTCCTCTTCTTCCTTCTCTGTCTCACCTACATACTCCATGACT

A human MuSK isoform 3 precursor protein sequence (NCBI Reference Sequence NP_001159753.1) is as follows:

| (SEQ ID NO: 103) | |
|---|---|
| 1 | MRELVNIPLV HILTLVAFSG TEKLPKAPVI TTPLETVDAL VEEVATFMCA |
| 51 | VESYPQPEIS WTRNKILIKL FDTRYSIREN GQLLTILSVE DSDDGIYCCT |
| 101 | ANNGVGGAVE SCGALQVKMK PKITRPPINV KIIEGLKAVL PCTTMGNPKP |
| 151 | SVSWIKGDSP LRENSRIAVL ESGSLRIHNV QKEDAGQYRC VAKNSLGTAY |
| 201 | SKVVKLEVEV FARILRAPES HNVITGSFVT LHCTATGIPV PTITWIENGN |
| 251 | AVSSGSIQES VKDRVIDSRL QLFITKPGLY TCIATNKHGE KFSTAKAAAT |
| 301 | ISIAEWREYC LAVKELFCAK EWLVMEEKTH RGLYRSEMHL LSVPECSKLP |
| 351 | SMHWDPTACA RLPHLAFPPM TSSKPSVDIP NLPSSSSSSF SVSPTYSMTV |
| 401 | 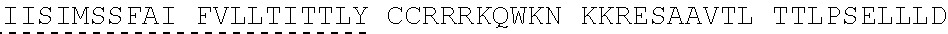 |
| 451 | RLHPNPMYQR MPLLLNPKLL SLEYPRNNIE YVRDIGEGAF GRVFQARAPG |
| 501 | LLPYEPFTMV AVKMLKEEAS ADMQADFQRE AALMAEFDNP NIVKLLGVCA |
| 551 | VGKPMCLLFE YMAYGDLNEF LRSMSPHTVC SLSHSDLSMR AQVSSPGPPP |
| 601 | LSCAEQLCIA RQVAAGMAYL SERKFVHRDL ATRNCLVGEN MVVKIADFGL |
| 651 | SRNIYSADYY KANENDAIPI RWMPPESIFY NRYTTESDVW AYGVVLWEIF |
| 701 | SYGLQPYYGM AHEEVIYYVR DGNILSCPEN CPVELYNLMR LCWSKLPADR |
| 751 | PSFTSIHRIL ERMCERAEGT VSV |

The signal peptide is indicated by single underline, the extracellular domain is indicated in bold font, and the transmembrane domain is indicated by dotted underline. This variant lacks an alternate in-frame exon in the middle portion of the coding region compared to variant 1. The encoded isoform 3 is shorter than isoform 1.

A processed MuSK isoform 3 polypeptide sequence (SEQ ID NO: 104) is as follows:

| (SEQ ID NO: 104) | |
|---|---|
| 1 | GTEKLPKAPV ITTPLETVDA LVEEVATFMC AVESYPQPEI SWTRNKILIK |
| 51 | LFDTRYSIRE NGQLLTILSV EDSDDGIYCC TANNGVGGAV ESCGALQVKM |
| 101 | KPKITRPPIN VKIIEGLKAV LPCTTMGNPK PSVSWIKGDS PLRENSRIAV |
| 151 | LESGSLRIHN VQKEDAGQYR CVAKNSLGTA YSKVVKLEVE VFARILRAPE |
| 201 | SHNVTFGSFV TLHCTATGIP VPTITWIENG NAVSSGSIQE SVKDRVIDSR |
| 251 | LQLFITKPGL YTCIATNKHG EKFSTAKAAA TISIAEWREY CLAVKELFCA |
| 301 | KEWLVMEEKT HRGLYRSEMH LLSVPECSKL PSMHWDPTAC ARLPHLAFPP |
| 351 | MTSSKPSVDI PNLPSSSSSS FSVSPTYSMT |

A nucleic acid sequence encoding the unprocessed precursor protein of human MuSK isoform 3 is shown below (SEQ ID NO: 105), corresponding to nucleotides 135-2453 of NCBI Reference Sequence NM_001166281.1. The signal sequence is indicated by solid underline and the transmembrane region by dotted underline.

ATGAGAGAGCTCGTCAACATTCCACTGGTACATATTCTTACTCTGGTTGCCTTCAGCGGAACTGAGAAACTTCCA
AAAGCTCCTGTCATCACCACTCCTCTTGAAACAGTGGATGCCTTAGTTGAAGAAGTGGCTACTTTCATGTGTGCA
GTGGAATCCTACCCCCAGCCTGAGATTTCCTGGACTAGAAATAAAATTCTCATTAAACTCTTTGACACCCGGTAC
AGCATCCGGGAGAATGGGCAGCTCCTCACCATCCTGAGTGTGGAAGACAGTGATGATGGCATTTACTGCTGCACG
GCCAACAATGGTGTGGGAGGAGCTGTGGAGAGTTGTGGAGCCCTGCAAGTGAAGATGAAACCTAAAATAACTCGT
CCTCCCATAAATGTGAAAATAATAGAGGGATTAAAAGCAGTCCTACCATGTACTACAATGGGTAATCCCAAACCA
TCAGTGTCTTGGATAAAGGGAGACAGCCCTCTCAGGGAAAATTCCCGAATTGCAGTTCTTGAATCTGGGAGCTTG
AGGATTCATAACGTACAAAAGGAAGATGCAGGACAGTATCGATGTGTGGCAAAAAACAGCCTCGGGACAGCATAT
TCCAAAGTGGTGAAGCTGGAAGTTGAGGTTTTTGCCAGGATCCTGCGGGCTCCTGAATCCCACAATGTCACCTTT
GGCTCCTTTGTGACCCTGCACTGTACAGCAACAGGCATTCCTGTCCCCACCATCACCTGGATTGAAAACGGAAAT
GCTGTTTCTTCTGGGTCCATTCAAGAGAGTGTGAAAGACCGAGTGATTGACTCAAGACTGCAGCTGTTTATCACC
AAGCCAGGACTCTACACATGCATAGCTACCAATAAGCATGGGGAGAAGTTCAGTACTGCCAAGGCTGCAGCCACC
ATCAGCATAGCAGAATGGAGAGAGTACTGCTTGGCAGTAAAGGAGCTCTTCTGCGCAAAAGAATGGCTGGTAATG
GAAGAGAAGACCCACAGAGGACTCTACAGATCCGAGATGCATTTGCTGTCCGTGCCAGAATGCAGCAAGCTTCCC
AGCATGCATTGGGACCCCACGGCCTGTGCCAGACTGCCACATCTAGCATTCCCACCAATGACGTCCTCAAAGCCA

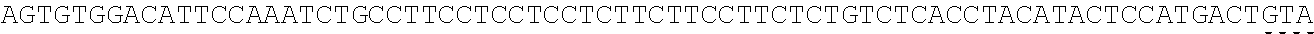

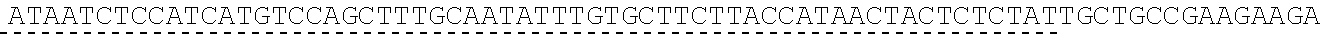

AAACAATGGAAAAATAAGAAAAGAGAATCAGCAGCAGTAACCCTCACCACACTGCCTTCTGAGCTCTTACTAGAT
AGACTTCATCCCAACCCCATGTACCAGAGGATGCCGCTCCTTCTGAACCCCAAATTGCTCAGCCTGGAGTATCCA
AGGAATAACATTGAATATGTGAGAGACATCGGAGAGGGAGCGTTTGGAAGGGTGTTTCAAGCAAGGGCACCAGGC
TTACTTCCCTATGAACCTTTCACTATGGTGGCAGTAAAGATGCTCAAAGAAGAAGCCTCGGCAGATATGCAAGCG
GACTTTCAGAGGGAGGCAGCCCTCATGGCAGAATTTGACAACCCTAACATTGTGAAGCTATTAGGAGTGTGTGCT
GTCGGGAAGCCAATGTGCCTGCTCTTTGAATACATGGCCTATGGTGACCTCAATGAGTTCCTCCGCAGCATGTCC
CCTCACACCGTGTGCAGCCTCAGTCACAGTGACTTGTCTATGAGGGCTCAGGTCTCCAGCCCTGGGCCCCCACCC
CTCTCCTGTGCTGAGCAGCTTTGCATTGCCAGGCAGGTGGCAGCTGGCATGGCTTACCTCTCAGAACGTAAGTTT
GTTCACCGAGATTTAGCCACCAGGAACTGCCTGGTGGGCGAGAACATGGTGGTGAAAATTGCCGACTTTGGCCTC
TCCAGGAACATCTACTCAGCAGACTACTACAAAGCTAATGAAAACGACGCTATCCCTATCCGTTGGATGCCACCA
GAGTCCATTTTTTATAACCGCTACACTACAGAGTCTGATGTGTGGGCCTATGGCGTGGTCCTCTGGGAGATCTTC
TCCTATGGCCTGCAGCCCTACTATGGGATGGCCCATGAGGAGGTCATTTACTACGTGCGAGATGGCAACATCCTC
TCCTGCCCTGAGAACTGCCCCGTGGAGCTGTACAATCTCATGCGTCTATGTTGGAGCAAGCTGCCTGCAGACAGA
CCCAGTTTCACCAGTATTCACCGAATTCTGGAACGCATGTGTGAGAGGGCAGAGGGAACTGTGAGTGTCTAA
(SEQ ID NO: 105)

A nucleic acid sequence encoding a processed extracellular domain of MuSK isoform 3 is shown below (SEQ ID NO: 106):

(SEQ ID NO: 106)
GGAACTGAGAAACTTCCAAAAGCTCCTGTCATCACCACTCCTCTTGAAA
CAGTGGATGCCTTAGTTGAAGAAGTGGCTACTTTCATGTGTGCAGTGGA
ATCCTACCCCCAGCCTGAGATTTCCTGGACTAGAAATAAAATTCTCATT
AAACTCTTTGACACCCGGTACAGCATCCGGGAGAATGGGCAGCTCCTCA
CCATCCTGAGTGTGGAAGACAGTGATGATGGCATTTACTGCTGCACGGC
CAACAATGGTGTGGGAGGAGCTGTGGAGAGTTGTGGAGCCCTGCAAGTG
AAGATGAAACCTAAAATAACTCGTCCTCCCATAAATGTGAAAATAATAG
AGGGATTAAAAGCAGTCCTACCATGTACTACAATGGGTAATCCCAAACC
ATCAGTGTCTTGGATAAAGGGAGACAGCCCTCTCAGGGAAAATTCCCGA
ATTGCAGTTCTTGAATCTGGGAGCTTGAGGATTCATAACGTACAAAAGG
AAGATGCAGGACAGTATCGATGTGTGGCAAAAAACAGCCTCGGGACAGC
ATATTCCAAAGTGGTGAAGCTGGAAGTTGAGGTTTTTGCCAGGATCCTG
CGGGCTCCTGAATCCCACAATGTCACCTTTGGCTCCTTTGTGACCCTGC
ACTGTACAGCAACAGGCATTCCTGTCCCCACCATCACCTGGATTGAAAA
CGGAAATGCTGTTTCTTCTGGGTCCATTCAAGAGAGTGTGAAAGACCGA
GTGATTGACTCAAGACTGCAGCTGTTTATCACCAAGCCAGGACTCTACA
CATGCATAGCTACCAATAAGCATGGGGAGAAGTTCAGTACTGCCAAGGC
TGCAGCCACCATCAGCATAGCAGAATGGAGAGAGTACTGCTTGGCAGTA
AAGGAGCTCTTCTGCGCAAAAGAATGGCTGGTAATGGAAGAGAAGACCC
ACAGAGGACTCTACAGATCCGAGATGCATTTGCTGTCCGTGCCAGAATG
CAGCAAGCTTCCCAGCATGCATTGGGACCCCACGGCCTGTGCCAGACTG
CCACATCTAGCATTCCCACCAATGACGTCCTCAAAGCCAAGTGTGGACA
TTCCAAATCTGCCTTCCTCCTCCTCTTCTTCCTTCTCTGTCTCACCTAC
ATACTCCATGACT

In certain embodiments, the disclosure relates to heteromultimers that comprise at least one MuSK polypeptide, which includes fragments, functional variants, and modified forms thereof. Preferably, MuSK polypeptides for use in accordance with the disclosure (e.g., heteromultimers comprising a MuSK polypeptide and uses thereof) are soluble (e.g., an extracellular domain of MuSK). In other preferred embodiments, MuSK polypeptides for use in accordance with disclosure bind to and/or inhibit (antagonize) activity (e.g., Smad signaling) of one or more TGF-beta superfamily ligands. In some embodiments, heteromultimers of the disclosure comprise at least one MuSK polypeptide that is at least 70%, 75%, 80%, 85%, 90%, 91%, 92%, 93%, 94%, 95%, 96%, 97%, 98%, 99%, or 100% identical to the amino acid sequence of SEQ ID NOs: 95, 96, 99, 100, 103, and 104. In some embodiments, heteromultimers of the disclosure comprise at least one MuSK polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 21-49 (e.g., amino acid residues 21, 22, 23, 24, 25, 26, 27, 28, 29, 30, 31, 32, 33, 34, 35, 35, 36, 37, 38, 39, 40, 41, 42, 43, 44, 45, 46, 47, 48, or 49) of SEQ ID NO: 95, and ends at any one of amino acids 447-495 (e.g., amino acid residues 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, or 495) of SEQ ID NO: 95. In some embodiments, heteromultimers of the disclosure comprise of at least one MuSK polypeptide that is at least 70%, 75%, 80%, 85%, 90%, 91%, 92%, 93%, 94%, 95%, 96%, 97%, 98%, 99%, or 100% identical to amino acids of 21-495 of SEQ ID NO: 95. In some embodiments, heteromultimers of the disclosure comprise of at least one MuSK polypeptide that is at least 70%, 75%, 80%, 85%, 90%, 91%, 92%, 93%, 94%, 95%, 96%, 97%, 98%, 99%, or 100% identical to amino acids of 49-447 of SEQ ID NO: 95. In some embodiments, heteromultimers of the disclosure comprise of at least one MuSK polypeptide that is at least 70%, 75%, 80%, 85%, 90%, 91%, 92%, 93%, 94%, 95%, 96%, 97%, 98%, 99%, or 100% identical to amino acids of 210-495 of SEQ ID NO: 95. In some embodiments, heteromultimers of the disclosure comprise at least one MuSK polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 20-49 (e.g., amino acid residues 20, 21, 22, 23, 24, 25, 26, 27, 28, 29, 30, 31, 32, 33, 34, 35, 35, 36, 37, 38, 39, 40, 41, 42, 43, 44, 45, 46, 47, 48, or 49) of SEQ ID NO: 99, and ends at any one of amino acids 369-409 (e.g., amino acid residues 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, or 409) of SEQ ID NO: 99. In some embodiments, heteromultimers of the disclosure comprise of at least one MuSK polypeptide that is at least 70%, 75%, 80%, 85%, 90%, 91%, 92%, 93%, 94%, 95%, 96%, 97%, 98%, 99%, or 100% identical to amino acids of 20-409 of SEQ ID NO: 99. In some embodiments, heteromultimers of the disclosure comprise of at least one MuSK polypeptide that is at least 70%, 75%, 80%, 85%, 90%, 91%, 92%, 93%, 94%, 95%, 96%, 97%, 98%, 99%, or 100% identical to amino acids of 49-369 of SEQ ID NO: 99. In some embodiments, heteromultimers of the disclosure comprise of at least one MuSK polypeptide that is at least 70%, 75%, 80%, 85%, 90%, 91%, 92%, 93%, 94%, 95%, 96%, 97%, 98%, 99%, or 100% identical to amino acids of 210-409 of SEQ ID NO: 99. In some embodiments, heteromultimers of the disclosure comprise at least one MuSK polypeptide that is at least 70%, 75%, 80%, 85%, 90%, 91%, 92%, 93%, 94%, 95%, 96%, 97%, 98%, 99%, or 100% identical to a polypeptide that begins at any one of amino acids of 20-49 (e.g., amino acid residues 20, 21, 22, 23, 24, 25, 26, 27, 28, 29, 30, 31, 32, 33, 34, 35, 35, 36, 37, 38, 39, 40, 41, 42, 43, 44, 45, 46, 47, 48, or 49) of SEQ ID NO: 103, and ends at any one of amino acids 359-399 (e.g., amino acid residues 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, or 399) of SEQ ID NO: 103. In some embodiments, heteromultimers of the disclosure comprise of at least one MuSK polypeptide that is at least 70%, 75%, 80%, 85%, 90%, 91%, 92%, 93%, 94%, 95%, 96%, 97%, 98%, 99%, or 100% identical to amino acids of 20-399 of SEQ ID NO: 103. In some embodiments, heteromultimers of the disclosure comprise of at least one MuSK polypeptide that is at least 70%, 75%, 80%, 85%, 90%, 91%, 92%, 93%, 94%, 95%, 96%, 97%, 98%, 99%, or 100% identical to amino acids of 49-359 of SEQ ID NO: 103. In some embodiments, heteromultimers of the disclosure comprise of at least one MuSK polypeptide that is at least 70%, 75%, 80%, 85%, 90%, 91%, 92%, 93%, 94%, 95%, 96%, 97%, 98%, 99%, or 100% identical to amino acids of 210-399 of SEQ ID NO: 103.

In some embodiments, the present disclosure contemplates making functional variants by modifying the structure of a TGF-beta superfamily co-receptor (e.g., endoglin, betaglycan, Cripto-1, Cryptic, Cryptic family protein 1B, CRIM1, CRIM2, BAMBI, BMPER, RGM-A, RGM-B, hemojuvelin, and MuSK) for such purposes as enhancing therapeutic efficacy or stability (e.g., shelf-life and resistance to proteolytic degradation in vivo). Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide, or to bind to one or more TGF-beta superfamily ligands including, for example, BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty. In some embodiments, the present disclosure contemplates making functional variants by modifying the structure of the TGF-beta superfamily co-receptor polypeptide for such purposes as enhancing therapeutic efficacy or stability (e.g., increased shelf-life and/or increased resistance to proteolytic degradation).

In certain embodiments, the present disclosure contemplates specific mutations of a TGF-beta superfamily co-receptor polypeptide (e.g., endoglin, betaglycan, Cripto-1, Cryptic, Cryptic family protein 1B, CRIM1, CRIM2, BAMBI, BMPER, RGM-A, RGM-B, MuSK, and hemojuvelin) of the disclosure so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine or asparagine-X-serine (where “X” is any amino acid) which is specifically recognized by appropriate cellular glycosylation enzymes. The alteration may also be made by the addition of, or substitution by, one or more serine or threonine residues to the sequence of the polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a polypeptide is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. Removal of one or more carbohydrate moieties present on a polypeptide may be accomplished chemically and/or enzymatically. Chemical deglycosylation may involve, for example, exposure of a polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Enzymatic cleavage of carbohydrate moieties on polypeptides can be achieved by the use of a variety of endo- and exo-glycosidases as described by Thotakura et al. [Meth. Enzymol. (1987) 138:350]. The sequence of a polypeptide may be adjusted, as appropriate, depending on the type of expression system used, as mammalian, yeast, insect, and plant cells may all introduce differing glycosylation patterns that can be affected by the amino acid sequence of the peptide. In general, heteromultimers of the disclosure for use in humans may be expressed in a mammalian cell line that provides proper glycosylation, such as HEK293 or CHO cell lines, although other mammalian expression cell lines are expected to be useful as well.

The present disclosure further contemplates a method of generating mutants, particularly sets of combinatorial mutants of a TGF-beta superfamily co-receptor polypeptide (e.g., endoglin, betaglycan, Cripto-1, Cryptic, Cryptic family protein 1B, CRIM1, CRIM2, BAMBI, BMPER, RGM-A, RGM-B, MuSK, and hemojuvelin) of the present disclosure, as well as truncation mutants. Pools of combinatorial mutants are especially useful for identifying functionally active (e.g., ligand binding) TGF-beta superfamily co-receptor sequences. The purpose of screening such combinatorial libraries may be to generate, for example, polypeptides variants which have altered properties, such as altered pharmacokinetic or altered ligand binding. A variety of screening assays are provided below, and such assays may be used to evaluate variants. For example, TGF-beta co-receptor variants may be screened for ability to bind to a TGF-beta superfamily ligand (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty), to prevent binding of a TGF-beta superfamily ligand to a TGF-beta superfamily co-receptor, and/or to interfere with signaling caused by an TGF-beta superfamily ligand.

The activity of a TGF-beta superfamily heteromultimers of the disclosure also may be tested, for example in a cell-based or in vivo assay. For example, the effect of a heteromultimer on the expression of genes or the activity of proteins involved in muscle production in a muscle cell may be assessed. This may, as needed, be performed in the presence of one or more recombinant TGF-beta superfamily ligand proteins (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty), and cells may be transfected so as to produce a TGF-beta superfamily heteromultimer, and optionally, a TGF-beta superfamily ligand. Likewise, a heteromultimer of the disclosure may be administered to a mouse or other animal, and one or more measurements, such as muscle formation and strength may be assessed using art-recognized methods. Similarly, the activity of a heteromultimer, or variants thereof, may be tested in osteoblasts, adipocytes, and/or neuronal cells for any effect on growth of these cells, for example, by the assays as described herein and those of common knowledge in the art. A SMAD-responsive reporter gene may be used in such cell lines to monitor effects on downstream signaling.

Combinatorial-derived variants can be generated which have increased selectivity or generally increased potency relative to a reference TGF-beta superfamily heteromultimer. Such variants, when expressed from recombinant DNA constructs, can be used in gene therapy protocols. Likewise, mutagenesis can give rise to variants which have intracellular half-lives dramatically different than the corresponding unmodified TGF-beta superfamily heteromultimer. For example, the altered protein can be rendered either more stable or less stable to proteolytic degradation or other cellular processes which result in destruction, or otherwise inactivation, of an unmodified polypeptide. Such variants, and the genes which encode them, can be utilized to alter polypeptide complex levels by modulating the half-life of the polypeptide. For instance, a short half-life can give rise to more transient biological effects and, when part of an inducible expression system, can allow tighter control of recombinant polypeptide complex levels within the cell. In an Fc fusion protein, mutations may be made in the linker (if any) and/or the Fc portion to alter one or more activities of the TGF-beta superfamily heteromultimer including, for example, immunogenicity, half-life, and solubility.

A combinatorial library may be produced by way of a degenerate library of genes encoding a library of polypeptides which each include at least a portion of potential TGF-beta superfamily or co-receptor polypeptide sequences. For instance, a mixture of synthetic oligonucleotides can be enzymatically ligated into gene sequences such that the degenerate set of potential TGF-beta superfamily co-receptor encoding nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes can then be ligated into an appropriate vector for expression. The synthesis of degenerate oligonucleotides is well known in the art. See, e.g., Narang, S A (1983) Tetrahedron 39:3; Itakura et al. (1981) Recombinant DNA, Proc. 3rd Cleveland Sympos. Macromolecules, ed. AG Walton, Amsterdam: Elsevier pp 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins. See, e.g., Scott et al., (1990) Science 249:386-390; Roberts et al. (1992) PNAS USA 89:2429-2433; Devlin et al. (1990) Science 249: 404-406; Cwirla et al., (1990) PNAS USA 87: 6378-6382; as well as U.S. Pat. Nos. 5,223,409, 5,198,346, and 5,096,815.

Alternatively, other forms of mutagenesis can be utilized to generate a combinatorial library. For example, heteromultimers of the disclosure can be generated and isolated from a library by screening using, for example, alanine scanning mutagenesis [see, e.g., Ruf et al. (1994) Biochemistry 33:1565-1572; Wang et al. (1994) J. Biol. Chem. 269:3095-3099; Balint et al. (1993) Gene 137:109-118; Grodberg et al. (1993) Eur. J. Biochem. 218:597-601; Nagashima et al. (1993) J. Biol. Chem. 268:2888-2892; Lowman et al. (1991) Biochemistry 30:10832-10838; and Cunningham et al. (1989) Science 244:1081-1085], by linker scanning mutagenesis [see, e.g., Gustin et al. (1993) Virology 193:653-660; and Brown et al. (1992) Mol. Cell Biol. 12:2644-2652; McKnight et al. (1982) Science 232:316], by saturation mutagenesis [see, e.g., Meyers et al., (1986) Science 232:613]; by PCR mutagenesis [see, e.g., Leung et al. (1989) Method Cell Mol Biol 1:11-19]; or by random mutagenesis, including chemical mutagenesis [see, e.g., Miller et al. (1992) A Short Course in Bacterial Genetics, CSHL Press, Cold Spring Harbor, N.Y.; and Greener et al. (1994) Strategies in Mol Biol 7:32-34]. Linker scanning mutagenesis, particularly in a combinatorial setting, is an attractive method for identifying truncated (bioactive) forms of TGF-beta superfamily co-receptor polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of heteromultimers of the disclosure. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Preferred assays include TGF-beta superfamily ligand (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty) binding assays and/or TGF-beta superfamily ligand-mediated cell signaling assays.

In certain embodiments, heteromultimers of the disclosure may further comprise post-translational modifications in addition to any that are naturally present in the TGF-beta superfamily co-receptor polypeptide. Such modifications include, but are not limited to, acetylation, carboxylation, glycosylation, phosphorylation, lipidation, and acylation. As a result, the heteromultimers may comprise non-amino acid elements, such as polyethylene glycols, lipids, polysaccharide or monosaccharide, and phosphates. Effects of such non-amino acid elements on the functionality of a heteromultimer may be tested as described herein for other heteromultimer variants. When a polypeptide of the disclosure is produced in cells by cleaving a nascent form of the polypeptide, post-translational processing may also be important for correct folding and/or function of the protein. Different cells (e.g., CHO, HeLa, MDCK, 293, WI38, NIH-3T3 or HEK293) have specific cellular machinery and characteristic mechanisms for such post-translational activities and may be chosen to ensure the correct modification and processing of the TGF-beta superfamily co-receptor polypeptides as well as heteromultimers comprising the same.

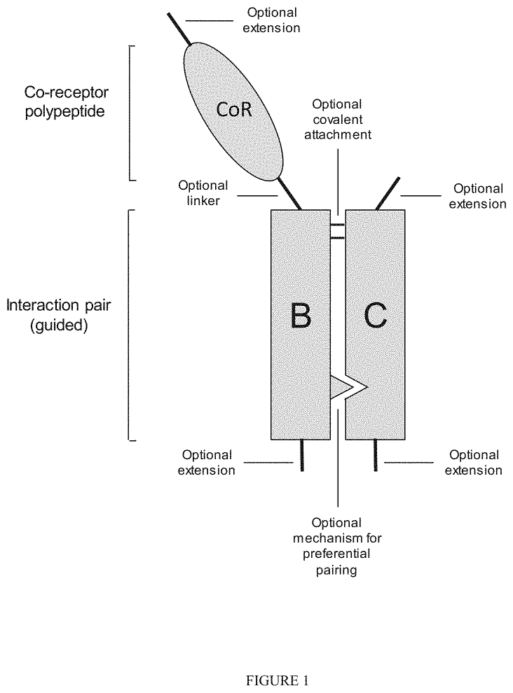

In certain aspects, the polypeptides disclosed herein may form heteromultimers comprising at least one TGF-beta superfamily co-receptor polypeptide. Preferably, polypeptides disclosed herein form heterodimers, although higher order heteromultimers are also included such as, but not limited to, heterotrimers, heterotetramers, and further oligomeric structures (see, e.g., FIG. 1). In some embodiments, TGF-beta superfamily co-receptor polypeptides of the present disclosure comprise at least one multimerization domain. As disclosed herein, the term “multimerization domain” refers to an amino acid or sequence of amino acids that promote covalent or non-covalent interaction between at least a first polypeptide and at least a second polypeptide. Polypeptides disclosed herein may be joined covalently or non-covalently to a multimerization domain. Preferably, a multimerization domain promotes interaction between a first polypeptide and a second polypeptide to promote heteromultimer formation (e.g., heterodimer formation), and optionally hinders or otherwise disfavors homomultimer formation (e.g., homodimer formation), thereby increasing the yield of desired heteromultimer (see, e.g., FIG. 1).

Many methods known in the art can be used to generate heteromultimers of the disclosure. For example, non-naturally occurring disulfide bonds may be constructed by replacing on a first polypeptide a naturally occurring amino acid with a free thiol-containing residue, such as cysteine, such that the free thiol interacts with another free thiol-containing residue on a second polypeptide such that a disulfide bond is formed between the first and second polypeptides. Additional examples of interactions to promote heteromultimer formation include, but are not limited to, ionic interactions such as described in Kjaergaard et al., WO2007147901; electrostatic steering effects such as described in Kannan et al., U.S. Pat. No. 8,592,562; coiled-coil interactions such as described in Christensen et al., U.S.20120302737; leucine zippers such as described in Pack & Plueckthun, (1992) Biochemistry 31: 1579-1584; and helix-turn-helix motifs such as described in Pack et al., (1993) Bio/Technology 11: 1271-1277. Linkage of the various segments may be obtained via, e.g., covalent binding such as by chemical cross-linking, peptide linkers, disulfide bridges, etc., or affinity interactions such as by avidin-biotin or leucine zipper technology.

In certain aspects, a multimerization domain may comprise one component of an interaction pair. In some embodiments, the polypeptides disclosed herein may form protein complexes comprising a first polypeptide covalently or non-covalently associated with a second polypeptide, wherein the first polypeptide comprises the amino acid sequence of a TGF-beta superfamily co-receptor polypeptide and the amino acid sequence of a first member of an interaction pair; and the second polypeptide comprises an amino acid sequence of a second member of an interaction pair. The interaction pair may be any two polypeptide sequences that interact to form a complex, particularly a heterodimeric complex although operative embodiments may also employ an interaction pair that can form a homodimeric complex. One member of the interaction pair may be fused to a TGF-beta superfamily co-receptor polypeptide as described herein, including for example, a polypeptide sequence comprising an amino acid sequence that is at least 70%, 75%, 80%, 85%, 90%, 91%, 92%, 93%, 94%, 95%, 96%, 97%, 98%, 99%, or 100% identical to the sequence of any one of SEQ ID NOs: 1, 2, 5, 6, 9, 10, 13, 14, 17, 18, 21, 22, 25, 26, 29, 30, 33, 34, 37, 38, 41, 42, 45, 46, 49, 50, 53, 54, 57, 58, 61, 62, 65, 66, 69, 70, 73, 74, 77, 78, 81, 82, 85, 86, 89, 90, 93, 95, 96, 99, 100, 103, and 104. An interaction pair may be selected to confer an improved property/activity such as increased serum half-life, or to act as an adaptor on to which another moiety is attached to provide an improved property/activity. For example, a polyethylene glycol moiety may be attached to one or both components of an interaction pair to provide an improved property/activity such as improved serum half-life.

The first and second members of the interaction pair may be an asymmetric pair, meaning that the members of the pair preferentially associate with each other rather than self-associate. Accordingly, first and second members of an asymmetric interaction pair may associate to form a heterodimeric complex (see, e.g., FIG. 1). Alternatively, the interaction pair may be unguided, meaning that the members of the pair may associate with each other or self-associate without substantial preference and thus may have the same or different amino acid sequences. Accordingly, first and second members of an unguided interaction pair may associate to form a homodimer complex or a heterodimeric complex. Optionally, the first member of the interaction pair (e.g., an asymmetric pair or an unguided interaction pair) associates covalently with the second member of the interaction pair. Optionally, the first member of the interaction pair (e.g., an asymmetric pair or an unguided interaction pair) associates non-covalently with the second member of the interaction pair.

As specific examples, the present disclosure provides fusion proteins comprising TGF-beta superfamily co-receptor polypeptides fused to a polypeptide comprising a constant domain of an immunoglobulin, such as a CH1, CH2, or CH3 domain of an immunoglobulin or an Fc domain. Fc domains derived from human IgG1, IgG2, IgG3, and IgG4 are provided herein. Other mutations are known that decrease either CDC or ADCC activity, and collectively, any of these variants are included in the disclosure and may be used as advantageous components of a heteromultimers of the disclosure. Optionally, the IgG1 Fc domain of SEQ ID NO: 208 has one or more mutations at residues such as Asp-265, Lys-322, and Asn-434 (numbered in accordance with the corresponding full-length IgG1). In certain cases, the mutant Fc domain having one or more of these mutations (e.g., Asp-265 mutation) has reduced ability of binding to the Fcγ receptor relative to a wildtype Fc domain. In other cases, the mutant Fc domain having one or more of these mutations (e.g., Asn-434 mutation) has increased ability of binding to the MHC class I-related Fc-receptor (FcRN) relative to a wildtype Fc domain.

An example of a native amino acid sequence that may be used for the Fc portion of human IgG1 (G1Fc) is shown below (SEQ ID NO: 208). Dotted underline indicates the hinge region, and solid underline indicates positions with naturally occurring variants. In part, the disclosure provides polypeptides comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NO: 208. Naturally occurring variants in G1Fc would include E134D and M136L according to the numbering system used in SEQ ID NO: 208 (see Uniprot P01857).

| (SEQ ID NO: 208) | |
|---|---|
| 1 | 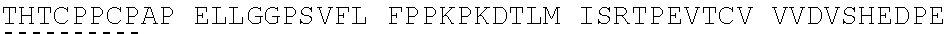 |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |

An example of a native amino acid sequence that may be used for the Fc portion of human IgG2 (G2Fc) is shown below (SEQ ID NO: 209). Dotted underline indicates the hinge region and double underline indicates positions where there are data base conflicts in the sequence (according to UniProt P01859). In part, the disclosure provides polypeptides comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NO: 209.

| (SEQ ID NO: 209) | |
|---|---|
| 1 | 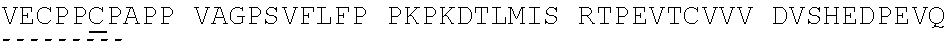 |
| 51 | FNWYVDGVEV HNAKTKPREE QFNSTFRVVS VLTVVHQDWL NGKEYKCKVS |
| 101 | NKGLPAPIEK TISKTKGQPR EPQVYTLPPS REEMTKNQVS LTCLVKGFYP |
| 151 | SDIAVEWESN GQPENNYKTT PPMLDSDGSF FLYSKLTVDK SRWQQGNVFS |
| 201 | CSVMHEALHN HYTQKSLSLS PGK |

Two examples of amino acid sequences that may be used for the Fc portion of human IgG3 (G3Fc) are shown below. The hinge region in G3Fc can be up to four times as long as in other Fc chains and contains three identical 15-residue segments preceded by a similar 17-residue segment. The first G3Fc sequence shown below (SEQ ID NO: 210) contains a short hinge region consisting of a single 15-residue segment, whereas the second G3Fc sequence (SEQ ID NO: 211) contains a full-length hinge region. In each case, dotted underline indicates the hinge region, and solid underline indicates positions with naturally occurring variants according to UniProt P01859. In part, the disclosure provides polypeptides comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NOs: 210 and 211.

| | (SEQ ID NO: 210) |
|---|---|
| 1 | 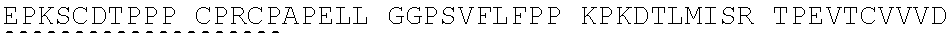 |
| 51 | VSHEDPEVQF KWYVDGVEVH NAKTKPREEQ YNSTFRVVSV LTVLHQDWLN |
| 101 | GKEYKCKVSN KALPAPIEKT ISKTKGQPRE PQVYTLPPSR EEMTKNQVSL |
| 151 | TCLVKGFYPS DIAVEWESSG QPENNYNTTP PMLDSDGSFF LYSKLTVDKS |
| 201 | RWQQGNIFSC SVMHEALHNR FTQKSLSLSP GK |
| | (SEQ ID NO: 211) |
| 1 | 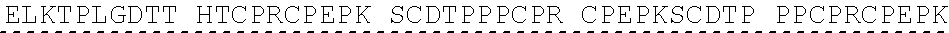 |
| 51 | 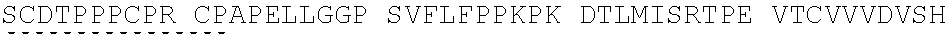 |
| 101 | EDPEVQFKWY VDGVEVHNAK TKPREEQYNS TFRVVSVLTV LHQDWLNGKE |
| 151 | YKCKVSNKAL PAPIEKTISK TKGQPREPQV YTLPPSREEM TKNQVSLTCL |
| 201 | VKGFYPSDIA VEWESSGQPE NNYNTTPPML DSDGSFFLYS KLTVDKSRWQ |
| 251 | QGNIFSCSVM HEALHNRFTQ KSLSLSPGK |

Naturally occurring variants in G3Fc (for example, see Uniprot P01860) include E68Q, P76L, E79Q, Y81F, D97N, N100D, T124A, S169N, S169del, F221Y when converted to the numbering system used in SEQ ID NO: 210, and the present disclosure provides fusion proteins comprising G3Fc domains containing one or more of these variations. In addition, the human immunoglobulin IgG3 gene (IGHG3) shows a structural polymorphism characterized by different hinge lengths [see Uniprot P01859]. Specifically, variant WIS is lacking most of the V region and all of the CH1 region. It has an extra interchain disulfide bond at position 7 in addition to the 11 normally present in the hinge region. Variant ZUC lacks most of the V region, all of the CH1 region, and part of the hinge. Variant OMM may represent an allelic form or another gamma chain subclass. The present disclosure provides additional fusion proteins comprising G3Fc domains containing one or more of these variants.

An example of a native amino acid sequence that may be used for the Fc portion of human IgG4 (G4Fc) is shown below (SEQ ID NO: 212). Dotted underline indicates the hinge region. In part, the disclosure provides polypeptides comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NO: 212.

| (SEQ ID NO: 212) | |
|---|---|
| 1 | 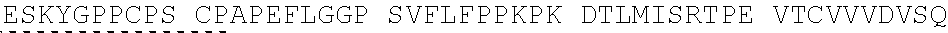 |
| 51 | EDPEVQFNWY VDGVEVHNAK TKPREEQFNS TYRVVSVLTV LHQDWLNGKE |
| 101 | YKCKVSNKGL PSSIEKTISK AKGQPREPQV YTLPPSQEEM TKNQVSLTCL |
| 151 | VKGFYPSDIA VEWESNGQPE NNYKTTPPVL DSDGSFFLYS RLTVDKSRWQ |
| 201 | EGNVFSCSVM HEALHNHYTQ KSLSLSLGK |

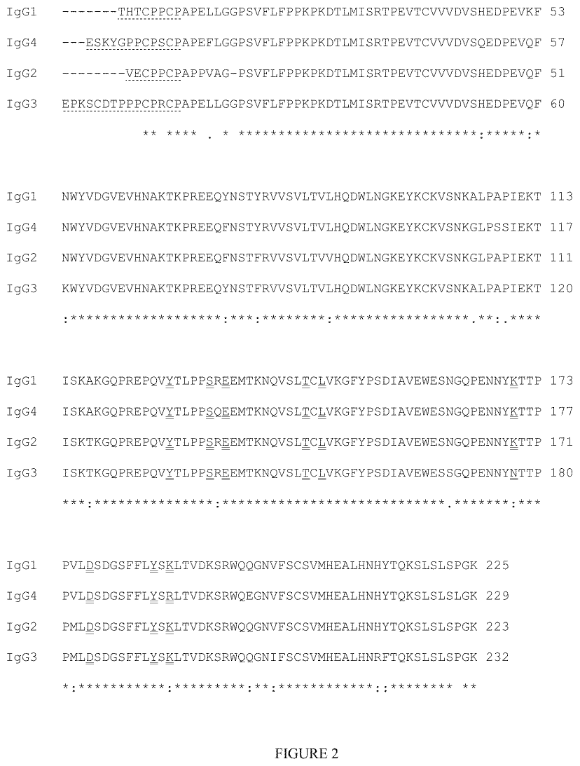

A variety of engineered mutations in the Fc domain are presented herein with respect to the G1Fc sequence (SEQ ID NO: 208), and analogous mutations in G2Fc, G3Fc, and G4Fc can be derived from their alignment with G1Fc in FIG. 2. Due to unequal hinge lengths, analogous Fc positions based on isotype alignment (FIG. 2) possess different amino acid numbers in SEQ ID NOs: 208, 209, 210, and 212. It can also be appreciated that a given amino acid position in an immunoglobulin sequence consisting of hinge, C_{H}2, and C_{H}3 regions (e.g., SEQ ID NOs: 208, 209, 210, 211, or 212) will be identified by a different number than the same position when numbering encompasses the entire IgG heavy-chain constant domain (consisting of the C_{H}1, hinge, C_{H}2, and C_{H}3 regions) as in the Uniprot database. For example, correspondence between selected C_{H}3 positions in a human G1Fc sequence (SEQ ID NO: 208), the human IgG1 heavy chain constant domain (Uniprot P01857), and the human IgG1 heavy chain is as follows.

Correspondence of C_{H}3 Positions in Different Numbering Systems

| G1Fc | IgG1 heavy chain | |
|---|---|---|
| (Numbering begins | constant domain | IgG1 heavy chain |
| at first threonine | (Numbering begins | (EU numbering scheme |
| in hinge region) | at C_{H}1) | of Kabat et al., 1991*) |
| Y127 | Y232 | Y349 |
| S132 | S237 | S354 |
| E134 | E239 | E356 |
| K138 | K243 | K360 |
| T144 | T249 | T366 |
| L146 | L251 | L368 |
| N162 | N267 | N384 |
| K170 | K275 | K392 |
| D177 | D282 | D399 |
| D179 | D284 | D401 |
| Y185 | Y290 | Y407 |
| K187 | K292 | K409 |
| H213 | H318 | H435 |
| K217 | K322 | K439 |

*Kabat et al. (eds) 1991; pp. 688-696 in Sequences of Proteins of Immunological Interest, 5^{th} ed., Vol. 1, NIH, Bethesda, MD.

A problem that arises in large-scale production of asymmetric immunoglobulin-based proteins from a single cell line is known as the “chain association issue”. As confronted prominently in the production of bispecific antibodies, the chain-association issue concerns 12′7 the challenge of efficiently producing a desired multichain protein from among the multiple combinations that inherently result when different heavy chains and/or light chains are produced in a single cell line [see, for example, Klein et al (2012) mAbs 4:653-663]. This problem is most acute when two different heavy chains and two different light chains are produced in the same cell, in which case there are a total of 16 possible chain combinations (although some of these are identical) when only one is typically desired. Nevertheless, the same principle accounts for diminished yield of a desired multichain fusion protein that incorporates only two different (asymmetric) heavy chains.

Various methods are known in the art that increase desired pairing of Fc-containing fusion polypeptide chains in a single cell line to produce a preferred asymmetric fusion protein at acceptable yields [see, for example, Klein et al (2012) mAbs 4:653-663; and Spiess et al (2015) Molecular Immunology 67(2A): 95-106]. Methods to obtain desired pairing of Fc-containing chains include, but are not limited to, charge-based pairing (electrostatic steering), “knobs-into-holes” steric pairing, SEEDbody pairing, and leucine zipper-based pairing. See, for example, Ridgway et al (1996) Protein Eng 9:617-621; Merchant et al (1998) Nat Biotech 16:677-681; Davis et al (2010) Protein Eng Des Sel 23:195-202; Gunasekaran et al (2010); 285:19637-19646; Wranik et al (2012) J Biol Chem 287:43331-43339; U.S. Pat. No. 5,932,448; WO 1993/011162; WO 2009/089004, and WO 2011/034605. As described herein, these methods may be used to generate heterodimers comprising a TGF-beta superfamily co-receptor. See FIG. 1.

For example, one means by which interaction between specific polypeptides may be promoted is by engineering protuberance-into-cavity (knob-into-holes) complementary regions such as described in Arathoon et al., U.S. Pat. No. 7,183,076 and Carter et al., U.S. Pat. No. 5,731,168. “Protuberances” are constructed by replacing small amino acid side chains from the interface of the first polypeptide (e.g., a first interaction pair) with larger side chains (e.g., tyrosine or tryptophan). Complementary “cavities” of identical or similar size to the protuberances are optionally created on the interface of the second polypeptide (e.g., a second interaction pair) by replacing large amino acid side chains with smaller ones (e.g., alanine or threonine). Where a suitably positioned and dimensioned protuberance or cavity exists at the interface of either the first or second polypeptide, it is only necessary to engineer a corresponding cavity or protuberance, respectively, at the adjacent interface.

At neutral pH (7.0), aspartic acid and glutamic acid are negatively charged and lysine, arginine, and histidine are positively charged. These charged residues can be used to promote heterodimer formation and at the same time hinder homodimer formation. Attractive interactions take place between opposite charges and repulsive interactions occur between like charges. In part, protein complexes disclosed herein make use of the attractive interactions for promoting heteromultimer formation (e.g., heterodimer formation), and optionally repulsive interactions for hindering homodimer formation (e.g., homodimer formation) by carrying out site directed mutagenesis of charged interface residues.

For example, the IgG1 CH3 domain interface comprises four unique charge residue pairs involved in domain-domain interactions: Asp356-Lys439′, Glu357-Lys370′, Lys392-Asp399′, and Asp399-Lys409′ [residue numbering in the second chain is indicated by (′)]. It should be noted that the numbering scheme used here to designate residues in the IgG1 CH3 domain conforms to the EU numbering scheme of Kabat. Due to the 2-fold symmetry present in the CH3-CH3 domain interactions, each unique interaction will represented twice in the structure (e.g., Asp-399-Lys409′ and Lys409-Asp399′). In the wild-type sequence, K409-D399′ favors both heterodimer and homodimer formation. A single mutation switching the charge polarity (e.g., K409E; positive to negative charge) in the first chain leads to unfavorable interactions for the formation of the first chain homodimer. The unfavorable interactions arise due to the repulsive interactions occurring between the same charges (negative-negative; K409E-D399′ and D399-K409E′). A similar mutation switching the charge polarity (D399K′; negative to positive) in the second chain leads to unfavorable interactions (K409′-D399K′ and D399K-K409′) for the second chain homodimer formation. But, at the same time, these two mutations (K409E and D399K′) lead to favorable interactions (K409E-D399K′ and D399-K409′) for the heterodimer formation.

The electrostatic steering effect on heterodimer formation and homodimer discouragement can be further enhanced by mutation of additional charge residues which may or may not be paired with an oppositely charged residue in the second chain including, for example, Arg355 and Lys360. The table below lists possible charge change mutations that can be used, alone or in combination, to enhance heteromultimer formation of the heteromultimers disclosed herein.

Examples of Pair-Wise Charged Residue Mutations
to Enhance Heterodimer Formation

| | | | Corresponding |
|---|---|---|---|
| Position in | Mutation in | Interacting position | mutation in |
| first chain | first chain | in second chain | second chain |
| Lys409 | Asp or Glu | Asp399′ | Lys, Arg, or His |
| Lys392 | Asp or Glu | Asp399′ | Lys, Arg, or His |
| Lys439 | Asp or Glu | Asp356′ | Lys, Arg, or His |
| Lys370 | Asp or Glu | Glu357′ | Lys, Arg, or His |
| Asp399 | Lys, Arg, or His | Lys409′ | Asp or Glu |
| Asp399 | Lys, Arg, or His | Lys392′ | Asp or Glu |
| Asp356 | Lys, Arg, or His | Lys439′ | Asp or Glu |
| Glu357 | Lys, Arg, or His | Lys370′ | Asp or Glu |

In some embodiments, one or more residues that make up the CH3-CH3 interface in a fusion protein of the instant application are replaced with a charged amino acid such that the interaction becomes electrostatically unfavorable. For example, a positive-charged amino acid in the interface (e.g., a lysine, arginine, or histidine) is replaced with a negatively charged amino acid (e.g., aspartic acid or glutamic acid). Alternatively, or in combination with the forgoing substitution, a negative-charged amino acid in the interface is replaced with a positive-charged amino acid. In certain embodiments, the amino acid is replaced with a non-naturally occurring amino acid having the desired charge characteristic. It should be noted that mutating negatively charged residues (Asp or Glu) to His will lead to increase in side chain volume, which may cause steric issues. Furthermore, His proton donor- and acceptor-form depends on the localized environment. These issues should be taken into consideration with the design strategy. Because the interface residues are highly conserved in human and mouse IgG subclasses, electrostatic steering effects disclosed herein can be applied to human and mouse IgG1, IgG2, IgG3, and IgG4. This strategy can also be extended to modifying uncharged residues to charged residues at the CH3 domain interface.

In part, the disclosure provides desired pairing of asymmetric Fc-containing polypeptide chains using Fc sequences engineered to be complementary on the basis of charge pairing (electrostatic steering). One of a pair of Fc sequences with electrostatic complementarity can be arbitrarily fused to the co-receptor polypeptide of the construct, with or without an optional linker, to generate a TGF-beta superfamily co-receptor receptor fusion polypeptide. This single chain can be coexpressed in a cell of choice along with the Fc sequence complementary to the first Fe to favor generation of the desired multichain construct (e.g., a TGF-beta superfamily heteromultimer). In this example based on electrostatic steering, SEQ ID NO: 200 [human G1Fc(E134K/D177K)] and SEQ ID NO: 201 [human G1Fc(K170D/K187D)] are examples of complementary Fc sequences in which the engineered amino acid substitutions are double underlined, and the TGF-beta superfamily co-receptor polypeptide of the construct can be fused to either SEQ ID NO: 200 or SEQ ID NO: 201, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 2) will generate complementary Fc pairs which may be used instead of the complementary hG1Fc pair below (SEQ ID NOs: 200 and 201).

| (SEQ ID NO: 200) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSRKEMTKNQ VSLTCLVKGF |
| 151 | YPSDIAVEWE SNGQPENNYK TTPPVLKSDG SFFLYSKLTV DKSRWQQGNV |
| 201 | FSCSVMHEAL HNHYTQKSLS LSPGK |
| (SEQ ID NO: 201) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYD TTPPVLDSDG SFFLYSDLTV DKSRWQQGNV |
| 201 | FSCSVMHEAL HNHYTQKSLS LSPGK |

In part, the disclosure provides desired pairing of asymmetric Fc-containing polypeptide chains using Fc sequences engineered for steric complementarity. In part, the disclosure provides knobs-into-holes pairing as an example of steric complementarity. One of a pair of Fc sequences with steric complementarity can be arbitrarily fused to the TGF-beta superfamily co-receptor polypeptide of the construct, with or without an optional linker, to generate a TGF-beta superfamily co-receptor fusion polypeptide. This single chain can be coexpressed in a cell of choice along with the Fc sequence complementary to the first Fc to favor generation of the desired multichain construct. In this example based on knobs-into-holes pairing, SEQ ID NO: 202 [human G1Fc(T144Y)] and SEQ ID NO: 203 [human G1Fc(Y185T)] are examples of complementary Fc sequences in which the engineered amino acid substitutions are double underlined, and the co-receptor polypeptide of the construct can be fused to either SEQ ID NO: 202 or SEQ ID NO: 203, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 2) will generate complementary Fc pairs which may be used instead of the complementary hG1Fc pair below (SEQ ID NOs: 202 and 203).

| (SEQ ID NO: 202) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLYCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |
| (SEQ ID NO: 203) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLTSKLTV DKSRWQQGNV |
| 201 | FSCSVMHEAL HNHYTQKSLS LSPGK |

An example of Fc complementarity based on knobs-into-holes pairing combined with an engineered disulfide bond is disclosed in SEQ ID NO: 204 [hG1Fc(S132C/T144W)] and SEQ ID NO: 205 [hGlFc(Y127C/T144S/L146A/Y185V)]. The engineered amino acid substitutions in these sequences are double underlined, and the TGF-beta superfamily co-receptor of the construct can be fused to either SEQ ID NO: 204 or SEQ ID NO: 205, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 2) will generate complementary Fc pairs which may be used instead of the complementary hG1Fc pair below (SEQ ID NOs: 204 and 205).

| (SEQ ID NO: 204) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PCREEMTKNQ VSLWCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |
| (SEQ ID NO: 205) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVCTLP PSREEMTKNQ VSLSCAVKGF |
| 151 | YPSDIAVEWE SNGQPENNYK TTPPVLDSDG SFFLVSKLTV DKSRWQQGNV |
| 201 | FSCSVMHEAL HNHYTQKSLS LSPGK |

In part, the disclosure provides desired pairing of asymmetric Fc-containing polypeptide chains using Fc sequences engineered to generate interdigitating β-strand segments of human IgG and IgA C_{H}3 domains. Such methods include the use of strand-exchange engineered domain (SEED) C_{H}3 heterodimers allowing the formation of SEEDbody fusion proteins [see, for example, Davis et al (2010) Protein Eng Design Sel 23:195-202]. One of a pair of Fc sequences with SEEDbody complementarity can be arbitrarily fused to the TGF-beta superfamily type co-receptor polypeptide of the construct, with or without an optional linker, to generate a TGF-beta superfamily fusion polypeptide. This single chain can be coexpressed in a cell of choice along with the Fc sequence complementary to the first Fc to favor generation of the desired multichain construct. In this example based on SEEDbody (Sb) pairing, SEQ ID NO: 206 [hG1Fc(Sb_{AG})] and SEQ ID NO: 207 [hG1Fc(Sb_{GA})] are examples of complementary IgG Fc sequences in which the engineered amino acid substitutions from IgA Fc are double underlined, and the TGF-beta superfamily co-receptor polypeptide of the construct can be fused to either SEQ ID NO: 206 or SEQ ID NO: 207, but not both. Given the high degree of amino acid sequence identity between native hG1Fc, native hG2Fc, native hG3Fc, and native hG4Fc, it can be appreciated that amino acid substitutions at corresponding positions in hG1Fc, hG2Fc, hG3Fc, or hG4Fc (see FIG. 2) will generate an Fc monomer which may be used in the complementary IgG-IgA pair below (SEQ ID NOs: 206 and 207).

| (SEQ ID NO: 206) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PFRPEVHLLP PSREEMTKNQ VSLTCLARGF |
| 151 | YPKDIAVEWE SNGQPENNYK TTPSRCEPSQ GTTTFAVTSK LTVDKSRWQQ |
| 201 | GNVFSCSVMH EALHNHYTQK TISLSPGK |
| (SEQ ID NO: 207) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PPSEELALNE LVTLTCLVKG |
| 151 | FYPSDIAVEW ESNGQELPRE KYLTWAPVLD SDGSFFLYSI LRVAAEDWKK |
| 201 | GDTFSCSVMH EALHNHYTQK SLDRSPGK |

In part, the disclosure provides desired pairing of asymmetric Fc-containing polypeptide chains with a cleavable leucine zipper domain attached at the C-terminus of the Fc C_{H}3 domains. Attachment of a leucine zipper is sufficient to cause preferential assembly of heterodimeric antibody heavy chains. See, e.g., Wranik et al (2012) J Biol Chem 287:43331-43339. As disclosed herein, one of a pair of Fc sequences attached to a leucine zipper-forming strand can be arbitrarily fused to the TGF-beta superfamily co-receptor polypeptide of the construct, with or without an optional linker, to generate a TGF-beta superfamily fusion polypeptide. This single chain can be coexpressed in a cell of choice along with the Fe sequence attached to a complementary leucine zipper-forming strand to favor generation of the desired multichain construct. Proteolytic digestion of the construct with the bacterial endoproteinase Lys-C post purification can release the leucine zipper domain, resulting in an Fc construct whose structure is identical to that of native Fc. In this example based on leucine zipper pairing, SEQ ID NO: 213 [hG1Fc-Ap1 (acidic)] and SEQ ID NO: 214 [hG1Fc-Bp1 (basic)] are examples of complementary IgG Fc sequences in which the engineered complimentary leucine zipper sequences are underlined, and the co-receptor polypeptide of the construct can be fused to either SEQ ID NO: 213 or SEQ ID NO: 214, but not both. Given the high degree of amino acid sequence identity between native hG1Fc, native hG2Fc, native hG3Fc, and native hG4Fc, it can be appreciated that leucine zipper-forming sequences attached, with or without an optional linker, to hG1Fc, hG2Fc, hG3Fc, or hG4Fc will generate an Fc monomer which may be used in the complementary leucine zipper-forming pair below (SEQ ID NOs: 213 and 214).

| (SEQ ID NO: 213) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGKGGSAQ LEKELQALEK ENAQLEWELQ |
| 251 | ALEKELAQGA T |
| (SEQ ID NO: 214) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGKGGSAQ LKKKLQALKK KNAQLKWKLQ |
| 251 | ALKKKLAQGA T |

In part, the disclosure provides desired pairing of asymmetric Fc-containing polypeptide chains by methods described above in combination with additional mutations in the Fc domain which facilitate purification of the desired heteromeric species. An example is complementarity of Fc domains based on knobs-into-holes pairing combined with an engineered disulfide bond, as disclosed in SEQ ID NOs: 204-205, plus additional substitution of two negatively charged amino acids (aspartic acid or glutamic acid) in one Fc-containing polypeptide chain and two positively charged amino acids (e.g., arginine) in the complementary Fc-containing polypeptide chain (SEQ ID NOs: 215-216). These four amino acid substitutions facilitate selective purification of the desired heteromeric fusion protein from a heterogeneous polypeptide mixture based on differences in isoelectric point or net molecular charge. The engineered amino acid substitutions in these sequences are double underlined below, and the TGFβ superfamily type I receptor polypeptide, type II receptor polypeptide, or co-receptor polypeptide of the construct can be fused to either SEQ ID NO: 215 or SEQ ID NO: 216, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 2) will generate complementary Fc pairs which may be used instead of the complementary hG1Fc pair below (SEQ ID NOs: 215-216).

| (SEQ ID NO: 215) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PCREEMTENQ VSLWCLVKGF |
| 151 | YPSDIAVEWE SNGQPENNYK TTPPVLDSDG SFFLYSKLTV DKSRWQQGNV |
| 201 | FSCSVMHEAL HNHYTQDSLS LSPGK |
| (SEQ ID NO: 216) | |
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVCTLP PSREEMTKNQ VSLSCAVKGF |
| 151 | YPSDIAVEWE SRGQPENNYK TTPPVLDSRG SFFLVSKLTV DKSRWQQGNV |
| 201 | FSCSVMHEAL HNHYTQKSLS LSPGK |

Another example involves complementarity of Fc domains based on knobs-into-holes pairing combined with an engineered disulfide bond, as disclosed in SEQ ID NOs: 204-205, plus a histidine-to-arginine substitution at position 213 in one Fc-containing polypeptide chain (SEQ ID NO: 217). This substitution (denoted H435R in the numbering system of Kabat et al.) facilitates separation of desired heteromer from undesirable homodimer based on differences in affinity for protein A. The engineered amino acid substitution is indicated by double underline, and the TGFβ superfamily co-receptor polypeptide of the construct can be fused to either SEQ ID NO: 217 or SEQ ID NO: 205, but not both. Given the high degree of amino acid sequence identity between native hG1Fc, native hG2Fc, native hG3Fc, and native hG4Fc, it can be appreciated that amino acid substitutions at corresponding positions in hG2Fc, hG3Fc, or hG4Fc (see FIG. 2) will generate complementary Fc pairs which may be used instead of the complementary hG1Fc pair of SEQ ID NO: 217 (below) and SEQ ID NO: 205.

| (SEQ ID NO: 217) | |
|---|---|
| 1 | THTCPPCPAP ELLGGPSVFL FPPKPKDTLM ISRTPEVTCV VVDVSHEDPE |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PCREEMTKNQ VSLWCLVKGF |
| 151 | YPSDIAVEWE SNGQPENNYK TTPPVLDSDG SFFLYSKLTV DKSRWQQGNV |
| 201 | FSCSVMHEAL HNRYTQKSLS LSPGK |

A variety of engineered mutations in the Fc domain are presented above with respect to the G1Fc sequence (SEQ ID NO: 208). Analogous mutations in G2Fc, G3Fc, and G4Fc can be derived from their alignment with G1Fc in FIG. 2. Due to unequal hinge lengths, analogous Fc positions based on isotype alignment (FIG. 2) possess different amino acid numbers in SEQ ID NOs: 208, 209, 210, and 212 as summarized in the following table.

Correspondence between C_{H}3 Positions for Human Fc Isotypes*

| IgG1 | IgG4 | IgG2 | IgG3 |
|---|---|---|---|
| SEQ ID | SEQ ID | SEQ ID | SEQ ID |
| NO: 208 | NO: 212 | NO: 209 | NO: 210 |
| Numbering | Numbering | Numbering | Numbering |
| begins at | begins at | begins at | begins at |
| THT . . . | ESK . . . | VEC . . . | EPK . . . |
| Y127 | Y131 | Y125 | Y134 |
| S132 | S136 | S130 | S139 |
| E134 | E138 | E132 | E141 |
| K138 | K142 | K136 | K145 |
| T144 | T148 | T142 | T151 |
| L146 | L150 | L144 | L153 |
| N162 | N166 | N160 | S169 |
| K170 | K174 | K168 | N177 |
| D177 | D181 | D175 | D184 |
| D179 | D183 | D177 | D186 |
| Y185 | Y189 | Y183 | Y192 |
| K187 | R191 | K185 | K194 |
| H213 | H217 | H211 | R220 |
| K217 | K221 | K215 | K224 |

*Numbering based on multiple sequence alignment shown in FIG. 2

It is understood that different elements of the fusion proteins (e.g., immunoglobulin Fc fusion proteins) may be arranged in any manner that is consistent with desired functionality. For example, a TGF-beta co-receptor polypeptide domain may be placed C-terminal to a heterologous domain, or alternatively, a heterologous domain may be placed C-terminal to a TGF-beta superfamily co-receptor polypeptide domain. The TGF-beta superfamily co-receptor domain and the heterologous domain need not be adjacent in a fusion protein, and additional domains or amino acid sequences may be included C- or N-terminal to either domain or between the domains.

For example, a TGF-beta superfamily co-receptor fusion protein may comprise an amino acid sequence as set forth in the formula A-B-C. The B portion corresponds to a TGF-beta superfamily co-receptor polypeptide domain. The A and C portions may be independently zero, one, or more than one amino acid, and both the A and C portions when present are heterologous to B. The A and/or C portions may be attached to the B portion via a linker sequence. A linker may be rich in glycine (e.g., 2-10, 2-5, 2-4, 2-3 glycine residues) or glycine and proline residues and may, for example, contain a single sequence of threonine/serine and glycines or repeating sequences of threonine/serine and/or glycines, e.g., GGG (SEQ ID NO: 158), GGGG (SEQ ID NO: 159), TGGGG (SEQ ID NO: 160), SGGGG (SEQ ID NO: 161), TGGG (SEQ ID NO: 162), or SGGG (SEQ ID NO: 163) singlets, or repeats. In certain embodiments, a TGF-beta superfamily co-receptor fusion protein comprises an amino acid sequence as set forth in the formula A-B-C, wherein A is a leader (signal) sequence, B consists of a TGF-beta superfamily co-receptor polypeptide domain, and C is a polypeptide portion that enhances one or more of in vivo stability, in vivo half-life, uptake/administration, tissue localization or distribution, formation of protein complexes, and/or purification. In certain embodiments, a TGF-beta superfamily co-receptor fusion protein comprises an amino acid sequence as set forth in the formula A-B-C, wherein A is a TPA leader sequence, B consists of a TGF-beta superfamily co-receptor polypeptide domain, and C is an immunoglobulin Fc domain. Preferred fusion proteins comprise the amino acid sequence set forth in any one of SEQ ID NOs: 500, 501, 504, 505, and 508-555.

In some embodiments, heteromultimers of the present disclosure further comprise one or more heterologous portions (domains) so as to confer a desired property. For example, some fusion domains are particularly useful for isolation of the fusion proteins by affinity chromatography. Well-known examples of such fusion domains include, but are not limited to, polyhistidine, Glu-Glu, glutathione S-transferase (GST), thioredoxin, protein A, protein G, an immunoglobulin heavy-chain constant region (Fc), maltose binding protein (MBP), or human serum albumin. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS6) fusion partners. As another example, a fusion domain may be selected so as to facilitate detection of the ligand trap polypeptides. Examples of such detection domains include the various fluorescent proteins (e.g., GFP) as well as “epitope tags,” which are usually short peptide sequences for which a specific antibody is available. Well-known epitope tags for which specific monoclonal antibodies are readily available include FLAG, influenza virus haemagglutinin (HA), and c-myc tags. In some cases, the fusion domains have a protease cleavage site, such as for factor Xa or thrombin, which allows the relevant protease to partially digest the fusion proteins and thereby liberate the recombinant proteins therefrom. The liberated proteins can then be isolated from the fusion domain by subsequent chromatographic separation.

In certain embodiments, TGF-beta superfamily co-receptor polypeptides of the present disclosure contain one or more modifications that are capable of stabilizing the polypeptides. For example, such modifications enhance the in vitro half-life of the polypeptides, enhance circulatory half-life of the polypeptides, and/or reduce proteolytic degradation of the polypeptides. Such stabilizing modifications include, but are not limited to, fusion proteins (including, for example, fusion proteins comprising a co-receptor polypeptide domain and a stabilizer domain), modifications of a glycosylation site (including, for example, addition of a glycosylation site to a polypeptide of the disclosure), and modifications of carbohydrate moiety (including, for example, removal of carbohydrate moieties from a polypeptide of the disclosure). As used herein, the term “stabilizer domain” not only refers to a fusion domain (e.g., an immunoglobulin Fc domain) as in the case of fusion proteins, but also includes nonproteinaceous modifications such as a carbohydrate moiety, or nonproteinaceous moiety, such as polyethylene glycol.

In preferred embodiments, heteromultimers to be used in accordance with the methods described herein are isolated polypeptide complexes. As used herein, an isolated protein (or protein complex) or polypeptide (or polypeptide complex) is one which has been separated from a component of its natural environment. In some embodiments, a heteromultimer complex of the disclosure is purified to greater than 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC). Methods for assessment of antibody purity are well known in the art [See, e.g., Flatman et al., (2007) J. Chromatogr. B 848:79-87]. In some embodiments, heteromultimer preparations of the disclosure are substantially free of TGF-beta superfamily co-receptor polypeptide homomultimers. For example, in some embodiments, heteromultimer preparations comprise less than about 10%, 9%, 8%, 7%, 5%, 4%, 3%, 2%, or less than 1% of TGF-beta superfamily co-receptor polypeptide homomultimers.

In certain embodiments, TGFβ superfamily co-receptor polypeptides as well as heteromultimer complexes thereof, of the disclosure can be produced by a variety of art-known techniques. For example, polypeptides of the disclosure can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (see, e.g., Advanced ChemTech Model 396; Milligen/Biosearch 9600). Alternatively, the polypeptides and complexes of the disclosure, including fragments or variants thereof, may be recombinantly produced using various expression systems [e.g., E. coli, Chinese Hamster Ovary (CHO) cells, COS cells, baculovirus] as is well known in the art. In a further embodiment, the modified or unmodified polypeptides of the disclosure may be produced by digestion of recombinantly produced full-length TGFβ superfamily co-receptor polypeptides by using, for example, a protease, e.g., trypsin, thermolysin, chymotrypsin, pepsin, or paired basic amino acid converting enzyme (PACE). Computer analysis (using a commercially available software, e.g., MacVector, Omega, PCGene, Molecular Simulation, Inc.) can be used to identify proteolytic cleavage sites.

### 3. Nucleic Acids Encoding TGFβ Superfamily Co-Receptor Polypeptides

In certain embodiments, the present disclosure provides isolated and/or recombinant nucleic acids encoding TGF superfamily co-receptors (including fragments, functional variants, and fusion proteins thereof) disclosed herein. For example, SEQ ID NO: 3 encodes a naturally occurring human endoglin isoform 1 precursor polypeptide, while SEQ ID NO: 4 encodes a mature, extracellular domain of endoglin isoform 1. The subject nucleic acids may be single-stranded or double stranded. Such nucleic acids may be DNA or RNA molecules. These nucleic acids may be used, for example, in methods for making TGF-beta superfamily heteromultimers of the present disclosure.

In certain embodiments, nucleic acids encoding TGFβ superfamily-receptor polypeptides of the present disclosure are understood to include nucleic acids of any one of SEQ ID NOs: 3, 4, 7, 8, 11, 12, 15, 16, 19, 20, 23, 24, 27, 28, 31, 32, 35, 36, 39, 40, 43, 44, 47, 48, 51, 52, 55, 56, 59, 60, 63, 64, 67, 68, 71, 72, 75, 76, 79, 80, 83, 84, 87, 88, 91, 92, 94, 97, 98, 101, 102, 105, and 106 as well as variants thereof. Variant nucleotide sequences include sequences that differ by one or more nucleotide substitutions, additions, or deletions including allelic variants, and therefore, will include coding sequences that differ from the nucleotide sequence designated in any one of SEQ ID NOs: 3, 4, 7, 8, 11, 12, 15, 16, 19, 20, 23, 24, 27, 28, 31, 32, 35, 36, 39, 40, 43, 44, 47, 48, 51, 52, 55, 56, 59, 60, 63, 64, 67, 68, 71, 72, 75, 76, 79, 80, 83, 84, 87, 88, 91, 92, 94, 97, 98, 101, 102, 105, and 106.

In certain embodiments, TGFβ superfamily co-receptor polypeptides of the present disclosure are encoded by isolated or recombinant nucleic acid sequences that are at least 70%, 75%, 80%, 85%, 90%, 91%, 92%, 93%, 94%, 95%, 96%, 97%, 98%, 99%, or 100% identical to SEQ ID NOs: 3, 4, 7, 8, 11, 12, 15, 16, 19, 20, 23, 24, 27, 28, 31, 32, 35, 36, 39, 40, 43, 44, 47, 48, 51, 52, 55, 56, 59, 60, 63, 64, 67, 68, 71, 72, 75, 76, 79, 80, 83, 84, 87, 88, 91, 92, 94, 97, 98, 101, 102, 105, and 106. One of ordinary skill in the art will appreciate that nucleic acid sequences that are at least 70%, 75%, 80%, 85%, 90%, 91%, 92%, 93%, 94%, 95%, 96%, 97%, 98%, 99%, or 100% identical to the sequences complementary to SEQ ID NOs: 3, 4, 7, 8, 11, 12, 15, 16, 19, 20, 23, 24, 27, 28, 31, 32, 35, 36, 39, 40, 43, 44, 47, 48, 51, 52, 55, 56, 59, 60, 63, 64, 67, 68, 71, 72, 75, 76, 79, 80, 83, 84, 87, 88, 91, 92, 94, 97, 98, 101, 102, 105, and 106 are also within the scope of the present disclosure. In further embodiments, the nucleic acid sequences of the disclosure can be isolated, recombinant, and/or fused with a heterologous nucleotide sequence or in a DNA library.

In other embodiments, nucleic acids of the present disclosure also include nucleotide sequences that hybridize under highly stringent conditions to the nucleotide sequence designated in SEQ ID NOs: 3, 4, 7, 8, 11, 12, 15, 16, 19, 20, 23, 24, 27, 28, 31, 32, 35, 36, 39, 40, 43, 44, 47, 48, 51, 52, 55, 56, 59, 60, 63, 64, 67, 68, 71, 72, 75, 76, 79, 80, 83, 84, 87, 88, 91, 92, 94, 97, 98, 101, 102, 105, and 106, the complement sequence of SEQ ID NOs: 3, 4, 7, 8, 11, 12, 15, 16, 19, 20, 23, 24, 27, 28, 31, 32, 35, 36, 39, 40, 43, 44, 47, 48, 51, 52, 55, 56, 59, 60, 63, 64, 67, 68, 71, 72, 75, 76, 79, 80, 83, 84, 87, 88, 91, 92, 94, 97, 98, 101, 102, 105, and 106, or fragments thereof. One of ordinary skill in the art will understand readily that appropriate stringency conditions which promote DNA hybridization can be varied. For example, one could perform the hybridization at 6.0 x sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the nucleic acids as set forth in SEQ ID NOs: 3, 4, 7, 8, 11, 12, 15, 16, 19, 20, 23, 24, 27, 28, 31, 32, 35, 36, 39, 40, 43, 44, 47, 48, 51, 52, 55, 56, 59, 60, 63, 64, 67, 68, 71, 72, 75, 76, 79, 80, 83, 84, 87, 88, 91, 92, 94, 97, 98, 101, 102, 105, and 106 due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In certain embodiments, the recombinant nucleic acids of the present disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and will vary with the host cell used.

In certain aspects of the present disclosure, the subject nucleic acid is provided in an expression vector comprising a nucleotide sequence encoding a TGFβ superfamily co-receptor polypeptide and operably linked to at least one regulatory sequence. Regulatory sequences are art-recognized and are selected to direct expression of the TGFβ superfamily co-receptor polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding a TGFβ superfamily co-receptor polypeptide. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid of the present disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant TGFβ superfamily co-receptor polypeptide include plasmids and other vectors. For instance, suitable vectors include plasmids of the following types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see, e.g., Molecular Cloning A Laboratory Manual, 3rd Ed., ed. by Sambrook, Fritsch and Maniatis (Cold Spring Harbor Laboratory Press, 2001). In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the ß-gal containing pBlueBac III).

In a preferred embodiment, a vector will be designed for production of the subject TGFβ superfamily co-receptor polypeptides in CHO cells, such as a Pcmv-Script vector (Stratagene, La Jolla, Calif.), pcDNA4 vectors (Invitrogen, Carlsbad, Calif.) and pCI-neo vectors (Promega, Madison, Wis.). As will be apparent, the subject gene constructs can be used to cause expression of the subject TGFβ superfamily co-receptor polypeptides in cells propagated in culture, e.g., to produce proteins, including fusion proteins or variant proteins, for purification.

This disclosure also pertains to a host cell transfected with a recombinant gene including a coding sequence for one or more of the subject TGF superfamily co-receptor polypeptides. The host cell may be any prokaryotic or eukaryotic cell. For example, a TGFβ superfamily co-receptor polypeptide of the disclosure may be expressed in bacterial cells such as E. coli, insect cells (e.g., using a baculovirus expression system), yeast, or mammalian cells [e.g. a Chinese hamster ovary (CHO) cell line]. Other suitable host cells are known to those skilled in the art.

Accordingly, the present disclosure further pertains to methods of producing the subject TGFβ superfamily co-receptor polypeptides. For example, a host cell transfected with an expression vector encoding a TGF superfamily co-receptor polypeptide can be cultured under appropriate conditions to allow expression of the TGFβ superfamily co-receptor polypeptide to occur. The polypeptide may be secreted and isolated from a mixture of cells and medium containing the polypeptide. Alternatively, the TGFβ superfamily co-receptor polypeptide may be isolated from a cytoplasmic or membrane fraction obtained from harvested and lysed cells. A cell culture includes host cells, media and other byproducts. Suitable media for cell culture are well known in the art. The subject polypeptides can be isolated from cell culture medium, host cells, or both, using techniques known in the art for purifying proteins, including ion-exchange chromatography, gel filtration chromatography, ultrafiltration, electrophoresis, immunoaffinity purification with antibodies specific for particular epitopes of the TGFβ superfamily co-receptor polypeptides and affinity purification with an agent that binds to a domain fused to TGFβ superfamily co-receptor polypeptides (e.g., a protein A column may be used to purify a TGFβ superfamily co-receptor-Fc fusion protein). In some embodiments, the TGFβ superfamily co-receptor polypeptide is a fusion protein containing a domain which facilitates its purification.

In some embodiments, purification is achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. A TGFβ superfamily co-receptor-Fc fusion protein may be purified to a purity of >90%, >95%, >96%, >98%, or >99% as determined by size exclusion chromatography and >90%, >95%, >96%, >98%, or >99% as determined by SDS PAGE. The target level of purity should be one that is sufficient to achieve desirable results in mammalian systems, particularly non-human primates, rodents (mice), and humans.

In another embodiment, a fusion gene coding for a purification leader sequence, such as a poly-(His)/enterokinase cleavage site sequence at the N-terminus of the desired portion of the recombinant TGFβ co-receptor polypeptide, can allow purification of the expressed fusion protein by affinity chromatography using a Ni²⁺ metal resin. The purification leader sequence can then be subsequently removed by treatment with enterokinase to provide the purified TGFβ superfamily co-receptor polypeptide. See, e.g., Hochuli et al. (1987) J Chromatography 411:177; and Janknecht et al. (1991) PNAS USA 88:8972.

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence. See, e.g., Current Protocols in Molecular Biology, eds. Ausubel et al., John Wiley & Sons: 1992.

### 4. Screening Assays

In certain aspects, the present disclosure relates to the use of TGFβ superfamily co-receptor heteromultimers which are agonists or antagonists of TGFβ superfamily receptors. Compounds identified through this screening can be tested to assess their ability to modulate tissues such as bone, cartilage, muscle, fat, and/or neurons, to assess their ability to modulate tissue growth in vivo or in vitro. These compounds can be tested, for example, in animal models.

There are numerous approaches to screening for therapeutic agents for modulating tissue growth by targeting TGFβ superfamily ligand signaling (e.g., SMAD signaling). In certain embodiments, high-throughput screening of compounds can be carried out to identify agents that perturb TGFβ superfamily receptor-mediated effects on a selected cell line. In certain embodiments, the assay is carried out to screen and identify compounds that specifically inhibit or reduce binding of a TGF-beta superfamily co-receptor heteromultimer to its binding partner, such as a TGFβ superfamily ligand (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). Alternatively, the assay can be used to identify compounds that enhance binding of a TGF-beta superfamily co-receptor heteromultimer to its binding partner such as a TGFβ superfamily ligand. In a further embodiment, the compounds can be identified by their ability to interact with a TGF-beta superfamily co-receptor heteromultimer of the disclosure.

A variety of assay formats will suffice and, in light of the present disclosure, those not expressly described herein will nevertheless be comprehended by one of ordinary skill in the art. As described herein, the test compounds (agents) of the invention may be created by any combinatorial chemical method. Alternatively, the subject compounds may be naturally occurring biomolecules synthesized in vivo or in vitro. Compounds (agents) to be tested for their ability to act as modulators of tissue growth can be produced, for example, by bacteria, yeast, plants or other organisms (e.g., natural products), produced chemically (e.g., small molecules, including peptidomimetics), or produced recombinantly. Test compounds contemplated by the present invention include non-peptidyl organic molecules, peptides, polypeptides, peptidomimetics, sugars, hormones, and nucleic acid molecules. In certain embodiments, the test agent is a small organic molecule having a molecular weight of less than about 2,000 Daltons.

The test compounds of the disclosure can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be optionally derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S-transferase (GST), photoactivatible crosslinkers or any combinations thereof.

In many drug-screening programs which test libraries of compounds and natural extracts, high-throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the drug on the molecular target as may be manifest in an alteration of binding affinity between a TGF-beta superfamily co-receptor heteromultimer and its binding partner (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty).

Merely to illustrate, in an exemplary screening assay of the present disclosure, the compound of interest is contacted with an isolated and purified TGF-beta superfamily co-receptor heteromultimer which is ordinarily capable of binding to a TGF-beta superfamily ligand, as appropriate for the intention of the assay. To the mixture of the compound and TGF-beta superfamily co-receptor heteromultimer is then added to a composition containing the appropriate TGF-beta superfamily ligand (e.g., BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, glial cell-derived neurotrophic factor (GDNF), neurturin, artemin, persephin, MIS, and Lefty). Detection and quantification of heteromultimer-superfamily ligand complexes provides a means for determining the compound's efficacy at inhibiting (or potentiating) complex formation between the TGF-beta superfamily co-receptor heteromultimer and its binding protein. The efficacy of the compound can be assessed by generating dose-response curves from data obtained using various concentrations of the test compound. Moreover, a control assay can also be performed to provide a baseline for comparison. For example, in a control assay, isolated and purified TGF-beta superfamily ligand is added to a composition containing the TGF-beta superfamily co-receptor heteromultimer, and the formation of heteromultimer-ligand complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system.

Binding of a TGF-beta superfamily co-receptor heteromultimer to another protein may be detected by a variety of techniques. For instance, modulation of the formation of complexes can be quantitated using, for example, detectably labeled proteins such as radiolabeled (e.g., ³²P, ³⁵ S, ¹⁴C or ³H), fluorescently labeled (e.g., FITC), or enzymatically labeled TGF-beta superfamily co-receptor heteromultimer and/or its binding protein, by immunoassay, or by chromatographic detection.

In certain embodiments, the present disclosure contemplates the use of fluorescence polarization assays and fluorescence resonance energy transfer (FRET) assays in measuring, either directly or indirectly, the degree of interaction between a TGF-beta superfamily co-receptor heteromultimer and its binding protein. Further, other modes of detection, such as those based on optical waveguides (see, e.g., PCT Publication WO 96/26432 and U.S. Pat. No. 5,677,196), surface plasmon resonance (SPR), surface charge sensors, and surface force sensors, are compatible with many embodiments of the disclosure.

Moreover, the present disclosure contemplates the use of an interaction trap assay, also known as the “two-hybrid assay,” for identifying agents that disrupt or potentiate interaction between a TGF-beta superfamily heteromultimer and its binding partner. See, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In a specific embodiment, the present disclosure contemplates the use of reverse two-hybrid systems to identify compounds (e.g., small molecules or peptides) that dissociate interactions between a TGF-beta superfamily heteromultimer and its binding protein [see, e.g., Vidal and Legrain, (1999) Nucleic Acids Res 27:919-29; Vidal and Legrain, (1999) Trends Biotechnol 17:374-81; and U.S. Pat. Nos. 5,525,490; 5,955,280; and 5,965,368].

In certain embodiments, the subject compounds are identified by their ability to interact with a TGF-beta superfamily co-receptor heteromultimer of the disclosure. The interaction between the compound and the TGF-beta superfamily co-receptor heteromultimer may be covalent or non-covalent. For example, such interaction can be identified at the protein level using in vitro biochemical methods, including photo-crosslinking, radiolabeled ligand binding, and affinity chromatography. See, e.g., Jakoby W B et al. (1974) Methods in Enzymology 46:1. In certain cases, the compounds may be screened in a mechanism-based assay, such as an assay to detect compounds which bind to a TGF-beta superfamily co-receptor heteromultimer. This may include a solid-phase or fluid-phase binding event. Alternatively, the gene encoding a TGF-beta superfamily co-receptor heteromultimer can be transfected with a reporter system (e.g., β-galactosidase, luciferase, or green fluorescent protein) into a cell and screened against the library preferably by high-throughput screening or with individual members of the library. Other mechanism-based binding assays may be used; for example, binding assays which detect changes in free energy. Binding assays can be performed with the target fixed to a well, bead or chip or captured by an immobilized antibody or resolved by capillary electrophoresis. The bound compounds may be detected usually using colorimetric endpoints or fluorescence or surface plasmon resonance.

### 5. Exemplary Therapeutic Uses

In aspects embodiments, a TGF-beta superfamily co-receptor heteromultimer, or combination of TGF-beta superfamily co-receptor heteromultimers, of the present disclosure can be administered to a patient in need thereof, particularly to treat or prevent a TGF-beta superfamily-associated disorder or condition. In some embodiments, the present invention provides methods of treating a disorder or condition in a patient in need thereof by administering to the patient a therapeutically effective amount of a TGF-beta superfamily co-receptor heteromultimer, or combination of TGF-beta superfamily co-receptor heteromultimers, as described herein. In some embodiments, the present invention provides methods of preventing a disorder or condition in a patient in need thereof by administering to the patient a therapeutically effective amount of a TGF-beta superfamily co-receptor heteromultimer, or combination of TGF-beta superfamily co-receptor heteromultimers, as described herein. In some embodiments, the present invention provides methods of delaying the progression or onset a disorder or condition in a patient in need thereof by administering to the patient a therapeutically effective amount of a TGF-beta superfamily co-receptor heteromultimer, or combination of TGF-beta superfamily co-receptor heteromultimers, as described herein. In some embodiments, the present invention provides methods of treating one or more complications of a disorder or condition in a patient in need thereof by administering to the patient a therapeutically effective amount of a TGF-beta superfamily co-receptor heteromultimer, or combination of TGF-beta superfamily co-receptor heteromultimers, as described herein. In some embodiments, the disorder or condition is one or more of anemia, a thalassemia, myelodysplastic syndrome (MDS), sickle cell disease, and a bone-related disorder (e.g., a bone-related disorder associated with one or more of low bone density, low bone strength, and/or low bone growth). In some embodiments, the methods of the disclosure relate to increasing bone growth in a patient in need thereof. In some embodiments, the methods of the disclosure relate to increasing bone strength in a patient in need thereof. In some embodiments, the methods of the disclosure relate to increasing bone density (e.g., bone mineral density) in a patient in need thereof. In some embodiments, the methods of the disclosure relate to increasing red blood cell levels in a patient in need thereof. In some embodiments, the methods of the disclosure relate to increasing hemoglobin levels in a patient in need thereof. Optionally, any of the TGF-beta superfamily co-receptor heteromultimers of the present disclosure can potentially be employed individually or in combination for therapeutic uses disclosed herein. These methods are particularly aimed at therapeutic and prophylactic treatments of mammals including, for example, rodents, primates, and humans.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample. The term “treating” as used herein includes amelioration or elimination of the condition once it has been established. In either case, prevention or treatment may be discerned in the diagnosis provided by a physician or other health care provider and the intended result of administration of the therapeutic agent.

In certain embodiments, a TGF-beta superfamily co-receptor heteromultimer, or combinations of TGF-beta superfamily co-receptor heteromultimers, of the present disclosure may be used in methods of inducing bone and/or cartilage formation, preventing bone loss, increasing bone mineralization, preventing the demineralization of bone, and/or increasing bone density. TGF-beta superfamily co-receptor heteromultimers may be useful in patients who are diagnosed with subclinical low bone density, as a protective measure against the development of osteoporosis.

In some embodiments, a TGF-beta superfamily co-receptor heteromultimer, or combinations of TGF-beta superfamily co-receptor heteromultimers, of the present disclosure may find medical utility in the healing of bone fractures and cartilage defects in humans and other animals. The subject methods and compositions may also have prophylactic use in closed as well as open fracture reduction and also in the improved fixation of artificial joints. De novo bone formation induced by an osteogenic agent is useful for repair of craniofacial defects that are congenital, trauma-induced, or caused by oncologic resection, and is also useful in cosmetic plastic surgery. Further, methods and compositions of the invention may be used in the treatment of periodontal disease and in other tooth repair processes. In certain cases, a TGF-beta superfamily co-receptor heteromultimer, or combinations of TGF-beta superfamily co-receptor heteromultimers, may provide an environment to attract bone-forming cells, stimulate growth of bone-forming cells, or induce differentiation of progenitors of bone-forming cells. TGF-beta superfamily co-receptor heteromultimers of the disclosure may also be useful in the treatment of osteoporosis. Further, TGF-beta superfamily co-receptor heteromultimers may be used in repair of cartilage defects and prevention/reversal of osteoarthritis.

In some embodiments, methods and compositions of the disclosure can be applied to conditions characterized by or causing bone loss, such as osteoporosis (including secondary osteoporosis), hyperparathyroidism, mineral bone disorder, sex hormone deprivation or ablation (e.g. androgen and/or estrogen), glucocorticoid treatment, rheumatoid arthritis, severe burns, hyperparathyroidism, hypercalcemia, hypocalcemia, hypophosphatemia, osteomalacia (including tumor-induced osteomalacia), hyperphosphatemia, vitamin D deficiency, hyperparathyroidism (including familial hyperparathyroidism) and pseudohypoparathyroidism, tumor metastases to bone, bone loss as a consequence of a tumor or chemotherapy, tumors of the bone and bone marrow (e.g., multiple myeloma), ischemic bone disorders, periodontal disease and oral bone loss, Cushing's disease, Paget's disease, thyrotoxicosis, chronic diarrheal state or malabsorption, renal tubular acidosis, or anorexia nervosa. Methods and compositions of the invention may also be applied to conditions characterized by a failure of bone formation or healing, including non-union fractures, fractures that are otherwise slow to heal, fetal and neonatal bone dysplasias (e.g., hypocalcemia, hypercalcemia, calcium receptor defects and vitamin D deficiency), osteonecrosis (including osteonecrosis of the jaw) and osteogenesis imperfecta. Additionally, the anabolic effects will cause such antagonists to diminish bone pain associated with bone damage or erosion. As a consequence of the anti-resorptive effects, such antagonists may be useful to treat disorders of abnormal bone formation, such as osteoblastic tumor metastases (e.g., associated with primary prostate or breast cancer), osteogenic osteosarcoma, osteopetrosis, progressive diaphyseal dysplasia, endosteal hyperostosis, osteopoikilosis, and melorheostosis. Other disorders that may be treated include fibrous dysplasia and chondrodysplasias.

In another specific embodiment, the disclosure provides a therapeutic method and composition for repairing fractures and other conditions related to cartilage and/or bone defects or periodontal diseases. The invention further provides therapeutic methods and compositions for wound healing and tissue repair. The types of wounds include, but are not limited to, burns, incisions and ulcers. See, e.g., PCT Publication No. WO 84/01106. Such compositions comprise a therapeutically effective amount of at least one of the TGF-beta superfamily co-receptor heteromultimers of the disclosure in admixture with a pharmaceutically acceptable vehicle, carrier, or matrix.

In some embodiments, a TGF-beta superfamily co-receptor heteromultimer, or combinations of TGF-beta superfamily co-receptor heteromultimers, of the disclosure can be applied to conditions causing bone loss such as osteoporosis, hyperparathyroidism, Cushing's disease, thyrotoxicosis, chronic diarrheal state or malabsorption, renal tubular acidosis, or anorexia nervosa. It is commonly appreciated that being female, having a low body weight, and leading a sedentary lifestyle are risk factors for osteoporosis (loss of bone mineral density, leading to fracture risk). However, osteoporosis can also result from the long-term use of certain medications. Osteoporosis resulting from drugs or another medical condition is known as secondary osteoporosis. In Cushing's disease, the excess amount of cortisol produced by the body results in osteoporosis and fractures. The most common medications associated with secondary osteoporosis are the corticosteroids, a class of drugs that act like cortisol, a hormone produced naturally by the adrenal glands. Although adequate levels of thyroid hormones are needed for the development of the skeleton, excess thyroid hormone can decrease bone mass over time. Antacids that contain aluminum can lead to bone loss when taken in high doses. Other medications that can cause secondary osteoporosis include phenytoin (Dilantin) and barbiturates that are used to prevent seizures; methotrexate (Rheumatrex, Immunex, Folex PFS), a drug for some forms of arthritis, cancer, and immune disorders; cyclosporine (Sandimmune, Neoral), a drug used to treat some autoimmune diseases and to suppress the immune system in organ transplant patients; luteinizing hormone-releasing hormone agonists (Lupron, Zoladex), used to treat prostate cancer and endometriosis; heparin (Calciparine, Liquaemin), an anticlotting medication; and cholestyramine (Questran) and colestipol (Colestid), used to treat high cholesterol. Bone loss resulting from cancer therapy is widely recognized and termed cancer therapy-induced bone loss (CTIBL). Bone metastases can create cavities in the bone that may be corrected by treatment with a TGF-beta superfamily co-receptor heteromultimer. Bone loss can also be caused by gum disease, a chronic infection in which bacteria located in gum recesses produce toxins and harmful enzymes.

In a further embodiment, the present disclosure provides methods and therapeutic agents for treating diseases or disorders associated with abnormal or unwanted bone growth. For example, patients with the congenital disorder fibrodysplasia ossificans progressiva (FOP) are afflicted by progressive ectopic bone growth in soft tissues spontaneously or in response to tissue trauma, with a major impact on quality of life. Additionally, abnormal bone growth can occur after hip replacement surgery and thus ruin the surgical outcome. This is a more common example of pathological bone growth and a situation in which the subject methods and compositions may be therapeutically useful. The same methods and compositions may also be useful for treating other forms of abnormal bone growth (e.g., pathological growth of bone following trauma, burns or spinal cord injury), and for treating or preventing the undesirable conditions associated with the abnormal bone growth seen in connection with metastatic prostate cancer or osteosarcoma.

In certain embodiments, a TGF-beta superfamily co-receptor heteromultimer, or combinations of TGF-beta superfamily co-receptor heteromultimers, of the disclosure may be used to promote bone formation in patients with cancer. Patients having certain tumors are at high risk for bone loss due to tumor-induced bone loss, bone metastases, and therapeutic agents. Generally, DEXA scans are employed to assess changes in bone density, while indicators of bone remodeling may be used to assess the likelihood of bone metastases. Serum markers may be monitored. Bone specific alkaline phosphatase (BSAP) is an enzyme that is present in osteoblasts. Blood levels of BSAP are increased in patients with bone metastasis and other conditions that result in increased bone remodeling. Osteocalcin and procollagen peptides are also associated with bone formation and bone metastases. Increases in BSAP have been detected in patients with bone metastasis caused by prostate cancer, and to a lesser degree, in bone metastases from breast cancer. BMP7 levels are high in prostate cancer that has metastasized to bone, but not in bone metastases due to bladder, skin, liver, or lung cancer. Type I carboxy-terminal telopeptide (ICTP) is a crosslink found in collagen that is formed during to the resorption of bone. Since bone is constantly being broken down and reformed, ICTP will be found throughout the body. However, at the site of bone metastasis, the level will be significantly higher than in an area of normal bone. ICTP has been found in high levels in bone metastasis due to prostate, lung, and breast cancer. Another collagen crosslink, Type I N-terminal telopeptide (NTx), is produced along with ICTP during bone turnover. The amount of NTx is increased in bone metastasis caused by many different types of cancer including lung, prostate, and breast cancer. Also, the levels of NTx increase with the progression of the bone metastasis. Therefore, this marker can be used to both detect metastasis as well as measure the extent of the disease. Other markers of resorption include pyridinoline and deoxypyridinoline. Any increase in resorption markers or markers of bone metastases indicate the need for therapy with a TGF-beta superfamily co-receptor heteromultimer, or combinations of TGF-beta superfamily co-receptor heteromultimers, in a patient.

A TGF-beta superfamily co-receptor heteromultimer, or combinations of TGF-beta superfamily co-receptor heteromultimers, of the disclosure may be conjointly administered with other bone-active pharmaceutical agents. Conjoint administration may be accomplished by administration of a single co-formulation, by simultaneous administration, or by administration at separate times. TGF-beta superfamily co-receptor heteromultimer complexes may be particularly advantageous if administered with other bone-active agents. A patient may benefit from conjointly receiving a TGF-beta superfamily co-receptor heteromultimer complex and taking calcium supplements, vitamin D, appropriate exercise and/or, in some cases, other medication. Examples of other medications incude, bisphosphonates (alendronate, ibandronate and risedronate), calcitonin, estrogens, parathyroid hormone and raloxifene. The bisphosphonates (alendronate, ibandronate and risedronate), calcitonin, estrogens and raloxifene affect the bone remodeling cycle and are classified as anti-resorptive medications. Bone remodeling consists of two distinct stages: bone resorption and bone formation. Anti-resorptive medications slow or stop the bone-resorbing portion of the bone-remodeling cycle but do not slow the bone-forming portion of the cycle. As a result, new formation continues at a greater rate than bone resorption, and bone density may increase over time. Teriparatide, a form of parathyroid hormone, increases the rate of bone formation in the bone remodeling cycle. Alendronate is approved for both the prevention (5 mg per day or 35 mg once a week) and treatment (10 mg per day or 70 mg once a week) of postmenopausal osteoporosis. Alendronate reduces bone loss, increases bone density and reduces the risk of spine, wrist and hip fractures. Alendronate also is approved for treatment of glucocorticoid-induced osteoporosis in men and women as a result of long-term use of these medications (i.e., prednisone and cortisone) and for the treatment of osteoporosis in men. Alendronate plus vitamin D is approved for the treatment of osteoporosis in postmenopausal women (70 mg once a week plus vitamin D), and for treatment to improve bone mass in men with osteoporosis. Ibandronate is approved for the prevention and treatment of postmenopausal osteoporosis. Taken as a once-a-month pill (150 mg), ibandronate should be taken on the same day each month. Ibandronate reduces bone loss, increases bone density and reduces the risk of spine fractures. Risedronate is approved for the prevention and treatment of postmenopausal osteoporosis. Taken daily (5 mg dose) or weekly (35 mg dose or 35 mg dose with calcium), risedronate slows bone loss, increases bone density and reduces the risk of spine and non-spine fractures. Risedronate also is approved for use by men and women to prevent and/or treat glucocorticoid-induced osteoporosis that results from long-term use of these medications (i.e., prednisone or cortisone). Calcitonin is a naturally occurring hormone involved in calcium regulation and bone metabolism. In women who are more than 5 years beyond menopause, calcitonin slows bone loss, increases spinal bone density, and may relieve the pain associated with bone fractures. Calcitonin reduces the risk of spinal fractures. Calcitonin is available as an injection (50-100 IU daily) or nasal spray (200 IU daily).

A patient may also benefit from conjointly receiving a TGF-beta superfamily co-receptor heteromultimer, or combinations of TGF-beta superfamily co-receptor heteromultimers, and additional bone-active medications. Estrogen therapy (ET)/hormone therapy (HT) is approved for the prevention of osteoporosis. ET has been shown to reduce bone loss, increase bone density in both the spine and hip, and reduce the risk of hip and spinal fractures in postmenopausal women. ET is administered most commonly in the form of a pill or skin patch that delivers a low dose of approximately 0.3 mg daily or a standard dose of approximately 0.625 mg daily and is effective even when started after age 70. When estrogen is taken alone, it can increase a woman's risk of developing cancer of the uterine lining (endometrial cancer). To eliminate this risk, healthcare providers prescribe the hormone progestin in combination with estrogen (hormone replacement therapy or HT) for those women who have an intact uterus. ET/HT relieves menopause symptoms and has been shown to have a beneficial effect on bone health. Side effects may include vaginal bleeding, breast tenderness, mood disturbances and gallbladder disease. Raloxifene, 60 mg a day, is approved for the prevention and treatment of postmenopausal osteoporosis. It is from a class of drugs called Selective Estrogen Receptor Modulators (SERMs) that have been developed to provide the beneficial effects of estrogens without their potential disadvantages. Raloxifene increases bone mass and reduces the risk of spine fractures. Data are not yet available to demonstrate that raloxifene can reduce the risk of hip and other non-spine fractures. Teriparatide, a form of parathyroid hormone, is approved for the treatment of osteoporosis in postmenopausal women and men who are at high risk for a fracture. This medication stimulates new bone formation and significantly increases bone mineral density. In postmenopausal women, fracture reduction was noted in the spine, hip, foot, ribs and wrist. In men, fracture reduction was noted in the spine, but there were insufficient data to evaluate fracture reduction at other sites. Teriparatide is self-administered as a daily injection for up to 24 months.

In certain aspects, a TGF-beta superfamily co-receptor heteromultimer, or combinations of TGF-beta superfamily co-receptor heteromultimers, of the present disclosure can be used to increase red blood cell levels, treat or prevent an anemia, and/or treat or prevent ineffective erythropoiesis in a subject in need thereof. In certain aspects, a TGF-beta superfamily co-receptor heteromultimer, or combinations of TGF-beta superfamily co-receptor heteromultimers, of the present disclosure may be used in combination with conventional therapeutic approaches for increasing red blood cell levels, particularly those used to treat anemias of multifactorial origin. Conventional therapeutic approaches for increasing red blood cell levels include, for example, red blood cell transfusion, administration of one or more EPO receptor activators, hematopoietic stem cell transplantation, immunosuppressive biologics and drugs (e.g., corticosteroids). In certain embodiments, a TGF-beta superfamily co-receptor heteromultimer, or combinations of TGF-beta superfamily co-receptor heteromultimers, of the present disclosure can be used to treat or prevent ineffective erythropoiesis and/or the disorders associated with ineffective erythropoiesis in a subject in need thereof. In certain aspects, a TGF-beta superfamily co-receptor heteromultimer, or combinations of TGF-beta superfamily co-receptor heteromultimers, of the present disclosure can be used in combination with conventional therapeutic approaches for treating or preventing an anemia or ineffective erythropoiesis disorder, particularly those used to treat anemias of multifactorial origin.

In certain embodiments, a TGF-beta superfamily co-receptor heteromultimer, or combinations of TGF-beta superfamily co-receptor heteromultimers, optionally combined with an EPO receptor activator, may be used to increase red blood cell, hemoglobin, or reticulocyte levels in healthy individuals and selected patient populations. Examples of appropriate patient populations include those with undesirably low red blood cell or hemoglobin levels, such as patients having an anemia, and those that are at risk for developing undesirably low red blood cell or hemoglobin levels, such as those patients who are about to undergo major surgery or other procedures that may result in substantial blood loss. In one embodiment, a patient with adequate red blood cell levels is treated with a TGF-beta superfamily co-receptor heteromultimer, or combinations of TGF-beta superfamily co-receptor heteromultimers, to increase red blood cell levels, and then blood is drawn and stored for later use in transfusions.

One or more TGF-beta superfamily co-receptor heteromultimers of the disclosure, optionally combined with an EPO receptor activator, may be used to increase red blood cell levels, hemoglobin levels, and/or hematocrit levels in a patient having an anemia. When observing hemoglobin and/or hematocrit levels in humans, a level of less than normal for the appropriate age and gender category may be indicative of anemia, although individual variations are taken into account. For example, a hemoglobin level from 10-12.5 g/dl, and typically about 11.0 g/dl is considered to be within the normal range in health adults, although, in terms of therapy, a lower target level may cause fewer cardiovascular side effects [see, e.g., Jacobs et al. (2000) Nephrol Dial Transplant 15, 15-19]. Alternatively, hematocrit levels (percentage of the volume of a blood sample occupied by the cells) can be used as a measure for anemia. Hematocrit levels for healthy individuals range from about 41-51% for adult males and from 35-45% for adult females. In certain embodiments, a patient may be treated with a dosing regimen intended to restore the patient to a target level of red blood cells, hemoglobin, and/or hematocrit. As hemoglobin and hematocrit levels vary from person to person, optimally, the target hemoglobin and/or hematocrit level can be individualized for each patient.

Anemia is frequently observed in patients having a tissue injury, an infection, and/or a chronic disease, particularly cancer. In some subjects, anemia is distinguished by low erythropoietin levels and/or an inadequate response to erythropoietin in the bone marrow [see, e.g., Adamson (2008) Harrison's Principles of Internal Medicine, 17th ed.; McGraw Hill, N.Y., pp 628-634]. Potential causes of anemia include, for example, blood loss, nutritional deficits (e.g. reduced dietary intake of protein), medication reaction, various problems associated with the bone marrow, and many diseases. More particularly, anemia has been associated with a variety of disorders and conditions that include, for example, bone marrow transplantation; solid tumors (e.g., breast cancer, lung cancer, and colon cancer); tumors of the lymphatic system (e.g., chronic lymphocyte leukemia, non-Hodgkins lymphoma, and Hodgkins lymphoma); tumors of the hematopoietic system (e.g., leukemia, a myelodysplastic syndrome and multiple myeloma); radiation therapy; chemotherapy (e.g., platinum containing regimens); inflammatory and autoimmune diseases, including, but not limited to, rheumatoid arthritis, other inflammatory arthritides, systemic lupus erythematosis (SLE), acute or chronic skin diseases (e.g., psoriasis), inflammatory bowel disease (e.g., Crohn's disease and ulcerative colitis); acute or chronic renal disease or failure, including idiopathic or congenital conditions; acute or chronic liver disease; acute or chronic bleeding; situations where transfusion of red blood cells is not possible due to patient allo- or auto-antibodies and/or for religious reasons (e.g., some Jehovah's Witnesses); infections (e.g., malaria and osteomyelitis); hemoglobinopathies including, for example, sickle cell disease (anemia), thalassemias; drug use or abuse (e.g., alcohol misuse); pediatric patients with anemia from any cause to avoid transfusion; and elderly patients or patients with underlying cardiopulmonary disease with anemia who cannot receive transfusions due to concerns about circulatory overload [see, e.g., Adamson (2008) Harrison's Principles of Internal Medicine, 17th ed.; McGraw Hill, N.Y., pp 628-634]. In some embodiments, one or more TGF-beta superfamily co-receptor heteromultimers of the disclosure could be used to treat or prevent anemia associated with one or more of the disorders or conditions disclosed herein.

Many factors can contribute to cancer-related anemia. Some are associated with the disease process itself and the generation of inflammatory cytokines such as interleukin-1, interferon-gamma, and tumor necrosis factor [Bron et al. (2001) Semin Oncol 28(Suppl 8):1-6]. Among its effects, inflammation induces the key iron-regulatory peptide hepcidin, thereby inhibiting iron export from macrophages and generally limiting iron availability for erythropoiesis [see, e.g., Ganz (2007) J Am Soc Nephrol 18:394-400]. Blood loss through various routes can also contribute to cancer-related anemia. The prevalence of anemia due to cancer progression varies with cancer type, ranging from 5% in prostate cancer up to 90% in multiple myeloma. Cancer-related anemia has profound consequences for patients, including fatigue and reduced quality of life, reduced treatment efficacy, and increased mortality. In some embodiments, one or more TGF-beta superfamily co-receptor heteromultimers of the disclosure, optionally combined with an EPO receptor activator, could be used to treat a cancer-related anemia.

A hypoproliferative anemia can result from primary dysfunction or failure of the bone marrow. Hypoproliferative anemias include: anemia of chronic disease, anemia associated with hypometabolic states, and anemia associated with cancer. In each of these types, endogenous erythropoietin levels are inappropriately low for the degree of anemia observed. Other hypoproliferative anemias include: early-stage iron-deficient anemia, and anemia caused by damage to the bone marrow. In these types, endogenous erythropoietin levels are appropriately elevated for the degree of anemia observed. Prominent examples would be myelosuppression caused by cancer and/or chemotherapeutic drugs or cancer radiation therapy. A broad review of clinical trials found that mild anemia can occur in 100% of patients after chemotherapy, while more severe anemia can occur in up to 80% of such patients [see, e.g., Groopman et al. (1999) J Natl Cancer Inst 91:1616-1634]. Myelosuppressive drugs include, for example: 1) alkylating agents such as nitrogen mustards (e.g., melphalan) and nitrosoureas (e.g., streptozocin); 2) antimetabolites such as folic acid antagonists (e.g., methotrexate), purine analogs (e.g., thioguanine), and pyrimidine analogs (e.g., gemcitabine); 3) cytotoxic antibiotics such as anthracyclines (e.g., doxorubicin); 4) kinase inhibitors (e.g., gefitinib); 5) mitotic inhibitors such as taxanes (e.g., paclitaxel) and vinca alkaloids (e.g., vinorelbine); 6) monoclonal antibodies (e.g., rituximab); and 7) topoisomerase inhibitors (e.g., topotecan and etoposide). In addition, conditions resulting in a hypometabolic rate can produce a mild-to-moderate hypoproliferative anemia. Among such conditions are endocrine deficiency states. For example, anemia can occur in Addison's disease, hypothyroidism, hyperparathyroidism, or males who are castrated or treated with estrogen. In some embodiments, one or more TGF-beta superfamily co-receptor heteromultimers of the disclosure, optionally combined with an EPO receptor activator, could be used to treat a hyperproliferative anemia.

Anemia resulting from acute blood loss of sufficient volume, such as from trauma or postpartum hemorrhage, is known as acute post-hemorrhagic anemia. Acute blood loss initially causes hypovolemia without anemia since there is proportional depletion of RBCs along with other blood constituents. However, hypovolemia will rapidly trigger physiologic mechanisms that shift fluid from the extravascular to the vascular compartment, which results in hemodilution and anemia. If chronic, blood loss gradually depletes body iron stores and eventually leads to iron deficiency. In some embodiments, one or more TGF-beta superfamily co-receptor heteromultimers of the disclosure, optionally combined with an EPO receptor activator, could be used to treat anemia resulting from acute blood loss.

Iron-deficiency anemia is the final stage in a graded progression of increasing iron deficiency which includes negative iron balance and iron-deficient erythropoiesis as intermediate stages. Iron deficiency can result from increased iron demand, decreased iron intake, or increased iron loss, as exemplified in conditions such as pregnancy, inadequate diet, intestinal malabsorption, acute or chronic inflammation, and acute or chronic blood loss. With mild-to-moderate anemia of this type, the bone marrow remains hypoproliferative, and RBC morphology is largely normal; however, even mild anemia can result in some microcytic hypochromic RBCs, and the transition to severe iron-deficient anemia is accompanied by hyperproliferation of the bone marrow and increasingly prevalent microcytic and hypochromic RBCs [see, e.g., Adamson (2008) Harrison's Principles of Internal Medicine, 17th ed.; McGraw Hill, N.Y., pp 628-634]. Appropriate therapy for iron-deficiency anemia depends on its cause and severity, with oral iron preparations, parenteral iron formulations, and RBC transfusion as major conventional options. In some embodiments, one or more TGF-beta superfamily co-receptor heteromultimers of the disclosure, optionally combined with an EPO receptor activator, could be used to treat a chronic iron-deficiency.

Myelodysplastic syndrome (MDS) is a diverse collection of hematological conditions characterized by ineffective production of myeloid blood cells and risk of transformation to acute myelogenous leukemia. In MDS patients, blood stem cells do not mature into healthy red blood cells, white blood cells, or platelets. MDS disorders include, for example, refractory anemia, refractory anemia with ringed sideroblasts, refractory anemia with excess blasts, refractory anemia with excess blasts in transformation, refractory cytopenia with multilineage dysplasia, and myelodysplastic syndrome associated with an isolated 5q chromosome abnormality. As these disorders manifest as irreversible defects in both quantity and quality of hematopoietic cells, most MDS patients are afflicted with chronic anemia. Therefore, MDS patients eventually require blood transfusions and/or treatment with growth factors (e.g., erythropoietin or G-CSF) to increase red blood cell levels. However, many MDS patients develop side-effects due to frequency of such therapies. For example, patients who receive frequent red blood cell transfusion can exhibit tissue and organ damage from the buildup of extra iron. Accordingly, one or more TGF-beta superfamily heteromultimer complexes of the disclosure, may be used to treat patients having MDS. In certain embodiments, patients suffering from MDS may be treated using one or more TGF-beta superfamily heteromultimers of the disclosure, optionally in combination with an EPO receptor activator. In other embodiments, patients suffering from MDS may be treated using a combination of one or more TGF-beta superfamily co-receptor heteromultimers of the disclosure and one or more additional therapeutic agents for treating MDS including, for example, thalidomide, lenalidomide, azacitadine, decitabine, erythropoietins, deferoxamine, antithymocyte globulin, and filgrastrim (G-CSF).

Originally distinguished from aplastic anemia, hemorrhage, or peripheral hemolysis on the basis of ferrokinetic studies [see, e.g., Ricketts et al. (1978) Clin Nucl Med 3:159-164], ineffective erythropoiesis describes a diverse group of anemias in which production of mature RBCs is less than would be expected given the number of erythroid precursors (erythroblasts) present in the bone marrow [Tanno et al. (2010) Adv Hematol 2010:358283]. In such anemias, tissue hypoxia persists despite elevated erythropoietin levels due to ineffective production of mature RBCs. A vicious cycle eventually develops in which elevated erythropoietin levels drive massive expansion of erythroblasts, potentially leading to splenomegaly (spleen enlargement) due to extramedullary erythropoiesis [see, e.g., Aizawa et al. (2003) Am J Hematol 74:68-72], erythroblast-induced bone pathology [see, e.g., Di Matteo et al. (2008) J Biol Regul Homeost Agents 22:211-216], and tissue iron overload, even in the absence of therapeutic RBC transfusions [see, e.g., Pippard et al. (1979) Lancet 2:819-821]. Thus, by boosting erythropoietic effectiveness, one or more TGF-beta superfamily heteromultimers of the present disclosure may break the aforementioned cycle and thus alleviate not only the underlying anemia but also the associated complications of elevated erythropoietin levels, splenomegaly, bone pathology, and tissue iron overload. In some embodiments, one or more TGF-beta superfamily co-receptor heteromultimers of the present disclosure can be used to treat or prevent ineffective erythropoiesis, including anemia and elevated EPO levels as well as complications such as splenomegaly, erythroblast-induced bone pathology, iron overload, and their attendant pathologies. With splenomegaly, such pathologies include thoracic or abdominal pain and reticuloendothelial hyperplasia. Extramedullary hematopoiesis can occur not only in the spleen but potentially in other tissues in the form of extramedullary hematopoietic pseudotumors [see, e.g., Musallam et al. (2012) Cold Spring Harb Perspect Med 2:a013482]. With erythroblast-induced bone pathology, attendant pathologies include low bone mineral density, osteoporosis, and bone pain [see, e.g., Haidar et al. (2011) Bone 48:425-432]. With iron overload, attendant pathologies include hepcidin suppression and hyperabsorption of dietary iron [see, e.g., Musallam et al. (2012) Blood Rev 26(Suppl 1):S16-S19], multiple endocrinopathies and liver fibrosis/cirrhosis [see, e.g., Galanello et al. (2010) Orphanet J Rare Dis 5:11], and iron-overload cardiomyopathy [Lekawanvijit et al., 2009, Can J Cardiol 25:213-218].

The most common causes of ineffective erythropoiesis are the thalassemia syndromes, hereditary hemoglobinopathies in which imbalances in the production of intact alpha- and beta-hemoglobin chains lead to increased apoptosis during erythroblast maturation [see, e.g., Schrier (2002) Curr Opin Hematol 9:123-126]. Thalassemias are collectively among the most frequent genetic disorders worldwide, with changing epidemiologic patterns predicted to contribute to a growing public health problem in both the U.S. and globally [Vichinsky (2005) Ann NY Acad Sci 1054:18-24]. Thalassemia syndromes are named according to their severity. Thus, α-thalassemias include α-thalassemia minor (also known as α-thalassemia trait; two affected α-globin genes), hemoglobin H disease (three affected α-globin genes), and α-thalassemia major (also known as hydrops fetalis; four affected α-globin genes). β-Thalassemias include β-thalassemia minor (also known as β-thalassemia trait; one affected β-globin gene), β-thalassemia intermedia (two affected β-globin genes), hemoglobin E thalassemia (two affected β-globin genes), and β-thalassemia major (also known as Cooley's anemia; two affected β-globin genes resulting in a complete absence of β-globin protein). β-Thalassemia impacts multiple organs, is associated with considerable morbidity and mortality, and currently requires life-long care. Although life expectancy in patients with β-thalassemia has increased in recent years due to use of regular blood transfusions in combination with iron chelation, iron overload resulting both from transfusions and from excessive gastrointestinal absorption of iron can cause serious complications such as heart disease, thrombosis, hypogonadism, hypothyroidism, diabetes, osteoporosis, and osteopenia [see, e.g., Rund et al. (2005) N Engl J Med 353:1135-1146]. In certain embodiments, one or more TGF-beta superfamily co-receptor heteromultimers of the disclosure, optionally combined with an EPO receptor activator, can be used to treat or prevent a thalassemia syndrome.

In some embodiments, one or more TGF-beta superfamily co-receptor heteromultimers of the disclosure, optionally combined with an EPO receptor activator, can be used for treating disorders of ineffective erythropoiesis besides thalassemia syndromes. Such disorders include siderblastic anemia (inherited or acquired); dyserythropoietic anemia (types I and II); sickle cell anemia; hereditary spherocytosis; pyruvate kinase deficiency; megaloblastic anemias, potentially caused by conditions such as folate deficiency (due to congenital diseases, decreased intake, or increased requirements), cobalamin deficiency (due to congenital diseases, pernicious anemia, impaired absorption, pancreatic insufficiency, or decreased intake), certain drugs, or unexplained causes (congenital dyserythropoietic anemia, refractory megaloblastic anemia, or erythroleukemia); myelophthisic anemias including; congenital erythropoietic porphyria; and lead poisoning.

In certain embodiments, one or more TGF-beta superfamily co-receptor heteromultimers of the disclosure may be used in combination with supportive therapies for ineffective erythropoiesis. Such therapies include transfusion with either red blood cells or whole blood to treat anemia. In chronic or hereditary anemias, normal mechanisms for iron homeostasis are overwhelmed by repeated transfusions, eventually leading to toxic and potentially fatal accumulation of iron in vital tissues such as heart, liver, and endocrine glands. Thus, supportive therapies for patients chronically afflicted with ineffective erythropoiesis also include treatment with one or more iron-chelating molecules to promote iron excretion in the urine and/or stool and thereby prevent, or reverse, tissue iron overload [see, e.g., Hershko (2006) Haematologica 91:1307-1312; Cao et al. (2011), Pediatr Rep 3(2):e17]. Effective iron-chelating agents should be able to selectively bind and neutralize ferric iron, the oxidized form of non-transferrin bound iron which likely accounts for most iron toxicity through catalytic production of hydroxyl radicals and oxidation products [see, e.g., Esposito et al. (2003) Blood 102:2670-2677]. These agents are structurally diverse, but all possess oxygen or nitrogen donor atoms able to form neutralizing octahedral coordination complexes with individual iron atoms in stoichiometries of 1:1 (hexadentate agents), 2:1 (tridentate), or 3:1 (bidentate) [Kalinowski et al. (2005) Pharmacol Rev 57:547-583]. In general, effective iron-chelating agents also are relatively low molecular weight (e.g., less than 700 daltons), with solubility in both water and lipids to enable access to affected tissues. Specific examples of iron-chelating molecules include deferoxamine, a hexadentate agent of bacterial origin requiring daily parenteral administration, and the orally active synthetic agents deferiprone (bidentate) and deferasirox (tridentate). Combination therapy consisting of same-day administration of two iron-chelating agents shows promise in patients unresponsive to chelation monotherapy and also in overcoming issues of poor patient compliance with dereroxamine alone [Cao et al. (2011) Pediatr Rep 3(2):e17; Galanello et al. (2010) Ann NY Acad Sci 1202:79-86].

As used herein, “combination”, “in combination with” or “conjoint administration” refers to any form of administration such that the second therapy is still effective in the body (e.g., the two compounds are simultaneously effective in the patient, which may include synergistic effects of the two compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, an individual who receives such treatment can benefit from a combined effect of different therapies. One or more TGF-beta superfamily co-receptor heteromultimers of the disclosure can be administered concurrently with, prior to, or subsequent to, one or more other additional agents or supportive therapies. In general, each therapeutic agent will be administered at a dose and/or on a time schedule determined for that particular agent. The particular combination to employ in a regimen will take into account compatibility of the antagonist of the present disclosure with the therapy and/or the desired therapeutic effect to be achieved.

In certain embodiments, one or more TGF-beta superfamily co-receptor heteromultimers of the disclosure may be used in combination with hepcidin or a hepcidin agonist for ineffective erythropoiesis. A circulating polypeptide produced mainly in the liver, hepcidin is considered a master regulator of iron metabolism by virtue of its ability to induce the degradation of ferroportin, an iron-export protein localized on absorptive enterocytes, hepatocytes, and macrophages. Broadly speaking, hepcidin reduces availability of extracellular iron, so hepcidin agonists may be beneficial in the treatment of ineffective erythropoiesis [see, e.g., Nemeth (2010) Adv Hematol 2010:750643]. This view is supported by beneficial effects of increased hepcidin expression in a mouse model of β-thalassemia [Gardenghi et al. (2010) J Clin Invest 120:4466-4477].

One or more TGF-beta superfamily co-receptor heteromultimers of the disclosure, optionally combined with an EPO receptor activator, would also be appropriate for treating anemias of disordered RBC maturation, which are characterized in part by undersized (microcytic), oversized (macrocytic), misshapen, or abnormally colored (hypochromic) RBCs.

In certain embodiments, the present disclosure provides methods of treating or preventing anemia in an individual in need thereof by administering to the individual a therapeutically effective amount of one or more TGF-beta superfamily co-receptor heteromultimers of the disclosure and an EPO receptor activator. In certain embodiments, one or more TGF-beta superfamily co-receptor heteromultimers of the disclosure may be used in combination with EPO receptor activators to reduce the required dose of these activators in patients that are susceptible to adverse effects of EPO. These methods may be used for therapeutic and prophylactic treatments of a patient.

One or more TGF-beta superfamily co-receptor heteromultimers of the disclosure may be used in combination with EPO receptor activators to achieve an increase in red blood cells, particularly at lower dose ranges of EPO receptor activators. This may be beneficial in reducing the known off-target effects and risks associated with high doses of EPO receptor activators. The primary adverse effects of EPO include, for example, an excessive increase in the hematocrit or hemoglobin levels and polycythemia. Elevated hematocrit levels can lead to hypertension (more particularly aggravation of hypertension) and vascular thrombosis. Other adverse effects of EPO which have been reported, some of which relate to hypertension, are headaches, influenza-like syndrome, obstruction of shunts, myocardial infarctions and cerebral convulsions due to thrombosis, hypertensive encephalopathy, and red cell blood cell aplasia. See, e.g., Singibarti (1994) J. Clin Investig 72(suppl 6), S36-S43; Horl et al. (2000) Nephrol Dial Transplant 15(suppl 4), 51-56; Delanty et al. (1997) Neurology 49, 686-689; and Bunn (2002) N Engl J Med 346(7), 522-523).

Provided that TGF-beta superfamily co-receptor heteromultimers of the present disclosure act by a different mechanism than EPO, these antagonists may be useful for increasing red blood cell and hemoglobin levels in patients that do not respond well to EPO. For example, a TGF-beta superfamily co-receptor heteromultimer of the present disclosure may be beneficial for a patient in which administration of a normal-to-increased dose of EPO (>300 IU/kg/week) does not result in the increase of hemoglobin level up to the target level. Patients with an inadequate EPO response are found in all types of anemia, but higher numbers of non-responders have been observed particularly frequently in patients with cancers and patients with end-stage renal disease. An inadequate response to EPO can be either constitutive (observed upon the first treatment with EPO) or acquired (observed upon repeated treatment with EPO).

In certain embodiments, the present disclosure provides methods for managing a patient that has been treated with, or is a candidate to be treated with, one or more TGF-beta superfamily co-receptor heteromultimers of the disclosure by measuring one or more hematologic parameters in the patient. The hematologic parameters may be used to evaluate appropriate dosing for a patient who is a candidate to be treated with the antagonist of the present disclosure, to monitor the hematologic parameters during treatment, to evaluate whether to adjust the dosage during treatment with one or more antagonist of the disclosure, and/or to evaluate an appropriate maintenance dose of one or more antagonists of the disclosure. If one or more of the hematologic parameters are outside the normal level, dosing with one or more TGF-beta superfamily co-receptor heteromultimers of the disclosure may be reduced, delayed or terminated.

Hematologic parameters that may be measured in accordance with the methods provided herein include, for example, red blood cell levels, blood pressure, iron stores, and other agents found in bodily fluids that correlate with increased red blood cell levels, using art-recognized methods. Such parameters may be determined using a blood sample from a patient. Increases in red blood cell levels, hemoglobin levels, and/or hematocrit levels may cause increases in blood pressure.

In one embodiment, if one or more hematologic parameters are outside the normal range or on the high side of normal in a patient who is a candidate to be treated with one or more TGF-beta co-receptor superfamily heteromultimers of the disclosure, then onset of administration of the one or more TGF-beta superfamily co-receptor heteromultimers of the disclosure may be delayed until the hematologic parameters have returned to a normal or acceptable level either naturally or via therapeutic intervention. For example, if a candidate patient is hypertensive or pre-hypertensive, then the patient may be treated with a blood pressure lowering agent in order to reduce the patient's blood pressure. Any blood pressure lowering agent appropriate for the individual patient's condition may be used including, for example, diuretics, adrenergic inhibitors (including alpha blockers and beta blockers), vasodilators, calcium channel blockers, angiotensin-converting enzyme (ACE) inhibitors, or angiotensin II receptor blockers. Blood pressure may alternatively be treated using a diet and exercise regimen. Similarly, if a candidate patient has iron stores that are lower than normal, or on the low side of normal, then the patient may be treated with an appropriate regimen of diet and/or iron supplements until the patient's iron stores have returned to a normal or acceptable level. For patients having higher than normal red blood cell levels and/or hemoglobin levels, then administration of the one or more TGF-beta superfamily co-receptor heteromultimers of the disclosure may be delayed until the levels have returned to a normal or acceptable level.

In certain embodiments, if one or more hematologic parameters are outside the normal range or on the high side of normal in a patient who is a candidate to be treated with one or more TGF-beta superfamily co-receptor heteromultimers of the disclosure, then the onset of administration may not be delayed. However, the dosage amount or frequency of dosing of the one or more TGF-beta superfamily co-receptor heteromultimers of the disclosure may be set at an amount that would reduce the risk of an unacceptable increase in the hematologic parameters arising upon administration of the one or more TGF-beta superfamily co-receptor heteromultimers of the disclosure. Alternatively, a therapeutic regimen may be developed for the patient that combines one or more TGF-beta superfamily co-receptor heteromultimers of the disclosure with a therapeutic agent that addresses the undesirable level of the hematologic parameter. For example, if the patient has elevated blood pressure, then a therapeutic regimen involving administration of one or more TGF-beta superfamily co-receptor heteromultimers of the disclosure and a blood pressure-lowering agent may be designed. For a patient having lower than desired iron stores, a therapeutic regimen of one or more TGF-beta superfamily co-receptor heteromultimers of the disclosure and iron supplementation may be developed.

In one embodiment, baseline parameter(s) for one or more hematologic parameters may be established for a patient who is a candidate to be treated with one or more TGF-beta superfamily co-receptor heteromultimers of the disclosure and an appropriate dosing regimen established for that patient based on the baseline value(s). Alternatively, established baseline parameters based on a patient's medical history could be used to inform an appropriate dosing regimen for a patient. For example, if a healthy patient has an established baseline blood pressure reading that is above the defined normal range it may not be necessary to bring the patient's blood pressure into the range that is considered normal for the general population prior to treatment with the one or more TGF-beta superfamily co-receptor heteromultimers of the disclosure. A patient's baseline values for one or more hematologic parameters prior to treatment with one or more TGF-beta superfamily co-receptor heteromultimers of the disclosure may also be used as the relevant comparative values for monitoring any changes to the hematologic parameters during treatment with the one or more TGF-beta superfamily co-receptor heteromultimers of the disclosure.

In certain embodiments, one or more hematologic parameters are measured in patients who are being treated with a one or more TGF-beta superfamily co-receptor heteromultimers of the disclosure. The hematologic parameters may be used to monitor the patient during treatment and permit adjustment or termination of the dosing with the one or more TGF-beta superfamily co-receptor heteromultimers of the disclosure or additional dosing with another therapeutic agent. For example, if administration of one or more TGF-beta superfamily co-receptor heteromultimer complexes of the disclosure of the disclosure results in an increase in blood pressure, red blood cell level, or hemoglobin level, or a reduction in iron stores, then the dose of the one or more TGF-beta superfamily co-receptor heteromultimers of the disclosure may be reduced in amount or frequency in order to decrease the effects of the one or more TGF-beta superfamily co-receptor heteromultimers of the disclosure on the one or more hematologic parameters. If administration of one or more TGF-beta superfamily co-receptor heteromultimers of the disclosure results in a change in one or more hematologic parameters that is adverse to the patient, then the dosing of the one or more TGF-beta superfamily co-receptor heteromultimers of the disclosure may be terminated either temporarily, until the hematologic parameter(s) return to an acceptable level, or permanently. Similarly, if one or more hematologic parameters are not brought within an acceptable range after reducing the dose or frequency of administration of the one or more TGF-beta superfamily co-receptor heteromultimers of the disclosure, then the dosing may be terminated. As an alternative, or in addition to, reducing or terminating the dosing with the one or more TGF-beta superfamily co-receptor heteromultimers of the disclosure, the patient may be dosed with an additional therapeutic agent that addresses the undesirable level in the hematologic parameter(s), such as, for example, a blood pressure-lowering agent or an iron supplement. For example, if a patient being treated with one or more TGF-beta superfamily co-receptor heteromultimers of the disclosure has elevated blood pressure, then dosing with the one or more TGF-beta superfamily co-receptor heteromultimers of the disclosure may continue at the same level and a blood pressure-lowering agent is added to the treatment regimen, dosing with the one or more TGF-beta superfamily co-receptor heteromultimers of the disclosure may be reduced (e.g., in amount and/or frequency) and a blood pressure-lowering agent is added to the treatment regimen, or dosing with the one or more TGF-beta superfamily co-receptor heteromultimers of the disclosure may be terminated and the patient may be treated with a blood pressure-lowering agent.

### 6. Pharmaceutical Compositions

In certain aspects, TGF-beta superfamily co-receptor single-arm heteromultimer complexes of the present disclosure can be administered alone or as a component of a pharmaceutical formulation (also referred to as a therapeutic composition or pharmaceutical composition). A pharmaceutical formation refers to a preparation which is in such form as to permit the biological activity of an active ingredient (e.g., an agent of the present disclosure) contained therein to be effective and which contains no additional components which are unacceptably toxic to a subject to which the formulation would be administered. The subject compounds may be formulated for administration in any convenient way for use in human or veterinary medicine. For example, one or more agents of the present disclosure may be formulated with a pharmaceutically acceptable carrier. A pharmaceutically acceptable carrier refers to an ingredient in a pharmaceutical formulation, other than an active ingredient, which is generally nontoxic to a subject. A pharmaceutically acceptable carrier includes, but is not limited to, a buffer, excipient, stabilizer, and/or preservative. In general, pharmaceutical formulations for use in the present disclosure are in a pyrogen-free, physiologically-acceptable form when administered to a subject. Therapeutically useful agents other than those described herein, which may optionally be included in the formulation as described above, may be administered in combination with the subject agents in the methods of the present disclosure.

In certain embodiments, compositions will be administered parenterally [e.g., by intravenous (I.V.) injection, intraarterial injection, intraosseous injection, intramuscular injection, intrathecal injection, subcutaneous injection, or intradermal injection]. Pharmaceutical compositions suitable for parenteral administration may comprise one or more agents of the disclosure in combination with one or more pharmaceutically acceptable sterile isotonic aqueous or nonaqueous solutions, dispersions, suspensions or emulsions, or sterile powders which may be reconstituted into sterile injectable solutions or dispersions just prior to use. Injectable solutions or dispersions may contain antioxidants, buffers, bacteriostats, suspending agents, thickening agents, or solutes which render the formulation isotonic with the blood of the intended recipient. Examples of suitable aqueous and nonaqueous carriers which may be employed in the pharmaceutical formulations of the present disclosure include water, ethanol, polyols (e.g., glycerol, propylene glycol, polyethylene glycol, etc.), vegetable oils (e.g., olive oil), injectable organic esters (e.g., ethyl oleate), and suitable mixtures thereof. Proper fluidity can be maintained, for example, by the use of coating materials (e.g., lecithin), by the maintenance of the required particle size in the case of dispersions, and by the use of surfactants.

In some embodiments, a therapeutic method of the present disclosure includes administering the pharmaceutical composition systemically, or locally, from an implant or device. Further, the pharmaceutical composition may be encapsulated or injected in a form for delivery to a target tissue site (e.g., bone marrow or muscle). In certain embodiments, compositions of the present disclosure may include a matrix capable of delivering one or more of the agents of the present disclosure to a target tissue site (e.g., bone marrow or muscle), providing a structure for the developing tissue and optimally capable of being resorbed into the body. For example, the matrix may provide slow release of one or more agents of the present disclosure. Such matrices may be formed of materials presently in use for other implanted medical applications.

The choice of matrix material may be based on one or more of biocompatibility, biodegradability, mechanical properties, cosmetic appearance, and interface properties. The particular application of the subject compositions will define the appropriate formulation. Potential matrices for the compositions may be biodegradable and chemically defined calcium sulfate, tricalciumphosphate, hydroxyapatite, polylactic acid, and polyanhydrides. Other potential materials are biodegradable and biologically well-defined including, for example, bone or dermal collagen. Further matrices are comprised of pure proteins or extracellular matrix components. Other potential matrices are non-biodegradable and chemically defined including, for example, sintered hydroxyapatite, bioglass, aluminates, or other ceramics. Matrices may be comprised of combinations of any of the above mentioned types of material including, for example, polylactic acid and hydroxyapatite or collagen and tricalciumphosphate. The bioceramics may be altered in composition (e.g., calcium-aluminate-phosphate) and processing to alter one or more of pore size, particle size, particle shape, and biodegradability.

In certain embodiments, pharmaceutical compositions of the present disclosure can be administered topically. “Topical application” or “topically” means contact of the pharmaceutical composition with body surfaces including, for example, the skin, wound sites, and mucous membranes. The topical pharmaceutical compositions can have various application forms and typically comprises a drug-containing layer, which is adapted to be placed near to or in direct contact with the tissue upon topically administering the composition. Pharmaceutical compositions suitable for topical administration may comprise one or more TGFβ superfamily co-receptor single-arm heteromultimer complexes of the disclosure in combination formulated as a liquid, a gel, a cream, a lotion, an ointment, a foam, a paste, a putty, a semi-solid, or a solid. Compositions in the liquid, gel, cream, lotion, ointment, foam, paste, or putty form can be applied by spreading, spraying, smearing, dabbing or rolling the composition on the target tissue. The compositions also may be impregnated into sterile dressings, transdermal patches, plasters, and bandages. Compositions of the putty, semi-solid or solid forms may be deformable. They may be elastic or non-elastic (e.g., flexible or rigid). In certain aspects, the composition forms part of a composite and can include fibers, particulates, or multiple layers with the same or different compositions.

Topical compositions in the liquid form may include pharmaceutically acceptable solutions, emulsions, microemulsions, and suspensions. In addition to the active ingredient(s), the liquid dosage form may contain an inert diluent commonly used in the art including, for example, water or other solvent, a solubilizing agent and/or emulsifier [e.g., ethyl alcohol, isopropyl alcohol, ethyl carbonate, ethyl acetate, benzyl alcohol, benzyl benzoate, propylene glycol, or 1,3-butylene glycol, an oil (e.g., cottonseed, groundnut, corn, germ, olive, castor, and sesame oil), glycerol, tetrahydrofuryl alcohol, a polyethylene glycol, a fatty acid ester of sorbitan, and mixtures thereof].

Topical gel, cream, lotion, ointment, semi-solid or solid compositions may include one or more thickening agents, such as a polysaccharide, synthetic polymer or protein-based polymer. In one embodiment of the invention, the gelling agent herein is one that is suitably nontoxic and gives the desired viscosity. The thickening agents may include polymers, copolymers, and monomers of vinylpyrrolidones, methacrylamides, acrylamides N-vinylimidazoles, carboxy vinyls, vinyl esters, vinyl ethers, silicones, polyethyleneoxides, polyethyleneglycols, vinylalcohols, sodium acrylates, acrylates, maleic acids, NN-dimethylacrylamides, diacetone acrylamides, acrylamides, acryloyl morpholine, pluronic, collagens, polyacrylamides, polyacrylates, polyvinyl alcohols, polyvinylenes, polyvinyl silicates, polyacrylates substituted with a sugar (e.g., sucrose, glucose, glucosamines, galactose, trehalose, mannose, or lactose), acylamidopropane sulfonic acids, tetramethoxyorthosilicates, methyltrimethoxyorthosilicates, tetraalkoxyorthosilicates, trialkoxyorthosilicates, glycols, propylene glycol, glycerine, polysaccharides, alginates, dextrans, cyclodextrin, celluloses, modified celluloses, oxidized celluloses, chitosans, chitins, guars, carrageenans, hyaluronic acids, inulin, starches, modified starches, agarose, methylcelluloses, plant gums, hylaronans, hydrogels, gelatins, glycosaminoglycans, carboxymethyl celluloses, hydroxyethyl celluloses, hydroxy propyl methyl celluloses, pectins, low-methoxy pectins, cross-linked dextrans, starch-acrylonitrile graft copolymers, starch sodium polyacrylate, hydroxyethyl methacrylates, hydroxyl ethyl acrylates, polyvinylene, polyethylvinylethers, polymethyl methacrylates, polystyrenes, polyurethanes, polyalkanoates, polylactic acids, polylactates, poly(3-hydroxybutyrate), sulfonated hydrogels, AMPS (2-acrylamido-2-methyl-1-propanesulfonic acid), SEM (sulfoethylmethacrylate), SPM (sulfopropyl methacrylate), SPA (sulfopropyl acrylate), N,N-dimethyl-N-methacryloxyethyl-N-(3-sulfopropyl)ammonium betaine, methacryllic acid amidopropyl-dimethyl ammonium sulfobetaine, SPI (itaconic acid-bis(1-propyl sulfonizacid-3) ester di-potassium salt), itaconic acids, AMBC (3-acrylamido-3-methylbutanoic acid), beta-carboxyethyl acrylate (acrylic acid dimers), and maleic anhydride-methylvinyl ether polymers, derivatives thereof, salts thereof, acids thereof, and combinations thereof. In certain embodiments, pharmaceutical compositions of present disclosure can be administered orally, for example, in the form of capsules, cachets, pills, tablets, lozenges (using a flavored basis such as sucrose and acacia or tragacanth), powders, granules, a solution or a suspension in an aqueous or non-aqueous liquid, an oil-in-water or water-in-oil liquid emulsion, or an elixir or syrup, or pastille (using an inert base, such as gelatin and glycerin, or sucrose and acacia), and/or a mouth wash, each containing a predetermined amount of a compound of the present disclosure and optionally one or more other active ingredients. A compound of the present disclosure and optionally one or more other active ingredients may also be administered as a bolus, electuary, or paste.

In solid dosage forms for oral administration (e.g., capsules, tablets, pills, dragees, powders, and granules), one or more compounds of the present disclosure may be mixed with one or more pharmaceutically acceptable carriers including, for example, sodium citrate, dicalcium phosphate, a filler or extender (e.g., a starch, lactose, sucrose, glucose, mannitol, and silicic acid), a binder (e.g. carboxymethylcellulose, an alginate, gelatin, polyvinyl pyrrolidone, sucrose, and acacia), a humectant (e.g., glycerol), a disintegrating agent (e.g., agar-agar, calcium carbonate, potato or tapioca starch, alginic acid, a silicate, and sodium carbonate), a solution retarding agent (e.g. paraffin), an absorption accelerator (e.g. a quaternary ammonium compound), a wetting agent (e.g., cetyl alcohol and glycerol monostearate), an absorbent (e.g., kaolin and bentonite clay), a lubricant (e.g., a talc, calcium stearate, magnesium stearate, solid polyethylene glycols, sodium lauryl sulfate), a coloring agent, and mixtures thereof. In the case of capsules, tablets, and pills, the pharmaceutical formulation (composition) may also comprise a buffering agent. Solid compositions of a similar type may also be employed as fillers in soft and hard-filled gelatin capsules using one or more excipients including, e.g., lactose or a milk sugar as well as a high molecular-weight polyethylene glycol.

Liquid dosage forms for oral administration of the pharmaceutical composition may include pharmaceutically acceptable emulsions, microemulsions, solutions, suspensions, syrups, and elixirs. In addition to the active ingredient(s), the liquid dosage form may contain an inert diluent commonly used in the art including, for example, water or other solvent, a solubilizing agent and/or emulsifier [e.g., ethyl alcohol, isopropyl alcohol, ethyl carbonate, ethyl acetate, benzyl alcohol, benzyl benzoate, propylene glycol, or 1,3-butylene glycol, an oil (e.g., cottonseed, groundnut, corn, germ, olive, castor, and sesame oil), glycerol, tetrahydrofuryl alcohol, a polyethylene glycol, a fatty acid ester of sorbitan, and mixtures thereof]. Besides inert diluents, the oral formulation can also include an adjuvant including, for example, a wetting agent, an emulsifying and suspending agent, a sweetening agent, a flavoring agent, a coloring agent, a perfuming agent, a preservative agent, and combinations thereof.

Suspensions, in addition to the active compounds, may contain suspending agents including, for example, an ethoxylated isostearyl alcohol, polyoxyethylene sorbitol, a sorbitan ester, microcrystalline cellulose, aluminum metahydroxide, bentonite, agar-agar, tragacanth, and combinations thereof.

Prevention of the action and/or growth of microorganisms may be ensured by the inclusion of various antibacterial and antifungal agents including, for example, paraben, chlorobutanol, and phenol sorbic acid.

In certain embodiments, it may be desirable to include an isotonic agent including, for example, a sugar or sodium chloride into the compositions. In addition, prolonged absorption of an injectable pharmaceutical form may be brought about by the inclusion of an agent that delay absorption including, for example, aluminum monostearate and gelatin.

It is understood that the dosage regimen will be determined by the attending physician considering various factors which modify the action of the one or more of the agents of the present disclosure. In the case of a TGF-beta superfamily co-receptor single-arm heteromultimer complex that promotes red blood cell formation, various factors may include, but are not limited to, the patient's red blood cell count, hemoglobin level, the desired target red blood cell count, the patient's age, the patient's sex, the patient's diet, the severity of any disease that may be contributing to a depressed red blood cell level, the time of administration, and other clinical factors. The addition of other known active agents to the final composition may also affect the dosage. Progress can be monitored by periodic assessment of one or more of red blood cell levels, hemoglobin levels, reticulocyte levels, and other indicators of the hematopoietic process.

In certain embodiments, the present disclosure also provides gene therapy for the in vivo production of one or more of the agents of the present disclosure. Such therapy would achieve its therapeutic effect by introduction of the agent sequences into cells or tissues having one or more of the disorders as listed above. Delivery of the agent sequences can be achieved, for example, by using a recombinant expression vector such as a chimeric virus or a colloidal dispersion system. Preferred therapeutic delivery of one or more of agent sequences of the disclosure is the use of targeted liposomes.

Various viral vectors which can be utilized for gene therapy as taught herein include adenovirus, herpes virus, vaccinia, or an RNA virus (e.g., a retrovirus). The retroviral vector may be a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Preferred targeting is accomplished by using an antibody. Those of skill in the art will recognize that specific polynucleotide sequences can be inserted into the retroviral genome or attached to a viral envelope to allow target specific delivery of the retroviral vector containing one or more of the agents of the present disclosure.

Alternatively, tissue culture cells can be directly transfected with plasmids encoding the retroviral structural genes (gag, pol, and env), by conventional calcium phosphate transfection. These cells are then transfected with the vector plasmid containing the genes of interest. The resulting cells release the retroviral vector into the culture medium.

Another targeted delivery system for one or more of the agents of the present disclosure is a colloidal dispersion system. Colloidal dispersion systems include, for example, macromolecule complexes, nanocapsules, microspheres, beads, and lipid-based systems including oil-in-water emulsions, micelles, mixed micelles, and liposomes. In certain embodiments, the preferred colloidal system of this disclosure is a liposome. Liposomes are artificial membrane vesicles which are useful as delivery vehicles in vitro and in vivo. RNA, DNA, and intact virions can be encapsulated within the aqueous interior and be delivered to cells in a biologically active form. See, e.g., Fraley, et al. (1981) Trends Biochem. Sci., 6:77. Methods for efficient gene transfer using a liposome vehicle are known in the art. See, e.g., Mannino, et al. (1988) Biotechniques, 6:682, 1988.

The composition of the liposome is usually a combination of phospholipids, which may include a steroid (e.g.cholesterol). The physical characteristics of liposomes depend on pH, ionic strength, and the presence of divalent cations. Other phospholipids or other lipids may also be used including, for example a phosphatidyl compound (e.g., phosphatidylglycerol, phosphatidylcholine, phosphatidylserine, phosphatidylethanolamine, a sphingolipid, a cerebroside, and a ganglioside), egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

### EXEMPLIFICATION

The invention now being generally described, it will be more readily understood by reference to the following examples, which are included merely for purposes of illustration of certain embodiments of the present invention, and are not intended to limit the invention.

### Example 1. Generation of a Single-Arm Endoglin-Fc Heterodimer

Applicants envision construction of a soluble single-arm endoglin-Fc heterodimeric complex comprising a monomeric Fc polypeptide with a short N-terminal extension and a second polypeptide in which a ligand-binding domain of human endoglin is fused to a separate Fc domain with a linker positioned between the ligand-binding domain and this second Fc domain. The individual constructs are referred to as monomeric Fc polypeptide and endoglin-Fc fusion polypeptide, respectively, and the sequences for each are provided below. Applicants also envision similar single-arm endoglin-Fc heterodimeric complexes comprising ligand-binding domains of endoglin isoforms 2 or 3 (SEQ ID Nos: 6 or 10).

A methodology for promoting formation of endoglin-Fc:Fc heteromeric complexes rather than endoglin-Fc: endoglin-Fc or Fc:Fc homodimeric complexes is to introduce alterations in the amino acid sequence of the Fc domains to guide the formation of asymmetric heteromeric complexes. Many different approaches to making asymmetric interaction pairs using Fc domains are described in this disclosure.

In one approach, illustrated in the endoglin-Fc and monomeric Fc polypeptide sequences of SEQ ID NOs: 500-501 and 502-503, respectively, one Fc domain is altered to introduce cationic amino acids at the interaction face, while the other Fc domain is altered to introduce anionic amino acids at the interaction face. The endoglin-Fc fusion polypeptide and monomeric Fe polypeptide each employ the tissue plasminogen activator (TPA) leader:

(SEQ ID NO: 300)
MDAMKRGLCCVLLLCGAVFVSP

The endoglin-Fc polypeptide sequence (SEQ ID NO: 500) is shown below:

| (SEQ ID NO: 500) | |
|---|---|
| 1 | MDAMKRGLCC VLLLCGAVFV SPGASETVHC DLQPVGPERG EVTYTTSQVS KGCVAQAPNA |
| 61 | ILEVHVIFLE FRTGPSQLEL TLQASKQNGT WPREVLLVLS VNSSVFLHLQ ALGIPLHLAY |
| 121 | NSSLVTFQEP PGVNTTELPS FPKTQILEWA AERGPITSAA ELNDPQSILL RLGOAQGSLS |
| 181 | FCMLEASQDM GRTLEWRPRT PALVRGCHLE GVAGHKEAHI LRVLPGHSAG PRTVTVKVEL |
| 241 | SCAPGDLDAV LILQGPPYVS WLIDANHNMQ IWTTGEYSFK IFPEKNIRGF KLPDTPQGLL |
| 301 | GEARMLNASI VASFVELPLA SIVSLHASSC GGRLQTSPAP IQTTPPKDTC SPELLMSLIQ |
| 361 | TKCADDAMTL VLKKELVAHL KCTITGLTFW DPSCEAEDRG DKFVIRSAYS SCGMQVSASM |
| 421 | ISNEAVVNIL SSSSPQRKKV HCLNMDSLSF QLGLYISPHF LQASNTIEPG QQSFVQVRVS |
| 481 | PSVSEFLLQL DSCHLDLGPE GGTVELIQGR AAKGNCVSLL SPSPEGDPRF SFLLHFYTVP |
| 541 | IPKTGTLSCT VALRPKTGSQ DOEVHRTVFM RLNIISPDLS GCTSKGTGGG THTCPPCPAP |
| 601 | ELLGGPSVFL FPPKPKDTLM ISRTPEVTCV VVDVSHEDPE VKFNWYVDGV EVHNAKTKPR |
| 661 | EEQYNSTYRV VSVLTVLHQD WLNGKEYKCK VSNKALPAPI EKTISKAKGQ PREPQVYTLP |
| 721 | PSRKEMTKNQ VSLTCLVKGF YPSDIAVEWE SNGQPENNYK TTPPVLKSDG SFFLYSKLTV |
| 781 | DKSPWQQGNV FSCSVMHEAL HNHYTQKSLS LSPGK |

The leader (signal) sequence and linker are underlined. To promote formation of the endoglin-Fc:Fc heterodimer rather than either of the possible homodimeric complexes (endoglin-Fc:endoglin-Fc or Fc:Fc), two amino acid substitutions (replacing acidic amino acids with lysine) can be introduced into the Fc domain of the endoglin fusion protein as indicated by double underline above. The amino acid sequence of SEQ ID NO: 500 may optionally be provided with the C-terminal lysine (K) removed.

The mature endoglin-Fc fusion polypeptide (SEQ ID NO: 501) is as follows and may optionally be provided with the C-terminal lysine removed.

| (SEQ ID NO: 501) | |
|---|---|
| 1 | ETVHCDLQPV GPERGEVTYT TSQVSKGCVA QAPNAILEVH VLFLEFPTGP SQLELTLQAS |
| 61 | KQNGTWPREV LLVLSVNSSV FLHLQALGIP LHLAYNSSLV TFQEPPGVNT TELPSFPKTQ |
| 121 | ILEWAAERGP ITSAAELNDP QSILLRLGQA QGSLSFCMLE ASQDMGRTLE WRPRTPALVR |
| 181 | GCHLEGVAGH KEAHILRVLP GHSAGPRTVT VKVELSCAPG DLDAVLILQG PPYVSWLIDA |
| 241 | NHNMQIWTTG EYSFKIFPEK NIRGFKLPDT PQGLLGEARM LNASIVASFV ELPLASIVSL |
| 301 | HASSCGGRLQ TSPAPIQTTP PKDTCSPELL MSLIQTKCAD DAMTLVLKKE LVAHLKCTIT |
| 361 | GLTFWDPSCE AEDRGDKFVL RSAYSSCGMQ VSASMISNEA VVNILSSSSP QRKKVECLNM |
| 421 | DSLSFQLGLY LSPHFLQASN TIEPGQQSFV QVRVSPSVSE FLLQLDSCHL DLGPEGGTVE |
| 481 | LIQGRAAKGN CVSLLSPSPE GDPRFSFLLH FYTVPIPKTG TLSCTVALRP KTGSQDQEVH |
| 541 | RTVFMRLNII SPDLSGCTSK GTGGGTHTCP PCPAPELLGG PSVFLFPPKP KDTLMISRTP |
| 601 | EVTCVVVDVS HEDPEVKFNW YVDGVEVHNA KTKPREEQYN STYRVVSVLT VLHQDWLNGK |
| 661 | EYKCKVSNKA LPAPIEKTIS KAKGQPREPQ VYTLPPSRKE MTKNQVSLTC LVKGFYPSDI |
| 721 | AVEWESNGQP ENNYKTTPPV LKSDGSFFLY SKLTVDKSRW QQGNVFSCSV MHEALENHYT |
| 781 | QKSLSLSPCK |

The complementary human G1Fc polypeptide (SEQ ID NO: 502) employs the TPA leader and is as follows:

```
(SEQ ID NO: 502)
  1 MDAMKRGLCC VLLLCGAVFV SPGASNTKVD
      KRVTGGGTHT CPPCPAPELL
  51 GGPSVFLFPP KPKDTLMISR TPEVTCVVVD
        VSHEDPEVKF NWYVDGVEVH
101 NAKTKPREEQ YNSTYRVVSV LTVLHQDWLN
        GKEYKCKVSN KALPAPIEKT
151 ISKAKGQPRE PQVYTLPPSR EEMTKNQVSL
        TCLVKGFYPS DIAVEWESNG
201 QPENNYDTTP PVLDSDGSFF LYSDLTVDKS
        RWQQGNVFSC SVMHEALHNH
251 YTQKSLSLSP GK
```

The leader sequence is underlined, and an optional N-terminal extension of the Fc polypeptide is indicated by double underline. To promote formation of the endoglin-Fc:Fc heterodimer rather than either of the possible homodimeric complexes, two amino acid substitutions (replacing lysines with anionic residues) can be introduced into the monomeric Fc polypeptide as indicated by double underline above. The amino acid sequence of SEQ ID NO: 502 may optionally be provided with the C-terminal lysine removed.

The sequence of the mature monomeric Fc polypeptide is as follows (SEQ ID NO: 503) and may optionally be provided with the C-terminal lysine removed.

| (SEQ ID NO: 503) | |
|---|---|
| 1 | SNTKVDKRVT GGGTHTCPPC PAPELLGGPS VFLFPPKPKD TLMISRTPEV |
| 51 | TCVVVDVSHE DPEVKFNWYV DGVEVHNAKT KPREEQYNST YRVVSVLTVL |
| 101 | HQDWLNGKEY KCKVSNKALP APIEKTISKA KGQPREPQVY TLPPSREEMT |
| 151 | KNQVSLTCLV KGFYPSDIAV EWESNGQPEN NYDTTPPVLD SDGSFFLYSD |
| 201 | LTVDKSRWQQ GNVFSCSVMH EALHNHYTQK SLSLSPGK |

The endoglin-Fc fusion polypeptide and monomeric Fc polypeptide of SEQ ID NO: 501 and SEQ ID NO: 503, respectively, may be co-expressed and purified from a CHO cell line to give rise to a single-arm heteromeric protein complex comprising endoglin-Fc:Fc.

In another approach to promote the formation of heteromultimer complexes using asymmetric Fc fusion polypeptides, the Fc domains are altered to introduce complementary hydrophobic interactions and an additional intermolecular disulfide bond, as illustrated in the endoglin-Fc and monomeric Fc polypeptide sequences of SEQ ID NOs: 504-505 and 506-507, respectively.

The endoglin-Fc polypeptide sequence (SEQ ID NO: 504) employs the TPA leader and is shown below:

```
(SEQ ID NO: 504)
    1 MDAMKRGLCC VLLLCGAVFV SPGASETVHC
        DLQPVGPERG EVTYTTSQVS KGCVAQAPNA
  61 ILEVHVLFLE FPTGPSQLEL TLQASKQNGT
        WPREVLLVLS VNSSVFLHLQ ALGIPLHLAY
121 NSSLVTFQEP PGVNTTELPS FPKTQILEWA
        AERGPITSAA ELNDPQSILL RLGQAQGSLS
181 FCMLEASQDM GRTLEWRPRT PALVRGCHLE
        GVAGHKEAHI LRVLPGHSAG PRTVTVKVEL
241 SCAPGDLDAV LILQGPPYVS WLIDANHNMQ
        IWTTGEYSFK IFPEKNIRGF KLPDTPQGLL
301 GEARMLNASI VASFVELPLA SIVSLHASSC
        GGRLQTSPAP IQTTPPKDTC SPELLMSLIQ
361 TKCADDAMTL VLKKELVAHL KCTITGLTFW
        DPSCEAEDRG DKFVLRSAYS SCGMQVSASM
421 ISNEAVVNIL SSSSPQRKKV HCLNMDSLSF
        QLGLYLSPHF LQASNTIEPG QQSFVQVRVS
481 PSVSEFLLQL DSCHLDLGPE GGTVELIQGR
        AAKGNCVSLL SPSPEGDPRF SFLLHFYTVP
541 IPKTGTLSCT VALRPKTGSQ DQEVHRTVFM
        RLNIISPDLS GCTSKGTGGG THTCPPCPAP
601 ELLGGPSVFL FPPKPKDTLM ISRTPEVTCV
        VVDVSHEDPE VKFNWYVDGV EVHNAKTKPR
661 EEQYNSTYRV VSVLTVLHQD WLNGKEYKCK
        VSNKALPAPI EKTISKAKGQ PREPQVYTLP
721 PCREEMTKNQ VSLWCLVKGF YPSDIAVEWE
        SNGQPENNYK TTPPVLDSDG SFFLYSKLTV
781 DKSRWQQGNV FSCSVMHEAL HNHYTQKSLS
        LSPGK
```

The leader sequence and linker are underlined. To promote formation of the endoglin-Fc:Fc heterodimer rather than either of the possible homodimeric complexes, two amino acid substitutions (replacing a serine with a cysteine and a threonine with a trytophan) can be introduced into the Fe domain of the fusion protein as indicated by double underline above. The amino acid sequence of SEQ ID NO: 504 may optionally be provided with the C-terminal lysine removed.

The mature endoglin-Fc fusion polypeptide is as follows:

```
(SEQ ID NO: 505)
    1 ETVHCDLQPV GPERGEVTYT TSQVSKGCVA
        QAPNAILEVH VLFLEFPTGP SQLELTLQAS
  61 KQNGTWPREV LLVLSVNSSV FLHLQALGIP
        LHLAYNSSLV TFQEPPGVNT TELPSFPKTQ
121 ILEWAAERGP ITSAAELNDP QSILLRLGQA
        QGSLSFCMLE ASQDMGRTLE WRPRTPALVR
181 GCHLEGVAGH KEAHILRVLP GHSAGPRTVT
        VKVELSCAPG DLDAVLILQG PPYVSWLIDA
241 NHNMQIWTTG EYSFKIFPEK NIRGFKLPDT
        PQGLLGEARM LNASIVASFV ELPLASIVSL
301 HASSCGGRLQ TSPAPIQTTP PKDTCSPELL
        MSLIQTKCAD DAMTLVLKKE LVAHLKCTIT
361 GLTFWDPSCE AEDRGDKFVL RSAYSSCGMQ
        VSASMISNEA VVNILSSSSP QRKKVHCLNM
421 DSLSFQLGLY LSPHFLQASN TIEPGQQSFV
        QVRVSPSVSE FLLQLDSCHL DLGPEGGTVE
481 LIQGRAAKGN CVSLLSPSPE GDPRFSFLLH
        FYTVPIPKTG TLSCTVALRP KTGSQDQEVH
541 RTVFMRLNII SPDLSGCTSK GTGGGTHTCP
        PCPAPELLGG PSVFLFPPKP KDTLMISRTP
601 EVTCVVVDVS HEDPEVKFNW YVDGVEVHNA
        KTKPREEQYN STYRVVSVLT VLHQDWLNGK
661 EYKCKVSNKA LPAPIEKTIS KAKGQPREPQ
        VYTLPPCREE MTKNQVSLWC LVKGFYPSDI
721 AVEWESNGQP ENNYKTTPPV LDSDGSFFLY
        SKLTVDKSRW QQGNVFSCSV MHEALHNHYT
781 QKSLSLSPGK
```

The complementary form of monomeric Fc polypeptide (SEQ ID NO: 506) uses the TPA leader and is as follows.

```
(SEQ ID NO: 506)
    1 MDAMKRGLCC VLLLCGAVFV SPGASNTKVD
        KRVTGGGTHT CPPCPAPELL
  51 GGPSVFLFPP KPKDTLMISR TPEVTCVVVD
        VSHEDPEVKF NWYVDGVEVH
101 NAKTKPREEQ YNSTYRVVSV LTVLHQDWLN
        GKEYKCKVSN KALPAPIEKT
151 ISKAKGQPRE PQVCTLPPSR EEMTKNQVSL
        SCAVKGFYPS DIAVEWESNG
201 QPENNYKTTP PVLDSDGSFF LVSKLTVDKS
        RWQQGNVFSC SVMHEALHNH
251 YTQKSLSLSP GK
```

The leader sequence is underlined, and an optional N-terminal extension of the Fc polypeptide is indicated by double underline. To promote formation of the endoglin-Fc:Fc heterodimer rather than either of the possible homodimeric complexes, four amino acid substitutions can be introduced into the monomeric Fc polypeptide as indicated by double underline above. The amino acid sequence of SEQ ID NO: 506 may optionally be provided with the C-terminal lysine removed.

The mature monomeric Fe polypeptide sequence (SEQ ID NO: 507) is as follows and may optionally be provided with the C-terminal lysine removed.

```
(SEQ ID NO: 507)
    1 SNTKVDKRVT GGGTHTCPPC PAPELLGGPS
        VFLFPPKPKD TLMISRTPEV
  51 TCVVVDVSHE DPEVKFNWYV DGVEVHNAKT
        KPREEQYNST YRVVSVLTVL
101 HQDWLNGKEY KCKVSNKALP APIEKTISKA
        KGQPREPQVC TLPPSREEMT
151 KNQVSLSCAV KGFYPSDIAV EWESNGQPEN
        NYKTTPPVLD SDGSFFLVSK
201 LTVDKSRWQQ GNVFSCSVMH EALHNHYTQK
        SLSLSPGK
```

The endoglin-Fc fusion polypeptide and monomeric Fc polypeptide of SEQ ID NO: 505 and SEQ ID NO: 507, respectively, may be co-expressed and purified from a CHO cell line to give rise to a single-arm heteromeric protein complex comprising endoglin-Fc:Fc.

Purification of various endoglin-Fc:Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 2. Generation of a Single-Arm Cripto-Fc Heterodimer

Applicants envision construction of a soluble single-arm Cripto-Fc heterodimeric complex comprising a monomeric Fc polypeptide with a short N-terminal extension and a second polypeptide in which a ligand-binding domain of human Cripto-1 is fused to a separate Fc domain with a linker positioned between a ligand-binding domain and this second Fc domain. The individual constructs are referred to as monomeric Fc polypeptide and Cripto-Fc fusion polypeptide, respectively, and the sequences for each are provided below. Applicants also envision additional single-arm Cripto-Fc heterodimeric complexes comprising a ligand-binding domain of Cripto-1 isoform 2 (SEQ ID NO: 18).

Formation of a single-arm Cripto-Fc heterodimer may be guided by approaches similar to those described for single-arm endoglin-Fc heterodimer in Example 1. In a first approach, illustrated in the Cripto-Fc and monomeric Fc polypeptide sequences of SEQ ID NOs: 508-509 and 502-503, respectively, one Fc domain is altered to introduce cationic amino acids at the interaction face, while the other Fc domain is altered to introduce anionic amino acids at the interaction face.

The Cripto-Fc fusion polypeptide employs the TPA leader and is as follows:

| (SEQ ID NO: 508) | |
|---|---|
| 1 | MDAMKRGLCC VLLLCGAVFV SPGASPPNPR TCVFFEAPGV RGSTKTLGEL LDTGTELPRA |
| 61 | IRCLYSRCCF GIWNLTQDRA QVEMQGCRDS DEPGCESLHC DPSPRAHPSP GSTLFTCSCG |
| 121 | TDFCNANYSH LPPPGSPGTP GSQGPQAAPG ESIWMALTGG GTHTCPPCPA PELLGGPSVF |
| 181 | LFPPKPKDTL MISRTPEVTC VVVDVSHEDP EVKFNWYVDG VEVHNAKTKP REEQYKSTYR |
| 241 | VVSVLTVLHQ DWLNGKEYKC KVSNKALPAP IEKTISKAKG QPREPQVYTL PPSRKEMTKN |
| 301 | QVSLTCLVKG FYPSDIAVEW ESNGQPENNY KTTPPVLKSD GSFFLYSKLT VDKSRWQQGN |
| 361 | VFSCSVMHEA LHNHYTQKSL SLSPGKTGGG THTCPPCPAP ELLGGPSVFL FPPKPKDTLM |
| 421 | ISRTPEVTCV VVDVSHEDPE VKFNWYVDGV EVHNAKTKPR EEQYNSTYRV VSVLTVLHQD |
| 481 | WLNGKEYKCK VSNKALPAPI EKTISKAKGQ PREPQVYTLP PSRKEMTKNQ VSLTCLVKGF |
| 541 | YPSDIAVEWE SNGQPENNYK TTPPVLKSDG SFFLYSKLTV DKSRWQQGNV FSCSVMHEAL |
| 601 | HNHYTQKSLS LSPGK |

The leader and linker sequences are underlined. To promote formation of the Cripto-Fc:Fc heterodimer rather than either of the possible homodimeric complexes (Cripto-Fc:Cripto-Fc or Fc:Fc), two amino acid substitutions (replacing anionic residues with lysines) can be introduced into the Fc domain of the fusion polypeptide as indicated by double underline above. The amino acid sequence of SEQ ID NO: 508 may optionally be provided with the C-terminal lysine removed.

The mature Cripto-Fc fusion polypeptide sequence is as follows (SEQ ID NO: 509) and may optionally be provided with the C-terminal lysine removed.

```
(SEQ ID NO: 509)
    1 PPNPRTCVFF EAPGVRGSTK TLGELLDTGT
        ELPRAIRCLY SRCCFGIWNL TQDRAQVEMQ
  61 GCRDSDEPGC ESLHCDPSPR AHPSPGSTLF
        TCSCGTDFCN ANYSHLPPPG SPGTPGSQGP
121 QAAPGESIWM ALTGGGTHTC PPCPAPELLG
        GPSVFLFPPK PKDTLMISRT PEVTCVVVDV
181 SHEDPEVKFN WYVDGVEVHN AKTKPPEEQY
        NSTYRVVSVL TVLHQDWLNG KEYKCKVSNK
241 ALPAPIEKTI SKAKGQPREP QVYTLPPSRK
        EMTKNQVSLT CLVKGFYPSD IAVEWESNGQ
301 PENNYKTTPP VLKSDGSFEL YSKLTVDKSR
        WQQGNVFSCS VMHEALHNHY TQKSLSLSPG
361 KTGGGTHTCP PCPAPELLGG PSVFLFPPKP
        KDTLMISRTP EVTCVVVDVS HEDPEVKFNW
421 YVDGVEVHNA KTKPREEQYN STYRVVSVLT
        VLHQDWLNGK EYKCKVSNKA LPAPIEKTIS
481 KAKGQPREPQ VYTLPPSRKE MTKNQVSLTC
        LVKGFYPSDI AVEWESNGQP ENNYKTTPPV
541 LKSDGSFFLY SKLTVDKSRW QQGNVFSCSV
        MHEALHNHYT QKSLSLSPGK
```

As described in Example 1, the complementary form of monomeric human G1Fc polypeptide (SEQ ID NO: 502) employs the TPA leader and incorporates an optional N-terminal extension. To promote formation of the Cripto-Fc:Fc heterodimer rather than either of the possible homodimeric complexes, two amino acid substitutions (replacing lysines with anionic residues) can be introduced into the monomeric Fc polypeptide as indicated. The amino acid sequence of SEQ ID NO: 502 may optionally be provided with the C-terminal lysine removed. The mature monomeric Fc polypeptide (SEQ ID NO: 503) may optionally be provided with the C-terminal lysine removed.

The Cripto-Fc fusion polypeptide and monomeric Fe polypeptide of SEQ ID NO: 509 and SEQ ID NO: 503, respectively, may be co-expressed and purified from a CHO cell line to give rise to a single-arm heteromeric protein complex comprising Cripto-Fc:Fc.

In another approach to promoting the formation of heteromultimer complexes using asymmetric Fc fusion polypeptides, the Fc domains are altered to introduce complementary hydrophobic interactions and an additional intermolecular disulfide bond as illustrated in the Cripto-Fc and Fc polypeptide sequences of SEQ ID NOs: 510-511 and 506-507, respectively.

The Cripto-Fc fusion polypeptide (SEQ ID NO: 510) uses the TPA leader and is as follows:

| (SEQ ID NO: 510) | |
|---|---|
| 1 | MDAMKRGLCC VLLLCGAVFV SPGASPPNRR TCVFFEADGV RGSTKTLGEL LDTGTELPRA |
| 61 | IRCLYSRCCF GIWNLTQDRA QVEMQGCRDS DEPGCESLHC DPSPRAHPSP GSTLFTCSCG |
| 121 | TDFCNANYSH LPPPGSPGTD GSQGPQAAPG ESIWMALTGG GTHTCPPCPA PELLGGPSVF |
| 181 | LFPPKPKDTL MISRTPEVTC VVVDVSHEDP EVKFNWYVDG VEVHNAKTKP REEQYKSTYR |
| 241 | VVSVLTVLHQ DWLNGKEYKC KVSNKALPAP IEKTISKAKG QPREPQVYTL PPSRKEMTKN |
| 301 | QVSLTCLVKG FYPSDIAVEW ESNGQPENNY KTTPPVLKSD GSFFLYSKLT VDKSRWQQGN |
| 361 | VFSCSVMHEA LHNHYTQKSL SLSPGKTGGG THTCPPCDAD ELLGGPSVFL FPPKPKDTLM |
| 421 | ISRTPEVTCV VVDVSHEDPE VKFNWYVDGV EVHNAKTKPR EEQYNSTYRV VSVLTVLHQD |
| 481 | WLNGKEYKCK VSNKALPAPI EKTISKAKGQ PREPQVYTLP PCREEMTKNQ VSLWCLVKGF |
| 541 | YTSDIAVEWE SNGQPENNYK TTPPVLDSDG SFFLYSKLTV DKSRWQQGNV FSCSVMHEAL |
| 601 | HNHYTQKSLS LSPGK |

The leader sequence and linker are underlined. To promote formation of the Cripto-Fc:Fc heterodimer rather than either of the possible homodimeric complexes, two amino acid substitutions (replacing a serine with a cysteine and a threonine with a tryptophan) can be introduced into the Fc domain of the Cripto fusion polypeptide as indicated by double underline above. The amino acid sequence of SEQ ID NO: 510 may optionally be provided with the C-terminal lysine removed.

The mature Cripto-Fc fusion polypeptide (SEQ ID NO: 511) is as follows and may optionally be provided with the C-terminal lysine removed.

```
(SEQ ID NO: 511
    1 PPNRRTCVFF EAPGVRGSTK TLGELLDTGT
        ELPRAIRCLY SRCCFGIWNL TQDRAQVEMQ
  61 GCRDSDEPGC ESLHCDPSPR AHPSPGSTLF
        TCSCGTDFCN ANYSHLPPPG SPGTPGSQGP
121 QAAPGESIWM ALTGGGTHTC PPCPAPELLG
        GPSVFLFPPK PKDTLMISRT PEVTCVVVDV
181 SHEDPEVKFN WYVDGVEVHN AKTKPREEQY
        NSTYRVVSVL TVLHQDWLNG KEYKCKVSNK
241 ALPAPIEKTI SKAKGQPREP QVYTLPPSRK
        EMTKNQVSLT CLVKGFYPSD IAVEWESNGQ
301 PENNYKTTPP VLKSDGSFFL YSKLTVDKSR
        WQQGNVFSCS VMHEALHNHY TQKSLSLSPG
361 KTGGGTHTCP PCPAPELLGG PSVFLFPPKP
        KDTLMISRTP EVTCVVVDVS HEDPEVKFNW
421 YVDGVEVHNA KTKPREEQYN STYRVVSVLT
        VLHQDWLNGK EYKCKVSNKA LPAPIEKTIS
481 KAKGQPPEPQ VYTLPPCREE MTKNQVSLWC
        LVKGFYPSDI AVEWESNGQP ENNYKTTPPV
541 LDSDGSFFLY SKLTVDKSRW QQGNVFSCSV
        MHEALHNHYT QKSLSLSPGK
```

As described in Example 1, the complementary form of monomeric G1Fc polypeptide (SEQ ID NO: 506) employs the TPA leader and incorporates an optional N-terminal extension. To promote formation of the Cripto-Fc:Fc heterodimer rather than either of the possible homodimeric complexes, four amino acid substitutions can be introduced into the monomeric Fc polypeptide as indicated. The amino acid sequence of SEQ ID NO: 506 and the mature Fc polypeptide (SEQ ID NO: 507) may optionally be provided with the C-terminal lysine removed.

The Cripto-Fc fusion polypeptide and monomeric Fc polypeptide of SEQ ID NO: 511 and SEQ ID NO: 507, respectively, may be co-expressed and purified from a CHO cell line to give rise to a single-arm heteromeric protein complex comprising Cripto-Fc:Fc.

Purification of various Cripto-Fc:Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 3. Generation of a Single-Arm Cryptic-Fc Heterodimer

Applicants envision construction of a soluble single-arm Cryptic-Fc heterodimeric complex comprising a monomeric Fc polypeptide with a short N-terminal extension and a second polypeptide in which a ligand-binding domain of human Cryptic is fused to a separate Fc domain with a linker positioned between a ligand-binding domain and this second Fc domain. The individual constructs are referred to as monomeric Fc polypeptide and Cryptic-Fc fusion polypeptide, respectively, and the sequences for each are provided below. Applicants also envision additional single-arm Cryptic-Fc heterodimeric complexes comprising a ligand-binding domain of Cryptic isoforms 2 or 3 (SEQ ID NOs: 26 or 30).

Formation of a single-arm Cryptic-Fc heterodimer may be guided by approaches similar to those described for single-arm endoglin-Fc heterodimer in Example 1. In a first approach, illustrated in the Cryptic-Fc and monomeric Fc polypeptide sequences of SEQ ID NOs: 512-513 and 502-503, respectively, one Fc domain is altered to introduce cationic amino acids at the interaction face, while the other Fc domain is altered to introduce anionic amino acids at the interaction face.

The Cryptic-Fc fusion polypeptide employs the TPA leader and is as follows:

| (SEQ ID NO: 512) | |
|---|---|
| 1 | 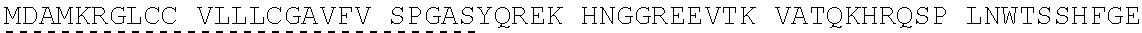 |
| 61 | VTGSAEGWGP EEPLPYSPAF GEGASARPRC CPNGGTCVLG SFCVCPAHFT GRYCEHDQRR |
| 121 | SECGALEHGA WTLRACHLCR CIFGALHCLP LQTPDRCDPK DFLASHAHGT GGGTHTCPPC |
| 181 | PAPELLGGPS VFLFPPKPKD TLMISRTPEV TCVVVDVSHE DPEVKFNWYV DGVEVHNAKT |
| 241 | KTREEQYNST YRVVSVLTVL HQDWLNGKEY KCKVSNKALP APIEKTISKA KGQPREPQVY |
| 301 | TLPPSRKEMT KNQVSLTCLV KGFYPSDIAV EWESNGQPEN NYKTTPPVLK SDGSFFLYSK |
| 361 | LTVDKSRWQQ GNVFSCSVMH EALHNHYTQK SLSLSPGK |

The leader and linker sequences are underlined. To promote formation of the Cryptic-Fc:Fc heterodimer rather than either of the possible homodimeric complexes (Cryptic-Fc:Cryptic-Fc or Fc:Fc), two amino acid substitutions (replacing anionic residues with lysines) can be introduced into the Fc domain of the fusion polypeptide as indicated by double underline above. The amino acid sequence of SEQ ID NO: 512 may optionally be provided with the C-terminal lysine removed.

The mature Cryptic-Fc fusion polypeptide sequence is as follows (SEQ ID NO: 513) and may optionally be provided with the C-terminal lysine removed.

```
(SEQ ID NO: 513)
    1 YQREKHNGGR EEVTKVATQK HRQSPLNWTS
        SHFGEVTGSA EGWGPEEPLP YSRAFGEGAS
  61 ARPRCCRNGG TCVLGSFCVC PAHFTGRYCE
        HDQRRSECGA LEHGAWTLRA CHLCRCIFGA
121 LHCLPLQTPD RCDPKDFLAS HAHGTGGGTH
        TCPPCPAPEL LGGPSVFLFP PKPKDTLMIS
181 RTPEVTCVVV DVSHEDPEVK FNWYVDGVEV
        HNAKTKPREE QYNSTYRVVS VLTVLHQDWL
241 NGKEYKCKVS NKALPAPIEK TISKAKGQPR
        EPQVYTLPPS RKEMTKNQVS LTCLVKGFYP
301 SDIAVEWESN GQPENNYKTT PPVLKSDGSF
        FLYSKLTVDK SRWQQGNVFS CSVMHEALHN
361 HYTQKSLSLS PGK
```

As described in Example 1, the complementary form of monomeric human G1Fc polypeptide (SEQ ID NO: 502) employs the TPA leader and incorporates an optional N-terminal extension. To promote formation of the Cryptic-Fc:Fc heterodimer rather than either of the possible homodimeric complexes, two amino acid substitutions (replacing lysines with anionic residues) can be introduced into the monomeric Fc polypeptide as indicated. The amino acid sequence of SEQ ID NO: 502 may optionally be provided with the C-terminal lysine removed. The mature monomeric Fc polypeptide (SEQ ID NO: 503) may optionally be provided with the C-terminal lysine removed.

The Cryptic-Fc fusion polypeptide and monomeric Fc polypeptide of SEQ ID NO: 513 and SEQ ID NO: 503, respectively, may be co-expressed and purified from a CHO cell line to give rise to a single-arm heteromeric protein complex comprising Cryptic-Fc:Fc.

In another approach to promoting the formation of heteromultimer complexes using asymmetric Fc fusion polypeptides, the Fc domains are altered to introduce complementary hydrophobic interactions and an additional intermolecular disulfide bond as illustrated in the Cryptic-Fc and Fc polypeptide sequences of SEQ ID NOs: 514-515 and 506-507, respectively.

The Cryptic-Fc fusion polypeptide (SEQ ID NO: 514) uses the TPA leader and is as follows:

| (SEQ ID NO: 514) | |
|---|---|
| 1 | MDAMKRGLCC VLLLCGAVFV SPGASYQREK HNGGREEVTK VATQKHRQSP LVWTSSEFGE |
| 61 | VTGSAEGWGP EEPLPYSRAF GEGASAPPRC CRNGGTCVLG SFCVCPAHFT GRYCEHDQRR |
| 121 | 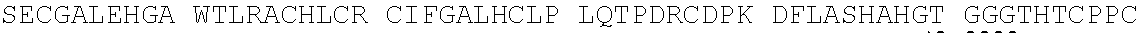 |
| 181 | PAPELLGGPS VFLFPPKPKD TLMISRTPEV TCVVVDVSHE DPEVKFNWYV DGVEVHNAKT |
| 241 | KPREEQYNST YRVVSVLTVL HQDWLNGKEY KCKVSNKALP APIEKTISKA KGQPREPQVY |
| 301 | TLPPCREEMT KNQVSLWCLV KGFYPSDIAV EWESNGQPEN NYKTTPPVLD SDGSFFLYSK |
| 361 | LTVDKSRWQQ GNVFSCSVMH EALHNHYTQK SLSLSPGK |

The leader sequence and linker are underlined. To promote formation of the Cryptic-Fc:Fc heterodimer rather than either of the possible homodimeric complexes, two amino acid substitutions (replacing a serine with a cysteine and a threonine with a tryptophan) can be introduced into the Fc domain of the Cryptic fusion polypeptide as indicated by double underline above. The amino acid sequence of SEQ ID NO: 514 may optionally be provided with the C-terminal lysine removed.

The mature Cryptic-Fc fusion polypeptide (SEQ ID NO: 515) is as follows and may optionally be provided with the C-terminal lysine removed.

```
(SEQ ID NO: 515)
    1 YQREKHNGGR EEVTKVATQK HRQSPLNWTS
        SHFGEVTGSA EGWGPEEPLP YSRAFGEGAS
  61 ARPRCCRNGG TCVLGSFCVC PAHFTGRYCE
        HDQRRSECGA LEHGAWTLRA CHLCRCIFGA
121 LHCLPLQTPD RCDPKDFLAS HAHGTGGGTH
        TCPPCPAPEL LGGPSVFLFP PKPKDTLMIS
181 RTPEVTCVVV DVSHEDPEVK FNWYVDGVEV
        HNAKTKPREE QYNSTYRVVS VLTVLHQDWL
241 NGKEYKCKVS NKALPAPIEK TISKAKGQPR
        EPQVYTLPPC REEMTKNQVS LWCLVKGFYP
301 SDIAVEWESN GQPENNYKTT PPVLDSDGSF
        FLYSKLTVDK SRWQQGNVFS CSVMHEALHN
361 HYTQKSLSLS PGK
```

As described in Example 1, the complementary form of monomeric G1Fc polypeptide (SEQ ID NO: 506) employs the TPA leader and incorporates an optional N-terminal extension. To promote formation of the Cryptic-Fc:Fc heterodimer rather than either of the possible homodimeric complexes, four amino acid substitutions can be introduced into the monomeric Fc polypeptide as indicated. The amino acid sequence of SEQ ID NO: 506 and the mature Fc polypeptide (SEQ ID NO: 507) may optionally be provided with the C-terminal lysine removed.

The Cryptic-Fc fusion polypeptide and monomeric Fc polypeptide of SEQ ID NO: 515 and SEQ ID NO: 507, respectively, may be co-expressed and purified from a CHO cell line to give rise to a single-arm heteromeric protein complex comprising Cryptic-Fc:Fc.

Purification of various Cryptic-Fc:Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 4. Generation of a Single-Arm Cryptic1B-Fc Heterodimer

Applicants envision construction of a soluble single-arm Cryptic1B-Fc heterodimeric complex comprising a monomeric Fc polypeptide with a short N-terminal extension and a second polypeptide in which a ligand-binding domain of human Cryptic family protein 1B is fused to a separate Fc domain with a linker positioned between a ligand-binding domain and this second Fc domain. The individual constructs are referred to as monomeric Fc polypeptide and Cryptic1B-Fc fusion polypeptide, respectively, and the sequences for each are provided below.

Formation of a single-arm Cryptic1B-Fc heterodimer may be guided by approaches similar to those described for single-arm endoglin-Fc heterodimer in Example 1. In a first approach, illustrated in the Cryptic1B-Fc and monomeric Fc polypeptide sequences of SEQ ID NOs: 516-517 and 502-503, respectively, one Fc domain is altered to introduce cationic amino acids at the interaction face, while the other Fc domain is altered to introduce anionic amino acids at the interaction face.

The Cryptic1B-Fc fusion polypeptide employs the TPA leader and is as follows:

| (SEQ ID NO: 516) | |
|---|---|
| 1 | MDAMKRGLCC VLLLCGAVFV SPGASYQREK HNGGREEVTK VATQKHRQSP LNWTSSHFGE |
| 61 | VTGSAEGWGP EEPLPYSWAF GEGASARPRC CRNGGTCVLG SFCVCPAHFT GRYCEHDQRR |
| 121 | 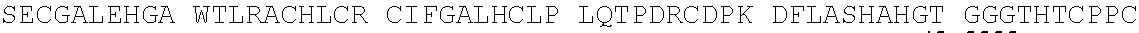 |
| 181 | PAPELLGGPS VFLFPPKPKD TLMISRTPEV TCVVVDVSHE DPEVKFNWYV DGVEVHNAKT |
| 241 | KPREEQYNST YRVVSVLTVL HQDWLNGKEY KCKVSNKALP APIEKTISKA KGQPREPQVY |
| 301 | TLPPSRKEMT KNQVSLTCLV KGFYPSDIAV EWESNGQPEN NYKTTPPVLK SDGSFFLYSK |
| 361 | LTVDKSRWQQ GNVFSCSVMH EALHNHYTQK SLSLSPGK |

The leader and linker sequences are underlined. To promote formation of the Cryptic1B-Fc:Fc heterodimer rather than either of the possible homodimeric complexes (Cryptic1B-Fc:Cryptic1B-Fc or Fc:Fc), two amino acid substitutions (replacing anionic residues with lysines) can be introduced into the Fc domain of the fusion polypeptide as indicated by double underline above. The amino acid sequence of SEQ ID NO: 516 may optionally be provided with the C-terminal lysine removed.

The mature Cryptic1B-Fc fusion polypeptide sequence is as follows (SEQ ID NO: 517) and may optionally be provided with the C-terminal lysine removed.

```
(SEQ ID NO: 517)
    1 YQREHENGGR EEVTKVATQK HRQSPLNWTS
        SHFGEVTGSA EGWGPEEPLP YSWAFGEGAS
  61 ARPRCCRNGG TCVLGSFCVC PAHFTGRYCE
        HDQRRSECGA LEHGAWTLRA CHLCRCIFGA
121 LHCLPLQTPD RCDPKDFLAS HAHGTGGGTH
        TCPPCPAPEL LGGPSVFLFP PKPKDTLMIS
181 RTPEVTCVVV DVSHEDPEVK FNWYVDGVEV
        HNAKTKPREE QNSTYRVVS VLTVLHQDWL
241 NGKEYKCKVS NKALPAPIEK TISKAKGQPR
        EPQVYTLPPS RKEMTKMQVS LTCLVKGFYP
301 SDIAVEWESN GQPENNYKTT PPVLKSDGSF
        FLYSKLTVDK SRWQQGNVFS CSVMHEALHN
361 HYTQKSLSLS PGK
```

As described in Example 1, the complementary form of monomeric human G1F polypeptide (SEQ ID NO: 502) employs the TPA leader and incorporates an optional N-terminal extension. To promote formation of the Cryptic1B-Fc:Fc heterodimer rather than either of the possible homodimeric complexes, two amino acid substitutions (replacing lysines with anionic residues) can be introduced into the monomeric Fc polypeptide as indicated. The amino acid sequence of SEQ ID NO: 502 may optionally be provided with the C-terminal lysine removed. The mature monomeric Fc polypeptide (SEQ ID NO: 503) may optionally be provided with the C-terminal lysine removed.

The Cryptic1B-Fc fusion polypeptide and monomeric Fc polypeptide of SEQ ID NO: 517 and SEQ ID NO: 503, respectively, may be co-expressed and purified from a CHO cell line to give rise to a single-arm heteromeric protein complex comprising CrypticB-Fc:Fc.

In another approach to promoting the formation of heteromultimer complexes using asymmetric Fc fusion polypeptides, the Fc domains are altered to introduce complementary hydrophobic interactions and an additional intermolecular disulfide bond as illustrated in the Cryptic1B-Fc and Fc polypeptide sequences of SEQ ID NOs: 518-519 and 506-507, respectively.

The Cryptic1B-Fc fusion polypeptide (SEQ ID NO: 518) uses the TPA leader and is as follows:

| (SEQ ID NO: 518) | |
|---|---|
| 1 | 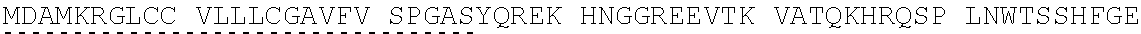 |
| 61 | VTGSAEGWGP EEPLPYSWAF GEGASARPRC CRNGGTCVLG SFCVCPAHFT GRYCEHDQRR |
| 121 | SECGALEHGA WTLRACHLCR CIFGALHCLP LQTPDRCDPK DFLASHAHGT GGGTHTCPPC |
| 181 | PAPELLGGPS VFLFPPKPKD TLMISRTPEV TCVVVDVSHE DPEVKFNWYV DGVEVHNAKT |
| 241 | KPREEQYNST YRVVSVLTVL HQDWLNGKEY KCKVSNKALP APIEKTISKA KGQPREPQVY |
| 301 | TLPPCREEMT KNQVSLWCLV KGFYPSDIAV EWESNGQPEN NYKTTPPVLD SDGSFFLYSK |
| 361 | LTVDKSRWQQ GNVFSCSVMH EALHNHYTQK SLSLSPGK |

The leader sequence and linker are underlined. To promote formation of the Cryptic1B-Fc:Fc heterodimer rather than either of the possible homodimeric complexes, two amino acid substitutions (replacing a seine with a cysteine and a threonine with a tryptophan) can be introduced into the Fc domain of the Cryptic1B fusion polypeptide as indicated by double underline above. The amino acid sequence of SEQ ID NO: 518 may optionally be provided with the C-terminal lysine removed.

The mature Cryptic1B-Fc fusion polypeptide (SEQ ID NO: 519) is as follows and may optionally be provided with the C-terminal lysine removed.

```
(SEQ ID NO: 519)
    1 YQREKHNGGR EEVTKVATQK HRQSPLNWTS
        SHFGEVTGSA EGWGPEEPLP YSWAFGEGAS
  61 ARPRCCRNGG TCVLGSFCVC PAHFTGRYCE
        HDQRRSECGA LEHGAWTLRA CHLCRCIFGA
121 LHCLPLQTPD RCDPKDFLAS HAHGTGGGTH
        TCPPCPAPEL LGGPSVFLFP PKPKDTLMIS
131 RTPEVTCVVV DVSHEDPEVK FNWYVDGVEV
        HNAKTKPREE QYNSTYRVVS VLTV1KQDWL
241 NGKEYKCKVS NKALPAPIEK TISKAKGQPR
        EPQVYTLPPC REEMTKNQVS LWCLVKGFYP
301 SDIAVEWESN GQPENNYKTT PPVLDSDGSF
        FLYSKLTVDK SRWQQGNVFS CSVMHEALHN
361 HYTQKSLSLS PGK
```

As described in Example 1, the complementary form of monomeric G1Fc polypeptide (SEQ ID NO: 506) employs the TPA leader and incorporates an optional N-terminal extension. To promote formation of the Cryptic1B-Fc:Fc heterodimer rather than either of the possible homodimeric complexes, four amino acid substitutions can be introduced into the monomeric Fc polypeptide as indicated. The amino acid sequence of SEQ ID NO: 506 and the mature Fc polypeptide (SEQ ID NO: 507) may optionally be provided with the C-terminal lysine removed.

The Cryptic1B-Fc fusion polypeptide and monomeric Fc polypeptide of SEQ ID NO: 519 and SEQ ID NO: 507, respectively, may be co-expressed and purified from a CHO cell line to give rise to a single-arm heteromeric protein complex comprising CrypticB-Fc:Fc.

Purification of various Cryptic1B-Fc:Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 5. Generation of a Single-Arm CRIM1-Fc Heterodimer

Applicants envision construction of a soluble single-arm CRIM1-Fc heterodimeric complex comprising a monomeric Fc polypeptide with a short N-terminal extension and a second polypeptide in which a ligand-binding domain of human CRIM1 is fused to a separate Fc domain with a linker positioned between a ligand-binding domain and this second Fc domain. The individual constructs are referred to as monomeric Fc polypeptide and CRIM1-Fc fusion polypeptide, respectively, and the sequences for each are provided below.

Formation of a single-arm CRIM1-Fc heterodimer may be guided by approaches similar to those described for single-arm endoglin-Fc heterodimer in Example 1. In a first approach, illustrated in the CRIM1-Fc and monomeric Fc polypeptide sequences of SEQ ID NOs: 520-521 and 502-503, respectively, one Fc domain is altered to introduce cationic amino acids at the interaction face, while the other Fc domain is altered to introduce anionic PGP211,TRE amino acids at the interaction face.

The CRIM1-Fc fusion polypeptide employs the TPA leader and is as follows:

| (SEQ ID NO: 520) | |
|---|---|
| 1 | MDAMKRGLCC VLLLCGAVFV SPGASLVCLP CDESKCEEPR NCPGSIVQGV CGCCYTCASQ |
| 61 | RNESCGGTFG IYGTCDRGLR CVIRPPLNGD SLTEYEAGVC EDENWTDDQL LGFKPCNENL |
| 121 | IAGCNIINGK CECNTIRTCS NPFEFPSQDM CLSALKRIEE EKPDCSKARC EVQFSPRCPE |
| 181 | DSVLIEGYAP PGECCPLPSR CVCNPAGCLR KVCQPGNLNI LVSKASGKPG ECCDLYECKP |
| 241 | VFGVDCRTVE CPPVQQTACP PDSYETQVRL TADGCCTLPT CECLSGLCGF PVCEVGSTPR |
| 301 | IVSRGDGTPG KCCDVFECVN DTKPACVFNN VEYYDGDMFR MDNCRFCRCQ GGVAICFTAQ |
| 361 | CGEINCERYY VPEGECCPVC EDPVYPFNNP AGCYANGLIL AHGDPWREDD CTFCQCVNGE |
| 421 | RHCVATVCGQ TCTNPVKVPG ECCPVCEEPT IITVDPPACG ELSNCTLTGK DCINGFKRDH |
| 481 | NGCRTCQCIN TEELCSERKQ GCTLNCPFGF LTDAQNCEIC ECRPRPKKCR PIICDKYCPL |
| 541 | GLLKNKHGCD ICRCKKCPEL SCSKICPLGF QQDSHGCLIC KCREASASAG PPILSGTCLT |
| 601 | VDGHHHKNEE SWHDGCRECY CLNGREMCAL ITCPVPACGN PTIHPGQCCP SCADDFVVQK |
| 661 | PELSTPSICH APGGEYFVEG ETWNIDSCTQ CTCHSGRVLC ETEVCPPLLC QNPSPTQDSC |
| 721 | CPQCTDQPFR PSLSRNNSVP NYCKNDEGDI FLAAESWKPD VCTSCICIDS VISCFSESCP |
| 781 | SVSCERPVLR KGQCCPYCIE DTIPKKVVCH FSGKAYADEE RWDLDSCTHC YCLQGQTLCS |
| 841 | TVSCPPLPCV EPINVEGSCC PMCPEMYVPE PTNIPIEKTN HRGEVDLEVP LWPTPSENDI |
| 901 | 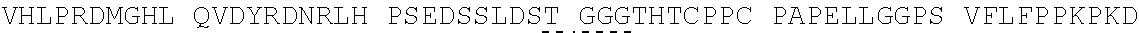 |
| 961 | TLMISRTPEV TCVVVDVSHE DPEVKFNWYV DGVEVHNAKT KPREEQYNST YRVVSVLTVL |
| 1021 | HQDWLNGKEY KCKVSNKALP APIEKTISKA KGQPREPQVY TLPPSRKEMT KNQVSLTCLV |
| 1081 | KGFYPSDIAV EWESNGQPEN NYKTTPPVLK SDGSFFLYSK LTVDKSRWQQ GNVFSCSVMH |
| 1141 | EALHNHYTQK SLSLSPGK |

The leader and linker sequences are underlined. To promote formation of the CRIM1-Fc:Fc heterodimer rather than either of the possible homodimeric complexes (CRIM1-Fc:CRIM1-Fc or Fc:Fc), two amino acid substitutions (replacing anionic residues with lysines) can be introduced into the Fc domain of the fusion polypeptide as indicated by double underline above. The amino acid sequence of SEQ ID NO: 520 may optionally be provided with the C-terminal lysine removed.

The mature CRIM1-Fc fusion polypeptide sequence is as follows (SEQ ID NO: 521) and may optionally be provided with the C-terminal lysine removed.

```
(SEQ ID NO: 521)
      1 LVCLPCDESK CEEPRNCPGS IVQGVCGCCY
          TCASQRNESC GGTFGIYGTC DRGLRCVIRP
    61 PLNGDSLTEY EAGVCEDENW TDDQLLGFKP
          CNENLIAGCN IINGKCECNT IRTCSNPFEF
  121 PSQDMCLSAL KRIEEEKPDC SKARCEVQFS
          PRCPEDSVLI EGYAPPGECC PLPSRCVCNP
  181 AGCLRKVCQP GNLNILVSKA SGKPGECCDL
          YECKPVFGVD CRTVECPPVQ QTACPPDSYE
  241 TQVPLTADGC CTLPTCECLS GLCGFPVCEV
          GSTPRIVSRG DGTPGKCCDV FECVNDTKPA
  301 CVFNNVEYYD GDMFRMDNCR FCRCQGGVAI
          CFTAQCGEIN CERYYVPEGE CCPVCEDPVY
  361 PFNNPAGCYA NGLILAHGDR WREDDCTFCQ
          CVNGERHCVA TVCGQTCTNP VKVPGECCPV
  421 CEEPTIITVD PPACGELSNC TLTGKDCING
          FKRDHKGCRT CQCINTEELC SERKQGCTLN
  481 CPFGELTDAQ NCEICECRPR PKKCRPIICD
          KYCPLGLLKN KHGCDICRCK KCPELSCSKI
  541 CPLGFQQDSH GCLICKCREA SASAGPPILS
          GTCLTVDGHH HKNEESWHDG CRECYCLNGR
  601 EMCALITCPV PACGNPTIHP GQCCPSCADD
          FVVQKPELST PSICHAPGGE YFVEGETWNI
  661 DSCTQCTCHS GRVLCETEVC PPILCQNPSP
          TQDSCCPQCT DQPFRPSLSR NNSVPKYCKN
  721 DEGDIFLAAE SWKPDVCTSC ICIDSVISCF
          SESCPSVSCE RPVLRKGQCC PYCIEDTIPK
  781 KVVCHFSGKA YADEERWDLD SCTHCYCLQG
          QTLCSTVSCP PLPCVEPINV EGSCCPMCPE
  841 MYVPEPTNIP IEKTNERGEV DLEVPLWPTP
          SENDIVHLPR DMGELQVDYR DNRLHPSEDS
  901 SLDSTGGGTH TCPPCPAPEL LGGPSVFLFP
          PKPKDTLMIS RTPEVTCVVV DVSHEDPEVK
  961 FNWYVDGVEV HNAKTKPREE OYNSTYRVVS
          VITVLHQDWL NGKEYKCKVS NKALPAPIEK
1021 TISKAKGQPR EPQVYTLPPS RKEMTKNQVS
          LTCLVKGFYP SDIAVEWESN GQPENNYKTT
1081 PPVLKSDGSF FLYSKLTVDK SRWQQGNVFS
          CSVMHEALHN HYTQKSLSLS PGK
```

As described in Example 1, the complementary form of monomeric human G1Fc polypeptide (SEQ ID NO: 502) employs the TPA leader and incorporates an optional N-terminal extension. To promote formation of the CRIM1-Fc:Fc heterodimer rather than either of the possible homodimeric complexes, two amino acid substitutions (replacing lysines with anionic residues) can be introduced into the monomeric Fc polypeptide as indicated. The amino acid sequence of SEQ ID NO: 502 may optionally be provided with the C-terminal lysine removed. The mature monomeric Fc polypeptide (SEQ ID NO: 503) may optionally be provided with the C-terminal lysine removed.

The CRIM1-Fc fusion polypeptide and monomeric Fc polypeptide of SEQ ID NO: 521 and SEQ ID NO: 503, respectively, may be co-expressed and purified from a CHO cell line to give rise to a single-arm heteromeric protein complex comprising CRIM1-Fc:Fc.

In another approach to promoting the formation of heteromultimer complexes using asymmetric Fc fusion polypeptides, the Fc domains are altered to introduce complementary hydrophobic interactions and an additional intermolecular disulfide bond as illustrated in the CRIM1-Fc and Fc polypeptide sequences of SEQ ID NOs: 522-523 and 506-507, respectively.

The CRIM1-Fc fusion polypeptide (SEQ ID NO: 522) uses the TPA leader and is as follows:

| (SEQ ID NO: 522) | |
|---|---|
| 1 | MDAMKRGLCC VLLLCGAVFV SPGASLVCLP CDESKCEEPR NCPGSIVQGV CGCCYTCASQ |
| 61 | RNESCGGTFG IYGTCDRGLR CVIRPPLNGD SLTEYEAGVC EDENWTDDQL LGFKPCNENL |
| 121 | IAGCNIINGK CECNTIRTCS NPFEFPSQDM CLSALKRIEE EKPDCSKARC EVQFSPRCPE |
| 181 | DSVLIEGYAP PGECCPLPSR CVCNPAGCLR KVCQPGNLNI LVSKASGKPG ECCDLYECKP |
| 241 | VFGVDCRTVE CPPVQQTACP PDSYETQVRL TADGCCTLPT CECLSGLCGF PVCEVGSTPR |
| 301 | IVSRGDGTPG KCCDVFECVN DTKPACVFNN VEYYDGDMFR MDNCRFCRCQ GGVAICFTAQ |
| 361 | CGEINCERYY VPEGECCPVC EDPVYPFNNP AGCYANGLIL AHGDRWREDD CTFCQCVNGE |
| 421 | RHCVATVCGQ TCTNPVKVPG ECCPVCEEPT IITVDPPACG ELSNCTLTGK DCINGFKRDH |
| 481 | NGCPTCQCIN TEELCSERKQ GCTLNCPFGF LTDAQNCEIC ECRPRPKKCR PIICDKYCPL |
| 541 | GLLKNKHGCD ICRCKKCPEL SCSKICPLGF QQDSHGCLIC KCREASASAG PPILSGTCLT |
| 601 | VDGHHHKNEE SWHDGCRECY CLNGREMCAL ITCPVPACGN PTIHPGQCCP SCADDFVVQK |
| 661 | PELSTPSICH APGGEYFVEG ETWNIDSCTQ CTCHSGPVLC ETEVCPPLLC QNPSRTQDSC |
| 721 | CPQCTDQPFR PSLSRNNSVP NYCKNDEGDI FLAAESWKPD VCTSCICIDS VISCFSESCP |
| 781 | SVSCERPVLR KGQCCPYCIE DTIPKKVVCH FSGKAYADEE RWDLDSCTHC YCLQGQTLCS |
| 841 | TVSCPPLPCV EPINVEGSCC PMCPEMYVPE PTNIPIEKTN HRGEVDLEVP LWPTPSENDI |
| 901 | VHLPRDMGHL QVDYRDNRLH PSEDSSLDST GGGTHTCPPC PAPELLGGPS VFLFPPKPKD |
| 961 | TLMISRTPEV TCVVVDVSHE DPEVKFNWYV DGVEVENAKT KPREEOYNST YRVVSYLTVL |
| 1021 | 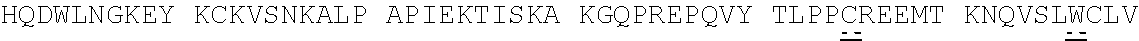 |
| 1081 | KGFYPSDIAV EWESNGQPEN NYKTTPPVLD SDGSFFLYSK LTVDKSRWQQ GNVFSCSVMH |
| 1141 | EALHNHYTQK SLSLSPGK |

The leader sequence and linker are underlined. To promote formation of the CRIM1-Fc:Fc heterodimer rather than either of the possible homodimeric complexes, two amino acid substitutions (replacing a serine with a cysteine and a threonine with a tryptophan) can be introduced into the Fc domain of the CRIM1 fusion polypeptide as indicated by double underline above. The amino acid sequence of SEQ ID NO: 522 may optionally be provided with the C-terminal lysine removed.

The mature CRIM1-Fc fusion polypeptide (SEQ ID NO: 523) is as follows and may optionally be provided with the C-terminal lysine removed.

```
(SEQ ID NO: 523)
      1 LVCLPCDESK CEEPRNCPGS IVQGVCGCCY
          TCASQRNESC GGTFGIYGTC DRGLRCVIRP
    61 PLNGDSLTEY EAGVCEDENW TDDQLLGFKP
          CNENLIAGCN IINGKCECNT IRTCSNPFEF
  121 PSQDMCLSAL KRIEEEKPDC SKARCEVQFS
          PRCPEDSVLI EGYAPPGECC PLPSRCVCNP
  181 AGCLRKVCQP GNLNILVSKA SGKPGECCDL
          YECKPVFGVD CPTVECPPVQ QTACPPDSYE
  241 TQVRLTADGC CTLPTCECLS GLCGFPVCEV
          GSTPRIVSRG DGTPGKCCDV FECVNDTKPA
  301 CVFNNVEYYD GDMFRMDNCR FCRCQGGVAI
          CFTAQCGEIN CERYYVPEGE CCPVCEDPVY
  361 PFNNPAGCYA NGLILAHGDR WREDDCTFCQ
          CVNGERHCVA TVCGQTCTNP VKVPGECCPV
  421 CEEPTIITVD PPACGELSNC TLTGKDCING
          FKRDHNGCRT CQCINTEELC SERKQGCTLN
  481 CPFGFLTDAQ NCEICECRPR PKKCRPIICD
          KYCPLGLLKN KHGCDICRCK KCPELSCSKI
  541 CPLGFQQDSH GCLICKCREA SASAGPPILS
          GTCLTVDGHH KKNEESWHDG CRECYCLNGR
  601 EMCALITCPV PACGNPTIHP GQCCPSCADD
          FVVQKPELST PSICHAPGGE YFVEGETWNI
  661 DSCTQCTCHS GRVLCETEVC PPLLCQNPSR
          TQDSCCPQCT DQPFRPSLSR NNSVPNYCKN
  721 DEGDIFLAAE SWKPDVCTSC ICIDSVISCF
          SESCPSVSCE RPVLRKGQCC PYCIEDTIPK
  781 KVVCHFSGFA YADEEPWDLD SCTHCYCLQG
          QTLCSTVSCP PLPCVEPINV EGSCCPMCPE
  841 MYVPEPTNIP IEKTNHRGEV DLEVPLWPTP
          SENDIVHLPR DMGHLQVDYR DNRLHPSEDS
  901 SLDSTGGGTH TCPPCPAPEL LGGPSVFLFP
          PKPKDTLMIS RTPEVTCVVV DVSHEDPEVK
  961 FNWYVDGVEV KNAKTKPREE QYNSTYPVVS
          VLTVLHQDWL NGKEYKCKVS NKALPAPIEK
1021 TISKAKGQPR EPQVYTLPPC REEMTKNQVS
          LWCLVKGFYP SDIAVEWESN GQPENNYKTT
1081 PPVLDSDGSF FLYSKLTVDK SRWQQGNVFS
          CSVMHEALHN HYTQKSLSLS PGIK
```

As described in Example 1, the complementary form of monomeric G1Fc polypeptide (SEQ ID NO: 506) employs the TPA leader and incorporates an optional N-terminal extension. To promote formation of the CRIM1-Fc:Fc heterodimer rather than either of the possible homodimeric complexes, four amino acid substitutions can be introduced into the monomeric Fc polypeptide as indicated. The amino acid sequence of SEQ ID NO: 506 and the mature Fc polypeptide (SEQ ID NO: 507) may optionally be provided with the C-terminal lysine removed.

The CRIM1-Fc fusion polypeptide and monomeric Fc polypeptide of SEQ ID NO: 523 and SEQ ID NO: 507, respectively, may be co-expressed and purified from a CHO cell line to give rise to a single-arm heteromeric protein complex comprising CRIM1-Fc:Fc.

Purification of various CRIM1-Fc:Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 6. Generation of a Single-Arm CRIM2-Fc Heterodimer

Applicants envision construction of a soluble single-arm CRIM2-Fc heterodimeric complex comprising a monomeric Fc polypeptide with a short N-terminal extension and a second polypeptide in which a ligand-binding domain of human CRIM2 is fused to a separate Fc domain with a linker positioned between a ligand-binding domain and this second Fc domain. The individual constructs are referred to as monomeric Fc polypeptide and CRIM2-Fc fusion polypeptide, respectively, and the sequences for each are provided below. Applicants also envision additional single-arm CRIM2-Fc heterodimeric complexes comprising a ligand-binding domain of CRIM2 isoform 2 (SEQ ID NO: 46).

Formation of a single-arm CRIM2-Fc heterodimer may be guided by approaches similar to those described for single-arm endoglin-Fc heterodimer in Example 1. In a first approach, illustrated in the CRIM2-Fc and monomeric Fc polypeptide sequences of SEQ ID NOs: 524-525 and 502-503, respectively, one Fc domain is altered to introduce cationic amino acids at the interaction face, while the other Fc domain is altered to introduce anionic amino acids at the interaction face.

The CRIM2-Fc fusion polypeptide employs the TPA leader and is as follows:

```
(SEQ ID NO: 524)
      1 MDAMKRGLCC VLLLCGAVFV SPGASGAVPR
          EPPGQQTTAH SSVLAGNSQE QWHPLREWLG
    61 RLEAAVMELR EQNKDLQTRV RQLESCECHP
          ASPQCWGLGR AWPEGARWEP DACTACVCQD
  121 GAAHCGPQAH LPHCRGCSQN GQTYGNGETF
          SPDACTTCRC LTGAVQCQGP SCSELNCLES
  181 CTPPGECCPI CCTEGGSHWE HGQEWTTPGD
          PCRICRCLEG HIQCRQRECA SLCPYPARPL
  241 PGTCCPVCDG CFLNGREHRS GEPVGSGDPC
          SHCRCANGSV QCEPLPCPPV PCRHPGKIPG
  301 QCCPVCDGCE YQGHQYQSQE TFRLQERGLC
          VRCSCQAGEV SCEEQECPVT PCALPASGRQ
  361 LCPACELDGE EFAEGVQWEP DGRPCTACVC
          QDGVPKCGAV LCPPAPCQHP TQPPGACCPS
  421 CDSCTYHSQV YANGQNFTDA DSPCHACHCQ
          DGTVTCSLVD CPPTTCARPQ SGPGQCCPRC
  481 PDCILEEEVF VDGESFSHPR DPCQECRCQE
          GHAHCQPRPC PRAPCAHPLP GTCCPNDCSG
  541 CAFGGKEYPS GADFPHPSDP CRLCRCLSGN
          VQCLARPCVP LPCPEPVLLP GECCPQCPAP
  601 AGCPRPGAAH ARHQEYESPP GDPCRRCLCL
          DGSVSCQRLP CPPAPCAHPR QGPCCPSCDG
  661 CLYQGKEFAS GERFPSPTAA CHLCLCWEGS
          VSCEPKACAP ALCPFPARGD CCPDCDGCEY
  721 LGESYLSNQE FPDPREPCNL CTCLGGFVTC
          GRRPCEPPGC SHPLIPSGHC CPTCQGCRYH
  781 GVTTASGETL PDPLDPTCSL CTCQEGSMRC
          QKKPCPPALC PHPSPGPCFC PVCHSCLSQG
  841 REHQDGEEFE GPAGSCEWCR CQAGQVSCVR
          LQCPPLPCKL QVTERGSCCP RCRGCLAHGE
  901 EEPEGSRWVP PDSACSSCVC HEGVVTCARI
          QCISSCAQPR QGPHDCCPQC SDCEHEGRKY
  961 EPGESFQPGA DPCEVCICEP QPEGPPSLRC
          HRRQCPSLVG CPPSQLLPPG PQHCCPTCAE
1021 ALSNCSEGLL GSELAPPDPC YTCQCQDLTW
          LCIHQACPEL SCPLSERHTP PGSCCPVCRA
1081 PTQSCVHQGR EVASGERWTV DTCTSCSCMA
          GTVRCQSQRC SPLSCGPDKA PALSPGSCCP
1141 RCLPRPASCM AFGDPHYRTF DGPLLHFQGS
          CSYVLAKDCH SGDFSVHVTN DDRGRSGVAW
1201 TQEVAVLLGD MAVRLLQDGA VTVDGHPVAL
          PFLQEPLLYV ELRGHTVILH AQPGLQVLWD
1261 GQSQVEVSVP GSYQGRTCGL CGNFNGFAQD
          DLQGPEGLLL PSEAAFGNSW QVSEGLWPGR
1321 PCSAGREVDP CRAAGYRARR EANARCGVLK
          SSPFSRCHAV VPPEPFFAAC VYDLCACGPG
1381 SSADACLCDA LEAYASHCRQ AGVTPTWRGP
          TLCVVGCPLE RGFVFDECGP PCPRTCFNQH
1441 IPLGELAAHC VRPCVPGCQC PAGLVEHEAH
          CIPPEACPQV LLTGDQPLGA RPSPSREPQE
1501 TPTGGGTHTC PPCPAPELLG GPSVFLFPPK
          PKDTIMISRT PEVTCVVVDV SHEDPEVKFN
1561 WYVDGVEVHN AKTKPREEQY NSTYRVVSVL
          TVLHQDWLNG KEYKCKVSNK ALPAPIEKTI
1621 SKAKGQPREP QVYTLPPSRK EMTKNQVSLT
          CLVKGFYPSD IAVEWESNGQ PENNYKTTPP
1681 VLKSDGSFFL YSKLTVDKSR WQQGNVFSCS
          VMHEALHNHY TQKSLSLSPG K
```

The leader and linker sequences are underlined. To promote formation of the CRIM2-Fc:Fc heterodimer rather than either of the possible homodimeric complexes (CRIM2-Fc:CRIM2-Fc or Fc:Fc), two amino acid substitutions (replacing anionic residues with lysines) can be introduced into the Fc domain of the fusion polypeptide as indicated by double underline above. The amino acid sequence of SEQ ID NO: 524 may optionally be provided with the C-terminal lysine removed.

The mature CRIM2-Fc fusion polypeptide sequence is as follows (SEQ ID NO: 525) and may optionally be provided with the C-terminal lysine removed.

```
(SEQ ID NO: 525)
      1 GAVPREPPGQ QTTAHSSVLA GNSQEQWHPL
          PEWLGRLEAA VMELREQNKD LQTRVRQLES
    61 CECHPASPQC WGLGRAWPEG ARWEPDACTA
          CVCQDGAAHC GPQAHLPHCR GCSQNGQTYG
  121 NGETFSPDAC TTCRCLTGAV QCQGPSCSEL
          NCLESCTPPG ECCPICCTEG GSHWEHGQEW
  181 TTPGDPCRIC RCLEGHIQCR QRECASLCPY
          PARPLPGTCC PVCDGCFLNG REHRSGEPVG
  241 SGDPCSHCRC ANGSVQCEPL PCPPVPCRHP
          GKIPGQCCPV CDGCEYQGHQ YQSQETFRLQ
  301 ERGLCVRCSC QAGEVSCEEQ ECPVTPCALP
          ASGRQLCPAC ELDGEEFAEG VQWEPDGRPC
  361 TACVCQDGVP KCGAVLCPPA PCQHPTQPPG
          ACCPSCDSCT YHSQVYANGQ NFTDADSPCH
  421 ACHCQDGTVT CSLVDCPPTT CARPQSGPGQ
          CCPRCPDCIL EEEVFVDGES FSHPRDPCQE
  481 CRCQEGHAHC QPRPCPRAPC AHPLPGTCCP
          NDCSGCAFGG KEYPSGADFP HPSDPCRLCR
  541 CLSGNVQCLA RRCVPLPCPE PVLLPGECCP
          QCPAPAGCPR PGAAHARHQE YFSPPGDPCR
  601 RCLCLDGSVS CQRLPCPPAP CAHPRQGPCC
          PSCDGCLYQG KEFASGERFP SPTAACHLCL
  661 CWEGSVSCEP KACAPALCPF PARGDCCPDC
          DGCEYLGESY LSNQEFPDPR EPCNLCTCLG
  721 GFVTCGRRPC EPPGCSHPLI PSGHCCPTCQ
          GCRYHGVTTA SGETLPDPLD PTCSLCTCQE
  781 GSMRCQKKPC PPALCPHPSP GPCFCPVCHS
          CLSQGREHQD GEEFEGPAGS CEWCPCQAGQ
  841 VSCVRLQCPP LPCKLQVTER GSCCPRCRGC
          LAHGEEHPEG SPWVPPDSAC SSCVCHEGVV
  901 TCARIQCISS CAQPRQGPHD CCPQCSDCEH
          EGRKYEPGES FQPGADPCEV CICEPQPEGP
  961 PSLRCHRRQC PSLVGCPPSQ LLPPGPQHCC
          PTCAEALSNC SEGLLGSELA PPDPCYTCQC
1021 QDLTWLCIHQ ACPELSCPLS ERHTPPGSCC
          PVCRAPTQSC VHQGREVASG ERWTVDTCTS
1081 CSCMAGTVRC QSQRCSPLSC GPDKAPALSP
          GSCCPRCLPR PASCMAFGDP HYRTFDGRIL
1141 HFQGSCSYVL AKDCHSGDFS VHVTNDDRGR
          SGVAWTQEVA VLLGDMAVRL LQDGAVTVDG
1201 HPVALPFLQE PLLYVELRGH TVILHAQPGL
          QVLWDGQSQV EVSVPGSYQG RTCGLCGNFN
1261 GFAQDDLQGP EGLLLPSEAA FGNSWQVSEG
          LWPGRPCSAG REVDPCRAAG YRARREANAR
1321 CGVLKSSPFS RCHAVVPPEP FFAACVYDLC
          ACGPGSSADA CLCDALEAYA SHCRQAGVTP
1381 TWRGPTLCVV GCPLERGFVF DECGPPCPRT
          CFNQHIPLGE LAAHCVRPCV PGCQCPAGLV
1441 EHEAHCIPPE ACPQVLLTGD QPLGARPSPS
          REPQETPTGG GTHTCPPCPA PELLGGPSVF
1501 LFPPKPKDTL MISRTPEVTC VVVDVSHEDP
          EVKFNWYVDG VEVHNAKTKP REEQYNSTYR
1561 VVSVLTVLHQ DWLNGKEYKC KVSNKALPAP
          IEKTISKAKG QPREPQVYTL PPSRKEMTKN
1621 QVSLTCLVKG FYPSDIAVEW ESNGQPENNY
          KTTPPVLKSD GSFFLYSKLT VDKSRWQQGN
1681 VFSCSVMHEA LHNHYTQKSL SLSPGK
```

As described in Example 1, the complementary form of monomeric human G1Fc polypeptide (SEQ ID NO: 502) employs the TPA leader and incorporates an optional N-terminal extension. To promote formation of the CRIM2-Fc:Fc heterodimer rather than either of the possible homodimeric complexes, two amino acid substitutions (replacing lysines with anionic residues) can be introduced into the monomeric Fc polypeptide as indicated. The amino acid sequence of SEQ ID NO: 502 may optionally be provided with the C-terminal lysine removed. The mature monomeric Fc polypeptide (SEQ ID NO: 503) may optionally be provided with the C-terminal lysine removed.

The CRIM2-Fc fusion polypeptide and monomeric Fc polypeptide of SEQ ID NO: 525 and SEQ ID NO: 503, respectively, may be co-expressed and purified from a CHO cell line to give rise to a single-arm heteromeric protein complex comprising CRIM2-Fc:Fc.

In another approach to promoting the formation of heteromultimer complexes using asymmetric Fc fusion polypeptides, the Fc domains are altered to introduce complementary hydrophobic interactions and an additional intermolecular disulfide bond as illustrated in the CRIM2-Fc and Fc polypeptide sequences of SEQ ID NOs: 526-527 and 506-507, respectively.

The CRIM2-Fc fusion polypeptide (SEQ ID NO: 526) uses the TPA leader and is as follows:

| (SEQ ID NO: 526) | |
|---|---|
| 1 | MDAMKRGLCC VLLLCGAVFV SPGASGAVPR EPPGQQTTAH SSVLAGNSQE QWHPLREWLG |
| 61 | RLEAAVMELR EQNKDLQTRV RQLESCECHP ASPQCWGLGR AWPEGARWEP DACTACVCQD |
| 121 | GAAHCGPQAH LPHCRGCSQN GQTYGNGETF SPDACTTCRC LTGAVQCQGP SCSELNCLES |
| 181 | CTPPGECCPI CCTEGGSHWE HGQEWTTPGD PCRICRCLEG HIQCPQRECA SLCPYPARPL |
| 241 | PGTCCPVCDG CFLNGREHRS GEPVGSGDPC SHCRCANGSV QCEPLPCPPV PCRHPGKIPG |
| 301 | QCCPVCDGCE YQGHQYQSQE TFRLQERGLC VRCSCQAGEV SCEEQECPVT PCALPASGRQ |
| 361 | LCPACELDGE EFAEGVQWEP DGRPCTACVC QDGVPKCGAV LCPPAPCQHP TQPPGACCPS |
| 421 | CDSCTYHSQV YANGQNFTDA DSPCHACHCQ DGTVTCSLVD CPPTTCARPQ SGPGQCCPRC |
| 481 | PDCILEEEVF VDGESFSHPR DPCQECRCQE GHAHCQPRPC PRAPCAHPLP GTCCPNDCSG |
| 541 | CAFGGKEYPS GADFPHPSDP CRLCRCLSGN VQCLARRCVP LPCPEPVLLP GECCPQCPAP |
| 601 | AGCPRPGAAH ARHQEYFSPP GDPCRRCLCL DGSVSCQRLP CPPAPCAHPR QGPCCPSCDG |
| 661 | CLYQGKEFAS GERFPSPTAA CHLCLCWEGS VSCEPKACAP ALCPFPARGD CCPDCDGCEY |
| 721 | LGESYLSNQE FPDPREPCNL CTCLGGFVTC GRRPCEPPGC SHPLIPSGHC CPTCQGCRYH |
| 781 | GVTTASGETL PDPLDPTCSL CTCQEGSMRC QKKPCPPALC RHPSPGPCFC PVCHSCLSQG |
| 841 | REHQDGEEFE GPAGSCEWCR CQAGQVSCVR LQCPPLPCKL QVTERGSCCP RCRGCLAHGE |
| 901 | EHPEGSRWVP PDSACSSCVC HEGVVTCARI QCISSCAQPR QGPHDCCPQC SDCEHEGRKY |
| 961 | EPGESFQPGA DPCEVCICEP QPEGPPSLRC HRRQCPSLVG CPPSQLLPPG PQHCCPTCAE |
| 1021 | ALSNCSEGLL GSELAPPDPC YTCQCQDLTW LCIHQACPEL SCPLSERHTP PGSCCPVCRA |
| 1081 | PTQSCVHQGR AFGDPHYRTF DGRLLHFQGS CSYVLAKDCH SGDFSVHVTN DDRGRSGVAW |
| 1141 | RCLPRPASCM AFGDPHYRTF DGRLLHFQGS CSYVLAKDCH SGDFSVHVTN DDRGRSGVAW |
| 1201 | TQEVAVLLGD MAVRLLQDGA VTVDGHPVAL PFLQEPLLYV ELRGHTVILH AQPGLQVLWD |
| 1261 | GQSQVEVSVP GSYQGRTCGL CGNFNGFAQD DLQGPEGLLL PSEAAFGNSW QVSEGLWPGR |
| 1321 | PCSAGREVDP CRAAGYRARR EANARCGVLK SSPFSRCHAV VPPEPFFAAC VYDLCACGPG |
| 1381 | SSADACLCDA LEAYASHCRQ AGVTPTWRGP TLCVVGCPLE RGFVFDECGP PCPRTCFNQH |
| 1441 | IPLGELAAHC VRPCVPGCQV PAGLVEHEAH CIPPEACPQV LLTGDQPLGA RPSPSREPQE |
| 1501 | TPTGGGTHTC PPCPAPELLG GPSVFLFPPK PKDTLMISRT PEVTCVVVDV SHEDPEVKFN |
| 1561 | WYVDGVEVHN AKTKPREEQY NSTYRVVSVL TVLHQDWLNG KEYKCKVSNK ALPAPIEKTI |
| 1621 | SKAKGQPREP QVYTLPPCRE EMTKNQVSLW CLVKGFYPSD IAVEWESNGQ PENNYKTTPP |
| 1681 | VLDSDGSFFL YSKLTVDKSR WQQGNVFSCS VMHEALHNHY TQKSLSLSPG K |

The leader sequence and linker are underlined. To promote formation of the CRIM2-Fc:Fc heterodimer rather than either of the possible homodimeric complexes, two amino acid substitutions (replacing a serine with a cysteine and a threonine with a tryptophan) can be introduced into the Fc domain of the CRIM2 fusion polypeptide as indicated by double underline above. The amino acid sequence of SEQ ID NO: 526 may optionally be provided with the C-terminal lysine removed.

The mature CRIM2-Fc fusion polypeptide (SEQ ID NO: 527) is as follows and may optionally be provided with the C-terminal lysine removed.

| (SEQ ID NO: 527) | |
|---|---|
| 1 | GAVPREPPGQ QTTAHSSVLA GNSQEQWHPL WEWLGRLEAA VMELPEQNKD LQTRVRQLES |
| 61 | CECHPASPQC WGLGRAWPEG ARWEPDACTA CVCQDGAAHC GPQAHLPHCR GCSQNGQTYG |
| 121 | NGETFSPDAC TTCRCLTGAV QCQGPSCSEL NCLESCTPPG ECCPICCTEG GSHWEHGQEW |
| 181 | TTPGDPCRIC RCLEGHIQCR QRECASLCPY PARPLPGTCC PVCDGCFLNG REHRSGEPVG |
| 241 | SGHPCSHCRC ANGSVQCEPL PCPPVPCRHP GKIPGQCCPV CDGCEYQGHQ YQSQETFRLQ |
| 301 | ERGLCVRCSC QAGEVSCEEQ ECPVTPCALP ASGRQLCPAC ELDGEEFAEG VQWEPDGRPC |
| 361 | TACVCQDGVR KCGAVLCPPA PCQHPTQPPG ACCPSCDSCT YHSQVYANGQ NFTDADSPCH |
| 421 | ACHCQDGTVT CSLVDCPPTT CARPQSGPGQ CCPRCPDCIL EEEVFVDGES FSHPRDPCQE |
| 481 | CRCQEGHAHC QPRPCPRAPC AHPLPGTCCP NDCSGCAFGG KEYPSGADFP HPSDPCRLCR |
| 541 | CLSGNVQCLA RRCVPLPCPE PVLLPGECCP QCPAPAGCPR PGAAHARHQE YFSPPGDPCR |
| 601 | RCLCLDGSVS CQRLPCPPAP CAHPRQGPCC PSCDGCLYQG KEFASGERFP SPTAACHLCL |
| 661 | CWEGSVSCEP KACAPALCPF PARGDCCPDC DGCEYLGESY LSNQEFPDPR EPCNLCTCLG |
| 721 | GFVTCGRRPC EPPGCSHPLI PSGHCCPTCQ GCRYHGVTTA SGETLPDPLD PTCSLCTCQE |
| 781 | GSMRCQKKPC PPALCPHPSP GPCFCPVCHS CLSQGREHQD GEEFEGPAGS CEWCRCQAGQ |
| 841 | VSCVRLQCPP LPCKLQVTEP GSCCPRCRGC LAHGEEHPEG SRWVPPDSAC SSCVCHEGVV |
| 901 | TCARIQCISS CAQPRQGPKD CCPQCSDCEH EGRKYEPGES FQPGADPCEV CICEPQPEGP |
| 961 | PSLRCHRRQC PSLVGCPPSQ LLPPGPQHCC PTCAEALSNC SEGLLGSELA PPDPCYTCQC |
| 1021 | QDLTWLCIHQ ACPELSCPLS ERHTPPGSCC PVCRAPTQSC VHQGREVASG ERWTVDTCTS |
| 1081 | CSCMAGTVRC QSQRCSPLSC GPDKAPALSP GSCCPRCLPR PASCMAFGDP HYRTFDGRLL |
| 1141 | HFQGSCSYVL AKDCHSGDFS VHVTNDDRGR SGVAWTQEVA VLLGDMAVRL LQDGAVTVDG |
| 1201 | HPVALPFLQE PLLYVELRGH TVILHAQPGL QVLWDGQSQV EVSVPGSYQG RTCGLCGNFN |
| 1261 | GFAQDDLQGP EGLLLPSEAA FGNSWQVSEG LWPGRPCSAG REVDPCRAAG YRARREANAR |
| 1321 | CGVLKSSPFS RCHAVVPPEP FFAACVYDLC ACGPGSSADA CLCDALEAYA SHCRQAGVTP |
| 1381 | TWRGPTLCVV GCPLERGFVF DECGPPCPRT CFNQHIPLGE LAAHCVRPCV PGCQCPAGLV |
| 1441 | EHEAHCIPPE ACPQVLLTGD QPLGARPSPS REPQETPTGG GTHTCPPCPA RELLGGPSVF |
| 1501 | LFPPKPKDTL MISRTPEVTC VVVDVSHEDP EVKFNWYVDG VEVHNAKTKP REEQYNSTYR |
| 1561 | VVSVLTVLHQ DWLNGKEYKC KVSNKALPAP IEKTISKAKG QPREPQVYTL PPCREEMTKN |
| 1621 | QVSLWCLVKG FYPSDIAVEW ESNGQPENNY KTTPPVLDSD GSFFLYSKLT VDKSRWQQGN |
| 1681 | VFSCSVMHEA LHNHYTQKSL SLSPGK |

As described in Example 1, the complementary form of monomeric G1Fe polypeptide (SEQ ID NO: 506) employs the TPA leader and incorporates an optional N-terminal extension. To promote formation of the CRIM2-Fc:Fc heterodimer rather than either of the possible homodimeric complexes, four amino acid substitutions can be introduced into the monomeric Fe polypeptide as indicated. The amino acid sequence of SEQ ID NO: 506 and the mature Fe polypeptide (SEQ ID NO: 507) may optionally be provided with the C-terminal lysine removed.

The CRIM2-Fc fusion polypeptide and monomeric Fe polypeptide of SEQ ID NO: 527 and SEQ ID NO: 507, respectively, may be co-expressed and purified from a CHO cell line to give rise to a single-arm heteromeric protein complex comprising CRIM2-Fc:Fc.

Purification of various CRIM2-Fc:Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 7. Generation of a Single-Arm BAMBI-Fc Heterodimer

Applicants envision construction of a soluble single-arm BAMBI-Fc heterodimeric complex comprising a monomeric Fc polypeptide with a short N-terminal extension and a second polypeptide in which a ligand-binding domain of human BAMBI is fused to a separate Fc domain with a linker positioned between a ligand-binding domain and this second Fc domain. The individual constructs are referred to as monomeric Fc polypeptide and BAMBI-Fc fusion polypeptide, respectively, and the sequences for each are provided below.

Formation of a single-arm BAMBI-Fc heterodimer may be guided by approaches similar to those described for single-arm endoglin-Fc heterodimer in Example 1. In a first approach, illustrated in the BAMBI-Fc and monomeric Fc polypeptide sequences of SEQ ID NOs: 528-529 and 502-503, respectively, one Fc domain is altered to introduce cationic amino acids at the interaction face, while the other Fc domain is altered to introduce anionic amino acids at the interaction face.

The BAMBI-Fc fusion polypeptide employs the TPA leader and is as follows:

| (SEQ ID NO: 528) | |
|---|---|
| 1 | MDAMKRGLCC VLLLCGAVFV SPGASVLLTK GEIRCYCDAA HCVATGYMCK SELSACFSRL |
| 61 | LDPQNSNSPL THGCLDSLAS TTDICQAKQA RNHSGTTIPT LECCHEDMCN YRGLHDVLSP |
| 121 | PRGEASGQGN RYQHDGSRNL ITKVQELTSS KELWFRATGG GTHTCPPCPA PELLGGPSVF |
| 181 | LFPPKPKDTL MISRTPEVTC VVVDVSHEDP EVKFNWYVDG VEVHNAKTKP REEQYNSTYR |
| 241 | VVSVLTVLHQ DWLNGKEYKC KVSNKALPAP IEKTISKAKG QPREPQVYTL PPSRKEMTKN |
| 301 | QVSLTCLVKG FYPSDIAVEW ESNGQPENNY KTTPPVLKSD GSFFLYSKLT VDKSRWQQGN |
| 361 | VFSCSVMHEA LHNHYTQKSL SLSPGK |

The leader and linker sequences are underlined. To promote formation of the BAMBI-Fc:Fc heterodimer rather than either of the possible homodimeric complexes (BAMBI-Fc:BAMBI-Fc or Fc:Fc), two amino acid substitutions (replacing anionic residues with lysines) can be introduced into the Fc domain of the fusion polypeptide as indicated by double underline above. The amino acid sequence of SEQ ID NO: 528 may optionally be provided with the C-terminal lysine removed.

The mature BAMBI-Fc fusion polypeptide sequence is as follows (SEQ ID NO: 529) and may optionally be provided with the C-terminal lysine removed.

| (SEQ ID NO: 529) | |
|---|---|
| 1 | VLLTKGEIRC YCDAAHCVAT GYMCKSELSA CFSRLLDPQN SNSPLTHGCL DSLASTTDIC |
| 61 | QAKQARNHSG TTIPTLECCH EDMCNYRGLH DVLSPPRGEA SGQGNRYQHD GSRNLITKVQ |
| 121 | ELTSSKELWF RATGGGTHTC PPCPAPELLG GPSVFLFPPK PKDTLMISRT PEVTCVVVDV |
| 181 | SHEDPEVKFN WYVDGVEVHN AKTKPREEQY NSTYRVVSVL TVLHQDWLNG KEYKCKVSNK |
| 241 | ALPAPIEKTI SKAKGQPREP QVYTLPPSRK EMTKNQVSLT CLVKGFYPSD IAVEWESNGQ |
| 301 | PENNYKTTPP VLKSDGSFFL YSKLTVDKSR WQQGNVFSCS VMHEALHNHY TQKSLSLSPG |
| 361 | K |

As described in Example 1, the complementary form of monomeric human G1Fc polypeptide (SEQ ID NO: 502) employs the TPA leader and incorporates an optional N-terminal extension. To promote formation of the BAMBI-Fc:Fc heterodimer rather than either of the possible homodimeric complexes, two amino acid substitutions (replacing lysines with anionic residues) can be introduced into the monomeric Fc polypeptide as indicated. The amino acid sequence of SEQ ID NO: 502 may optionally be provided with the C-terminal lysine removed. The mature monomeric Fc polypeptide (SEQ ID NO: 503) may optionally be provided with the C-terminal lysine removed.

The BAMBI-Fc fusion polypeptide and monomeric Fc polypeptide of SEQ ID NO: 529 and SEQ ID NO: 503, respectively, may be co-expressed and purified from a CHO cell line to give rise to a single-arm heteromeric protein complex comprising BAMBI-Fc:Fc.

In another approach to promoting the formation of heteromultimer complexes using asymmetric Fc fusion polypeptides, the Fc domains are altered to introduce complementary hydrophobic interactions and an additional intermolecular disulfide bond as illustrated in the BAMBI-Fc and Fc polypeptide sequences of SEQ ID NOs: 530-531 and 506-507, respectively.

The BAMBI-Fc fusion polypeptide (SEQ ID NO: 530) uses the TPA leader and is as follows:

| (SEQ ID NO: 530) | |
|---|---|
| 1 | MDAMKRGLCC VLLLCGAVFV SPGASVLLTK GEIRCYCDAA HCVATGYMCK SELSACFSRL |
| 61 | LDPQNSNSPL THGCLDSLAS TTDICQAKQA RNHSGTTIPT LECCHEDMCN YRGLHDVLSP |
| 121 | PRGEASGQGN RYQHDGSRNL ITKVQELTSS KELWFRATGG GTHTCPPCPA PELLGGPSVF |
| 181 | LFPPKPKDTL MISRTPEVTC VVVDVSHEDP EVKFNWYVDG VEVHNAKTKP REEQYNSTYR |
| 241 | VVSVLTVLHQ DWLNGKEYKC KVSNKALPAP IEKTISKAKG QPREPQVYTL PPCRKEMTKN |
| 301 | QVSLWCLVKG FYPSDIAVEW ESNGQPENNY KTTPPVLDSD GSFFLYSKLT VDKSRWQQGN |
| 361 | VFSCSVMHEA LHNHYTQKSL SLSPGK |

The leader sequence and linker are underlined. To promote formation of the BAMBI-Fc:Fc heterodimer rather than either of the possible homodimeric complexes, two amino acid substitutions (replacing a serine with a cysteine and a threonine with a tryptophan) can be introduced into the Fc domain of the BAMBI fusion polypeptide as indicated by double underline above. The amino acid sequence of SEQ ID NO: 530 may optionally be provided with the C-terminal lysine removed.

The mature BAMBI-Fc fusion polypeptide (SEQ ID NO: 531) is as follows and may optionally be provided with the C-terminal lysine removed.

| (SEQ ID NO: 531) | |
|---|---|
| 1 | VLLTKGEIRC YCDAAHCVAT GYMCKSELSA CFSRLLDPQN SNSPLTHGCL DSLASTTDIC |
| 61 | QAKQARNHSG TTIPTLECCH EDMCNYRGLH DVLSPPRGEA SGQGNRYQHD GSRNLITKVQ |
| 121 | ELTSSKELWF RATGGGTHTC PPCPAPELLG GPSVFLFPPK PKDTLMISRT PEVTCVVVDV |
| 181 | SHEDPEVKFN WYVDGVEVHN AKTKPREEQY NSTYRVVSVL TVLHQDWLNG KEYKCKVSNK |
| 241 | ALPAPIEKTI SKAKGQPREP QVYTLPPCRE EMTKNQVSLW CLVKGFYPSD IAVEWESNGQ |
| 301 | PENNYKTTPP VLDSDGSFFL YSKLTVDKSR WQQGNVFSCS VMHEALHNHY TQKSLSLSPG |
| 361 | K |

As described in Example 1, the complementary form of monomeric G1Fc polypeptide (SEQ ID NO: 506) employs the TPA leader and incorporates an optional N-terminal extension. To promote formation of the BAMBI-Fc:Fc heterodimer rather than either of the possible homodimeric complexes, four amino acid substitutions can be introduced into the monomeric Fc polypeptide as indicated. The amino acid sequence of SEQ ID NO: 506 and the mature Fc polypeptide (SEQ ID NO: 507) may optionally be provided with the C-terminal lysine removed.

The BAMBI-Fc fusion polypeptide and monomeric Fc polypeptide of SEQ ID NO: 531 and SEQ ID NO: 507, respectively, may be co-expressed and purified from a CHO cell line to give rise to a single-arm heteromeric protein complex comprising BAMBI-Fc:Fc.

Purification of various BAMBI-Fc:Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 8. Generation of a Single-Arm BMPER-Fc Heterodimer

Applicants envision construction of a soluble single-arm BMPER-Fc heterodimeric complex comprising a monomeric Fc polypeptide with a short N-terminal extension and a second polypeptide in which a ligand-binding domain of human BMPER is fused to a separate Fc domain with a linker positioned between a ligand-binding domain and this second Fc domain. The individual constructs are referred to as monomeric Fc polypeptide and BMPER-Fc fusion polypeptide, respectively, and the sequences for each are provided below.

Formation of a single-arm BMPER-Fc heterodimer may be guided by approaches similar to those described for single-arm endoglin-Fc heterodimer in Example 1. In a first approach, illustrated in the BMPER-Fc and monomeric Fc polypeptide sequences of SEQ ID NOs: 532-533 and 502-503, respectively, one Fc domain is altered to introduce cationic amino acids at the interaction face, while the other Fc domain is altered to introduce anionic amino acids at the interaction face.

The BMPER-Fc fusion polypeptide employs the TPA leader and is as follows:

| (SEQ ID NO: 532) | |
|---|---|
| 1 | MDAMKRGLCC VLLLCGAVFV SPGASSSFLT GSVAKCENEG EVLQIPFITD NPCIMCVCLN |
| 61 | KEVTCKREKC PVLSRDCALA IKQRGACCEQ CKGCTYEGNT YKSSFKWQSP AEPCVLRQCQ |
| 121 | EGVVTESGVR CVVHCKNPLE HLGMCCPTCP GCVFEGVQYQ EGEEFQPEGS KCTKCSCTGG |
| 181 | RTQCVREVCP ILSCPQHLSH IPPGQCCPKC LGQRKVFDLP FGSCLFRSDV YDNGSSFLYD |
| 241 | NCTACTCRDS TVVCKRKCSH PGGCDQGQEG CCEECLLRVP PEDIKVCKFG NKIFQDGEMW |
| 381 | SSINCTICAC VKGRTECRNK QCIPISSCPQ GKILNRKGCC PICTEKPGVC TVFGDPHYNT |
| 361 | FDGRTFNFQG TCQYVITKDC SSPASPFQVL VKNDARRTRS FSWTKSVELV LGESRVSLQQ |
| 421 | HLTVRWNGSR IALPCRAPHF HIDLDGYLLK VTTKAGLEIS WDGDSFVEVM AAPHLKGKLC |
| 481 | GLCGNYNGHK RDDLIGGDGN FKFDVDDFAE SWRVESNEFC NRPQRKPVPE LCQGTVKVKL |
| 541 | RAHRECQKLK SWEFQTCHST VDYATFYRSC VTDMCECPVH KNCYCESFLA YTRACQREGI |
| 601 | KVHWEPQQNC AATQCKHGAV YDTCGPGCIK TCDNWNEIGP CNKPCVAGCH CPANLVLHKG |
| 661 | RCIKPVICPQ RTGGGTHTCP PCPAPELLGG PSVFLFPPKP KDTLMISRTP EVTCVVVDVS |
| 721 | HEDPEVKFNW YVDGVEVHNA KTKPREEQYN STYRVVSVLT VLHQDWLNGK EYKCKVSNKA |
| 781 | LPAPIEKTIS KAKGQPREPQ VYTLPPSRKE MTKNQVSLTC LVKGFYPSDI AVEWESNGQP |
| 841 | ENNYKTTPPV LKSDGSFFLY SKLTVDKSRW QQGNVFSCSV MHEALHNHYT QKSLSLSPGK |

The leader and linker sequences are underlined. To promote formation of the BMPER-Fc:Fc heterodimer rather than either of the possible homodimeric complexes (BMPER-Fc:BMPER-Fc or Fc:Fc), two amino acid substitutions (replacing anionic residues with lysines) can be introduced into the Fe domain of the fusion polypeptide as indicated by double underline above. The amino acid sequence of SEQ ID NO: 532 may optionally be provided with the C-terminal lysine removed.

The mature BMPER-Fc fusion polypeptide sequence is as follows (SEQ ID NO: 533) and may optionally be provided with the C-terminal lysine removed.

| (SEQ ID NO: 533) | |
|---|---|
| 1 | SSFLTGSVAK CENEGEVLQI PFITDNPCIM CVCLNKEVTC KREKCPVLSR DCALAIKQRG |
| 61 | ACCEQCKGCT YEGNTYNSSF KWQSPAEPCV LRQCQEGVVT ESGVRCVVHC KNPLEHLGMC |
| 121 | CPTCPGCVFE GVQYQEGEEF QPEGSKCTKC SCTGGRTQCV REVCPILSCP QHLSHIPPGQ |
| 181 | CCPKCLGQRK VFDLPFGSCL FRSDVYDNGS SFLYDNCTAC TCRDSTVVCK RKCSHPGGCD |
| 241 | QGQEGCCEEC LLRVPPEDIK VCKFGNKIFQ DGEMWSSINC TICACVKGRT ECRNKQCIPI |
| 301 | SSCPQGKILN RKGCCPICTE KPGVCTVFGD PHYNTFDGRT FNFQGTCQYV LTKDCSSPAS |
| 361 | PFQVLVKNDA RRTRSFSWTK SVELVLGESR VSLQQHLTVR WNGSRIALPC RAPHFHIDLD |
| 421 | GYLLKVTTKA GLEISWDGDS FVEVMAAPHL KGKLCGLCGN YNGHKRDDLI GGDGNFKFDV |
| 481 | DDFAESWRVE SNEFCNRPQR KPVPELCQGT VKVKLRAHRE CQKLKSWEFQ TCHSTVDYAT |
| 541 | FYRSCVTDMC ECPVHKNCYC ESFLAYTRAC QREGIKVHWE PQQNCAATQC KHGAVYDTCG |
| 601 | PGCIKTCDNW NEIGPCNKPC VAGCHCPANL VLHKGRCIKP VLCPQRTGGG THTCPPCPAP |
| 661 | ELLGGPSVFL FPPKPKDTLM ISRTPEVTCV VVDVSHEDPE VKFNWYVDGV EVHNAKTKPR |
| 721 | EEQYNSTYRV VSVLTVLHQD WLNGKEYKCK VSNKALPAPI EKTISKAKGQ PREPQVYTLP |
| 781 | PSRKEMTKNQ VSLTCLVKGF YPSDIAVEWE SNGQPENNYK TTPPVLKSDG SFFLYSKLTV |
| 841 | DKSRWQQGNV FSCSVMHEAL HNHYTQKSLS LSPGK |

As described in Example 1, the complementary form of monomeric human G1Fe polypeptide (SEQ ID NO: 502) employs the TPA leader and incorporates an optional N-terminal extension. To promote formation of the BMPER-Fc:Fc heterodimer rather than either of the possible homodimeric complexes, two amino acid substitutions (replacing lysines with anionic residues) can be introduced into the monomeric Fe polypeptide as indicated. The amino acid sequence of SEQ ID NO: 502 may optionally be provided with the C-terminal lysine removed. The mature monomeric Fe polypeptide (SEQ ID NO: 503) may optionally be provided with the C-terminal lysine removed.

The BMPER-Fc fusion polypeptide and monomeric Fc polypeptide of SEQ ID NO: 533 and SEQ ID NO: 503, respectively, may be co-expressed and purified from a CHO cell line to give rise to a single-arm heteromeric protein complex comprising BMPER-Fc:Fc.

In another approach to promoting the formation of heteromultimer complexes using asymmetric Fc fusion polypeptides, the Fc domains are altered to introduce complementary hydrophobic interactions and an additional intermolecular disulfide bond as illustrated in the BMPER-Fc and Fc polypeptide sequences of SEQ ID NOs: 534-535 and 506-507, respectively.

The BMPER-Fc fusion polypeptide (SEQ ID NO: 534) uses the TPA leader and is as follows:

| (SEQ ID NO: 534) | |
|---|---|
| 1 | MDAMKRGLCC VLLLCGAVFV SPGASSSFLT GSVAKCENEG EVLQIPFITD NPCIMCVCLN |
| 61 | KEVTCKREKC PVLSRDCALA IKQRGACCEQ CKGCTYEGNT YNSSFKWQSP AEPCVLRQCQ |
| 121 | EGVVTESGVR CVVHCKNPLE HLGMCCPTCP GCVFEGVQYQ EGEEFQPEGS KCTKCSCTGG |
| 181 | RTQCVREVCP ILSCPQHLSH IPPGQCCPKC LGQRKVFDLP FGSCLFRSDV YDNGSSFLYD |
| 241 | NCTACTCRDS TVVCKRKCSH PGGCDQGQEG CCEECLLRVP PEDIKVCKFG NKIFQDGEMW |
| 381 | SSINCTICAC VKGRTECRNK QCIPISSCPQ GKILNRKGCC PICTEKPGVC TVFGDPHYNT |
| 361 | FDGRTFNFQG TCQYVLTKDC SSPASPFQVL VKNDARRTRS FSWTKSVELV LGESRVSLQQ |
| 421 | HLTVRWNGSR IALPCRAPHF HIDLDGYLLK VTTKAGLEIS WDGDSFVEVM AAPHLKGKLC |
| 481 | GLCGNYNGHK RDDLIGGDGN FKFDVDDFAE SWRVESNEFC NRPQRKPVPE LCQGTVKVKL |
| 541 | RAHRECQKLK SWEFQTCHST VDYATFYRSC VTDMCECPVH KNCYCESFLA YTRACQREGI |
| 601 | KVHWEPQQNC AATQCKHGAV YDTCGPGCIK TCDNWNEIGP CNKPCVAGCH CPANLVLHKG |
| 661 | RCIKPVLCPQ RTGGGTHTCP PCPAPELLGG PSVFLFPPKP KDTLMISRTP EVTCVVVDVS |
| 721 | HEDPEVKFNW YVDGVEVHNA KTKPREEQYN STYRVVSVLT VLHQDWLNGK EYKCKVSNKA |
| 781 | LPAPIEKTIS KAKGQPREPQ VYTLPPCREE MTKNQVSLWC LVKGFYPSDI AVEWESNGQP |
| 841 | ENNYKTTPPV LKSDGSFFLY SKLTVDKSRW QQGNVFSCSV MHEALHNHYT QKSLSLSPGK |

The leader sequence and linker are underlined. To promote formation of the BMPER-Fc:Fc heterodimer rather than either of the possible homodimeric complexes, two amino acid substitutions (replacing a serine with a cysteine and a threonine with a tryptophan) can be introduced into the Fc domain of the BMPER fusion polypeptide as indicated by double underline above. The amino acid sequence of SEQ ID NO: 534 may optionally be provided with the C-terminal lysine removed.

The mature BMPER-Fc fusion polypeptide (SEQ ID NO: 535) is as follows and may optionally be provided with the C-terminal lysine removed.

| (SEQ ID NO: 535) | |
|---|---|
| 1 | SSFLTGSVAK CENEGEVLQI PFITDNPCIM CVCLNKEVTC KREKCPVLSR DCALAIKQRG |
| 61 | ACCEQCKGCT YEGNTYNSSF KWQSPAEPCV LRQCQEGVVT ESGVRCVVHC KNPLEHLGMC |
| 121 | CPTCPGCVFE GVQYQEGEEF QPEGSKCTKC SCTGGRTQCV REVCPILSCP QHLSHIPPGQ |
| 181 | CCPKCLGQRK VFDLPFGSCL FRSDVYDNGS SFLYDNCTAC TCRDSTVVCK RKCSHPGGCD |
| 241 | QGQEGCCEEC LLRVPPEDIK VCKFGNKIFQ DGEMWSSINC TICACVKGRT ECRNKQCIPI |
| 301 | SSCPQGKILN RKGCCPICTE KPGVCTVFGD PHYNTFDGRT FNFQGTCQYV LTKDCSSPAS |
| 361 | PFQVLVKNDA RRTRSFSWTK SVELVLGESR VSLQQHLTVR WNGSRIALPC RAPHFHIDLD |
| 421 | GYLLKVTTKA GLEISWDGDS FVEVMAAPHL KGKLCGLCGN YNGHKRDDLI GGDGNFKFDV |
| 481 | DDFAESWRVE SNEFCNRPQR KPVPELCQGT VKVKLRAHRE CQKLKSWEFQ TCHSTVDYAT |
| 541 | FYRSCVTDMC ECPVHKNCYC ESFLAYTRAC QREGIKVHWE PQQNCAATQC KHGAVYDTCG |
| 601 | PGCIKTCDNW NEIGPCNKPC VAGCHCPANL VLHKGRCIKP VLCPQRTGGG THTCPPCPAP |
| 661 | ELLGGPSVFL FPPKPKDTLM ISRTPEVTCV VVDVSHEDPE VKFNWYVDGV EVHNAKTKPR |
| 721 | EEQYNSTYRV VSVLTVLHQD WLNGKEYKCK VSNKALPAPI EKTISKAKGQ PREPQVYTLP |
| 781 | PSRKEMTKNQ VSLTCLVKGF YPSDIAVEWE SNGQPENNYK TTPPVLDSDG SFFLYSKLTV |
| 841 | DKSRWQQGNV FSCSVMHEAL HNHYTQKSLS LSPGK |

As described in Example 1, the complementary form of monomeric G1Fc polypeptide (SEQ ID NO: 506) employs the TPA leader and incorporates an optional N-terminal extension. To promote formation of the BMPER-Fc:Fc heterodimer rather than either of the possible homodimeric complexes, four amino acid substitutions can be introduced into the monomeric Fc polypeptide as indicated. The amino acid sequence of SEQ ID NO: 506 and the mature Fc polypeptide (SEQ ID NO: 507) may optionally be provided with the C-terminal lysine removed.

The BMPER-Fc fusion polypeptide and monomeric Fc polypeptide of SEQ ID NO: 535 and SEQ ID NO: 507, respectively, may be co-expressed and purified from a CHO cell line to give rise to a single-arm heteromeric protein complex comprising BMPER-Fc:Fc.

Purification of various BMPER-Fc:Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 9. Generation of a Single-Arm RGMB-Fc Heterodimer

Applicants envision construction of a soluble single-arm RGMB-Fc heterodimeric complex comprising a monomeric Fc polypeptide with a short N-terminal extension and a second polypeptide in which a ligand-binding domain of human RGM-B is fused to a separate Fc domain with a linker positioned between a ligand-binding domain and this second Fc domain. The individual constructs are referred to as monomeric Fc polypeptide and RGMB-Fc fusion polypeptide, respectively, and the sequences for each are provided below.

Formation of a single-arm RGMB-Fc heterodimer may be guided by approaches similar to those described for single-arm endoglin-Fc heterodimer in Example 1. In a first approach, illustrated in the RGMB-Fc and monomeric Fc polypeptide sequences of SEQ ID NOs: 536-537 and 502-503, respectively, one Fc domain is altered to introduce cationic amino acids at the interaction face, while the other Fc domain is altered to introduce anionic amino acids at the interaction face.

The RGMB-Fc fusion polypeptide employs the TPA leader and is as follows:

| (SEQ ID NO: 536) | |
|---|---|
| 1 | MDAMKRGLCC VLLLCGAVFV SPGASGDCQQ PAQCRIQKCT TDFVSLTSHL NSAVDGFDSE |
| 61 | FCKALRAYAG CTQRTSKACR GNLVYHSAVL GISDLMSQRN CSKDGPTSST NPEVTHDPCN |
| 121 | YHSHAGAREH RRGDQNPPSY LFCGLFGDPH LRTFKDNFQT CKVEGAWPLI DNNYLSVQVT |
| 181 | NVPVVPGSSA TATNKITIIF KAHHECTDQK VYQAVTDDLP AAFVDGTTSG GDSDAKSLRI |
| 241 | VERESGHYVE MHARYIGTTV FVRQVGRYLT LAIRMPEDLA MSYEESQDLQ LCVNGCPLSE |
| 301 | RIDDGQGQVS AILGHSLPRT SLVQAWPGYT LETANTQCHE KMPVKDIYFQ SCVFDLLTTG |
| 361 | DANFTAAAHS ALEDVEALHP RKERWHIFPS STGGGTHTCP PCPAPELLGG PSVFLFPPKP |
| 421 | KDTLMISRTP EVTCVVVDVS HEDPEVKFMW YVDGVEVENA KTKPREEQYN STYRVVSVLT |
| 481 | VLHQDWLNGK EYKCKVSNKA LPAPIEKTIS KAKGQPREPQ VYTLPPSRKE MTKNQVSLTC |
| 541 | LVKGFYPSDI AVEWESNGQP ENNYKTTPPV LKSDGSFFLY SKLTVDKSRW QQGNVFSCSV |
| 601 | MHEALHNHYT QKSLSLSPGK |

The leader and linker sequences are underlined. To promote formation of the RGMB-Fc:Fc heterodimer rather than either of the possible homodimeric complexes (RGMB-Fc:RGMB-Fc or Fc:Fc), two amino acid substitutions (replacing anionic residues with lysines) can be introduced into the Fc domain of the fusion polypeptide as indicated by double underline above. The amino acid sequence of SEQ ID NO: 536 may optionally be provided with the C-terminal lysine removed.

The mature RGMB-Fc fusion polypeptide sequence is as follows (SEQ ID NO: 537) and may optionally be provided with the C-terminal lysine removed.

| (SEQ ID NO: 537) | |
|---|---|
| 1 | GDCQQPAQCR IQKCTTDFVS LTSHLNSAVD GFDSEFCKAL RAYAGCTQRT SKACRGNLVY |
| 61 | HSAVLGISDL MSQRNCSKDG PTSSTNPEVT HDPCNYHSHA GAREHRRGDQ NPPSYLFCGL |
| 121 | FGDPHLRTFK DNFQTCKVEG AWPLIDNNYL SVQVTNVPVV PGSSATATNK ITIIFKAHHE |
| 181 | CTDQKVYQAV TDDLPAAFVD GTTSGGDSDA KSLRIVERES GHYVEMHARY IGTTVFVRQV |
| 241 | GRYLTLAIRM PEDLAMSYEE SQDLQLCVNG CPLSERIDDG QGQVSAILGH SLPRTSLVQA |
| 301 | WPGYTLETAN TQCHEKMPVK DIYFQSCVFD LLTTGDANFT AAAHSALEDV EALHPRKERW |
| 361 | HIFPSSTGGG THTCPPCPAP ELLGGPSVFL FPPKPKDTLM ISRTPEVTCV VVDVSHEDPE |
| 421 | VKFNWYVDGV EVHNAKTKPR EEQYNSTYRV VSVLTVLHQD WLNGKEYKCK VSNKALPAPI |
| 481 | EKTISKAKGQ PREPQVYTLP PSRKEMTKNQ VSLTCLVKGF YPSDIAVEWE SNGQPENNYK |
| 541 | TTPPVLKSDG SFFLYSKLTV DKSRWQQGNV FSCSVMHEAL HNHYTQKSLS LSPGK |

As described in Example 1, the complementary form of monomeric human G1Fc polypeptide (SEQ ID NO: 502) employs the TPA leader and incorporates an optional N-terminal extension. To promote formation of the RGMB-Fc:Fc heterodimer rather than either of the possible homodimeric complexes, two amino acid substitutions (replacing lysines with anionic residues) can be introduced into the monomeric Fc polypeptide as indicated. The amino acid sequence of SEQ ID NO: 502 may optionally be provided with the C-terminal lysine removed. The mature monomeric Fc polypeptide (SEQ ID NO: 503) may optionally be provided with the C-terminal lysine removed.

The RGMB-Fc fusion polypeptide and monomeric Fc polypeptide of SEQ ID NO: 537 and SEQ ID NO: 503, respectively, may be co-expressed and purified from a CHO cell line to give rise to a single-arm heteromeric protein complex comprising RGMB-Fc:Fc.

In another approach to promoting the formation of heteromultimer complexes using asymmetric Fc fusion polypeptides, the Fc domains are altered to introduce complementary hydrophobic interactions and an additional intermolecular disulfide bond as illustrated in the RGMB-Fc and Fc polypeptide sequences of SEQ ID NOs: 538-539 and 506-507, respectively.

The RGMB-Fc fusion polypeptide (SEQ ID NO: 538) uses the TPA leader and is as follows:

| (SEQ ID NO: 538) | |
|---|---|
| 1 | MDAMKRGLCC VLLLCGAVFV SPGASGDCQQ PAQCRIQKCT TDFVSLTSHL NSAVDGFDSE |
| 61 | FCKALRAYAG CTQRTSKACR GNLVYHSAVL GISDLMSQRN CSKDGPTSST NPEVTHDPCN |
| 121 | YHSHAGAREH RRGDQNPPSY LFCGLFGDPH LRTFKDNFQT CKVEGAWPLI DNNYLSVQVT |
| 181 | NVPVVPGSSA TATNKITIIF KAHHECTDQK VYQAVTDDLP AAFVDGTTSG GDSDAKSLRI |
| 241 | VERESGHYVE MHARYIGTTV FVRQVGRYLT LAIRMPEDLA MSYEESQDLQ LCVNGCPLSE |
| 301 | RIDDGQGQVS AILGHSLPRT SLVQAWPGYT LETANTQCHE KMPVKDIYFQ SCVFDLLTTG |
| 361 | DANFTAAAHS ALEDVEALHP RKERWHIFPS STGGGTHTCP PCPAPELLGG PSVFLFPPKP |
| 421 | KDTLMISRTP EVTCVVVDVS HEDPEVKFMW YVDGVEVEHA KTKPREEQYN STYRVVSVLT |
| 481 | VLHQDWLNGK EYKCKVSNKA LPAPIEKTIS KAKGQPREPQ VYTLPPCREE MTKNQVSLWC |
| 541 | LVKGFYPSDI AVEWESNGQP ENNYKTTPPV LDSDGSFFLY SKLTVDKSRW QQGNVFSCSV |
| 601 | MHEALHNHYT QKSLSLSPGK |

The leader sequence and linker are underlined. To promote formation of the RGMB-Fc:Fc heterodimer rather than either of the possible homodimeric complexes, two amino acid substitutions (replacing a serine with a cysteine and a threonine with a tryptophan) can be introduced into the Fc domain of the RGMB fusion polypeptide as indicated by double underline above. The amino acid sequence of SEQ ID NO: 538 may optionally be provided with the C-terminal lysine removed.

The mature RGMB-Fc fusion polypeptide (SEQ ID NO: 539) is as follows and may optionally be provided with the C-terminal lysine removed.

| (SEQ ID NO: 539) | |
|---|---|
| 1 | GDCQQPAQCR IQKCTTDFVS LTSHLNSAVD GFDSEFCKAL RAYAGCTQRT SKACRGNLVY |
| 61 | HSAVLGISDL MSQRNCSKDG PTSSTNPEVT HDPCNYHSHA GAREHRRGDQ NPPSYLFCGL |
| 121 | FGDPHLRTFK DNFQTCKVEG AWPLIDNNYL SVQVTNVPVV PGSSATATNK ITIIFKAHHE |
| 181 | CTDQKVYQAV TDDLPAAFVD GTTSGGDSDA KSLRIVERES GHYVEMHARY IGTTVFVRQV |
| 241 | GRYLTLAIRM PEDLAMSYEE SQDLQLCVNG CPLSERIDDG QGQVSAILGH SLPRTSLVQA |
| 301 | WPGYTLETAN TQCHEKMPVK DIYFQSCVFD LLTTGDANFT AAAHSALEDV EALHPRKERW |
| 361 | HIFPSSTGGG THTCPPCPAP ELLGGPSVFL FPPKPKDTLM ISRTPEVTCV VVDVSHEDPE |
| 421 | VKFNWYVDGV EVHNAKTKPR EEQYNSTYRV VSVLTVLHQD WLNGKEYKCK VSNKALPAPI |
| 481 | EKTISKAKGQ PREPQVYTLP PCREEMTKNQ VSLWCLVKGF YPSDIAVEWE SNGQPENNYK |
| 541 | TTPPVLKSDG SFFLYSKLTV DKSRWQQGNV FSCSVMHEAL HNHYTQKSLS LSPGK |

As described in Example 1, the complementary form of monomeric G1Fc polypeptide (SEQ ID NO: 506) employs the TPA leader and incorporates an optional N-terminal extension. To promote formation of the RGMB-Fc:Fc heterodimer rather than either of the possible homodimeric complexes, four amino acid substitutions can be introduced into the monomeric Fc polypeptide as indicated. The amino acid sequence of SEQ ID NO: 506 and the mature Fc polypeptide (SEQ ID NO: 507) may optionally be provided with the C-terminal lysine removed.

The RGMB-Fc fusion polypeptide and monomeric Fc polypeptide of SEQ ID NO: 539 and SEQ ID NO: 507, respectively, may be co-expressed and purified from a CHO cell line to give rise to a single-arm heteromeric protein complex comprising RGMB-Fc:Fc.

Purification of various RGMB-Fc:Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 10. Generation of a Single-Arm RGMA-Fc Heterodimer

Applicants envision construction of a soluble single-arm RGMA-Fc heterodimeric complex comprising a monomeric Fc polypeptide with a short N-terminal extension and a second polypeptide in which a ligand-binding domain of human RGM-A is fused to a separate Fc domain with a linker positioned between a ligand-binding domain and this second Fc domain. The individual constructs are referred to as monomeric Fc polypeptide and RGMA-Fc fusion polypeptide, respectively, and the sequences for each are provided below. Applicants also envision additional single-arm RGMA-Fc heterodimeric complexes comprising a ligand-binding domain of RGM-A isoforms 2 or 3 (SEQ ID NOs: 66 or 70).

Formation of a single-arm RGMA-Fc heterodimer may be guided by approaches similar to those described for single-arm endoglin-Fc heterodimer in Example 1. In a first approach, illustrated in the RGMA-Fc and monomeric Fc polypeptide sequences of SEQ ID NOs: 540-541 and 502-503, respectively, one Fc domain is altered to introduce cationic amino acids at the interaction face, while the other Fc domain is altered to introduce anionic amino acids at the interaction face.

The RGMA-Fc fusion polypeptide employs the TPA leader and is as follows:

| (SEQ ID NO: 540) | |
|---|---|
| 1 | MDAMKRGLCC VLLLCGAVFV SPGASCKILK CNSEFWSATS GSHAPASDDT PEFCAALRSY |
| 61 | ALCTRRTART CRGDLAYHSA VHGIEDLMSQ HNCSKDGPTS QPRLRTLPPA GDSQERSDSP |
| 121 | EICHYEKSFH KHSATPNYTH CGLFGDPHLR TFTDRFQTCK VQGAWPLIDN NYLNVQVTNT |
| 181 | PVLPGSAATA TSKLTIIFKN FQECVDQKVY QAEMDELPAA FVDGSKNGGD KHGANSLKIT |
| 241 | EKVSGQHVEI QAKYIGTTIV VRQVGRYLTF AVRMPEEVVN AVEDWDSQGL YLCLRGCPLN |
| 301 | QQIDFQAFHT NAEGTGARRL AAASPAPTAP ETFPYETAVA KCKEKLPVED LYYQACVFDL |
| 361 | LTTGDVNFTL AAYYALEDVK MLHSTGGGTH TCPPCPAPEL LGGPSVFLFP PKPKDTLMIS |
| 421 | RTPEVTCVVV DVSHEDPEVK FNWYVDGVEV HNAKTKPREE QYNSTRYVVS VLTVLHQDWL |
| 481 | NGKEYKCKVS NKALPAPIEK TISKAKGQPR EPQVYTLPPS RKEMTKNQVS LTCLVKGFYP |
| 541 | SDIAVEWESN GQPENNYKTT PPVLKSDGSF FLYSKLTVDK SRWQQGNVFS CSVMHEALHN |
| 601 | HYTQKSLSLS PGK |

The leader and linker sequences are underlined. To promote formation of the RGMA-Fc:Fc heterodimer rather than either of the possible homodimeric complexes (RGMA-Fc:RGMA-Fc or Fc:Fc), two amino acid substitutions (replacing anionic residues with lysines) can be introduced into the Fc domain of the fusion polypeptide as indicated by double underline above. The amino acid sequence of SEQ ID NO: 540 may optionally be provided with the C-terminal lysine removed.

The mature RGMA-Fc fusion polypeptide sequence is as follows (SEQ ID NO: 541) and may optionally be provided with the C-terminal lysine removed.

| (SEQ ID NO: 541) | |
|---|---|
| 1 | CKILKCNSEF WSATSGSHAP ASDDTPEFCA ALRSYALCTR RTARTCRGDL AYHSAVHGIE |
| 61 | DLMSQHNCSK DGPTSQPRLR TLPPAGDSQE RSDSPEICHY EKSFHKHSAT PNYTHCGLFG |
| 121 | DPHLRTFTDR FQTCKVQGAW PLIDNNYLNV QVTVTPVLPG SAATATSKLT IIFKNFQECV |
| 181 | DQKVYAQEMD ELPAAFVDGS KNGGDKHGAN SLKITEKVSG QHVEIQAKYI GTTIVVRQVG |
| 241 | RYLTFAVRMP EEVVNAVEDW DSQGLYLCLR GCPLNQQIDF QAFHTNAEGT GARRLAAASP |
| 301 | APTAPETFPY ETAVAKCKEK LPVEDLYYQA CVFDLLTTGD VNFTLAAYYA LEDMKMLHST |
| 361 | GGGTHTCPPC PAPELLGGPS VFLFPPKPKD TLMISRTPEV TCVVVDVSHE DPEVKFNWYV |
| 421 | DGVEVHNAKT KPREEQYNST YRVVSVLTVL HQDWLNGKEY KCKVSNKALP APIEKTISKA |
| 481 | KGQPREPQVY TLPPSRKEMT KNQVSLTCLV KGFYPSDIAV EWESNGQPEN NYKTTPPVLK |
| 541 | SDGSFFLYSK LTVDKSRWQQ GNVFSCSMNH EALHNHYTQK SLSLSPGK |

As described in Example 1, the complementary form of monomeric human G1Fc polypeptide (SEQ ID NO: 502) employs the TPA leader and incorporates an optional N-terminal extension. To promote formation of the RGMA-Fc:Fc heterodimer rather than either of the possible homodimeric complexes, two amino acid substitutions (replacing lysines with anionic residues) can be introduced into the monomeric Fc polypeptide as indicated. The amino acid sequence of SEQ ID NO: 502 may optionally be provided with the C-terminal lysine removed. The mature monomeric Fc polypeptide (SEQ ID NO: 503) may optionally be provided with the C-terminal lysine removed.

The RGMA-Fc fusion polypeptide and monomeric Fc polypeptide of SEQ ID NO: 541 and SEQ ID NO: 503, respectively, may be co-expressed and purified from a CHO cell line to give rise to a single-arm heteromeric protein complex comprising RGMA-Fc:Fc.

In another approach to promoting the formation of heteromultimer complexes using asymmetric Fc fusion polypeptides, the Fc domains are altered to introduce complementary hydrophobic interactions and an additional intermolecular disulfide bond as illustrated in the RGMA-Fc and Fc polypeptide sequences of SEQ ID NOs: 542-543 and 506-507, respectively.

The RGMA-Fc fusion polypeptide (SEQ ID NO: 542) uses the TPA leader and is as follows:

| (SEQ ID NO: 542) | |
|---|---|
| 1 | MDAMKRGLCC VLLLCGAVFV SPGASCKILK CNSEFWSATS GSHAPASDDT PEFCAALRSY |
| 61 | ALCTRRTART CRGDLAYHSA VHGIEDLMSQ HNCSKDGPTS QPRLRTLPPA GDSQERSDSP |
| 121 | EICHYEKSFH KHSATPNYTH CGLFGDPHLR TFTDRFQTCK VQGAWPLIDN NYLNVQVTNT |
| 181 | PVLPGSAATA TSKLTIIFKN FQECVDQKVY QAEMDELPAA FVDGSKNGGD KHGANSLKIT |
| 241 | EKVSGQHVEI QAKYIGTTIV VRQVGRYLTF AVRMPEEVVN AVEDWDSQGL YLCLRGCPLN |
| 301 | QQIDFQAFHT NAEGTGARRL AAASPAPTAP ETFPYETAVA KCKEKLPVED LYYQACVFDL |
| 361 | LTTGDVNFTL AAYYALEDVK MLHSTGGGTH TCPPCPAPEL LGGPSVFLFP PKPKDTLMIS |
| 421 | RTPEVTCVVV DVSHEDPEVK FNWYVDGVEV HNAKTKPREE QYNSTRYVVS VLTVLHQDWL |
| 481 | NGKEYKCKVS NKALPAPIEK TISKAKGQPR EPQVYTLPPC REEMTKNQVS LWCLVKGFYP |
| 541 | SDIAVEWESN GQPENNYKTT PPVLDSDGSF FLYSKLTVDK SRWQQGNVFS CSVMHEALHN |
| 601 | HYTQKSLSLS PGK |

The leader sequence and linker are underlined. To promote formation of the RGMA-Fc:Fc heterodimer rather than either of the possible homodimeric complexes, two amino acid substitutions (replacing a serine with a cysteine and a threonine with a tryptophan) can be introduced into the Fc domain of the RGMA fusion polypeptide as indicated by double underline above. The amino acid sequence of SEQ ID NO: 542 may optionally be provided with the C-terminal lysine removed.

The mature RGMA-Fc fusion polypeptide (SEQ ID NO: 543) is as follows and may optionally be provided with the C-terminal lysine removed.

| (SEQ ID NO: 543) | |
|---|---|
| 1 | CKILKCNSEF WSATSGSHAP ASDDTPEFCA ALRSYALCTR RTARTCRGDL AYHSAVHGIE |
| 61 | DLMSQHNCSK DGPTSQPRLR TLPPAGDSQE RSDSPEICHY EKSFHKHSAT PNYTHCGLFG |
| 121 | DPHLRTFTDR FQTCKVQGAW PLIDNNYLNV QVTVTPVLPG SAATATSKLT IIFKNFQECV |
| 181 | DQKVYAQEMD ELPAAFVDGS KNGGDKHGAN SLKITEKVSG QHVEIQAKYI GTTIVVRQVG |
| 241 | RYLTFAVRMP EEVVNAVEDW DSQGLYLCLR GCPLNQQIDF QAFHTNAEGT GARRLAAASP |
| 301 | APTAPETFPY ETAVAKCKEK LPVEDLYYQA CVFDLLTTGD VNFTLAAYYA LEDVKMLHST |
| 361 | GGGTHTCPPC PAPELLGGPS VFLFPPKPKD TLMISRTPEV TCVVVDVSHE DPEVKFNWYV |
| 421 | DGVEVHNAKT KPREEQYNST YRVVSVLTVL HQDWLNGKEY KCKVSNKALP APIEKTISKA |
| 481 | KGQPREPQVY TLPPCREEMT KNQVSLWCLV KGFYPSDIAV EWESNGQPEN NYKTTPPVLD |
| 541 | SDGSFFLYSK LTVDKSRWQQ GNVFSCSMNH EALHNHYTQK SLSLSPGK |

As described in Example 1, the complementary form of monomeric G1Fc polypeptide (SEQ ID NO: 506) employs the TPA leader and incorporates an optional N-terminal extension. To promote formation of the RGMA-Fc:Fc heterodimer rather than either of the possible homodimeric complexes, four amino acid substitutions can be introduced into the monomeric Fc polypeptide as indicated. The amino acid sequence of SEQ ID NO: 506 and the mature Fc polypeptide (SEQ ID NO: 507) may optionally be provided with the C-terminal lysine removed.

The RGMA-Fc fusion polypeptide and monomeric Fc polypeptide of SEQ ID NO: 543 and SEQ ID NO: 507, respectively, may be co-expressed and purified from a CHO cell line to give rise to a single-arm heteromeric protein complex comprising RGMA-Fc:Fc.

Purification of various RGMA-Fc:Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 11. Generation of a Single-Arm HEMO-Fc Heterodimer

Applicants envision construction of a soluble single-arm HEMO-Fc heterodimeric complex comprising a monomeric Fc polypeptide with a short N-terminal extension and a second polypeptide in which a ligand-binding domain of human hemojuvelin is fused to a separate Fc domain with a linker positioned between a ligand-binding domain and this second Fc domain. The individual constructs are referred to as monomeric Fc polypeptide and HEMO-Fc fusion polypeptide, respectively, and the sequences for each are provided below. Applicants also envision additional single-arm HEMO-Fc heterodimeric complexes comprising a ligand-binding domain of hemojuvelin isoforms 2 or 3 (SEQ ID NOs: 78 or 82).

Formation of a single-arm HEMO-Fc heterodimer may be guided by approaches similar to those described for single-arm endoglin-Fc heterodimer in Example 1. In a first approach, illustrated in the HEMO-Fc and monomeric Fc polypeptide sequences of SEQ ID NOs: 544-545 and 502-503, respectively, one Fc domain is altered to introduce cationic amino acids at the interaction face, while the other Fc domain is altered to introduce anionic amino acids at the interaction face.

The HEMO-Fc fusion polypeptide employs the TPA leader and is as follows:

| (SEQ ID NO: 544) | |
|---|---|
| 1 | MDAMKRGLCC VLLLCGAVFV SPGASQCKIL RCNAEYVSST LSLRGGGSSG ALRGGGGGGR |
| 61 | GGGVGSGGLC RALRSYALCT RRTARTCRGD LAFHSAVHGI EDLMIQHNCS RQGPTAPPPP |
| 121 | RGPALPGAGS GLPAPDPCDY EGRFSRLHGR PPGFLHCASF GDPHVRSFHH HFHTCRVQGA |
| 181 | WPLLDNDFLF VQATSSPMAL GANATATRKL TIIFKNMQEC IKQKVYQAEV DNLPVAFEDG |
| 241 | SINGGDRPGG SSLSIQTANP GNHVEIQAAY IGTTIIIRQT AGQLSFSIKV AEDVAMAFSA |
| 301 | EQDLQLCVGG CPPSQRLSRS ERNRRGAITI DTARRLCKEG LPVEDAYFHS CVFDVLISGD |
| 361 | PNFTVAAQAA LEDARAFLPD LEKLHLFPSD TGGGTHTCPP CPAPELLGGP SVFLFPPKPK |
| 421 | DTLMISRTPE VTCVVVDVSH EDPEVKFNWY VDGVEVHNAK TKPREEQYNS TYRVVSVLTV |
| 481 | LHQDWLNGKE YKCKVSNKAL PAPIEKTISK AKGQPREPQV YTLPPSRKEM TKNQVSLTCL |
| 541 | VKGFYPSDIA VEWESNGQPE NNYKTTPPVL KSDGSFFLYS KLTVDKSRWQ QGNVFSCSVM |
| 601 | HEALHNHYTQ KSLSLSPGK |

The leader and linker sequences are underlined. To promote formation of the HEMO-Fc:Fc heterodimer rather than either of the possible homodimeric complexes (HEMO-Fc:HEMO-Fc or Fc:Fc), two amino acid substitutions (replacing anionic residues with lysines) can be introduced into the Fe domain of the fusion polypeptide as indicated by double underline above. The amino acid sequence of SEQ ID NO: 544 may optionally be provided with the C-terminal lysine removed.

The mature HEMO-Fc fusion polypeptide sequence is as follows (SEQ ID NO: 545) and may optionally be provided with the C-terminal lysine removed.

| (SEQ ID NO: 545) | |
|---|---|
| 1 | QCKILRCNAE YVSSTLSLRG GGSSGALRGG GGGGRGGGVG SGGLCRALRS YALCTRRTAR |
| 61 | TCRGDLAFHS AVHGIEDLMI QHNCSRQGPT APPPPRGPAL PGAGSGLPAP DPCDYEGRFS |
| 121 | RLHGRPPGFL HCASFGDPHY RSFHHHFHTC RVQGAWPLLD NDFLFVQATS SPMALGANAT |
| 181 | ATRKLTIIFK NMQECIDQKV YQAEVDNLPV AFEDGSINGG DRPGGSSLSI QTANPGNHVE |
| 241 | IQAAYIGTTI IIRQTAGQLS FSIKVAEDVA MAFSAEQDLQ LCVGGCPPSQ RLSRSERNRR |
| 301 | GAITIDTARR LCKEGLPVED AYFHSCVFDV LISGDPNFTV AAQAALEDAR AFLPDLEKLH |
| 361 | LFPSDTGGGT HTCPPCPAPE LLGGPSVFLF PPKPKDTLMI SRTPEVTCVV VDVSHEDPEV |
| 421 | KFNWYVDGVE VHNAKTKPRE EQYNSTYRVV SVLTVLHQDW LNGKEYKCKV SNKALPAPIE |
| 481 | KTISKAKGQP REPQVYTLPP SRKEMTKNQV SLTCLVKGFY PSDIAVEWES NGQPENNYKT |
| 541 | TPPVLKSDGS FFLYSKLTVD KSRWQQGNVF SCSVMHEALH NHYTQKSLSL SPGK |

As described in Example 1, the complementary form of monomeric human G1Fc polypeptide (SEQ ID NO: 502) employs the TPA leader and incorporates an optional N-terminal extension. To promote formation of the HEMO-Fc:Fc heterodimer rather than either of the possible homodimeric complexes, two amino acid substitutions (replacing lysines with anionic residues) can be introduced into the monomeric Fc polypeptide as indicated. The amino acid sequence of SEQ ID NO: 502 may optionally be provided with the C-terminal lysine removed. The mature monomeric Fc polypeptide (SEQ ID NO: 503) may optionally be provided with the C-terminal lysine removed.

The HEMO-Fc fusion polypeptide and monomeric Fc polypeptide of SEQ ID NO: 545 and SEQ ID NO: 503, respectively, may be co-expressed and purified from a CHO cell line to give rise to a single-arm heteromeric protein complex comprising HEMO-Fc:Fc.

In another approach to promoting the formation of heteromultimer complexes using asymmetric Fc fusion polypeptides, the Fc domains are altered to introduce complementary hydrophobic interactions and an additional intermolecular disulfide bond as illustrated in the HEMO-Fc and Fc polypeptide sequences of SEQ ID NOs: 546-547 and 506-507, respectively.

The HEMO-Fc fusion polypeptide (SEQ ID NO: 546) uses the TPA leader and is as follows:

| (SEQ ID NO: 546) | |
|---|---|
| 1 | MDAMKRGLCC VLLLCGAVFV SPGASQCKIL RCNAEYVSST LSLRGGGSSG ALRGGGGGGR |
| 61 | GGGVGSGGLC RALRSYALCT RRTARTCRGD LAFHSAVHGI EDLMIQHNCS RQGPTAPPPP |
| 121 | RGPALPGAGS GLPAPDPCDY EGRFSRLHGR PPGFLHCASF GDPHVRSFHH HFHTCRVQGA |
| 181 | WPLLDNDFLF VQATSSPMAL GANATATRKL TIIFKNMQEC IKQKVYQAEV DNLPVAFEDG |
| 241 | SINGGDRPGG SSLSIQTANP GNHVEIQAAY IGTTIIIRQT AGQLSFSIKV AEDVAMAFSA |
| 301 | EQDLQLCVGG CPPSQRLSRS ERNRRGAITI DTARRLCKEG LPVEDAYFHS CVFDVLISGD |
| 361 | PNFTVAAQAA LEDARAFLPD LEKLHLFPSD TGGGTHTCPP CPAPELLGGP SVFLFPPKPK |
| 421 | DTLMISRTPE VTCVVVDVSH EDPEVKFNWY VDGVEVHNAK TKPREEQYNS TYRVVSVLTV |
| 481 | LHQDWLNGKE YKCKVSNKAL PAPIEKTISK AKGQPREPQV YTLPPCREEM TKNQVSLWCL |
| 541 | VKGFYPSDIA VEWESNGQPE NNYKTTPPVL DSDGSFFLYS KLTVDKSRWQ QGNVFSCSVM |
| 601 | HEALHNHYTQ KSLSLSPGK |

The leader sequence and linker are underlined. To promote formation of the HEMO-Fc:Fc heterodimer rather than either of the possible homodimeric complexes, two amino acid substitutions (replacing a serine with a cysteine and a threonine with a tryptophan) can be introduced into the Fc domain of the hemojuvelin fusion polypeptide as indicated by double underline above. The amino acid sequence of SEQ ID NO: 546 may optionally be provided with the C-terminal lysine removed.

The mature HEMO-Fc fusion polypeptide (SEQ ID NO: 547) is as follows and may optionally be provided with the C-terminal lysine removed.

| (SEQ ID NO: 547) | |
|---|---|
| 1 | QCKILRCNAE YVSSTLSLRG GGSSGALRGG GGGGRGGGVG SGGLCRALRS YALCTRRTAR |
| 61 | TCRGDLAFHS AVHGIEDLMI QHNCSRQGPT APPPPRGPAL PGAGSGLPAP DPCDYEGRFS |
| 121 | RLHGRPPGFL HCASFGDPHV RSFHHHFHTC RVQGAWPLLD NDFLFVQATS SPMALGANAT |
| 181 | ATRKLTIIFK NMQECIDQKV YQAEVDNLPV AFEDGSINGG DRPGGSSLSI QTANPGNHVE |
| 241 | IQAAYIGTTI IIRQTAGQLS FSIKVAEDVA MAFSAEQDLQ LCVGGCPPSQ RLSRSERNRR |
| 301 | GAITIDTARR LCKEGLPVED AYFHSCVFDV LISGDPNFTV AAQAALEDAR AFLPDLEKLH |
| 361 | LFPSDTGGGT HTCPPCPAPE LLGGPSVFLF PPKPKDTLMI SRTPEVTCVV VDVSHEDPEV |
| 421 | KFNWYVDGVE VHNAKTKPRE EQYNSTYRVV SVLTVLHQDW LNGKEYKCKV SNKALPAPIE |
| 481 | KTISKAKGQP REPQVYTLPP CREEMTKNQV SLWCLVKGFY PSDIAVEWES NGQPENNYKT |
| 541 | TPPVLDSDGS FFLYSKLTVD KSRWQQGNVF SCSVMHEALH NHYTQKSLSL SPGK |

As described in Example 1, the complementary form of monomeric G1Fc polypeptide (SEQ ID NO: 506) employs the TPA leader and incorporates an optional N-terminal extension. To promote formation of the HEMO-Fc:Fc heterodimer rather than either of the possible homodimeric complexes, four amino acid substitutions can be introduced into the monomeric Fc polypeptide as indicated. The amino acid sequence of SEQ ID NO: 506 and the mature Fc polypeptide (SEQ ID NO: 507) may optionally be provided with the C-terminal lysine removed.

The HEMO-Fc fusion polypeptide and monomeric Fc polypeptide of SEQ ID NO: 547 and SEQ ID NO: 507, respectively, may be co-expressed and purified from a CHO cell line to give rise to a single-arm heteromeric protein complex comprising HEMO-Fc:Fc.

Purification of various HEMO-Fc:Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 12. Generation of a Single-Arm BG-Fc Heterodimer

Applicants envision construction of a soluble single-arm BG-Fc heterodimeric complex comprising a monomeric Fc polypeptide with a short N-terminal extension and a second polypeptide in which a ligand-binding domain of human betaglycan is fused to a separate Fc domain with a linker positioned between a ligand-binding domain and this second Fc domain. The individual constructs are referred to as monomeric Fc polypeptide and BG-Fc fusion polypeptide, respectively, and the sequences for each are provided below. Applicants also envision additional single-arm BG-Fc heterodimeric complexes comprising a ligand-binding domain of betaglycan isoform 2 (SEQ ID NO: 90).

Formation of a single-arm BG-Fc heterodimer may be guided by approaches similar to those described for single-arm endoglin-Fc heterodimer in Example 1. In a first approach, illustrated in the BG-Fc and monomeric Fc polypeptide sequences of SEQ ID NOs: 548-549 and 502-503, respectively, one Fc domain is altered to introduce cationic amino acids at the interaction face, while the other Fc domain is altered to introduce anionic amino acids at the interaction face.

The BG-Fc fusion polypeptide employs the TPA leader and is as follows:

| (SEQ ID NO: 548) | |
|---|---|
| 1 | MDAMKRGLCC VLLLCGAVFV SPGASGPEPG ALCELSPVSA SHPVQALMES FTVLSGCASR |
| 61 | GTTGLPQEVH VLNLRTAGQG PGQLQREVTL HLNPISSVHI HHKSVVFLLN SPHPLVWHLK |
| 121 | TERLATGVSR LFLVSEGSVV QFSSANFSLT AETEERNFPH GNEELLNWAR KEYGAVTSFT |
| 181 | ELKIARNIYI KVGEDQVFPP KCNIGKNFLS LNYLAEYLQP KAAEGCVMSS QPQNEEVHII |
| 241 | ELITPNSNPY SAFQVDITID IRPSQEDLEV VKNLILILKC KKSVNWVIKS FDVKGSLKII |
| 301 | APNSIGFGKE SERSMTMTKS IRDDIPSTQG NLVKWALDNG YSPITSYTMA PVANRFHLRL |
| 361 | ENNAEEMGDE EVHTIPPELR ILLDPGALPA LQNPPIRGGE GQNGGLPFPF PDISRRVWNE |
| 421 | EGEDGLPRPK DPVIPSIQLF PGLREPEEVQ GSVDIALSVK CDNEKMIVAV EKDSFQASGY |
| 481 | SGMDVTLLDP TCKAKMNGTH FVLESPLNGC GTRPRWSALD GVVYYNSIVI QVPALGDSSG |
| 541 | WPDGYEDLES GDNGFPGDMD EGDASLFTRP EIVVFNCSLQ QVRNPSSFQE QPHGNITFNM |
| 601 | ELYNTDLFLV PSQGVFSVPE NGHVYVEVSV TKAEQELGFA IQTCFISPYS NPDRMSHYTI |
| 661 | IENICPKDES VKFYSPKRVH FPIPQADMDK KRFSFVFKPV FNTSLLFLQC ELTLCTKMEK |
| 721 | HPQKLPKCVP PDEACTSLDA SIIWAMMQNK KTFTKPLAVI HHEAESKEKG PSMKEPNPIS |
| 781 | PPIFHGLDTL TVTGGGTHTC PPCPAPELLG GPSVFLFPPK PKDTLMISRT PEVTCVVVDV |
| 841 | SHEDPEVKFN WYVDGVEVHN AKTKPREEQY NSTYRVVSVL TVLHQDWLNG KEYKCKVSNK |
| 901 | ALPAPIEKTI SKAKGQPREP QVYTLPPSRK EMTKNQVSLT CLVKGFYPSD IAVEWESNGQ |
| 961 | PENNYKTTPP VLKSDGSFFL YSKLTVDKSR WQQGNVFSCS VMHEALHNHY TQKSLSLSPG |
| 1021 | K |

The leader and linker sequences are underlined. To promote formation of the BG-Fc:Fc heterodimer rather than either of the possible homodimeric complexes (BG-Fc:BG-Fc or Fc:Fc), two amino acid substitutions (replacing anionic residues with lysines) can be introduced into the Fc domain of the fusion polypeptide as indicated by double underline above. The amino acid sequence of SEQ ID NO: 548 may optionally be provided with the C-terminal lysine removed.

The mature BG-Fc fusion polypeptide sequence is as follows (SEQ ID NO: 549) and may optionally be provided with the C-terminal lysine removed.

| (SEQ ID NO: 549) | |
|---|---|
| 1 | SGPEPGALCE LSPVSASHPV QALMESFTVL SGCASRGTTG LPQEVHVLNL RTAGQGPGQL |
| 61 | QREVTLHLNP ISSVHIHHKS VVFLLNSPHP LVWHLKTERL ATGVSRLFLV SEGSVVQFSS |
| 121 | ANFSLTAETE ERNFPHGNEH LLNWARKEYG AVTSFTELKI ARNIYIKVGE DQVFPPKCNI |
| 181 | GKNFLSLNYL AEYLQPKAAE GCVMSSQPQN EEVHIIELIT PNSNPYSAFQ VDITIDIRPS |
| 241 | QEDLEVVKNL ILILKCKKSV NWVIKSFDVK GSLKIIAPNS IGFGKESERS MTMTKSIRDD |
| 301 | IPSTQGNLVK WALDNGYSPI TSYTMAPVAN RFHLRLENNA EEMGDEEVHT IPPELRILLD |
| 361 | PGALPALQNP PIRGGEGQNG GLPFPFPDIS RRVWNEEGED GLPRPKDPVI PSIQLFPGLR |
| 421 | EPEEVQGSVD IALSVKCDNE KMIVAVEKDS FQASGYSGMD VTLLDPTCKA KMNGTHFVLE |
| 481 | SPLNGCGTRP RWSALDGVVY YNSIVIQVPA LGDSSGWPDG YEDLESGDNG FPGDMDEGDA |
| 541 | SLFTRPEIVV FNCSLQQVRN PSSFQEQPHG NITFNMELYN TDLFLVPSQG VFSVPENGHV |
| 601 | YVEVSVTKAE QELGFAIQTC FISPYSNPDR MSHYTIIENI CPKDESVKFY SPKRVHFPIP |
| 661 | QADMDKKRFS FVFKPVFNTS LLFLQCELTL CTKMEKHPQK LPKCVPPDEA CTSLDASIIW |
| 721 | AMMQNKKTFT KPLAVIHHEA ESKEKGPSMK EPNPISPPIF HGLDTLTVTG GGTHTCPPCP |
| 781 | APELLGGPSV FLFPPKPKDT LMISRTPEVT CVVVDVSHED PEVKFNWYVD GVEVHNAKTK |
| 841 | PREEQYNSTY RVVSVLTVLH QDWLNGKEYK CKVSNKALPA PIEKTISKAK GQPREPQVYT |
| 901 | LPPSRKEMTK NQVSLTCLVK GFYPSDIAVE WESNGQPENN YKTTPPVLKS DGSFFLYSKL |
| 961 | TVDKSRWQQG NVFSCSVMHE ALHNHYTQKS LSLSPGK |

As described in Example 1, the complementary form of monomeric human G1Fc polypeptide (SEQ ID NO: 502) employs the TPA leader and incorporates an optional N-terminal extension. To promote formation of the BG-Fc:Fc heterodimer rather than either of the possible homodimeric complexes, two amino acid substitutions (replacing lysines with anionic residues) can be introduced into the monomeric Fc polypeptide as indicated. The amino acid sequence of SEQ ID NO: 502 may optionally be provided with the C-terminal lysine removed. The mature monomeric Fc polypeptide (SEQ ID NO: 503) may optionally be provided with the C-terminal lysine removed.

The BG-Fc fusion polypeptide and monomeric Fc polypeptide of SEQ ID NO: 549 and SEQ ID NO: 503, respectively, may be co-expressed and purified from a CHO cell line to give rise to a single-arm heteromeric protein complex comprising BG-Fc:Fc.

In another approach to promoting the formation of heteromultimer complexes using asymmetric Fc fusion polypeptides, the Fc domains are altered to introduce complementary hydrophobic interactions and an additional intermolecular disulfide bond as illustrated in the BG-Fc and Fc polypeptide sequences of SEQ ID NOs: 550-551 and 506-507, respectively.

The BG-Fc fusion polypeptide (SEQ ID NO: 550) uses the TPA leader and is as follows:

| (SEQ ID NO: 550) | |
|---|---|
| 1 | MDAMKRGLCC VLLLCGAVFV SPGASGPEPG ALCELSPVSA SHPVQALMES FTVLSGCASR |
| 61 | GTTGLPQEVH VLNLRTAGQG PGQLQREVTL HLNPISSVHI HHKSVVFLLN SPHPLVWHLK |
| 121 | TERLATGVSR LFLVSEGSVV QFSSANFSLT AETEERNFPH GNEHLLNWAR KEYGAVTSFT |
| 181 | ELKIARNIYI KVGEDQVFPP KCNIGKNFLS LNYLAEYLQP KAAEGCVMSS QPQNEEVHII |
| 241 | ELITPNSNPY SAFQVDITID IRPSQEDLEV VKNLILILKC KKSVNWVIKS FDVKGSLKII |
| 301 | APNSIGFGKE SERSMTMTKS IRDDIPSTQG NLVKWALDNG YSPITSYTMA PVANRFHLRL |
| 361 | ENNAEEMGDE EVHTIPPELR ILLDPGALPA LQNPPIRGGE GQNGGLPFPF PDISRRVWNE |
| 421 | EGEDGLPRPK DPVIPSIQLF PGLREPEEVQ GSVDIALSVK CDNEKMIVAV EKDSFQASGY |
| 481 | SGMDVTLLDP TCKAKMNGTH FVLESPLNGC GTRPRWSALD GVVYYNSIVI QVPALGDSSG |
| 541 | WPDGYEDLES GDNGFPGDMD EGDASLFTRP EIVVFNCSLQ QVRNPSSFQE QPHGNITFNM |
| 601 | ELYNTDLFLV PSQGVFSVPE NGHVYVEVSV TKAEQELGFA IQTCFISPYS NPDRMSHYTI |
| 661 | IENICPKDES VKFYSPKRVH FPIPQADMDK KRFSFVFKPV FNTSLLFLQC ELTLCTKMEK |
| 721 | HPQKLPKCVP PDEACTSLDA SIIWAMMQNK KTFTKPLAVI HHEAESKEKG PSMKEPNPIS |
| 781 | PPIFHGLDTL TVTGGGTHTC PPCPAPELLG GPSVFLFPPK PKDTLMISRT PEVTCVVVDV |
| 841 | SHEDPEVKFN WYVDGVEVHN AKTKPREEQY NSTYRVVSVL TVLHQDWLNG KEYKCKVSNK |
| 901 | ALPAPIEKTI SKAKGQPREP QVYTLPPCRE EMTKNQVSLW CLVKGFYPSD IAVEWESNGQ |
| 961 | PENNYKTTPP VLDSDGSFFL YSKLTVDKSR WQQGNVFSCS VMHEALHNHY TQKSLSLSPG |
| 1021 | K |

The leader sequence and linker are underlined. To promote formation of the BG-Fc:Fc heterodimer rather than either of the possible homodimeric complexes, two amino acid substitutions (replacing a serine with a cysteine and a threonine with a tryptophan) can be introduced into the Fe domain of the betaglycan fusion polypeptide as indicated by double underline above. The amino acid sequence of SEQ ID NO: 550 may optionally be provided with the C-terminal lysine removed.

The mature BG-Fc fusion polypeptide (SEQ ID NO: 551) is as follows and may optionally be provided with the C-terminal lysine removed.

| (SEQ ID NO: 551) | |
|---|---|
| 1 | GPEPGALCEL SPVSASHPVQ ALMESFTVLS GCASRGTTGL PQEVHVLNLR TAGQGPGQLQ |
| 61 | REVTLHLNPI SSVHIHHKSV VFLLNSPHPL VWHLKTERLA TGVSRLFLVS EGSVVQFSSA |
| 121 | NFSLTAETEE RNFPHGNEHL LNWARKEYGA VTSFTELKIA RNIYIKVGED QVFPPKCNIG |
| 181 | KNFLSLNYLA EYLQPKAAEG CVMSSQPQNE EVHIIELITP NSNPYSAFQV DITIDIRPSQ |
| 241 | EDLEVVKNLI LILKCKKSVN WVIKSFDVKG SLKIIAPNSI GFGKESERSM TMTKSIRDDI |
| 301 | PSTQGNLVKW ALDNGYSPIT SYTMAPVANR FHLRLENNAE EMGDEEVHTI PPELRILLDP |
| 361 | GALPALQNPP IRGGEGQNGG LPFPFPDISR RVWNEEGEDG LPRPKDPVIP SIQLFPGLRE |
| 421 | PEEVQGSVDI ALSVKCDNEK MIVAVEKDSF QASGYSGMDV TLLDPTCKAK MNGTHFVLES |
| 481 | PLNGCGTRPR WSALDGVVYY NSIVIQVPAL GDSSGWPDGY EDLESGDNGF PGDMDEGDAS |
| 541 | LFTRPEIVVF NCSLQQVRNP SSFQEQPHGN ITFNMELYNT DLFLVPSQGV FSVPENGHVY |
| 601 | VEVSVTKAEQ ELGFAIQTCF ISPYSNPDRM SHYTIIENIC PKDESVKFYS PKRVHFPIPQ |
| 661 | ADMDKKRFSF VFKPVFNTSL LFLQCELTLC TKMEKHPQKL PKCVPPDEAC TSLDASIIWA |
| 721 | MMQNKKTFTK PLAVIHHEAE SKEKGPSMKE PNPISPPIFH GLDTLTVTGG GTHTCPPCPA |
| 781 | PELLGGPSVF LFPPKPKDTL MISRTPEVTC VVVDVSHEDP EVKFNWYVDG VEVHNAKTKP |
| 841 | REEQYNSTYR VVSVLTVLHQ DWLNGKEYKC KVSNKALPAP IEKTISKAKG QPREPQVYTL |
| 901 | PPCREEMTKN QVSLWCLVKG FYPSDIAVEW ESNGQPENNY KTTPPVLKSD GSFFLYSKLT |
| 961 | VDKSRWQQGN VFSCSVMHEA LHNHYTQKSL SLSPGK |

As described in Example 1, the complementary form of monomeric G1Fc polypeptide (SEQ ID NO: 506) employs the TPA leader and incorporates an optional N-terminal extension. To promote formation of the BG-Fc:Fc heterodimer rather than either of the possible homodimeric complexes, four amino acid substitutions can be introduced into the monomeric Fc polypeptide as indicated. The amino acid sequence of SEQ ID NO: 506 and the mature Fc polypeptide (SEQ ID NO: 507) may optionally be provided with the C-terminal lysine removed.

The BG-Fc fusion polypeptide and monomeric Fc polypeptide of SEQ ID NO: 551 and SEQ ID NO: 507, respectively, may be co-expressed and purified from a CHO cell line to give rise to a single-arm heteromeric protein complex comprising BG-Fc:Fc.

Purification of various BG-Fc:Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

### Example 13. Generation of a Single-Arm MUSK-Fc Heterodimer

Applicants envision construction of a soluble single-arm MUSK-Fc heterodimeric complex comprising a monomeric Fc polypeptide with a short N-terminal extension and a second polypeptide in which a ligand-binding domain of human MuSK is fused to a separate Fc domain with a linker positioned between a ligand-binding domain and this second Fc domain. The individual constructs are referred to as monomeric Fc polypeptide and MUSK-Fc fusion polypeptide, respectively, and the sequences for each are provided below. Applicants also envision additional single-arm MUSK-Fc heterodimeric complexes comprising a ligand-binding domain of MuSK isoforms 2 or 3 (SEQ ID NO: 100 or 104).

Formation of a single-arm MUSK-Fc heterodimer may be guided by approaches similar to those described for single-arm endoglin-Fc heterodimer in Example 1. In a first approach, illustrated in the MUSK-Fc and monomeric Fc polypeptide sequences of SEQ ID NOs: 552-553 and 502-503, respectively, one Fc domain is altered to introduce cationic amino acids at the interaction face, while the other Fc domain is altered to introduce anionic amino acids at the interaction face.

The MUSK-Fc fusion polypeptide employs the TPA leader and is as follows:

| (SEQ ID NO: 552) | |
|---|---|
| 1 | MDAMKRGLCC VLLLCGAVFV SPGASGTEKL PKAPVITTPL ETVDALVEEV ATFMCAVESY |
| 61 | PQPEISWTRN KILIKLFDTR YSIRENGQLL TILSVEDSDD GIYCCTANNG VGGAVESCGA |
| 121 | LQVKMKPKIT RPPINVKIIE GLKAVLPCTT MGNPKPSVSW IKGDSPLREN SRIAVIESGS |
| 181 | LRIHNVQKED AGQYRCVAKN SLGTAYSKVV KLEVEVFARI LRAPESHNVT FGSFVTLHCT |
| 241 | ATGIPVPTIT WIENGNAVSS GSIQESVKDR VIDSRLQLFI TKPGLYTCIA TNKHGEKFST |
| 301 | AKAAATISIA EWSKPQKDNK GYCAQYRGEV CNAVLAKDAL VFLNTSYADP EEAQELLVHT |
| 361 | AWNELKVVSP VCRPAAEALL CNHIFQECSP GVVPTPIPIC REYCLAVKEL FCAKEWLVME |
| 421 | EKTHRGLYRS EMHLLSVPEC SKLPSMHWDP TACARLPHLD YNKENLKTFP PMTSSKPSVD |
| 481 | IPNLPSSSSS SFSVSPTYSM TTGGGTHTCP PCPAPELLGG PSVFLFPPKP KDTLMISRTP |
| 541 | EVTCVVVDVS HEDPEVKFNW YVDGVEVHNA KTKPREEQYN STYRVVSVLT VLHQDWLNGK |
| 601 | EYKCKVSNKA LPAPIEKTIS KAKGQPPEPQ VYTLPPSRKE MTKNQVSLTC LVKGFYPSDI |
| 662 | AVEWESNGQP ENNYKTTPPV LKSDGSFFLY SKLTVDKSRW QQGNVFSCSV MHEALHNHYT |
| 721 | QKSLSLSPGK |

The leader and linker sequences are underlined. To promote formation of the MUSK-Fc:Fc heterodimer rather than either of the possible homodimeric complexes (MUSK-Fc:MUSK-Fc or Fc:Fc), two amino acid substitutions (replacing anionic residues with lysines) can be introduced into the Fc domain of the fusion polypeptide as indicated by double underline above. The amino acid sequence of SEQ ID NO: 552 may optionally be provided with the C-terminal lysine removed.

The mature MUSK-Fc fusion polypeptide sequence is as follows (SEQ ID NO: 553) and may optionally be provided with the C-terminal lysine removed.

| (SEQ ID NO: 553) | |
|---|---|
| 1 | GTEKLPKAPV ITTPLETVDA LVEEVATFMC AVESYPQPEI SWTRNKILIK LFDTRYSIRE |
| 61 | NGQLLTILSV EDSDDGIYCC TANNGVGGAV ESCGALQVKM KPKITRPPIN VKIIEGLKAV |
| 121 | LPCTTMGNPK PSVSWIKGDS PLRENSRIAV LESGSLRIHN VQKEDAGQYR CVAKNSLGTA |
| 181 | YSKVVKLEVE VFARILRAPE SHNVTFGSFV TLHCTATGIP VPTITWIENG NAVSSGSIQE |
| 241 | SVKDRVIDST LQLFITKPGL YTCIATNKHG EKFSTAKAAA TISIAEWSKP QKDNKGYCAQ |
| 301 | YRGEVCNAVL AKDALVFLNT SYADPEEAQE LLVHTAWNEL KVVSPVCRPA AEALLCNHIF |
| 361 | QECSPGVVPT PIPICREYCL AVKELFCAKE WLVMEEKTHR GLYRSEMHLL SVPECSKLPS |
| 421 | MHWDPTACAR LPHLDYNKEN LKTFPPMTSS KPSVDIPNLP SSSSSSFSVS PTYSMTTGGG |
| 481 | THTCPPCPAP ELLGGPSVFL FPPKPKDTLM ISRTPEVTCV VVDVSHEDPE VKFNWYVDGV |
| 541 | EVHNAKTKPR EEQYNSTYRV VSVLTVLHQD WLNGKEYKCK VSNKALPAPI EKTISKANGQ |
| 601 | PREPQVYTLP PSRKEMTKNQ VSLTCLVKGF YPSDIAVEWE SNGQPENNYK TTPPVLKSDG |
| 661 | SFFLYSKLTV DKSRWQQGNV FSCSVMHEAL HNHYTQKSLS LSPGK |

As described in Example 1, the complementary form of monomeric human G1Fc polypeptide (SEQ ID NO: 502) employs the TPA leader and incorporates an optional N-terminal extension. To promote formation of the MUSK-Fc:Fc heterodimer rather than either of the possible homodimeric complexes, two amino acid substitutions (replacing lysines with anionic residues) can be introduced into the monomeric Fc polypeptide as indicated. The amino acid sequence of SEQ ID NO: 502 may optionally be provided with the C-terminal lysine removed. The mature monomeric Fc polypeptide (SEQ ID NO: 503) may optionally be provided with the C-terminal lysine removed.

The MUSK-Fc fusion polypeptide and monomeric Fc polypeptide of SEQ ID NO: 553 and SEQ ID NO: 503, respectively, may be co-expressed and purified from a CHO cell line to give rise to a single-arm heteromeric protein complex comprising MUSK-Fc:Fc.

In another approach to promoting the formation of heteromultimer complexes using asymmetric Fc fusion polypeptides, the Fc domains are altered to introduce complementary hydrophobic interactions and an additional intermolecular disulfide bond as illustrated in the MUSK-Fc and Fc polypeptide sequences of SEQ ID NOs: 554-555 and 506-507, respectively.

The MUSK-Fc fusion polypeptide (SEQ ID NO: 554) uses the TPA leader and is as follows:

| (SEQ ID NO: 554) | |
|---|---|
| 1 | MDAMKRGLCC VLLLCGAVFV SPGASGTEKL PKAPVITTPL ETVDALVEEV ATFMCAVESY |
| 61 | PQPEISWTRN KILIKLFDTR YSIRENGQLL TILSVEDSDD GIYCCTANNG VGGAVESCGA |
| 121 | LQVKMKPKIT RPPINVKIIE GLKAVLPCTT MGNPKPSVSW IKGDSPLREN SRIAVIESGS |
| 181 | LRIHNVQKED AGQYRCVAKN SLGTAYSKVV KLEVEVFARI LRAPESHNVT FGSFVTLHCT |
| 241 | ATGIPVPTIT WIENGNAVSS GSIQESVKDR VIDSRLQLFI TKPGLYTCIA TNKHGEKFST |
| 301 | AKAAATISIA EWSKPQKDNK GYCAQYRGEV CNAVLAKDAL VFLNTSYADP EEAQELLVHT |
| 361 | AWNELKVVSP VCRPAAEALL CNHIFQECSP GVVPTPIPIC REYCLAVKEL FCAKEWLVME |
| 421 | EKTHRGLYRS EMHLLSVPEC SKLPSMHWDP TACARLPHLD YNKENLKTFP PMTSSKPSVD |
| 481 | IPNLPSSSSS SFSVSPTYSM TTGGGTHTCP PCPAPELLGG PSVFLFPPKP KDTLMISRTP |
| 541 | EVTCVVVDVS HEDPEVKFNW YVDGVEVHNA KTKPREEQYN STYRVVSVLT VLHQDWLNGK |
| 601 | EYKCKVSNKA LPAPIEKTIS KAKGQPPEPQ VYTLPPCREE MTKNQVSLWC LVKGFYPSDI |
| 662 | AVEWESNGQP ENNYKTTPPV LDSDGSFFLY SKLTVDKSRW QQGNVFSCSV MHEALHNHYT |
| 721 | QKSLSLPGK |

The leader sequence and linker are underlined. To promote formation of the MUSK-Fc:Fc heterodimer rather than either of the possible homodimeric complexes, two amino acid substitutions (replacing a serine with a cysteine and a threonine with a tryptophan) can be introduced into the Fc domain of the MuSK fusion polypeptide as indicated by double underline above. The amino acid sequence of SEQ ID NO: 554 may optionally be provided with the C-terminal lysine removed.

The mature MUSK-Fc fusion polypeptide (SEQ ID NO: 555) is as follows and may optionally be provided with the C-terminal lysine removed.

| (SEQ ID NO: 555) | |
|---|---|
| 1 | GTEKLPKAPV ITTPLETVDA LVEEVATFMC AVESYPQPEI SWTRNKILIK LFDTRYSIRE |
| 61 | NGQLLTILSV EDSDDGIYCC TANNGVGGAV ESCGALQVKM KPKITRPPIN VKIIEGLKAV |
| 121 | LPCTTMGNPK PSVSWIKGDS PLRENSRIAV LESGSLRIHN VQKEDAGQYR CVAKNSLGTA |
| 181 | YSKVVKLEVE VFARILRAPE SHNVTFGSFV TLHCTATGIP VPTITWIENG NAVSSGSIQE |
| 241 | SVKDRVIDSR LQLFITKPGL YTCIATNKHG EKFSTAKAAA TISIAEWSKP QKDNKGYCAQ |
| 301 | YRGEVCNAVL AKDALVFLNT SYADPEEAQE LLVHTAWNEL KVVSPVCRPA AEALLCNHIF |
| 361 | QECSPGVVPT PIPICREYCL AVKELFCAKE WLVMEEKTHR GLYRSEMHLL SVPECSKLPS |
| 421 | MHWDPTACAR LPHLDYNKEN LKTFPPMTSS KPSVDIPNLP SSSSSSFSVS PTYSMTTGGG |
| 481 | THTCPPCPAP ELLGGPSVFL FPPKPKDTLM ISRTPEVTCV VVDVSHEDPE VKFNWYVDGV |
| 541 | EVHNAKTKPR EEQYNSTYRV VSVLTVLHQD WLNGKEYKCK VSNKALPAPI EKTISKANGQ |
| 601 | PREPQVYTLP PCREEMTKNQ VSLWCLVKGF YPSDIAVEWE SNGQPENNYK TTPPVLKSDG |
| 661 | SFFLYSKLTV DKSRWQQGNV FSCSVMHEAL HNHYTQKSLS LSPGK |

As described in Example 1, the complementary form of monomeric G1Fc polypeptide (SEQ ID NO: 506) employs the TPA leader and incorporates an optional N-terminal extension. To promote formation of the MUSK-Fc:Fc heterodimer rather than either of the possible homodimeric complexes, four amino acid substitutions can be introduced into the monomeric Fc polypeptide as indicated. The amino acid sequence of SEQ ID NO: 506 and the mature Fc polypeptide (SEQ ID NO: 507) may optionally be provided with the C-terminal lysine removed.

The MUSK-Fc fusion polypeptide and monomeric Fe polypeptide of SEQ ID NO: 555 and SEQ ID NO: 507, respectively, may be co-expressed and purified from a CHO cell line to give rise to a single-arm heteromeric protein complex comprising MUSK-Fc:Fc.

Purification of various MUSK-Fc:Fc complexes could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

We claim:

1. A heteromultimer comprising a first polypeptide covalently or non-covalently associated with a second polypeptide, wherein:
a. the first polypeptide comprises the amino acid sequence of a first member of an interaction pair and the amino acid sequence of a TGFβ superfamily co-receptor polypeptide, wherein the TGFβ superfamily co-receptor polypeptide is selected from: endoglin, betaglycan, Cripto-1, Cryptic, Cryptic family protein 1B, Crim1, Crim2, BAMBI, BMPER, RGM-A, RGM-B, MuSK, and hemojuvelin polypeptides; and
b. the second polypeptide comprises the amino acid sequence of a second member of the interaction pair, and wherein the second polypeptide does not comprise a TGFβ superfamily co-receptor polypeptide.

2. The heteromultimer of claim 1, wherein the TGFβ superfamily co-receptor polypeptide is an endoglin polypeptide.

3. The heteromultimer of claim 1, wherein the TGFβ superfamily co-receptor polypeptide is a betaglycan polypeptide.

4. The heteromultimer of claim 1, wherein the TGFβ superfamily co-receptor polypeptide is a Cripto-1 polypeptide.

5. The heteromultimer of claim 1, wherein the TGFβ superfamily co-receptor polypeptide is a Cryptic polypeptide.

6. The heteromultimer of claim 1, wherein the TGFβ superfamily co-receptor polypeptide is a Cryptic family protein 1B polypeptide.

7. The heteromultimer of claim 1, wherein the TGFβ superfamily co-receptor polypeptide is a Crim1 polypeptide.

8. The heteromultimer of claim 1, wherein the TGFβ superfamily co-receptor polypeptide is a Crim2 polypeptide

9. The heteromultimer of claim 1, wherein the TGFβ superfamily co-receptor polypeptide is a BAMBI polypeptide.

10. The heteromultimer of claim 1, wherein the TGFβ superfamily co-receptor polypeptide is a BMPER polypeptide.

11. The heteromultimer of claim 1, wherein the TGFβ superfamily co-receptor polypeptide is an RGM-A polypeptide.

12. The heteromultimer of claim 1, wherein the TGFβ superfamily co-receptor polypeptide is an RGM-B polypeptide.

13. The heteromultimer of claim 1, wherein the TGFβ superfamily co-receptor polypeptide is a hemojuvelin polypeptide.

14. The heteromultimer of claim 1, wherein the TGFβ superfamily co-receptor polypeptide is a MuSK polypeptide.

15. The heteromultimer of claim 2, wherein the endoglin polypeptide comprises an amino acid sequence that is at least 70%, 75%, 80%, 85%, 90%, 91%, 92%, 93%, 94%, 95%, 96%, 97%, 98%, 99% or 100% identical to the sequence of any one of SEQ ID Nos: 2, 6, 10, 500, 501, 504, or 505.

16. The heteromultimer of claim 3, wherein the betaglycan polypeptide comprises an amino acid sequence that is at least 70%, 75%, 80%, 85%, 90%, 91%, 92%, 93%, 94%, 95%, 96%, 97%, 98%, 99% or 100% identical to the sequence of any one of SEQ ID Nos: 86, 90, 548, 549, 550, or 551.

17. The heteromultimer of claim 4, wherein the Cripto-1 polypeptide comprises an amino acid sequence that is at least 70%, 75%, 80%, 85%, 90%, 91%, 92%, 93%, 94%, 95%, 96%, 97%, 98%, 99% or 100% identical to the sequence of any one of SEQ ID Nos: 14, 18, 508, 509, 510, or 511.

18. The heteromultimer of claim 5, wherein the Cryptic polypeptide comprises an amino acid sequence that is at least 70%, 75%, 80%, 85%, 90%, 91%, 92%, 93%, 94%, 95%, 96%, 97%, 98%, 99% or 100% identical to the sequence of any one of SEQ ID Nos: 22, 26, 30, 512, 513, 514, or 515.

19. The heteromultimer of claim 6, wherein the Cryptic family protein 1B polypeptide comprises an amino acid sequence that is at least 70%, 75%, 80%, 85%, 90%, 91%, 92%, 93%, 94%, 95%, 96%, 97%, 98%, 99% or 100% identical to the sequence of any one of SEQ ID Nos: 34, 516, 517, 518, or 519.

20. The heteromultimer of claim 7, wherein the Crim1 polypeptide comprises an amino acid sequence that is at least 70%, 75%, 80%, 85%, 90%, 91%, 92%, 93%, 94%, 95%, 96%, 97%, 98%, 99% or 100% identical to the sequence of any one of SEQ ID Nos: 38, 520, 521, 522, or 523.

21. The heteromultimer of claim 8, wherein the Crim2 polypeptide comprises an amino acid sequence that is at least 70%, 75%, 80%, 85%, 90%, 91%, 92%, 93%, 94%, 95%, 96%, 97%, 98%, 99% or 100% identical to the sequence of any one of SEQ ID Nos: 42, 46, 524, 525, 526, or 527.

22. The heteromultimer of claim 9, wherein the BAMBI polypeptide comprises an amino acid sequence that is at least 70%, 75%, 80%, 85%, 90%, 91%, 92%, 93%, 94%, 95%, 96%, 97%, 98%, 99% or 100% identical to the sequence of any one of SEQ ID Nos: 50, 528, 529, 530, or 531.

23. The heteromultimer of claim 10, wherein the BMPER polypeptide comprises an amino acid sequence that is at least 70%, 75%, 80%, 85%, 90%, 91%, 92%, 93%, 94%, 95%, 96%, 97%, 98%, 99% or 100% identical to the sequence of any one of SEQ ID Nos: 54, 532, 533, 534, or 535.

24. The heteromultimer of claim 11, wherein the RGM-A polypeptide comprises an amino acid sequence that is at least 70%, 75%, 80%, 85%, 90%, 91%, 92%, 93%, 94%, 95%, 96%, 97%, 98%, 99% or 100% identical to the sequence of any one of SEQ ID Nos: 62, 66, 70, 540, 541, 542, or 543.

25. The heteromultimer of claim 13, wherein the hemojuvelin polypeptide comprises an amino acid sequence that is at least 70%, 75%, 80%, 85%, 90%, 91%, 92%, 93%, 94%, 95%, 96%, 97%, 98%, 99% or 100% identical to the sequence of any one of SEQ ID Nos: 74, 78, 82, 544, 545, 546, or 547.

26. The heteromultimer of claim 14, wherein the MuSK polypeptide comprises an amino acid sequence that is at least 70%, 75%, 80%, 85%, 90%, 91%, 92%, 93%, 94%, 95%, 96%, 97%, 98%, 99% or 100% identical to the sequence of any one of SEQ ID Nos: 74, 78, 82, 552, 553, 554, or 555.

27. The heteromultimer of claim 12, wherein the RGM-B polypeptide comprises an amino acid sequence that is at least 70%, 75%, 80%, 85%, 90%, 91%, 92%, 93%, 94%, 95%, 96%, 97%, 98%, 99% or 100% identical to the sequence of any one of SEQ ID Nos: 58, 536, 537, 538, or 539.

28. The heteromultimer of any one of claims 1-27, wherein the protein complex is a recombinant heterodimer.

29. The heteromultimer of any of claims 1-28, wherein the first member of an interaction pair comprises a first constant region from an IgG heavy chain.

30. The heteromultimer of any of claims 1-29, wherein the second member of an interaction pair comprises a second constant region from an IgG heavy chain.

31. The heteromultimer of claim 29, wherein the first constant region from an IgG heavy chain is a first immunoglobulin Fc domain.

32. The heteromultimer of claim 30, wherein the second constant region from an IgG heavy chain is a first immunoglobulin Fc domain.

33. The heteromultimer of claim 31, wherein the first constant region from an IgG heavy chain comprises an amino acid sequence that is at least 70%, 80%, 85%, 90%, 95%, 96%, 97%, 98%, 99% or 100% identical to a sequence selected from any one of SEQ ID NOs: 200-214.

34. The heteromultimer of claim 32, wherein the second constant region from an IgG heavy chain comprises an amino acid sequence that is at least 70%, 80%, 85%, 90%, 95%, 96%, 97%, 98%, 99% or 100% identical to a sequence selected from any one of SEQ ID NOs: 200-214.

35. The heteromultimer of any of claims 1-34, wherein the first polypeptide comprises an amino acid sequence that is at least 70%, 75%, 80%, 85%, 90%, 91%, 92%, 93%, 94%, 95%, 96%, 97%, 98%, 99% or 100% identical to a sequence selected from any one of SEQ ID NOs: 500, 501, 504, 505, 548, 549, 550, 551, 508, 509, 510, 511, 512, 513, 514, 515, 516, 517, 518, 519, 520, 521, 522, 523, 524, 525, 526, 527, 528, 529, 530, 531, 532, 533, 534, 535, 540, 541, 542, 543, 544, 545, 546, 547, 552, 553, 554, or 555.

36. The heteromultimer of any of claims 1-35, wherein the second polypeptide comprises an amino acid sequence that is at least 70%, 75%, 80%, 85%, 90%, 91%, 92%, 93%, 94%, 95%, 96%, 97%, 98%, 99% or 100% identical to a sequence selected from any one of SEQ ID NOs: 502, 503, 506, or 507.

37. The heteromultimer of any one of claims 1-36, wherein the first polypeptide and/or second polypeptide comprises one or more modified amino acid residues selected from: a glycosylated amino acid, a PEGylated amino acid, a farnesylated amino acid, an acetylated amino acid, a biotinylated amino acid, and an amino acid conjugated to a lipid moiety.

38. The heteromultimer of any one of claims 1-37, wherein the first polypeptide and/or second polypeptide is glycosylated and has a glycosylation pattern obtainable from expression of the polypeptide in a CHO cell.

39. The heteromultimer of any one of claims 1-38, wherein the heteromultimer has one or more of the following characteristics: i) binds to a TGF-beta superfamily ligand with a K_{D} of less than or equal to 10⁻⁷, 10⁻⁸, 10⁻⁹, or 10⁻¹⁰ M; and ii) inhibits a TGF-beta superfamily type I and/or type II receptor-mediated signaling transduction a cell.

40. The heteromultimer of any one of claims 1-39, wherein the heteromultimer binds to one or more of BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, GDNF, neurturin, artemin, persephin, MIS, and Lefty.

41. The protein complex of any one of claims 1-40, wherein the heteromultimer inhibits the activity of one or more TGF-beta superfamily ligands in a cell-based assay.

42. The heteromultimer of claim 41, wherein the TGF-beta superfamily ligand is selected from: BMP2, BMP2/7, BMP3, BMP4, BMP4/7, BMP5, BMP6, BMP7, BMP8a, BMP8b, BMP9, BMP10, GDF3, GDF5, GDF6/BMP13, GDF7, GDF8, GDF9b/BMP15, GDF11/BMP11, GDF15/MIC1, TGF-β1, TGF-β2, TGF-β3, activin A, activin B, activin C, activin E, activin AB, activin AC, activin AE, activin BC, activin BE, nodal, GDNF, neurturin, artemin, persephin, MIS, and Lefty.

43. A pharmaceutical preparation comprising the heteromultimer of any one of claims 1-42 and a pharmaceutically acceptable carrier.

44. A method for treating a patient having a TGF superfamily-associated condition comprising administering to a patient in need thereof an effective amount of the heteromultimer of any one of claims 1-42 or pharmaceutical preparation of claim 43.

45. The method of claim 44, wherein the TGFβ superfamily-associated condition is selected from the group: a muscle disorder, a red blood cell disorder, an anemia, a bone disorder, bone loss, a fibrotic disorder, chronic kidney disease, a metabolic disease, type II diabetes, obesity, and a cardiovascular disorder.
